(19) United States
(12) Patent Application Publication
(10) Pub. No.: US 2011/0105436 A1
(43) Pub. Date: May 5, 2011

(54) HETEROARYL COMPOUNDS, COMPOSITIONS, AND METHODS OF USE IN CANCER TREATMENT

(75) Inventors: Sandra Turcotte, Montreal (CA); Denise A. Chan, Palo Alto, CA (US); Patrick D. Sutphin, Boston, MA (US); Amato J. Giaccia, Stanford, CA (US); Michael P. Hay, Aukland (NZ); William A. Denny, Auckland (NZ); Muriel Marie Bonnet, Auckland (NZ)

(73) Assignees: AUCKLAND UNISERVICES LIMITED, Auckland (NZ); THE BOARD OF TRUSTEES OF THE LELAND STANFORD JUNIOR UNIVERSITY, PALO ALTO, CA (US)

(21) Appl. No.: 12/921,767

(22) PCT Filed: Mar. 10, 2009
(86) PCT No.: PCT/US09/36696

§ 371 Date: Jan. 5, 2011

Related U.S. Application Data
(60) Provisional application No. 61/035,358, filed on Mar. 10, 2008.

(51) Int. Cl. A61K 31/675; C07D 417/04; C07F 9/6558; C07D 417/14; C07D 413/14; C07D 413/04; C07D 401/04; A61K 31/4439; A61K 31/5377; A61K 31/4709; A61K 31/496; A61P 35/00

(52) U.S. Cl. 514/89; 546/270.4; 546/22; 544/124; 546/271.4; 546/167; 546/274.1; 544/364; 514/342; 514/338; 514/236.8; 514/340; 514/314; 514/341; 514/253.1

(57) ABSTRACT

Provided herein are novel heteroaryl compounds, compositions comprising the compounds, and methods of treatment or prevention comprising administration of the compounds. The compounds are effective in the targeting of cells defective in the von Hippel-Lindau gene and in inducing autophagic cell death. The methods are directed to treating or preventing diseases such as cancer, and in particular cancers resulting from von Hippel-Lindau disease. The compounds of the invention may be administered in combination with another therapeutic agent.

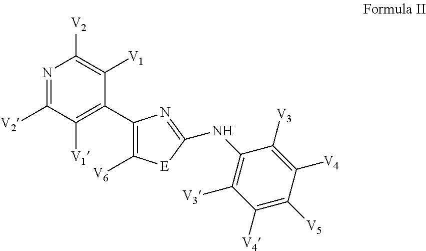

### CROSS-REFERENCE TO RELATED APPLICATION

This application claims the benefit of U.S. Provisional Application No. 61/035,358, filed Mar. 10, 2008, the disclosure of which is incorporated herein by reference in its entirety.

### STATEMENT REGARDING FEDERALLY SPONSORED RESEARCH OR DEVELOPMENT

This invention was made with Government support under contract CA082566, awarded by the National Cancer Institute. The government has certain rights in this invention.

### BACKGROUND OF THE INVENTION

Mutations and/or inactivation of the von Hippel-Lindau (VHL) tumor suppressor gene occur in the majority of clear cell renal carcinoma (CC-RCC) and is associated with metastatic disease (Motzer, R. J., et al., N. Engl. J. Med. 1996, 335, 865). The tumor suppressor function of VHL was demonstrated by the restoration of VHL function in VHL−/− RCC that resulted in significant inhibition of these cells to form tumors in nude mice (Iliopoulos, O., et al., Nat. Med. 1995, 1, 822). Functional studies indicate that pVHL, the protein product of VHL, is an E3 ubiquitin ligase that targets the α-subunit of the hypoxia-inducible factor (HIF) for proteasomal degradation under normoxia. In the presence of oxygen, hydroxylation on proline residues 564 and 402 by prolyl hydroxylases (PHDs) marks HIF-α for recognition and binding with pVHL, leading to degradation of HIF-α. Under hypoxic conditions, activity of the PHDs decrease, which prevents the recognition of HIF-α by pVHL (Ivan, M., et al. Science, 2001, 292, 464; Jaakkola, P., et al., Science, 2001, 292, 468; Chan, D. A., et al., J. Biol. Chem. 2002, 277, 40112). In cells that lack VHL, stabilized HIF-α binds HIF-β to activate the transcription of genes involved in several processes (Staller, P., et al., Nature, 2003, 425, 307; Gnarra, J. R., et al. Proc. Natl. Acad. Sci. USA, 1996, 93, 10589; Iliopoulos, O., et al., Proc. Natl. Acad. Sci. USA, 1996, 93, 10595; Knebelmann, B., et al., Cancer Res., 1998, 58, 226). In addition to its role in HIF regulation, pVHL has been implicated in a variety of processes including extracellular matrix assembly, regulation of microtubule stability, polyubiquitination of atypical PKC family members, regulation of fibronectin, and RNA polymerase II subunits (Hergovich, A., Nat. Cell Biol. 2003, 5, 64; Okuda, H., et al., J. Biol. Chem. 2001, 276, 43611; Ohh, M., et al., Mol. Cell. 1998, 1, 959; Na, X., et al., EMBO J. 2003, 22, 4249). It has also been reported that an acidic domain present in the N-terminal region of pVHL contributes to its tumor suppression function in a HIF-independent manner and may be relevant for the development of VHL-associated malignancies (Lolkema, M. P., et al., J. Biol. Chem. 2005, 280, 22205).

Defects in apoptosis that are observed in many solid tumor cells, including RCCs, increase the resistance of the tumor cells to chemotherapy, radiotherapy and molecularly targeted therapies. In contrast to apoptosis, autophagy regulates the turnover of organelles and long-lived proteins to ensure homeostasis. Under metabolic stress, autophagy is activated and promotes survival (Mathew, R., et al., Genes Dev. 2007, 21, 1367). It can also result in cell death if this proceeds to completion under persistert stress or genetic alterations. Studies have reported a deregulated level of autophagy in diverse diseases including neuronal degeneration, infectious disease, and cancer (Kondo, Y., et al., Nat. Rev. Cancer 2005, 5, 726).

Autophagy occurs in all eukaryotic cells from yeast to mammals. In response to a diverse number of stimuli, such as starvation, hypoxia, or high temperature, portions of the cytoplasm and organelles are sequestered in a double-membrane vesicle called an autophagosome. These vesicles undergo maturation by fusion with endosomes and/or lysosomes to become autolysosomes where hydrolases degrade their conterts (Klionsky, D. J. and S. D. Emr, Science 2000, 290, 1717). The induction of autophagy selectively in tumor cells has potertial for the treatment of cancer.

Westphal, G., et al., J. Praktische Chemie 1976, 318, 875 describe the synthesis of pyridyl thiazoles.

PCT International Publication No. WO01/64674 A1 describes 2,4-disubstituted thiazolyl derivatives purportedly useful in the prevention or treatment of diseases mediated through cytokines, particularly TNF-α and/or Interleukin-12.

PCT International Publication No. WO2007/031440 A2 describes 2-aniline-4-aryl substituted thiazole derivatives purportedly useful in the modulation of the α7 nicotinic receptor.

PCT International Publication No. WO00/33837 describes compounds, including substituted thiazoles, as inhibitors of membrane-associated tyrosine and threonine kinases.

US Patent Application Publication No. 2005/0176776 A1 relates to substituted thiazole derivatives purportedly useful in inhibiting mitotic kinesins, particularly KSP, and Kin I kinesin-related proteins, particularly MCAK. The compounds are suggested to be useful in treating cellular proliferative diseases.

US Patent Application Publication No. 2008/0039466 A1 and PCT International Publication Nos. WO2004/014903 A1, WO2004/096225 A2, WO2005/016323 A2, and WO2005/073225 A1 relate to substituted amino-aryl-thiazoles as tyrosine kinase inhibitors, specifically inhibitors of c-kit and c-kit pathway. The compounds are proposed to be candidates for treating diseases such as autoimmune diseases, inflammatory diseases, cancer, mastocytosis, diabetes, and cerebral ischemia.

PCT International Publication No. WO2005/063709 A1 relates to heterocyclyl moiety-containing amides as BCR-ABL tyrosine kinase inhibitors.

PCT International Publication No. WO2007/118149 A2 describe substituted thiazoles and thiophenes reportedly useful in targeting transcription factors NF-κB and AP-1 and translation initiation factor eIF-4E.

PCT International Publication Nos. WO2006/122011 A2, WO2006/135383 A2, and WO2007/103550 A2 and Severson, W. E., et al., J. Biomol. Screening 2007, 12, 33 describe compounds, including thiazole derivatives, as inhibitors of viral metabolism.

PCT International Publication No. WO2001/068645 A2 describes thiazole derivatives as cysteine protease inhibitors for the treatment of osteoporosis.

PCT International Publication No. WO2004/110350 A2 describes the use of aryl compounds, including substituted thiazoles, in the modulation of amyloid 0.

PCT International Publication No. WO2005/012295 A1 relates to substituted thiazoles as inhibitors of phosphotyrosine phosphatase IB in the treatment of diabetes.

Narayana, B., et al., Phosphorus, Sulfur and Silicon and the Related Elements 2007, 182, 7 describe thiazole derivatives as antifungals and antibacterials.

None of the above publications describes the use of heteroaryl compounds, such as substituted thiazoles, in the targeting of cells defective in the von Hippel-Lindau gene or in the treatment of patients afflicted with diseases associated with such defects. Furthermore, it is known that radiation and standard chemotherapy have been unsuccessful in the treatment of diseases such as renal cell carcinoma. There is therefore a need in the art for the development of novel compounds, compositions, and treatments that target cells defective in the von Hippel-Lindau gene and diseases associated with such defects.

### SUMMARY OF THE INVENTION

The present invention addresses these problems by providing novel heteroaryl compounds, compositions, and methods of treatment.

In one aspect, the invention provides compounds represented by structural formula II:

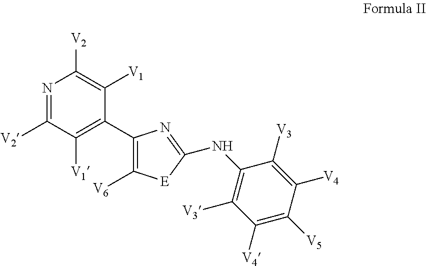

or a pharmaceutically acceptable salt, derivative, or prodrug thereof, wherein:

E is S, O, or N—V₇;

V₃, V₃′, V₄′, and V₅ are all hydrogen;

V₁, V₁′, V₂, V₂′, V₄, V₆, and V₇ are independently hydrogen, halo, R, OH, OR, OC(O)H, OC(O)R, OC(O)NH₂, OC(O)NHR, OC(O)NRR, OP(O)(OH)₂, OP(O)(OR)₂, NO₂, NH₂, NHR, NRR, N⁺(—O⁻)RR, NHC(O)H, NHC(O)R, NRC(O)R, NHC(O)NH₂, NHC(O)NRR, NRC(O)NHR, N²C(O)NHR, SH, SR, S(O)H, S(O)R, SO₂R, SO₂NH₂, SO₂NHR, SO₂NRR, CF₃, CN, CO₂H, CO₂R, CHO, C(O)R, C(O)NH₂, C(O)NHR, C(O)NRR, CONHSO₂H, C(O)NHSO₂R, C(O)NRSO₂R, cyclic C₃-C₇ alkylamino, imidazolyl, piperazinyl, morpholinyl, piperidinyl, azepanyl, pyrrolidinyl or azetidinyl;

wherein each imidazolyl, piperazinyl, morpholinyl, piperidinyl, azepanyl, pyrrolidinyl and azetidinyl is optionally and independently substituted by one or more R¹, halo, OH, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N⁺(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, CONHSO₂H, C(O)NHSO₂R¹ or C(O)NR¹SO₂R¹;

and wherein V₁ and V₂, taken together with the atoms to which they are attached, optionally form a cyclic structure having from 4 to 8 atoms in the ring;

each R is independently C₁₋₆ alkyl, C₂₋₆ alkenyl, C₂₋₆ alkynyl, C₃₋₇ cyclic alkyl, aryl, aralkyl, heteroaryl, or heteroaralkyl, and is optionally and independently substituted with halo, OH, R¹, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N⁺(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, C(O)NHSO₂R¹, C(O)NR¹SO₂R¹, cyclic C₃-C₇ alkylamino, imidazolyl, piperazinyl, morpholinyl, piperidinyl, azepanyl, pyrrolidinyl, or azetidinyl;

wherein each imidazolyl, piperazinyl, morpholinyl, piperidinyl, azepanyl, pyrrolidinyl and azetidinyl is optionally and independently substituted with one or more R¹, halo, OH, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N⁺(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, CONHSO₂H, C(O)NHSO₂R¹ or C(O)NR¹SO₂R¹;

and wherein each heteroaryl group contains one or more heteroatoms in its ring system, independently selected from O, N or S;

each R¹ is independently C₁₋₆ alkyl or C₂₋₆ alkenyl, optionally and independently substituted with halo, OH, OR², OC(O)R², OC(O)NH₂, OC(O)NHR², OC(O)NR²R², OP(O)(OH)₂, OP(O)(OR²)₂, NO₂, NH₂, NHR², NR²R², N⁺(—O⁻)R²R², NHC(O)R², NR²C(O)R², NHC(O)NH₂, NHC(O)NR²R², NR²C(O)NHR², SH, SR², S(O)H, S(O)R², SO₂R², SO₂NH₂, SO₂NHR², SO₂NR²R², CF₃, CN, CO₂H, CO₂R², CHO, C(O)R², C(O)NH₂, C(O)NHR², C(O)NR²R², C(O)NHSO₂R², C(O)NR²SO₂R², cyclic C₃-C₇ alkylamino, imidazolyl, piperazinyl, morpholinyl, piperidinyl, azepanyl, pyrrolidinyl or azetidinyl;

wherein each imidazolyl, piperazinyl, morpholinyl, piperidinyl, azepanyl, pyrrolidinyl and azetidinyl is optionally and independently substituted with one or more R², halo, OH, OR², OC(O)R², OC(O)NH₂, OC(O)NHR², OC(O)NR²R², OP(O)(OH)₂, OP(O)(OR²)₂, NO₂, NH₂, NHR², NR²R², N⁺(—O⁻)R²R², NHC(O)H, NHC(O)R², NR²C(O)R², NHC(O)NH₂, NHC(O)NR²R²₅NR²C(O)NHR², SH, SR², S(O)H, S(O)R², SO₂R², SO₂NH₂, SO₂NHR², SO₂NR²R², CF₃, CN, CO₂H, CO₂R², CHO, C(O)R², C(O)NH₂, C(O)NHR², C(O)NR²R², CONHSO₂H, C(O)NHSO₂R² or C(O)NR²SO₂R²;

each R² is independently C₁₋₆ alkyl, C₂₋₆ alkenyl, OH, OMe, NO₂, NH₂, CF₃, CN, CO₂H or SH;

provided that V₁, V₁′, V₂, V₂′, V₄, V₆, and V₇ are not all hydrogen; and

when E is S; V₁, V₁′, V₂′, and V₆ are all hydrogen; and V₂ is Cl; then V₄ is not Cl; and

when E is S and V₁, V₁′, V₂, V₂′, and V₆ are all hydrogen; then V₄ is not COOH, COOCH₃, C(O)CH₃, CF₃, CH₃, OH, OCH₃, SCH₃, CN, O-phenyl, Cl, Br, or NO₂; and

when E is S; V₁′, V₂′, V₄, and V₆ are all hydrogen; then V₁ and V₂, taken together with the ring to which they are attached, do not form

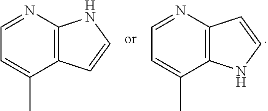

In another aspect, the invention provides compounds represented by structural formula II:

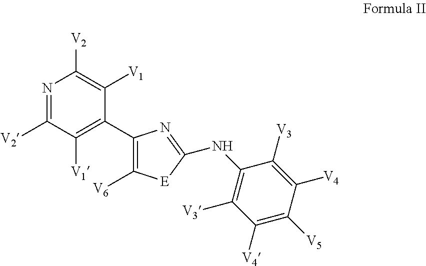

or a pharmaceutically acceptable salt, derivative, or prodrug thereof, wherein:

E is S, O, or N—V₇;

V₅ and V₆ are both hydrogen;

V₁, V₁′, V₂, V₂′, V₃, V₃′, V₄, V₄′, and V₇ are independently hydrogen, halo, R, OH, OR, OC(O)H, OC(O)R, OC(O)NH₂, OC(O)NHR, OC(O)NRR, OP(O)(OH)₂, OP(O)(OR)₂, NO₂, NH₂, NHR, NRR, N⁺(—O⁻)RR, NHC(O)H, NHC(O)R, NRC(O)R, NHC(O)NH₂, NHC(O)NRR, NRC(O)NHR, N²C(O)NHR, SH, SR, S(O)H, S(O)R, SO₂R, SO₂NH₂, SO₂NHR, SO₂NRR, CF₃, CN, CO₂H, CO₂R, CHO, C(O)R, C(O)NH₂, C(O)NHR, C(O)NRR, CONHSO₂H, C(O)NHSO₂R, C(O)NRSO₂R, cyclic C₃-C₇ alkylamino, imidazolyl, piperazinyl, morpholinyl, piperidinyl, azepanyl, pyrrolidinyl or azetidinyl;

wherein each imidazolyl, piperazinyl, morpholinyl, piperidinyl, azepanyl, pyrrolidinyl and azetidinyl is optionally and independently substituted by one or more R¹, halo, OH, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N⁺(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, CONHSO₂H, C(O)NHSO₂R¹ or C(O)NR¹SO₂R¹;

and wherein groups V₁ and V₂ and groups V₃ and V₄, taken together with the atoms to which they are attached, optionally and independently form a cyclic structure having from 4 to 8 atoms in the ring;

each R is independently C₁₋₆ alkyl, C₂₋₆ alkenyl, C₂₋₆ alkynyl, C₃₋₇ cyclic alkyl, aryl, aralkyl, heteroaryl, or heteroaralkyl, and is optionally and independently substituted with halo, OH, R¹, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N⁺(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, C(O)NHSO₂R¹, C(O)NR¹SO₂R¹, cyclic C₃-C₇ alkylamino, imidazolyl, piperazinyl, morpholinyl, piperidinyl, azepanyl, pyrrolidinyl, or azetidinyl;

wherein each imidazolyl, piperazinyl, morpholinyl, piperidinyl, azepanyl, pyrrolidinyl and azetidinyl is optionally and independently substituted with one or more R¹, halo, OH, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N⁺(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, CONHSO₂H, C(O)NHSO₂R¹ or C(O)NR¹SO₂R¹;

and wherein each heteroaryl group contains one or more heteroatoms in its ring system, independently selected from O, N or S;

each R¹ is independently C₁₋₆ alkyl or C₂₋₆ alkenyl, optionally and independently substituted with halo, OH, OR², OC(O)R², OC(O)NH₂, OC(O)NHR², OC(O)NR²R², OP(O)(OH)₂, OP(O)(OR²)₂, NO₂, NH₂, NHR², NR²R², N⁺(—O⁻)R²R², NHC(O)R², NR²C(O)R², NHC(O)NH₂, NHC(O)NR²R², NR²C(O)NHR², SH, SR², S(O)H, S(O)R², SO₂R², SO₂NH₂, SO₂NHR², SO₂NR²R², CF₃, CN, CO₂H, CO₂R², CHO, C(O)R², C(O)NH₂, C(O)NHR², C(O)NR²R², C(O)NHSO₂R², C(O)NR²SO₂R², cyclic C₃-C₇ alkylamino, imidazolyl, piperazinyl, morpholinyl, piperidinyl, azepanyl, pyrrolidinyl or azetidinyl;

wherein each imidazolyl, piperazinyl, morpholinyl, piperidinyl, azepanyl, pyrrolidinyl and azetidinyl is optionally and independently substituted with one or more R², halo, OH, OR², OC(O)R², OC(O)NH₂, OC(O)NHR², OC(O)NR²R², OP(O)(OH)₂, OP(O)(OR²)₂, NO₂, NH₂, NHR², NR²R², N⁺(—O⁻)R²R², NHC(O)H, NHC(O)R², NR²C(O)R², NHC(O)NH₂, NHC(O)NR²R², NR²C(O)NHR², SH, SR², S(O)H, S(O)R², SO₂R², SO₂NH₂, SO₂NHR², SO₂NR²R², CF₃, CN, CO₂H, CO₂R², CHO, C(O)R², C(O)NH₂, C(O)NHR², C(O)NR²R², CONHSO₂H, C(O)NHSO₂R² or C(O)NR²SO₂R²;

each R² is independently C₁₋₆ alkyl, C₂₋₆ alkenyl, OH, OMe, NO₂, NH₂, CF₃, CN, CO₂H or SH;

provided that V₁, V₁′, V₂, V₂′, V₃, V₃′, V₄, V₄′, and V₇ are not all hydrogen; and

when E is S; V₁, V₁′, and V₂′ are all hydrogen; and V₂ is Cl; then V₃ is not CH₃ and V₄ is not Cl; and

when E is S and V₃′ and V₄ are both hydrogen, then either V₃ or V₄′ is also hydrogen; and

when E is S and V₁, V₁′, V₂, V₂′, V₃, V₃′, and V₄′ are all hydrogen, then V₄ is not COOH, COOCH₃, C(O)CH₃, CF₃, CH₃, OH, OCH₃, SCH₃, CN, O-phenyl, Cl, Br, or NO₂; and

when E is S and V₁, V₁′, V₂, V₂′, V₃′, V₄, and V₄′ are all hydrogen, then V₃ is not CH₃, CH₂CH₃, F, Cl, NO₂, CF₃, OCH₃, OCH₂CH₃, or a substituted 1,4-dihydropyridyl ring; and

when E is S and V₁, V₁′, V₂, V₂′, V₃′, and V₄′ are all hydrogen; then when V₃ is Cl, V₄ is not Cl; when V₃ is CH₃, V₄ is not Cl; and V₃ and V₄, taken together with the ring to which they are attached, do not form

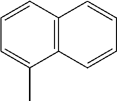

or

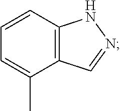

and

when E is S and V₁′, V₂′, V₃, V₃′, V₄, and V₄′ are all hydrogen, then V₁ and V₂, taken together with the ring to which they are attached, do not form

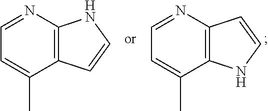

and

when V₃ and V₃′ are both hydrogen, then V₄ and V₄′ are not both CH₃.

In yet another aspect, the invention provides compounds represented by structural formula II:

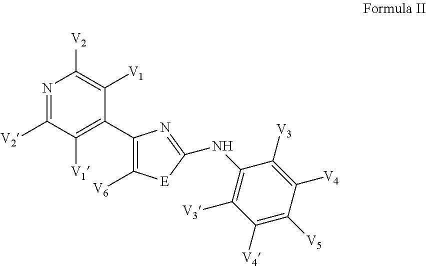

or a pharmaceutically acceptable salt, derivative, or prodrug thereof, wherein:

E is S, O, or N—V₇;

V₃′ and V₄′ are both hydrogen;

V₁, V₁′, V₂, V₂′, V₃, V₄, V₅, V₆, and V₇ are independently hydrogen, halo, R, OH, OR, OC(O)H, OC(O)R, OC(O)NH₂, OC(O)NHR, OC(O)NRR, OP(O)(OH)₂, OP(O)(OR)₂, NO₂, NH₂, NHR, NRR, N⁺(—O⁻)RR, NHC(O)H, NHC(O)R, NRC(O)R, NHC(O)NH₂, NHC(O)NRR, NRC(O)NHR, N²C(O)NHR, SH, SR, S(O)H, S(O)R, SO₂R, SO₂NH₂, SO₂NHR, SO₂NRR, CF₃, CN, CO₂H, CO₂R, CHO, C(O)R, C(O)NH₂, C(O)NHR, C(O)NRR, CONHSO₂H, C(O)NHSO₂R, C(O)NRSO₂R, cyclic C₃-C₇ alkylamino, imidazolyl, piperazinyl, morpholinyl, piperidinyl, azepanyl, pyrrolidinyl or azetidinyl;

wherein each imidazolyl, piperazinyl, morpholinyl, piperidinyl, azepanyl, pyrrolidinyl and azetidinyl is optionally and independently substituted by one or more R¹, halo, OH, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N⁺(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, CONHSO₂H, C(O)NHSO₂R¹ or C(O)NR¹SO₂R¹;

and wherein groups V₁ and V₂, groups V₃ and V₄, and groups V₄ and V₅, taken together with the atoms to which they are attached, optionally and independently form a cyclic structure having from 4 to 8 atoms in the ring;

each R is independently C₁₋₆ alkyl, C₂₋₆ alkenyl, C₂₋₆ alkynyl, C₃₋₇ cyclic alkyl, aryl, aralkyl, heteroaryl, or heteroaralkyl, and is optionally and independently substituted with halo, OH, R¹, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N⁺(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, C(O)NHSO₂R¹, C(O)NR¹SO₂R¹, cyclic C₃-C₇ alkylamino, imidazolyl, piperazinyl, morpholinyl, piperidinyl, azepanyl, pyrrolidinyl, or azetidinyl;

wherein each imidazolyl, piperazinyl, morpholinyl, piperidinyl, azepanyl, pyrrolidinyl and azetidinyl is optionally and independently substituted with one or more R¹, halo, OH, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N⁺(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, CONHSO₂H, C(O)NHSO₂R¹ or C(O)NR¹SO₂R¹;

and wherein each heteroaryl group contains one or more heteroatoms in its ring system, independently selected from O, N or S;

each R¹ is independently C₁₋₆ alkyl or C₂₋₆ alkenyl, optionally and independently substituted with halo, OH, OR², OC(O)R², OC(O)NH₂, OC(O)NHR², OC(O)NR²R², OP(O)(OH)₂, OP(O)(OR²)₂, NO₂, NH₂, NHR², NR²R², N⁺(—O⁻)R²R², NHC(O)R², NR²C(O)R², NHC(O)NH₂, NHC(O)NR²R², NR²C(O)NHR², SH, SR², S(O)H, S(O)R², SO₂R², SO₂NH₂, SO₂NHR², SO₂NR²R², CF₃, CN, CO₂H, CO₂R², CHO, C(O)R², C(O)NH₂, C(O)NHR², C(O)NR²R², C(O)NHSO₂R², C(O)NR²SO₂R², cyclic C₃-C₇ alkylamino, imidazolyl, piperazinyl, morpholinyl, piperidinyl, azepanyl, pyrrolidinyl or azetidinyl;

wherein each imidazolyl, piperazinyl, morpholinyl, piperidinyl, azepanyl, pyrrolidinyl and azetidinyl is optionally and independently substituted with one or more R², halo, OH, OR², OC(O)R², OC(O)NH₂, OC(O)NHR², OC(O)NR²R², OP(O)(OH)₂, OP(O)(OR²)₂, NO₂, NH₂, NHR², NR²R², N⁺(—O⁻)R²R², NHC(O)H, NHC(O)R², NR²C(O)R², NHC(O)NH₂, NHC(O)NR²R², NR²C(O)NHR², SH, SR², S(O)H, S(O)R², SO₂R², SO₂NH₂, SO₂NHR², SO₂NR²R², CF₃, CN, CO₂H, CO₂R², CHO, C(O)R², C(O)NH₂, C(O)NHR², C(O)NR²R², CONHSO₂H, C(O)NHSO₂R² or C(O)NR²SO₂R²;

each R² is independently C₁₋₆ alkyl, C₂₋₆ alkenyl, OH, OMe, NO₂, NH₂, CF₃, CN, CO₂H or SH;

provided that V₁, V₁′, V₂, V₂′, V₃, V₄, V₅, V₆, and V₇ are not all hydrogen; and when E is S; V₁, V₁′, and V₂′ are all hydrogen; and V₂ is Cl; then V₃ is not CH₃ when V₄ is Cl; and V₅ is not CH₃, OCH₃, or Cl; and

when E is S; V₁, V₁′, and V₂′ are all hydrogen; V₂ is H or NH₂; and V₄ is CF₃; then V₅ is not F; and

when E is S and V₁, V₁′, V₂, V₂′, V₃ and V₅ are all hydrogen, then V₄ is not COOH, COOCH₃, C(O)CH₃, CF₃, CH₃, OH, OCH₃, SCH₃, CN, O-phenyl, Cl, Br, or NO₂; and

when E is S and V₁, V₁′, V₂, V₂′, V₄ and V₅ are all hydrogen, then V₃ is not CH₃, CH₂CH₃, F, Cl, NO₂, CF₃, OCH₃, OCH₂CH₃, or a substituted 1,4-dihydropyridyl ring; and

when E is S and V₁, V₁′, V₂, V₂′, V₃ and V₄ are all hydrogen, then V₅ is not CH₃, CH₂CH₃, C(O)CH₃, CF₃, S(O)₂—NH₂, S(O)₂—NHR, N(CH₃)₂, N(CH₂CH₃)₂, N(i-Pr)phenyl, NO₂, OCH₂CH₃, NH—C(O)CH₃, NH₂, COOH, F, Cl, Br, I, OH, OCH₃, O-phenyl, O—C₇₋₈-alkyl, or C(O)NHC(i-Bu)C(O)NHCH₂CN; and

when E is S and V₁, V₁′, V₂, V₂′, and V₅ are all hydrogen; then when V₃ is C₁, V₄ is not Cl; when V₃ is CH₃, then V₄ is not Cl; and V₃ and V₄, taken together with the ring to which they are attached, do not form

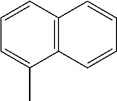

or

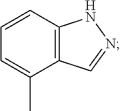

and

when E is S and V₁, V₁′, V₂, V₂′, and V₃ are all hydrogen; then when V₅ is O—C₅₋₈-alkyl, V₄ is not hydrogen, F, or CF₃; when V₅ is CH₃, V₄ is not CH₃; and V₄ and V₅, taken together with the rink to which they are attached, do not form

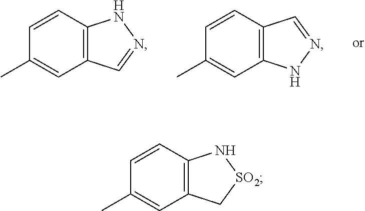

when E is S and V₁′, V₂′, V₃, V₄, and V₅ are all hydrogen, then V₁ and V₂, taken together with the ring to which they are attached, do not form

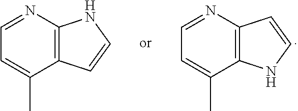

In some embodiments of the invention, V₄ is hydrogen; halo; R; OH; OR; CF₃; NO₂; NH₂; NHR; NRR; OP(O)(OH)₂; or OP(O)(OR)₂.

In specific embodiments, V₄ is hydrogen; halo; C₁₋₆ alkyl, optionally substituted with OH, NH₂, or N(CH₃)₂; OH; O—C₁₋₆ alkyl, optionally substituted with OH, NH₂, or N(CH₃)₂; CF₃; NO₂; NH₂; or OP(O)(OH)₂.

In some embodiments, V₆ is hydrogen; R; CHO; CO₂R; C(O)NH₂; C(O)NHR; or C(O)NRR.

In specific embodiments, V₆ is hydrogen; C₁₋₆ alkyl or C₂₋₄ alkenyl, optionally substituted with OH, OR¹, CO₂R¹, NH₂, NHR¹, or NR¹R¹; CHO; or CO₂R.

In more specific embodiments, V₆ is hydrogen.

In some embodiments, V₁ and V₂ are independently hydrogen, halo, R, OH, OR, CF₃, NO₂, NH₂, NHR, NRR, or, taken together with the atoms to which they are attached, form a 5- or 6-membered ring structure.

In other embodiments, V₁, V₁′, V₂, V₂′ are all hydrogen.

In certain embodiments, E is S or O.

In certain specific embodiments, E is S.

In another aspect, the invention provides a pharmaceutical composition comprising a compound as described above and a pharmacuetically acceptable carrier.

In certain embodiments, the pharmaceutical composition further comprises one or more chemotherapeutic agents.

In another aspect, the invention provides a method of treating or preventing a disease, comprising administering to a mammalian host a therapeutically-effective amount of a compound of Formula II or a pharmaceutically acceptable salt, derivative, or prodrug thereof:

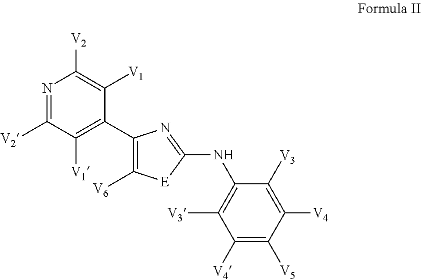

wherein:

E is S, O, or N—V₇;

V₁, V₁′, V₂, V₂′, V₃, V₃′, V₄, V₄′, V₅, V₆, and V₇ are independently hydrogen, halo, R, OH, OR, OC(O)H, OC(O)R, OC(O)NH₂, OC(O)NHR, OC(O)NRR, OP(O)(OH)₂, OP(O)(OR)₂, NO₂, NH₂, NHR, NRR, N′(—O⁻)RR, NHC(O)H, NHC(O)R, NRC(O)R, NHC(O)NH₂, NHC(O)NRR, NRC(O)NHR, N²C(O)NHR, SH, SR, S(O)H, S(O)R, SO₂R, SO₂NH₂, SO₂NHR, SO₂NRR, CF₃, CN, CO₂H, CO₂R, CHO, C(O)R, C(O)NH₂, C(O)NHR, C(O)NRR, CONHSO₂H, C(O)NHSO₂R, C(O)NRSO₂R, cyclic C₃-C₇ alkylamino, imidazolyl, piperazinyl, morpholinyl, piperidinyl, azepanyl, pyrrolidinyl or azetidinyl;

wherein each imidazolyl, piperazinyl, morpholinyl, piperidinyl, azepanyl, pyrrolidinyl and azetidinyl is optionally and independently substituted by one or more R¹, halo, OH, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N⁺(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, CONHSO₂H, C(O)NHSO₂R¹ or C(O)NR¹SO₂R¹;

and wherein groups V₁ and V₂, groups V₃ and V₄, and groups V₄ and V₅, taken together with the atoms to which they are attached, optionally and independently form a cyclic structure having from 4 to 8 atoms in the ring;

each R is independently C₁₋₆ alkyl, C₂₋₆ alkenyl, C₂₋₆ alkynyl, C₃₋₇ cyclic alkyl, aryl, aralkyl, heteroaryl, or heteroaralkyl, and is optionally and independently substituted with halo, OH, R¹, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N⁺(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, C(O)NHSO₂R¹, C(O)NR¹SO₂R¹, cyclic C₃-C₇ alkylamino, imidazolyl, piperazinyl, morpholinyl, piperidinyl, azepanyl, pyrrolidinyl, or azetidinyl;

wherein each imidazolyl, piperazinyl, morpholinyl, piperidinyl, azepanyl, pyrrolidinyl and azetidinyl is optionally and independently substituted with one or more R¹, halo, OH, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N⁺(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, CONHSO₂H, C(O)NHSO₂R¹ or C(O)NR¹SO₂R¹;

and wherein each heteroaryl group contains one or more heteroatoms in its ring system, independently selected from O, N or S;

each R¹ is independently C₁₋₆ alkyl or C₂₋₆ alkenyl, optionally and independently substituted with halo, OH, OR², OC(O)R², OC(O)NH₂, OC(O)NHR², OC(O)NR²R², OP(O)(OH)₂, OP(O)(OR²)₂, NO₂, NH₂, NHR², NR²R², N⁺(—O⁻)R²R², NHC(O)R², NR²C(O)R², NHC(O)NH₂, NHC(O)NR²R², NR²C(O)NHR², SH, SR², S(O)H, S(O)R², SO₂R², SO₂NH₂, SO₂NHR², SO₂NR²R², CF₃, CN, CO₂H, CO₂R², CHO, C(O)R², C(O)NH₂, C(O)NHR², C(O)NR²R², C(O)NHSO₂R², C(O)NR²SO₂R², cyclic C₃-C₇ alkylamino, imidazolyl, piperazinyl, morpholinyl, piperidinyl, azepanyl, pyrrolidinyl or azetidinyl;

wherein each imidazolyl, piperazinyl, morpholinyl, piperidinyl, azepanyl, pyrrolidinyl and azetidinyl is optionally and independently substituted with one or more R², halo, OH, OR², OC(O)R², OC(O)NH₂, OC(O)NHR², OC(O)NR²R², OP(O)(OH)₂, OP(O)(OR²)₂, NO₂, NH₂, NHR², NR²R², N⁺(—O⁻)R²R², NHC(O)H, NHC(O)R², NR²C(O)R², NHC(O)NH₂, NHC(O)NR²R², NR²C(O)NHR², SH, SR², S(O)H, S(O)R², SO₂R², SO₂NH₂, SO₂NHR², SO₂NR²R², CF₃, CN, CO₂H, CO₂R², CHO, C(O)R², C(O)NH₂, C(O)NHR², C(O)NR²R², CONHSO₂H, C(O)NHSO₂R² or C(O)NR²SO₂R²; and

each R² is independently C₁₋₆ alkyl, C₂₋₆ alkenyl, OH, OMe, NO₂, NH₂, CF₃, CN, CO₂H or SH.

In certain embodiments, the V₁, V₁′, V₂, V₂′, V₃, V₃′, V₄, V₄′, V₅, V₆, V₇, and E groups of the compound administered according to the methods of the invention have the specific definitions recited above.

In certain embodiments, the disease treated or prevented according to the methods of the invention is caused by a defect in the von Hippel-Lindau gene.

In certain embodiments, the compound administered according to the methods of the invention targets cells deficient in the von Hippel-Lindau gene.

In certain embodiments, the compound administered according to the methods of the invention induces autophagic cell death.

In certain embodiments, the disease is a cancer.

In specific embodiments, the cancer is a renal cell carcinoma, a pheochromocytoma, a hemangioblastoma of the central nervous system or retina, an endolymphatic sac tumor, a renal cyst, a pancreatic cyst, a neuroendocrine tumor, an endolymphatic sac tumor, or an epididymal or broad ligament cystadenoma.

In other specific embodiments, the cancer is a cancer of the retina, brain, spinal cord, ear, epidymis, broad ligament, adrenal gland, kidney, or pancreas.

In some embodiments, the methods further comprise the step of administering a therapeutically-effective amount of one or more chemotherapeutic agents to the mammalian host before, during, or after administration of the compound.

### LISTING OF DRAWINGS

FIG. 1. Effects of STF-62247 on VHL-deficient cells.

FIG. 2. Detection of autophagic vacuoles in RCCs following treatment with STF-62247.

FIG. 3. Role of ATG5 in autophagic cell death following treatment with STF-62247.

FIG. 4. Golgi trafficking and role of PI3K following treatment with STF-62247.

FIG. 5. Evaluation of autophagy in RCCs treated with STF-62247 analogs.

FIG. 6. Effects of STF-62247 on Golgi trafficking proteins.

FIG. 7. Screening assays.

FIG. 8. Transmission electron microscopy of control and STF-62247-treated RCC4 cells.

FIG. 9. Effects of STF-62247 and other treatments on VHL-deficient RCC4 and wild-type VHL cells.

FIG. 10. Knockdown of genes using siRNA.

### DETAILED DESCRIPTION OF THE INVENTION

The instant invention provides novel heteroaryl compounds, compositions, and methods of use in targeting cells defective in the von Hippel-Lindau (VHL) gene and diseases associated with such defects. The provided compounds induce cytotoxicity and reduce tumor growth of VHL-deficient cells compared to genetically matched cells with wild-type VHL. The compounds thus selectively induce cell death in VHL-deficient cells and therefore represent a novel strategy for targeted therapy. See also Sutphin, P., et al., Cancer Res. 2007, 67, 5896; Turcotte, S., et al., Cancer Cell. 2008, 14, 90; Turcotte, S., et al., Autophagy 2008, Oct. 1; 4(7):944-6. Epub 2008 Oct. 13.

Before further description of the invention, certain terms employed in the specification, examples and appended claims are, for convenience, collected here.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In some embodiments, a straight chain or branched chain alkyl has 30 or fewer carbon atoms in its backbone (e.g., C₁-C₃₀ for straight chains, C₃-C₃₀ for branched chains), and more specifically 20 or fewer. Likewise, some cycloalkyls have from 3-10 carbon atoms in their ring structure, and more specifically have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halo, a hydroxyl, a carbonyl (such as a keto, a carboxy, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a thio, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. “C₀-alkyl” indicates a hydrogen where the group is in a terminal position, or is a bond if internal. The terms “C_{2-y}-alkenyl” and “C_{2-y}-alkynyl” refer to substituted or unsubstituted unsaturated aliphatic groups analogous in length and possible substitution to the alkyls described above, but that contain at least one double or triple bond, respectively.

The term “alkylamino”, as used herein, refers to an amino group substituted with at least one alkyl group.

The term “alkylthio”, as used herein, refers to a thiol group substituted with an alkyl group and may be represented by the general formula alkyl-S—.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, cycloalkyl, heterocyclyl, aryl, or heteroaryl groups is contemplated.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, cycloalkyl, heterocyclyl, aryl, or heteroaryl groups is contemplated.

The term “amide”, as used herein, refers to a group

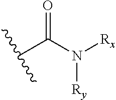

wherein Rₓ and R_{y} each independently represent a hydrogen or hydrocarbyl group, or Rₓ and R_{y} taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

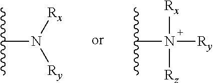

wherein Rₓ, R_{y}, and R_{z} each independently represent a hydrogen or a hydrocarbyl group, or Rₓ and R_{y} taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein includes substituted or unsubstituted single-ring aromatic groups in which each atom of the ring is carbon. In certain embodiments, the ring is a 5- to 7-membered ring, and in more specific embodiments is a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

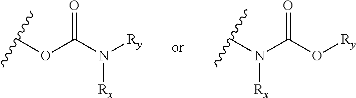

wherein Rₓ and R_{y} independently represent hydrogen or a hydrocarbyl group, or Rₓ and R_{y} taken together with the atoms to which they are attached complete a heterocycle having from 4 to 8 atoms in the ring structure.

The term “cycloalkyl” and “cyclic alkyl”, as used herein, refers to a non-aromatic saturated or unsaturated ring in which each atom of the ring is carbon. In certain embodiments, a cycloalkyl ring contains from 3 to 10 atoms, and in more specific embodiments from 5 to 7 atoms.

The term “carbonate” is art-recognized and refers to a group —OCO₂—R₄, wherein R₄ represents a hydrocarbyl group.

The term “carboxy”, as used herein, refers to a group represented by the formula —CO₂H.

The terms “halo” and “halogen” as used herein mean halogen and include chloro, fluoro, bromo, and iodo.

The terms “hetaralkyl” and “heteroaralkyl”, as used herein, refer to an alkyl group substituted with a hetaryl group.

The terms “heteroaryl” and “hetaryl” include substituted or unsubstituted aromatic single ring structures, in certain specific embodiments 4- to 8-membered rings or 5- to 7-membered rings, more specifically 5- to 6-membered rings, whose ring structures include at least one heteroatom, in some embodiments one to four heteroatoms, and in more specific embodiments one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Typical heteroatoms are nitrogen, oxygen, and sulfur.

The terms “heterocyclyl”, “heterocycle”, and “heterocyclic” refer to substituted or unsubstituted non-aromatic ring structures, in certain specific embodiments 3- to 10-membered rings, more specifically 3- to 7-membered rings, whose ring structures include at least one heteroatom, in some embodiments one to four heteroatoms, and in more specific embodiments one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, and in certain embodiments, six or fewer. A “lower alkyl”, for example, refers to an alkyl group that contains ten or fewer carbon atoms, and in specific embodiments six or fewer carbon atoms. In certain embodiments, the acyl, acyloxy, alkyl, alkenyl, alkynyl, and alkoxy substituents defined herein are respectively lower acyl, lower acyloxy, lower alkyl, lower alkenyl, lower alkynyl, and lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc., under conditions in which the compound is to be used. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents may include any substituents described herein, for example, a halogen, a hydroxyl, a carbonyl (such as a keto, a carboxy, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain may themselves be substituted, if appropriate.

Unless specifically described as “unsubstituted”, references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

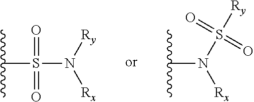

wherein Rₓ and R_{y} independently represent hydrogen or hydrocarbyl.

The term “sulfoxide” is art-recognized and refers to the group —S(O)—Rₓ, wherein Rₓ represents a hydrocarbyl.

The term “sulfonate” is art-recognized and refers to the group SO₃H, or a pharmaceutically acceptable salt thereof.

The term “sulfone” is art-recognized and refers to the group —S(O)₂—Rₓ, wherein Rₓ represents a hydrocarbyl.

### Compounds

In one aspect, the present invention provides novel compounds that target cells deficient in the von Hippel-Lindau gene. In certain embodiments, the compounds are represented by Formula II:

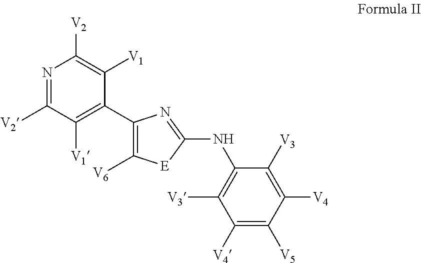

or a pharmaceutically acceptable salt, derivative, or prodrug thereof, wherein:

E is S, O, or N—V₇;

V₃, V₃′, V₄′, and V₅ are all hydrogen;

V₁, V₁′, V₂, V₂′, V₄, V₆, and V₇ are independently hydrogen, halo, R, OH, OR, OC(O)H, OC(O)R, OC(O)NH₂, OC(O)NHR, OC(O)NRR, OP(O)(OH)₂, OP(O)(OR)₂, NO₂, NH₂, NHR, NRR, N⁺(—O⁻)RR, NHC(O)H, NHC(O)R, NRC(O)R, NHC(O)NH₂, NHC(O)NRR, NRC(O)NHR, N²C(O)NHR, SH, SR, S(O)H, S(O)R, SO₂R, SO₂NH₂, SO₂NHR, SO₂NRR, CF₃, CN, CO₂H, CO₂R, CHO, C(O)R, C(O)NH₂, C(O)NHR, C(O)NRR, CONHSO₂H, C(O)NHSO₂R, C(O)NRSO₂R, cyclic C₃-C₇ alkylamino, imidazolyl, piperazinyl, morpholinyl, piperidinyl, azepanyl, pyrrolidinyl or azetidinyl;

wherein each imidazolyl, piperazinyl, morpholinyl, piperidinyl, azepanyl, pyrrolidinyl and azetidinyl is optionally and independently substituted by one or more R¹, halo, OH, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N⁺(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, CONHSO₂H, C(O)NHSO₂R¹ or C(O)NR¹SO₂R¹;

and wherein V₁ and V₂, taken together with the atoms to which they are attached, optionally form a cyclic structure having from 4 to 8 atoms in the ring;

each R is independently C₁₋₆ alkyl, C₂₋₆ alkenyl, C₂₋₆ alkynyl, C₃₋₇ cyclic alkyl, aryl, aralkyl, heteroaryl, or heteroaralkyl, and is optionally and independently substituted with halo, OH, R¹, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N⁺(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, C(O)NHSO₂R¹, C(O)NR¹SO₂R¹, cyclic C₃-C₇ alkylamino, imidazolyl, piperazinyl, morpholinyl, piperidinyl, azepanyl, pyrrolidinyl, or azetidinyl;

wherein each imidazolyl, piperazinyl, morpholinyl, piperidinyl, azepanyl, pyrrolidinyl and azetidinyl is optionally and independently substituted with one or more R¹, halo, OH, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N⁺(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, CONHSO₂H, C(O)NHSO₂R¹ or C(O)NR¹SO₂R¹;

and wherein each heteroaryl group contains one or more heteroatoms in its ring system, independently selected from O, N or S;

each R¹ is independently C₁₋₆ alkyl or C₂₋₆ alkenyl, optionally and independently substituted with halo, OH, OR², OC(O)R², OC(O)NH₂, OC(O)NHR², OC(O)NR²R², OP(O)(OH)₂, OP(O)(OR²)₂, NO₂, NH₂, NHR², NR²R², N⁺(—O⁻)R²R², NHC(O)R², NR²C(O)R², NHC(O)NH₂, NHC(O)NR²R², NR²C(O)NHR², SH, SR², S(O)H, S(O)R², SO₂R², SO₂NH₂, SO₂NHR², SO₂NR²R², CF₃, CN, CO₂H, CO₂R², CHO, C(O)R², C(O)NH₂, C(O)NHR², C(O)NR²R², C(O)NHSO₂R², C(O)NR²SO₂R², cyclic C₃-C₇ alkylamino, imidazolyl, piperazinyl, morpholinyl, piperidinyl, azepanyl, pyrrolidinyl or azetidinyl;

wherein each imidazolyl, piperazinyl, morpholinyl, piperidinyl, azepanyl, pyrrolidinyl and azetidinyl is optionally and independently substituted with one or more R², halo, OH, OR², OC(O)R², OC(O)NH₂, OC(O)NHR², OC(O)NR²R², OP(O)(OH)₂, OP(O)(OR²)₂, NO₂, NH₂, NHR², NR²R², N⁺(—O⁻)R²R², NHC(O)H, NHC(O)R², NR²C(O)R², NHC(O)NH₂, NHC(O)NR²R², NR²C(O)NHR², SH, SR², S(O)H, S(O)R², SO₂R², SO₂NH₂, SO₂NHR², SO₂NR²R², CF₃, CN, CO₂H, CO₂R², CHO, C(O)R², C(O)NH₂, C(O)NHR², C(O)NR²R², CONHSO₂H, C(O)NHSO₂R² or C(O)NR²SO₂R²;

each R² is independently C₁₋₆ alkyl, C₂₋₆ alkenyl, OH, OMe, NO₂, NH₂, CF₃, CN, CO₂H or SH;

provided that V₁, V₁′, V₂, V₂′, V₄, V₆, and V₇ are not all hydrogen; and

when E is S; V₁, V₁′, V₂′, and V₆ are all hydrogen; and V₂ is Cl; then V₄ is not Cl; and

when E is S; and V₁, V₁′, V₂, V₂′, and V₆ are all hydrogen; then V₄ is not COOH, COOCH₃, C(O)CH₃, CF₃, CH₃, OH, OCH₃, SCH₃, CN, O-phenyl, Cl, Br, or NO₂; and

when E is S; V₁′, V₂′, V₄, and V₆ are all hydrogen; then V₁ and V₂, taken together with the ring to which they are attached, do not form

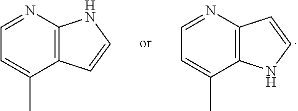

In some embodiments, R is independently C₁₋₆ alkyl, C₂₋₄ alkenyl, C₃₋₇ cyclic alkyl, C₄₋₈ aryl, or C₄₋₈ heteroaryl, and is optionally and independently substituted with halo, OH, R¹, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N′(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, C(O)NHSO₂R¹, C(O)NR¹SO₂R¹, cyclic C₃-C₇ alkylamino, imidazolyl, piperazinyl, morpholinyl, piperidinyl, azepanyl, pyrrolidinyl, or azetidinyl.

In some embodiments, V₄ is hydrogen; halo; R; OH; OR; CF₃; NO₂; NH₂; NHR; NRR; OP(O)(OH)₂; or OP(O)(OR)₂.

In more specific embodiments, V₄ is hydrogen; halo; C₁₋₆ alkyl, optionally substituted with OH, NH₂, or N(CH₃)₂; OH; O—C₁₋₆ alkyl, optionally substituted with OH, NH₂, or N(CH₃)₂; CF₃; NO₂; NH₂; or OP(O)(OH)₂.

In some embodiments, V₆ is hydrogen; R; CHO; CO₂R; C(O)NH₂; C(O)NHR; or C(O)NRR.

In more specific embodiments, V₆ is hydrogen; C₁₋₆ alkyl or C₂₋₄ alkenyl, optionally substituted with OH, OR¹, CO₂R¹, NH₂, NHR¹, or NR¹R¹; CHO; or CO₂R.

In even more specific embodiments, V₆ is hydrogen.

In some embodiments, V₁ and V₂ are independently hydrogen, halo, R, OH, OR, CF₃, NO₂, NH₂, NHR, NRR, or, taken together with the atoms to which they are attached, form a 5- or 6-membered ring structure.

In other embodiments, V₁, V₁′, V₂, V₂′ are all hydrogen.

In some embodiments, E is S or O.

In specific embodiments, E is S.

In certain embodiments, compounds of the invention are represented by Formula II, or a pharmaceutically acceptable salt, derivative, or prodrug thereof, wherein:

E is S, O, or N—V₇;

V₅ and V₆ are both hydrogen;

V₁, V₁′, V₂, V₂′, V₃, V₃′, V₄, V₄′, and V₇ are independently hydrogen, halo, R, OH, OR, OC(O)H, OC(O)R, OC(O)NH₂, OC(O)NHR, OC(O)NRR, OP(O)(OH)₂, OP(O)(OR)₂, NO₂, NH₂, NHR, NRR, N⁺(—O⁻)RR, NHC(O)H, NHC(O)R, NRC(O)R, NHC(O)NH₂, NHC(O)NRR, NRC(O)NHR, N²C(O)NHR, SH, SR, S(O)H, S(O)R, SO₂R, SO₂NH₂, SO₂NHR, SO₂NRR, CF₃, CN, CO₂H, CO₂R, CHO, C(O)R, C(O)NH₂, C(O)NHR, C(O)NRR, CONHSO₂H, C(O)NHSO₂R, C(O)NRSO₂R, cyclic C₃-C₇ alkylamino, imidazolyl, piperazinyl, morpholinyl, piperidinyl, azepanyl, pyrrolidinyl or azetidinyl;

wherein each imidazolyl, piperazinyl, morpholinyl, piperidinyl, azepanyl, pyrrolidinyl and azetidinyl is optionally and independently substituted by one or more R¹, halo, OH, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N⁺(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, CONHSO₂H, C(O)NHSO₂R¹ or C(O)NR¹SO₂R¹;

and wherein groups V₁ and V₂ and groups V₃ and V₄, taken together with the atoms to which they are attached, optionally and independently form a cyclic structure having from 4 to 8 atoms in the ring;

each R is independently C₁₋₆ alkyl, C₂₋₆ alkenyl, C₂₋₆ alkynyl, C₃₋₇ cyclic alkyl, aryl, aralkyl, heteroaryl, or heteroaralkyl, and is optionally and independently substituted with halo, OH, R¹, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N⁺(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, C(O)NHSO₂R¹, C(O)NR¹SO₂R¹, cyclic C₃-C₇ alkylamino, imidazolyl, piperazinyl, morpholinyl, piperidinyl, azepanyl, pyrrolidinyl, or azetidinyl;

wherein each imidazolyl, piperazinyl, morpholinyl, piperidinyl, azepanyl, pyrrolidinyl and azetidinyl is optionally and independently substituted with one or more R¹, halo, OH, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N⁺(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, CONHSO₂H, C(O)NHSO₂R¹ or C(O)NR¹SO₂R¹;

and wherein each heteroaryl group contains one or more heteroatoms in its ring system, independently selected from O, N or S;

each R¹ is independently C₁₋₆ alkyl or C₂₋₆ alkenyl, optionally and independently substituted with halo, OH, OR², OC(O)R², OC(O)NH₂, OC(O)NHR², OC(O)NR²R², OP(O)(OH)₂, OP(O)(OR²)₂, NO₂, NH₂, NHR², NR²R², N⁺(—O⁻)R²R², NHC(O)R², NR²C(O)R², NHC(O)NH₂, NHC(O)NR²R², NR²C(O)NHR², SH, SR², S(O)H, S(O)R², SO₂R², SO₂NH₂, SO₂NHR², SO₂NR²R², CF₃, CN, CO₂H, CO₂R², CHO, C(O)R², C(O)NH₂, C(O)NHR², C(O)NR²R², C(O)NHSO₂R², C(O)NR²SO₂R², cyclic C₃-C₇ alkylamino, imidazolyl, piperazinyl, morpholinyl, piperidinyl, azepanyl, pyrrolidinyl or azetidinyl;

wherein each imidazolyl, piperazinyl, morpholinyl, piperidinyl, azepanyl, pyrrolidinyl and azetidinyl is optionally and independently substituted with one or more R², halo, OH, OR², OC(O)R², OC(O)NH₂, OC(O)NHR², OC(O)NR²R², OP(O)(OH)₂, OP(O)(OR²)₂, NO₂, NH₂, NHR², NR²R², N⁺(—O⁻)R²R², NHC(O)H, NHC(O)R², NR²C(O)R², NHC(O)NH₂, NHC(O)NR²R², NR²C(O)NHR², SH, SR², S(O)H, S(O)R², SO₂R², SO₂NH₂, SO₂NHR², SO₂NR²R², CF₃, CN, CO₂H, CO₂R², CHO, C(O)R², C(O)NH₂, C(O)NHR², C(O)NR²R², CONHSO₂H, C(O)NHSO₂R² or C(O)NR²SO₂R²;

each R² is independently C₁₋₆ alkyl, C₂₋₆ alkenyl, OH, OMe, NO₂, NH₂, CF₃, CN, CO₂H or SH;

provided that V₁, V₁′, V₂, V₂′, V₃, V₃′, V₄, V₄′, and V₇ are not all hydrogen; and

when E is S; V₁, V₁′, and V₂′ are all hydrogen; and V₂ is Cl; then V₃ is not CH₃ and V₄ is not Cl; and

when E is S and V₃′ and V₄ are both hydrogen, then either V₃ or V₄′ is also hydrogen; and

when E is S and V₁, V₁′, V₂, V₂′, V₃, V₃′, and V₄′ are all hydrogen, then V₄ is not COOH, COOCH₃, C(O)CH₃, CF₃, CH₃, OH, OCH₃, SCH₃, CN, O-phenyl, Cl, Br, or NO₂; and

when E is S and V₁, V₁′, V₂, V₂′, V₃′, V₄, and V₄′ are all hydrogen, then V₃ is not CH₃, CH₂CH₃, F, Cl, NO₂, CF₃, OCH₃, OCH₂CH₃, or a substituted 1,4-dihydropyridyl ring; and

when E is S and V₁, V₁′, V₂, V₂′, V₃′, and V₄′ are all hydrogen; then when V₃ is C₁, V₄ is not Cl; and when V₃ is CH₃, V₄ is not Cl; and V₃ and V₄, taken together with the ring to which they are attached, do not form

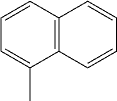

or

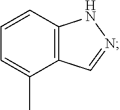

and

when E is S and V₁′, V₂′, V₃, V₃′, V₄, and V₄′ are all hydrogen, then V₁ and V₂, taken together with the ring to which they are attached, do not form

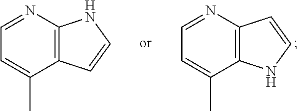

and

when V₃ and V₃′ are both hydrogen, then V₄ and V₄′ are not both CH₃.

In some embodiments, V₁ and V₂, taken together with the atoms to which they are attached, optionally and independently form a cyclic structure having from 4 to 8 atoms in the ring;

In some embodiments, R is independently C₁₋₆ alkyl, C₂₋₄ alkenyl, C₃₋₇ cyclic alkyl, C₄₋₈ aryl, or C₄₋₈ heteroaryl, and is optionally and independently substituted with halo, OH, R¹, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N′(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, C(O)NHSO₂R¹, C(O)NR¹SO₂R¹, cyclic C₃-C₇ alkylamino, imidazolyl, piperazinyl, morpholinyl, piperidinyl, azepanyl, pyrrolidinyl, or azetidinyl.

In some embodiments, V₃ and V₄ are independently hydrogen; halo; R; OH; OR; CF₃; NO₂; NH₂; NHR; NRR; OP(O)(OH)₂; OP(O)(OR)₂; or, taken together with the atoms to which they are attached, form a 5- or 6-membered ring.

In specific embodiments, V₃ and V₄ are independently hydrogen; halo; C₁₋₆ alkyl, optionally substituted with OH, NH₂, or N(CH₃)₂; OH; O—C₁₋₆ alkyl, optionally substituted with OH, NH₂, or N(CH₃)₂; CF₃; NO₂; NH₂; or OP(O)(OH)₂.

In some embodiments, V₄ is hydrogen; halo; R; OH; OR; CF₃; NO₂; NH₂; NHR; NRR; OP(O)(OH)₂; or OP(O)(OR)₂.

In specific embodiments, V₄ is hydrogen; halo; C₁₋₆ alkyl, optionally substituted with OH, NH₂, or N(CH₃)₂; OH; O—C₁₋₆ alkyl, optionally substituted with OH, NH₂, or N(CH₃)₂; CF₃; NO₂; NH₂; or OP(O)(OH)₂.

In some embodiments, V₁ and V₂ are independently hydrogen, halo, R, OH, OR, CF₃, NO₂, NH₂, NHR, NRR, or, taken together with the atoms to which they are attached, form a 5- or 6-membered ring structure.

In some embodiments, V₁, V₁′, V₂, V₂′ are all hydrogen.

In some embodiments, E is S or O.

In specific embodiments, E is S.

In other embodiments, compounds of the invention are represented by Formula II, or a pharmaceutically acceptable salt, derivative, or prodrug thereof, wherein:

E is S, O, or N—V₇;

V₃′ and V₄′ are both hydrogen;

V₁, V₁′, V₂, V₂′, V₃, V₄, V₅, V₆, and V₇ are independently hydrogen, halo, R, OH, OR, OC(O)H, OC(O)R, OC(O)NH₂, OC(O)NHR, OC(O)NRR, OP(O)(OH)₂, OP(O)(OR)₂, NO₂, NH₂, NHR, NRR, N⁺(—O⁻)RR, NHC(O)H, NHC(O)R, NRC(O)R, NHC(O)NH₂, NHC(O)NRR, NRC(O)NHR, N²C(O)NHR, SH, SR, S(O)H, S(O)R, SO₂R, SO₂NH₂, SO₂NHR, SO₂NRR, CF₃, CN, CO₂H, CO₂R, CHO, C(O)R, C(O)NH₂, C(O)NHR, C(O)NRR, CONHSO₂H, C(O)NHSO₂R, C(O)NRSO₂R, cyclic C₃-C₇ alkylamino, imidazolyl, piperazinyl, morpholinyl, piperidinyl, azepanyl, pyrrolidinyl or azetidinyl;

wherein each imidazolyl, piperazinyl, morpholinyl, piperidinyl, azepanyl, pyrrolidinyl and azetidinyl is optionally and independently substituted by one or more R¹, halo, OH, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N⁺(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, CONHSO₂H, C(O)NHSO₂R¹ or C(O)NR¹SO₂R¹;

and wherein groups V₁ and V₂, groups V₃ and V₄, and groups V₄ and V₅, taken together with the atoms to which they are attached, optionally and independently form a cyclic structure having from 4 to 8 atoms in the ring;

each R is independently C₁₋₆ alkyl, C₂₋₆ alkenyl, C₂₋₆ alkynyl, C₃₋₇ cyclic alkyl, aryl, aralkyl, heteroaryl, or heteroaralkyl, and is optionally and independently substituted with halo, OH, R¹, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N⁺(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, C(O)NHSO₂R¹, C(O)NR¹SO₂R¹, cyclic C₃-C₇ alkylamino, imidazolyl, piperazinyl, morpholinyl, piperidinyl, azepanyl, pyrrolidinyl, or azetidinyl;

wherein each imidazolyl, piperazinyl, morpholinyl, piperidinyl, azepanyl, pyrrolidinyl and azetidinyl is optionally and independently substituted with one or more R¹, halo, OH, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N⁺(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, CONHSO₂H, C(O)NHSO₂R¹ or C(O)NR¹SO₂R¹;

and wherein each heteroaryl group contains one or more heteroatoms in its ring system, independently selected from O, N or S;

each R¹ is independently C₁₋₆ alkyl or C₂₋₆ alkenyl, optionally and independently substituted with halo, OH, OR², OC(O)R², OC(O)NH₂, OC(O)NHR², OC(O)NR²R², OP(O)(OH)₂, OP(O)(OR²)₂, NO₂, NH₂, NHR², NR²R², N⁺(—O⁻)R²R², NHC(O)R², NR²C(O)R², NHC(O)NH₂, NHC(O)NR²R², NR²C(O)NHR², SH, SR², S(O)H, S(O)R², SO₂R², SO₂NH₂, SO₂NHR², SO₂NR²R², CF₃, CN, CO₂H, CO₂R², CHO, C(O)R², C(O)NH₂, C(O)NHR², C(O)NR²R², C(O)NHSO₂R², C(O)NR²SO₂R², cyclic C₃-C₇ alkylamino, imidazolyl, piperazinyl, morpholinyl, piperidinyl, azepanyl, pyrrolidinyl or azetidinyl;

wherein each imidazolyl, piperazinyl, morpholinyl, piperidinyl, azepanyl, pyrrolidinyl and azetidinyl is optionally and independently substituted with one or more R², halo, OH, OR², OC(O)R², OC(O)NH₂, OC(O)NHR², OC(O)NR²R², OP(O)(OH)₂, OP(O)(OR²)₂, NO₂, NH₂, NHR², NR²R², N⁺(—O⁻)R²R², NHC(O)H, NHC(O)R², NR²C(O)R², NHC(O)NH₂, NHC(O)NR²R², NR²C(O)NHR², SH, SR², S(O)H, S(O)R², SO₂R², SO₂NH₂, SO₂NHR², SO₂NR²R², CF₃, CN, CO₂H, CO₂R², CHO, C(O)R², C(O)NH₂, C(O)NHR², C(O)NR²R², CONHSO₂H, C(O)NHSO₂R² or C(O)NR²SO₂R²;

each R² is independently C₁₋₆ alkyl, C₂₋₆ alkenyl, OH, OMe, NO₂, NH₂, CF₃, CN, CO₂H or SH;

provided that V₁, V₁′, V₂, V₂′, V₃, V₄, V₅, V₆, and V₇ are not all hydrogen; and

when E is S; V₁, V₁′, and V₂′ are all hydrogen; and V₂ is Cl; then V₃ is not CH₃ when V₄ is Cl; and V₅ is not CH₃, OCH₃, or Cl; and

when E is S; V₁, V₁′, and V₂′ are all hydrogen; V₂ is H or NH₂; and V₄ is CF₃; then V₅ is not F; and

when E is S and V₁, V₁′, V₂, V₂′, V₃ and V₅ are all hydrogen, then V₄ is not COOH, COOCH₃, C(O)CH₃, CF₃, CH₃, OH, OCH₃, SCH₃, CN, O-phenyl, Cl, Br, or NO₂; and

when E is S and V₁, V₁′, V₂, V₂′, V₄ and V₅ are all hydrogen, then V₃ is not CH₃, CH₂CH₃, F, Cl, NO₂, CF₃, OCH₃, OCH₂CH₃, or a substituted 1,4-dihydropyridyl ring; and

when E is S and V₁, V₁′, V₂, V₂′, V₃ and V₄ are all hydrogen, then V₅ is not CH₃, CH₂CH₃, C(O)CH₃, CF₃, S(O)₂—NH₂, S(O)₂—NHR, N(CH₃)₂, N(CH₂CH₃)₂, N(i-Pr)phenyl, NO₂, OCH₂CH₃, NH—C(O)CH₃, NH₂, COOH, F, Cl, Br, I, OH, OCH₃, O-phenyl, O—C₇₋₈-alkyl, or C(O)NHC(i-Bu)C(O)NHCH₂CN; and

when E is S and V₁, V₁′, V₂, V₂′, and V₅ are all hydrogen; then when V₃ is C₁, V₄ is not Cl; when V₃ is CH₃, then V₄ is not Cl; and V₃ and V₄, taken together with the ring to which they are attached, do not form

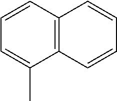

or

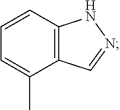

and

when E is S and V₁, V₁′, V₂, V₂′, and V₃ are all hydrogen; then when V₅ is O—C₅₋₈-alkyl, V₄ is not hydrogen, F, or CF₃; when V₅ is CH₃, V₄ is not CH₃; and V₄ and V₅, taken together with the ring to which they are attached, do not form

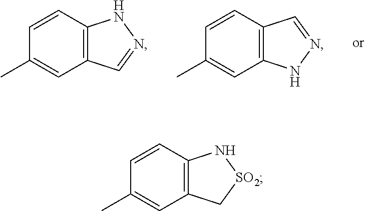

when E is S and V₁′, V₂′, V₃, V₄, and V₅ are all hydrogen, then V₁ and V₂, taken together with the ring to which they are attached, do not form

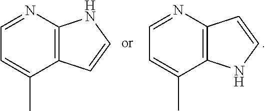

In some embodiments, R is independently C₁₋₆ alkyl, C₂₋₄ alkenyl, C₂₋₄ alkynyl, C₃₋₇ cyclic alkyl, C₄₋₈ aryl, or C₄₋₈ heteroaryl, and is optionally and independently substituted with halo, OH, R¹, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N⁺(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, C(O)NHSO₂R¹, C(O)NR¹SO₂R¹, cyclic C₃-C₇ alkylamino, imidazolyl, piperazinyl, morpholinyl, piperidinyl, azepanyl, pyrrolidinyl, or azetidinyl.

In some embodiments, V₃, V₄, and V₅ are independently hydrogen; halo; R; OH; OR; CF₃; NO₂; NH₂; NHR; NRR; OP(O)(OH)₂; or OP(O)(OR)₂; and groups V₃ and V₄ and groups V₄ and V₅, taken together with the atoms to which they are attached, optionally and independently form a 5- or 6-membered ring structure.

In specific embodiments, V₃, V₄, and V₅ are independently hydrogen; halo; C₁₋₆ alkyl, optionally substituted with OH, NH₂, or N(CH₃)₂; OH; O—C₁₋₆ alkyl, optionally substituted with OH, NH₂, or N(CH₃)₂; CF₃; NO₂; NH₂; or OP(O)(OH)₂.

In some embodiments, V₄ is hydrogen; halo; R; OH; OR; CF₃; NO₂; NH₂; NHR; NRR; OP(O)(OH)₂; or OP(O)(OR)₂.

In specific embodiments, V₄ is hydrogen; halo; C₁₋₆ alkyl, optionally substituted with OH, NH₂, or N(CH₃)₂; OH; O—C₁₋₆ alkyl, optionally substituted with OH, NH₂, or N(CH₃)₂; CF₃; NO₂; NH₂; or OP(O)(OH)₂.

In some embodiments, V₁ and V₂ are independently hydrogen, halo, R, OH, OR, CF₃, NO₂, NH₂, NHR, NRR, or, taken together with the atoms to which they are attached, form a 5- or 6-membered ring structure.

In other embodiments, V₁, V₁′, V₂, V₂′ are all hydrogen.

In some embodiments, E is S or O.

In more specific embodiments, E is S.

In even more specific embodiments of the invention, the compound is selected from the following list of compounds:
N-Phenyl-4-(4-pyridinyl)-1,3-thiazol-2-amine,
N-(3-Methylphenyl)-4-(4-pyridinyl)-1,3-thiazol-2-amine,
N-(3-Ethylphenyl)-4-(4-pyridinyl)-1,3-thiazol-2-amine,
N-(3-Isopropylphenyl)-4-(4-pyridinyl)-1,3-thiazol-2-amine,
N-(3-tert-Butylphenyl)-4-(4-pyridinyl)-1,3-thiazol-2-amine,
3-{[4-(4-Pyridinyl)-1,3-thiazol-2-yl]amino}phenol,
N-(3-Methoxyphenyl)-4-(4-pyridinyl)-1,3-thiazol-2-amine,
N-(3-Chlorophenyl)-4-(4-pyridinyl)-1,3-thiazol-2-amine,
4-(4-Pyridinyl)-N-[3-(trifluoromethyl)phenyl]-1,3-thiazol-2-amine,
N-(4-Methylphenyl)-4-(4-pyridinyl)-1,3-thiazol-2-amine,
2-(4-{[4-(4-Pyridinyl)-1,3-thiazol-2-yl]amino}phenyl)ethanol,
4-{[4-(4-Pyridinyl)-1,3-thiazol-2-yl]amino}phenol,
N-(4-Methoxyphenyl)-4-(4-pyridinyl)-1,3-thiazol-2-amine,
N-{4-[2-(Dimethylamino)ethoxy]phenyl}-4-(4-pyridinyl)-1,3-thiazol-2-amine,
N-(4-Chlorophenyl)-4-(4-pyridinyl)-1,3-thiazol-2-amine,
N-(4-Fluorophenyl)-4-(4-pyridinyl)-1,3-thiazol-2-amine,
N-(4-Nitrophenyl)-4-(4-pyridinyl)-1,3-thiazol-2-amine,
N-{4-[2-(Dimethylamino)ethyl]phenyl}-4-(4-pyridinyl)-1,3-thiazol-2-amine,
N¹-[4-(4-Pyridinyl)-1,3-thiazol-2-yl]-1,4-benzenediamine,
3-{[4-(4-Pyridinyl)-1,3-thiazol-2-yl]amino}phenyl Dihydrogen Phosphate,
4-{[4-(4-Pyridinyl)-1,3-thiazol-2-yl]amino}phenyl Dihydrogen Phosphate,
N-(3,4-Dimethylphenyl)-4-(4-pyridinyl)-1,3-thiazol-2-amine,
N-(3,5-Dimethylphenyl)-4-(4-pyridinyl)-1,3-thiazol-2-amine,
N-(2,3-Dihydro-1H-inden-5-yl)-4-(4-pyridinyl)-1,3-thiazol-2-amine,
2-Methyl-5-{[4-(4-pyridinyl)-1,3-thiazol-2-yl]amino}phenol,
2-Methoxy-5-{[4-(4-pyridinyl)-1,3-thiazol-2-yl]amino}phenol,
2-Methyl-4-{[4-(4-pyridinyl)-1,3-thiazol-2-yl]amino}phenol,
N-(3,4-Dimethoxyphenyl)-4-(4-pyridinyl)-1,3-thiazol-2-amine,
N-(3,5-Dimethoxyphenyl)-4-(4-pyridinyl)-1,3-thiazol-2-amine,
N-(1,3-Benzodioxol-5-yl)-4-(4-pyridinyl)-1,3-thiazol-2-amine,
N-(3,4-Dichlorophenyl)-4-(4-pyridinyl)-1,3-thiazol-2-amine,
4-(4-Pyridinyl)-N-(3,4,5-trimethoxyphenyl)-1,3-thiazol-2-amine,
N-(3-Methylphenyl)-4-(3-pyridinyl)-1,3-thiazol-2-amine,
4-{[4-(3-Pyridinyl)-1,3-thiazol-2-yl]amino}phenol,
N-(3-Methylphenyl)-4-(2-pyridinyl)-1,3-thiazol-2-amine,
4-{[4-(2-Pyridinyl)-1,3-thiazol-2-yl]amino}phenol,
5-Methyl-N-(3-methylphenyl)-4-(4-pyridinyl)-1,3-thiazol-2-amine,
N-(2,3-Dihydro-1H-inden-5-yl)-5-methyl-4-(4-pyridinyl)-1,3-thiazol-2-amine,
[2-(Phenylamino)-4-(4-pyridinyl)-1,3-thiazol-5-yl]methanol,
[2-[(3-Methylphenyl)amino]-4-(4-pyridinyl)-1,3-thiazol-5-yl]methanol,
[2-[(4-Methylphenyl)amino]-4-(4-pyridinyl)-1,3-thiazol-5-yl]methanol,
[2-(2,3-Dihydro-1H-inden-5-ylamino)-4-(4-pyridinyl)-1,3-thiazol-5-yl]methanol,
2-[(3-Methylphenyl)amino]-4-(4-pyridinyl)-1,3-thiazole-5-carbaldehyde,
Methyl (2E)-3-[2-[(3-Methylphenyl)amino]-4-(4-pyridinyl)-1,3-thiazol-5-yl]-2-propenoate,

Methyl 3-[2-[(3-Methylphenyl)amino]-4-(4-pyridinyl)-1,3-thiazol-5-yl]propanoate,
3-[2-[(3-Methylphenyl)amino]-4-(4-pyridinyl)-1,3-thiazol-5-yl]-1-propanol,
N-(3-Methylphenyl)-5-[3-(4-morpholinyl)propyl]-4-(4-pyridinyl)-1,3-thiazol-2-amine,
5-[(Dimethylamino)methyl]-N-(3-methylphenyl)-4-(4-pyridinyl)-1,3-thiazol-2-amine,
5-[(Diethylamino)methyl]-N-(3-methylphenyl)-4-(4-pyridinyl)-1,3-thiazol-2-amine,
N-(3-Methylphenyl)-5-(1-piperidinylmethyl)-4-(4-pyridinyl)-1,3-thiazol-2-amine,
N-(3-Methylphenyl)-5-(4-morpholinylmethyl)-4-(4-pyridinyl)-1,3-thiazol-2-amine,

Ethyl 2-[(3-methylphenyl)amino]-4-(4-pyridinyl)-1,3-thiazole-5-carboxylate,
Ethyl 2-(2,3-dihydro-1H-inden-5-ylamino)-4-(4-pyridinyl)-1,3-thiazole-5-carboxylate,
2-[(3-Methylphenyl)amino]-4-(4-pyridinyl)-1,3-thiazole-5-carboxamide,
N,N-Dimethyl-2-[(3-methylphenyl)amino]-4-(4-pyridinyl)-1,3-thiazole-5-carboxamide,
N-[2-(Dimethylamino)ethyl]-2-[(3-methylphenyl)amino]-4-(4-pyridinyl)-1,3-thiazole-5-carboxamide,
2-[(3-Methylphenyl)amino]-N-[3-(4-morpholinyl)propyl]-4-(4-pyridinyl)-1,3-thiazole-5-carboxamide,
N-Methyl-N-(3-methylphenyl)-4-(4-pyridinyl)-1,3-thiazol-2-amine,
N-Ethyl-N-(3-methylphenyl)-4-(4-pyridinyl)-1,3-thiazol-2-amine,
2-{3-Methyl[4-(4-pyridinyl)-1,3-thiazol-2-yl]anilino}ethanol,
N-[4-(4-pyridinyl)-1,3-thiazol-2-yl]benzamide,
3-Methyl-N-[4-(4-pyridinyl)-1,3-thiazol-2-yl]benzamide,
4-Methyl-N-[4-(4-pyridinyl)-1,3-thiazol-2-yl]benzamide,
4-Methoxy-N-[4-(4-pyridinyl)-1,3-thiazol-2-yl]benzamide,
N-(3-Methylbenzyl)-4-(4-pyridinyl)-1,3-thiazol-2-amine,
N-(3-Methylphenyl)-4-(4-pyridinyl)-1,3-oxazol-2-amine,
2-{[4-(4-Pyridinyl)-1,3-thiazol-2-yl]amino}phenol,
N-(2-Methoxyphenyl)-4-(4-pyridinyl)-1,3-thiazol-2-amine,
N-(2-Methylphenyl)-4-(4-pyridinyl)-1,3-thiazol-2-amine,
N-(2-Fluorophenyl)-4-(4-pyridinyl)-1,3-thiazol-2-amine,
N-[2-Chlorophenyl]-4-(4-pyridinyl)-1,3-thiazol-2-amine,
4-(4-Pyridinyl)-N-[2-(trifluoromethyl)phenyl]-1,3-thiazol-2-amine,
N-[2-Nitrophenyl]-4-(4-pyridinyl)-1,3-thiazol-2-amine,
N-(3-Fluorophenyl)-4-(4-pyridinyl)-1,3-thiazol-2-amine,
N-(3-Bromophenyl)-4-(4-pyridinyl)-1,3-thiazol-2-amine,
1-(3-{[4-(4-Pyridinyl)-1,3-thiazol-2-yl]amino}phenyl)ethanone,
3-{[4-(4-Pyridinyl)-1,3-thiazol-2-yl]amino}benzonitrile,
N-(3-Nitrophenyl)-4-(4-pyridinyl)-1,3-thiazol-2-amine,
1-(4-{[4-(4-Pyridinyl)-1,3-thiazol-2-yl]amino}phenyl)ethanone,
4-(4-Pyridinyl)-N-[4-(trifluoromethyl)phenyl]-1,3-thiazol-2-amine,
4-{[4-(4-Pyridinyl)-1,3-thiazol-2-yl]amino}benzonitrile,
N-(2,6-Dimethylphenyl)-4-(4-pyridinyl)-1,3-thiazol-2-amine,
N-(2,6-Difluorophenyl)-4-(4-pyridinyl)-1,3-thiazol-2-amine,
N-{4-[2-(Methyloxy)-4-pyridinyl]-1,3-thiazol-2-yl}-N-(3-methylphenyl)amine,
N-(3-Methylphenyl)-4-(2-methyl-4-pyridinyl)-1,3-thiazol-2-amine,
4-(2-Fluoro-4-pyridinyl)-N-(3-methylphenyl)-1,3-thiazol-2-amine,
4-(2-Chloro-4-pyridinyl)-N-(3-methylphenyl)-1,3-thiazol-2-amine,
4-(2-Bromo-4-pyridinyl)-N-(3-methylphenyl)-1,3-thiazol-2-amine,
N-[4-(2,6-Dichloro-4-pyridinyl)-1,3-thiazol-2-yl]-N-(3-methylphenyl)amine,
4-(2-Ethynyl-4-pyridinyl)-N-(3-methylphenyl)-1,3-thiazol-2-amine,
4-(2-Ethyl-4-pyridinyl)-N-(3-methylphenyl)-1,3-thiazol-2-amine,
3-(4-{2-[(3-Methylphenyl)amino]-1,3-thiazol-4-yl}-2-pyridinyl)-2-propyn-1-ol,
3-(4-{2-[(3-Methylphenyl)amino]-1,3-thiazol-4-yl}-2-pyridinyl)-1-propanol,
N-(3-Methylphenyl)-4-(3-methyl-4-pyridinyl)-1,3-thiazol-2-amine,
4-(3-Fluoro-4-pyridinyl)-N-(3-methylphenyl)-1,3-thiazol-2-amine,
4-(3-Chloro-4-pyridinyl)-N-(3-methylphenyl)-1,3-thiazol-2-amine,
4-(3-Bromo-4-pyridinyl)-N-(3-methylphenyl)-1,3-thiazol-2-amine,
N-(3-Methylphenyl)-4-(2-nitro-4-pyridinyl)-1,3-thiazol-2-amine,
N-[4-(2-Amino-4-pyridinyl)-1,3-thiazol-2-yl]-N-(3-methylphenyl)amine,
N-(4-{2-[(3-Methylphenyl)amino]-1,3-thiazol-4-yl}-2-pyridinyl)acetamide,
4-(3-Ethynyl-4-pyridinyl)-N-(3-methylphenyl)-1,3-thiazol-2-amine,
4-(3-Ethyl-4-pyridinyl)-N-(3-methylphenyl)-1,3-thiazol-2-amine,
3-(4-{2-[(3-Methylphenyl)amino]-1,3-thiazol-4-yl}-3-pyridinyl)-2-propyn-1-ol,
3-(4-{2-[(3-Methylphenyl)amino]-1,3-thiazol-4-yl}-3-pyridinyl)-1-propanol,
N-(3-Methylphenyl)-4-(1-oxido-4-pyridinyl)-1,3-thiazol-2-amine,
N-(3-Methylphenyl)-4-(4-quinolinyl)-1,3-thiazol-2-amine,
N-(3-Methylphenyl)-4-(2-phenyl-4-quinolinyl)-1,3-thiazol-2-amine,
N-(3-Methylphenyl)-4-(4-pyridinyl)-1H-imidazol-2-amine,
N-(3-Methylphenyl)-4-[2-(4-morpholinyl)-4-pyridinyl]-1,3-thiazol-2-amine,
N-(3-Methylphenyl)-4-[2-(4-methyl-1-piperazinyl)-4-pyridinyl]-1,3-thiazol-2-amine, and
N,N-Dimethyl-4-{2-[(3-methylphenyl)amino]-1,3-thiazol-4-yl}-2-pyridinamine.

As used herein, the compounds of the invention are defined to include pharmaceutically acceptable salts, derivatives, or prodrugs thereof. A “pharmaceutically acceptable salt, derivative, or prodrug” means any pharmaceutically acceptable salt, ester, salt of an ester, or other derivative of a compound of this invention, which, upon administration to a recipient, is capable of providing or provides (directly or indirectly) a compound of the invention.

Accordingly, this invention also provides prodrugs of the compounds of the invention, which are derivatives that are designed to enhance biological properties such as oral absorption, clearance, metabolism, or compartmental distribution. Such derivations are well known in the art.

As the skilled practitioner realizes, the compounds of the invention may be modified by appending appropriate functionalities to enhance selective biological properties. Such modifications are known in the art and include those which increase biological penetration into a given biological compartment (e.g., blood, lymphatic system, central nervous system), increase oral availability, increase solubility to allow administration by injection, alter metabolism, or alter rate of excretion.

Certain derivatives and prodrugs are those that increase the bioavailability of the compounds of the invention when such compounds are administered to an individual (e.g., by allowing an orally administered compound to be more readily absorbed into the blood), have more favorable clearance rates or metabolic profiles, or enhance delivery of the parent compound to a biological compartment (e.g., the brain or lymphatic system) relative to the parent species. Examples of prodrugs include derivatives in which a group that enhances aqueous solubility or active transport through the gut membrane is appended to the structure.

In some embodiments, the compounds of the invention are provided in the form of pharmaceutically acceptable salts. Compounds containing an amine may be basic in nature and accordingly may react with any number of inorganic and organic acids to form pharmaceutically acceptable acid addition salts. Acids commonly employed to form such salts include inorganic acids such as hydrochloric, hydrobromic, hydriodic, sulfuric and phosphoric acid, as well as organic acids such as para-toluenesulfonic, methanesulfonic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, β-hydroxybutyrate, glycollate, maleate, tartrate, methanesulfonate, propanesulfonates, naphthalene-1-sulfonate, naphthalene-2-sulfonate, mandelate, hippurate, gluconate, lactobionate, and the like salts. In certain specific embodiments, pharmaceutically acceptable acid addition salts include those formed with mineral acids such as hydrochloric acid and hydrobromic acid, and those formed with organic acids such as fumaric acid and maleic acid.

Compounds of the instant invention that are acidic in nature may accordingly react with any number of inorganic and organic bases to form pharmaceutically acceptable base salts. Specific bases include the mineral bases, such as NaOH and KOH, but one of skill in the art would appreciate that other bases may also be used. See Ando et al., Remington: The Science and Practice of Pharmacy, 20th ed. 700-720 (Alfonso R. Gennaro ed.), 2000.

The pharmaceutically acceptable addition salts of the compounds of the invention may also exist as various solvates, such as with water, methanol, ethanol, dimethylformamide, and the like. Mixtures of such solvates may also be prepared. The source of such solvate may be from the solvent of crystallization, inherent in the solvent of preparation or crystallization, or adventitious to such solvent.

### Methods for Preparing Compounds of the Invention

Compounds of the invention may usefully be prepared according to the following general schemes. The detailed use of these schemes in the synthesis of specific compounds is provided below in the Examples.

A convenient synthesis of the compounds may be achieved using the Hantzsch thiazole synthesis, combining pyridylbromoketones with phenylthioureas (Scheme 1). The pyridylbromoketones may be readily synthesized from the corresponding ketopyridine. Thioureas are commercially available or may be conveniently prepared from the corresponding aniline, either directly by the condensation of the appropriate aniline and NH₄SCN, or by reaction of benzoyl isothiocyanate and the appropriate aniline to give the benzoyl thiourea, followed by hydrolysis under basic conditions, to give the corresponding thiourea (Rasmussen, C. R. et al., Synthesis 1988, 456).

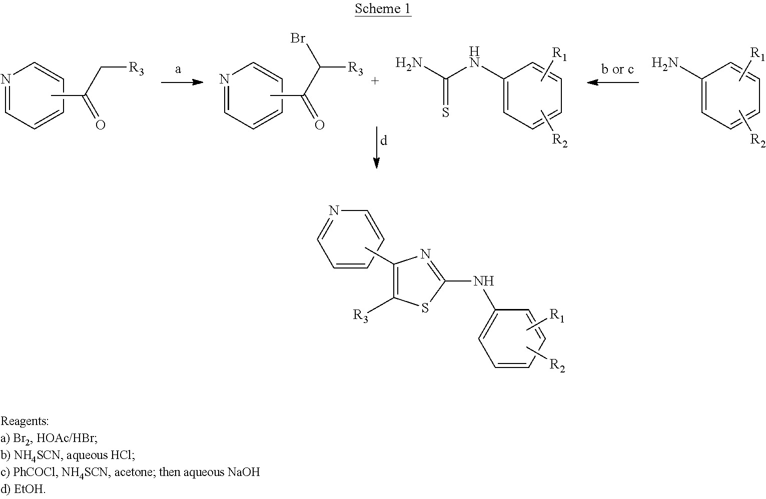

1-(4-Pyridyl)-2-bromoethanone 1 is commercially available or may be readily synthesized by treatment of 4-acetylpyridine with bromine (Scheme 2). Thiourea 2 is commercially available or may be conveniently prepared from aniline by treatment with NH₄SCN in acidic solution. Alternatively, reaction of benzoyl isothiocyanate and aniline, followed by hydrolysis under basic conditions, gave the phenylthiourea. Reaction of 1-(4-pyridyl)-2-bromoethanone 1 with phenylthiourea 2 in EtOH gives aminothiazole 3.

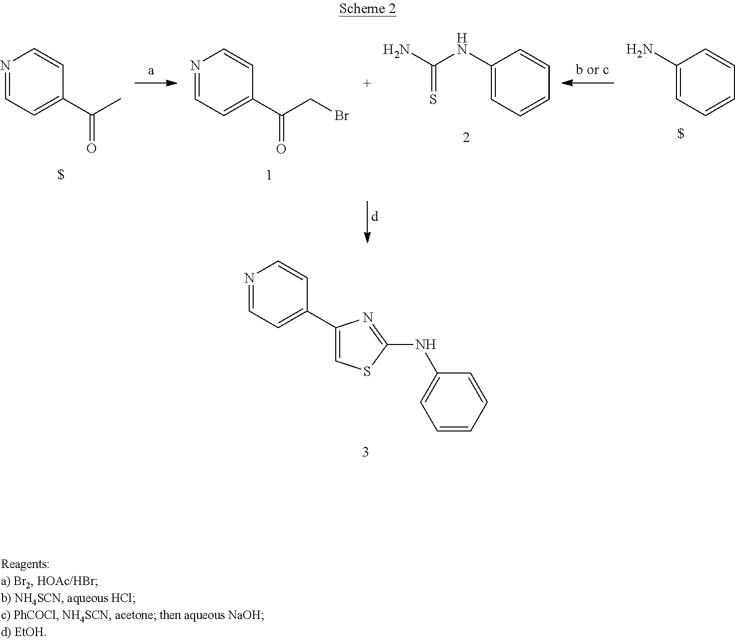

Similarly, reaction of bromide 1 with thioureas 4, 6, 8, 10, 12, 14, 16, 18, 20, 22, 24, 26, 28, 30, 32 and 34 gives aminothiazoles 5, 7, 9, 11, 13, 15, 17, 19, 21, 23, 25, 27, 29, 31, 33 and 35, respectively (Scheme 3). Alternatively, reaction of 4-(2-(dimethylamino)ethoxy)aniline with thiophosgene and iPr₂NEt in THF and subsequent reaction with NH₃ gives thiourea 28.

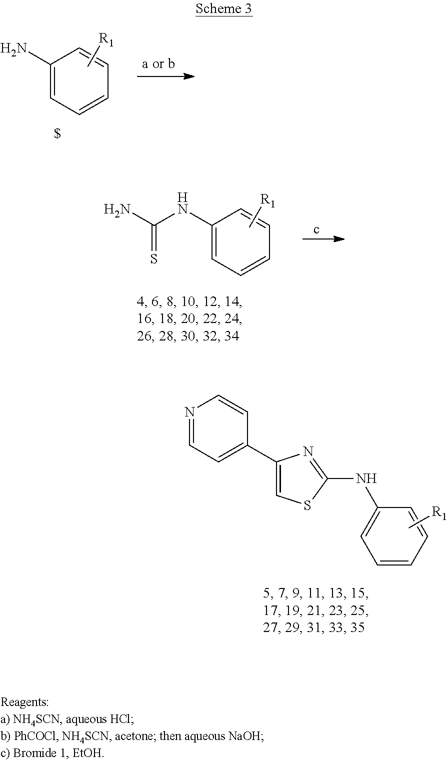

| R₁ | Thiourea | Aminothiazole |
|---|---|---|
| 3-Me | 4 | 5 |
| 3-Et | 6 | 7 |
| 3-iPr | 8 | 9 |
| 3-tBu | 10 | 11 |
| 3-OH | 12 | 13 |
| 3-OMe | 14 | 15 |
| 3-Cl | 16 | 17 |
| 3-CF₃ | 18 | 19 |
| 4-Me | 20 | 21 |
| 4-CH₂CH₂OH | 22 | 23 |
| 4-OH | 24 | 25 |
| 4-OMe | 26 | 27 |
| 4-OCH₂CH₂NMe₂ | 28 | 29 |
| 4-Cl | 30 | 31 |
| 4-F | 32 | 33 |
| 4-NO₂ | 34 | 35 |

Mesylation of aminothiazole 23 with methanesulfonyl chloride and subsequent displacement with dimethylamine gives amine 36 (Scheme 4).

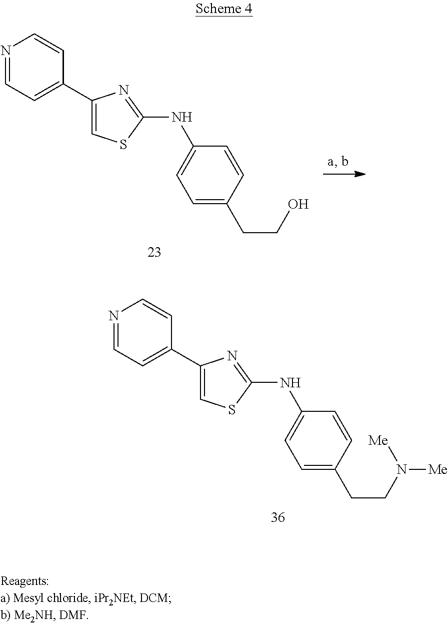

Catalytic hydrogenation of nitroaniline 35 with Pd/C gives diamine 37 (Scheme 5).

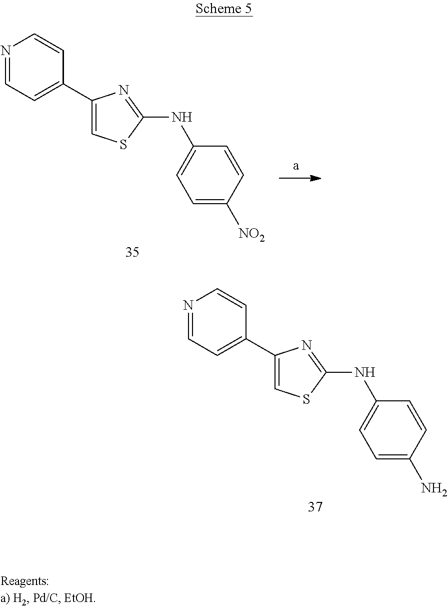

Reaction of phenols 13 and 25 with di-tertbutyl diisopropylphosphoramidite and tetrazole followed by oxidation with MCPBA gives the diesters 38 and 40, respectively. Hydrolysis of the diester gives the phosphates 39 and 41, respectively (Scheme 6).

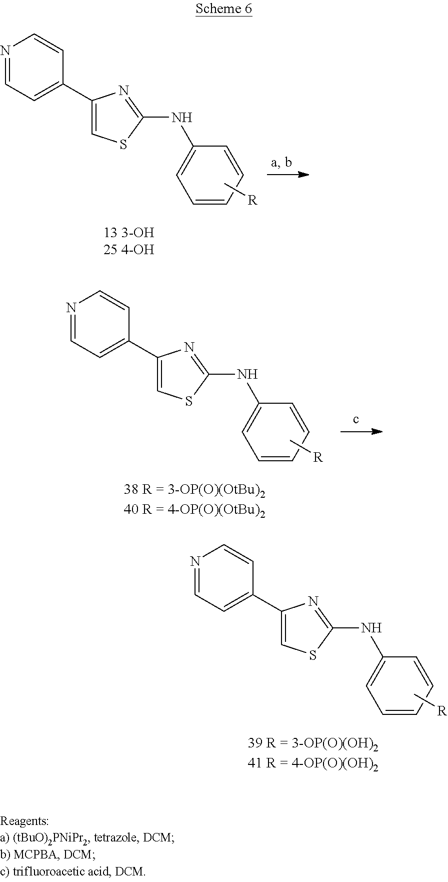

Reaction of bromide 1 with thioureas 42, 44, 46, 48, 50, 52, 54, 56, 58, 60 and 62 gives aminothiazoles 43, 45, 47, 49, 50, 51, 53, 55, 57, 59, 61 and 63, respectively (Scheme 7).

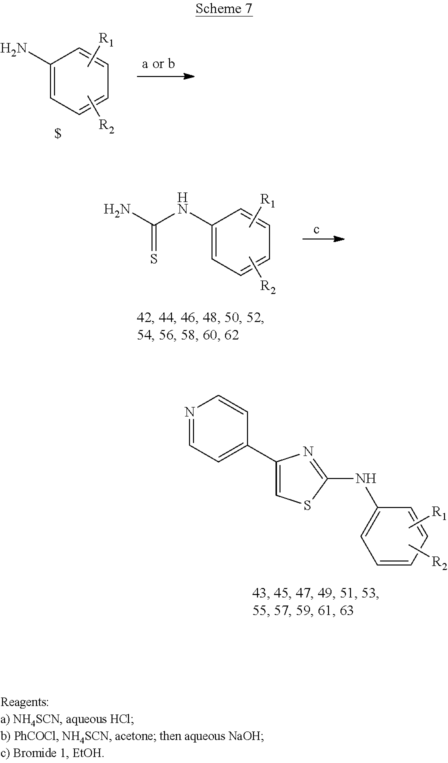

| R₁ | R₂ | Thiourea | Aminothiazole |
|---|---|---|---|
| 3-Me | 4-Me | 42 | 43 |
| 3-Me | 5-Me | 44 | 45 |
| 3-CH₂CH₂CH₂-4 | | 46 | 47 |
| 3-OH | 4-Me | 48 | 49 |
| 3-OH | 4-OMe | 50 | 51 |
| 3-Me | 4-OH | 52 | 53 |
| 3-OMe | 4-OMe | 54 | 55 |
| 3-OMe | 5-OMe | 56 | 57 |
| 3-OCH₂O-4 | | 58 | 59 |
| 3-Cl | 4-Cl | 60 | 61 |

| | | |
|---|---|---|
| 3,4,5-OMe₃ | 62 | 63 |

Treatment of 3-acetylpyridine with Br₂ gives 1-(3-pyridyl)-2-bromoethanone 64 which may be combined with phenylthioureas 4 and 24 to give aminothiazoles 65 and 66, respectively (Scheme 8).

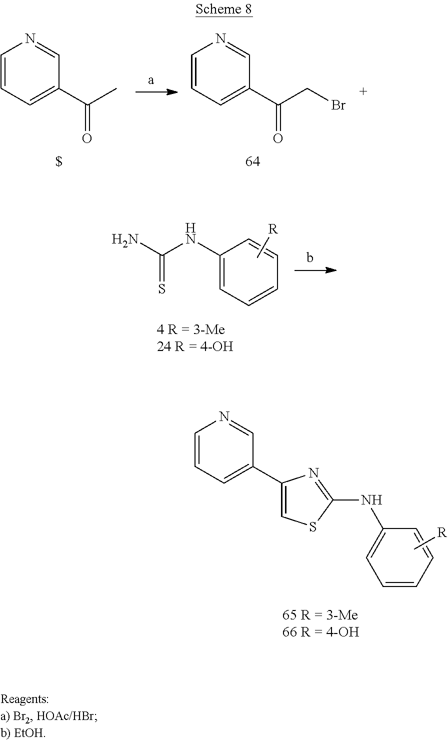

Similarly, treatment of 2-acetylpyridine with Br₂ gives 1-(2-pyridyl)-2-bromoethanone 67 which may be combined with phenylthioureas 4 and 24 to give aminothiazoles 68 and 69, respectively (Scheme 9).

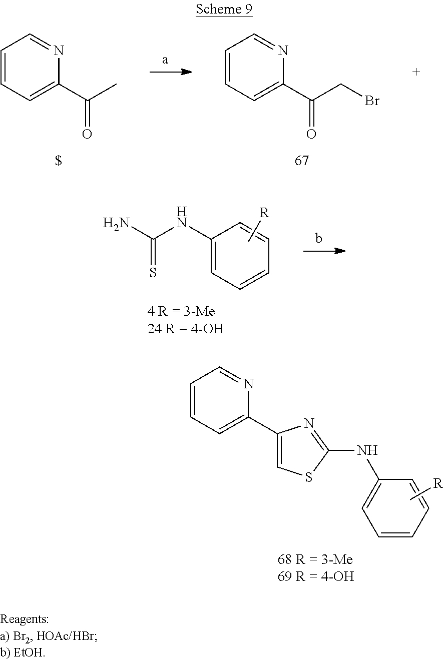

Treatment of 4-propionylpyridine with Br₂ gives 1-(4-pyridyl)-2-bromopropanone 70 which may be combined with phenylthioureas 4 and 46 to give aminothiazoles 71 and 72, respectively (Scheme 10).

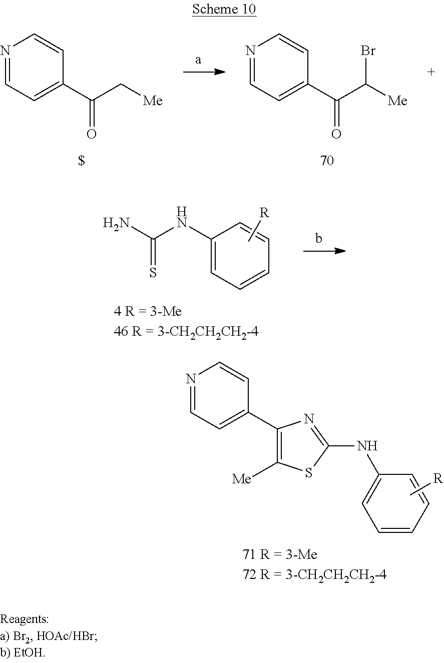

Reaction of aminothiazoles 3, 5, 21 and 47 with formaldehyde gives the alcohols 73-76 (Scheme 11).

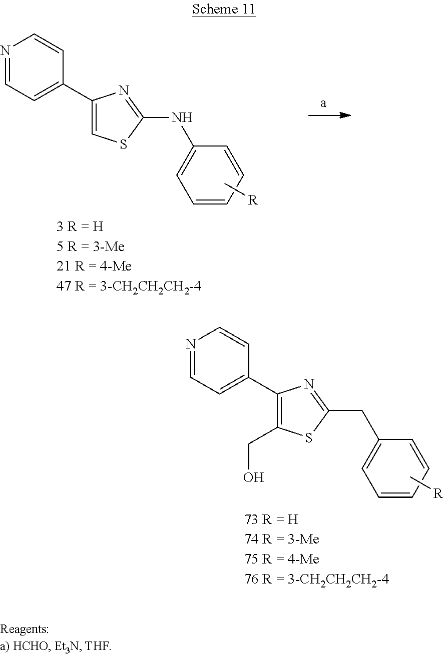

Oxidation of alcohol 74, with MnO₂ gives the aldehyde 77 which may undergo a Wittig reaction to give a mixture of E/Z isomers of the ester 78 (Scheme 12). Hydrogenation of the double bond over palladium catalyst gives the ester 79, which may be hydrolysed to acid 80. Reduction of acid 80 gives propanol 81 which may be activated with methane sulfonyl chloride and may undergo displacement with morpholine to give morpholide 82.

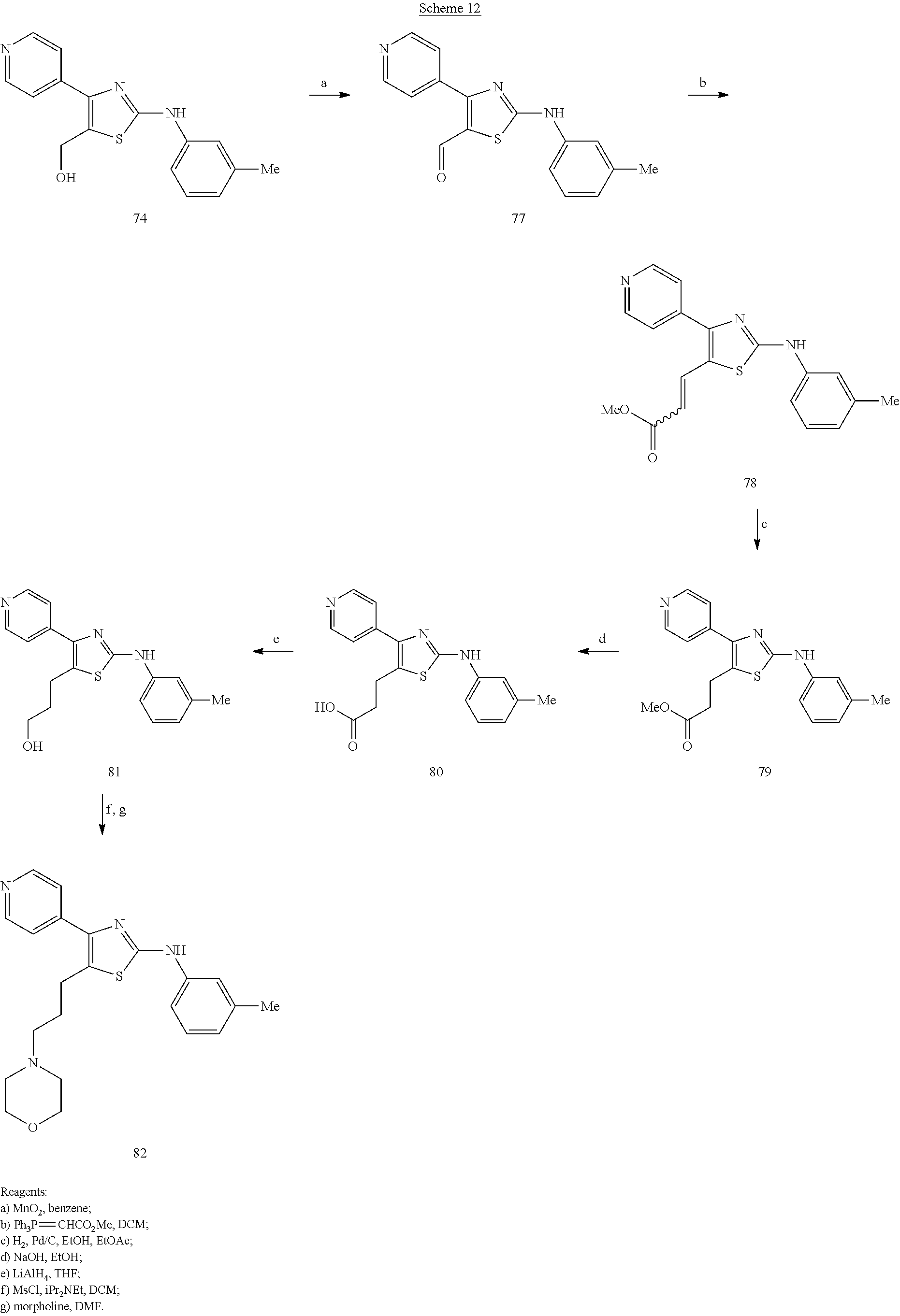

Reaction of alcohol 74 with methane sulfonyl chloride and subsequent reaction with a range of amines gave the amines 83-86 (Scheme 13).

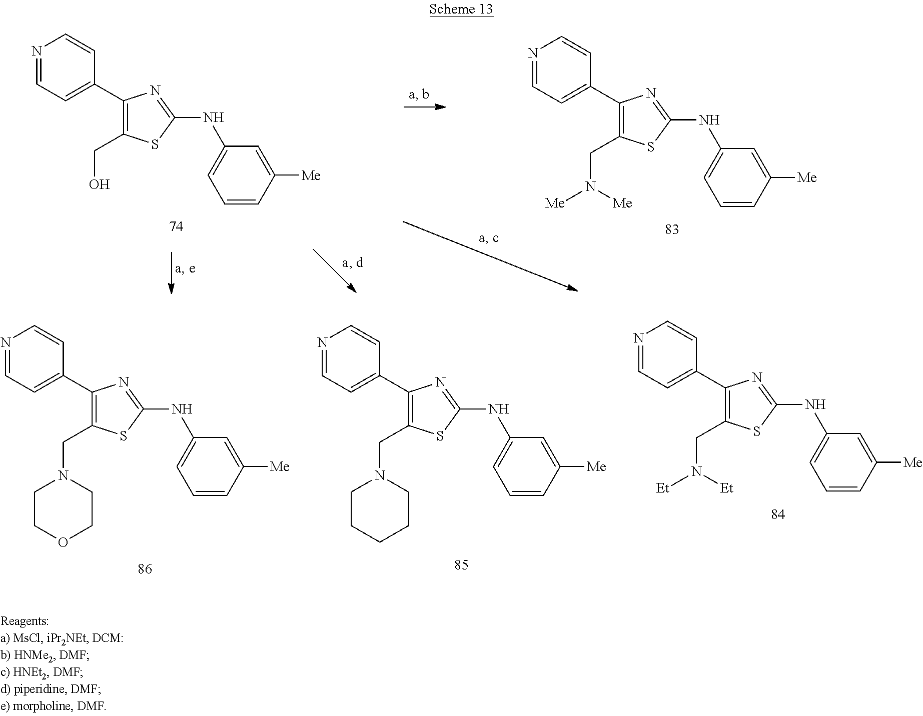

Treatment of ethyl isonicotinoylacetate with Br₂ in CHCl₃ gives bromoacetate 87, which may be combined with phenylthioureas 4 and 46, to give aminothiazoles 88 and 89, respectively (Scheme 14).

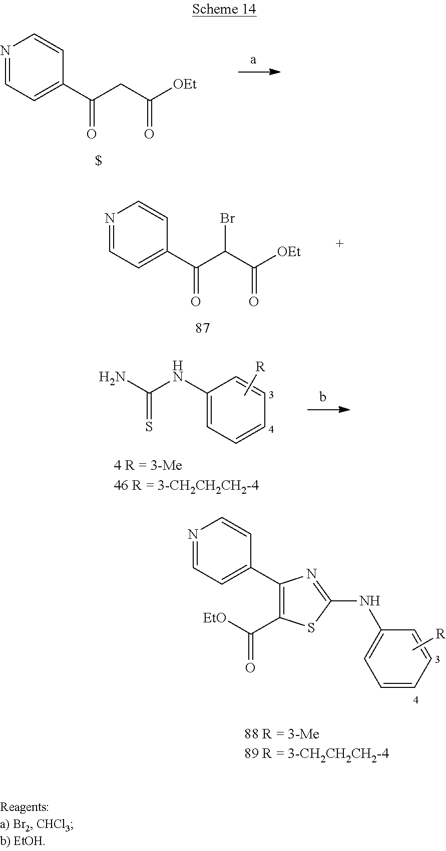

Hydrolysis of ester 88 gives acid 90, which may be converted into amides 91-94 (Scheme 15).

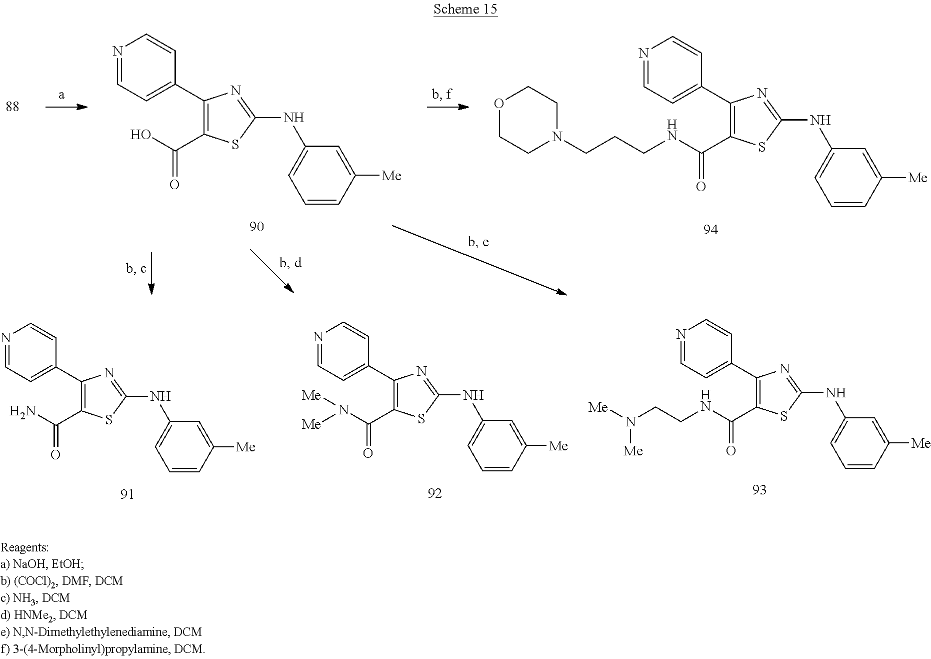

Reaction of aminothiazole 5 with sodium hydride and various alkyl iodides gives aminothiazoles 95-97 (Scheme 16).

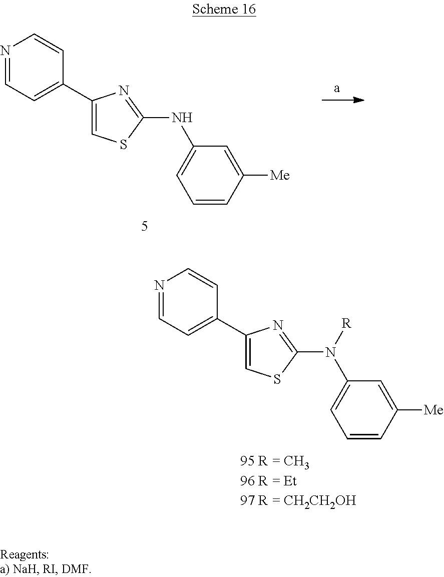

Treatment of bromoketone 1 with thiourea gives 2-aminothiazole 98 which may be condensed with various benzoyl chlorides to give amides 99-102 (Scheme 17). Reaction of amine 98 with 3-methylbenzaldehyde with subsequent reduction gives the benzylamine 103.

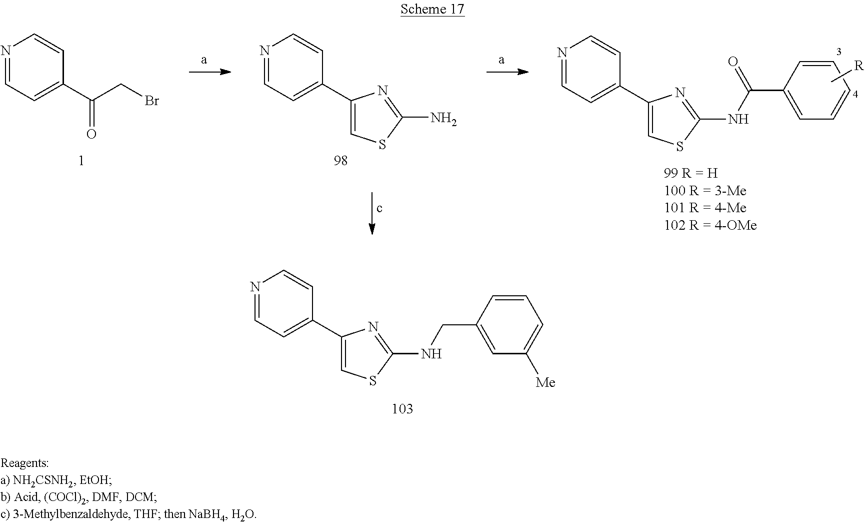

Condensation of bromide 1 with urea 104 gives oxazole 105 (Scheme 18).

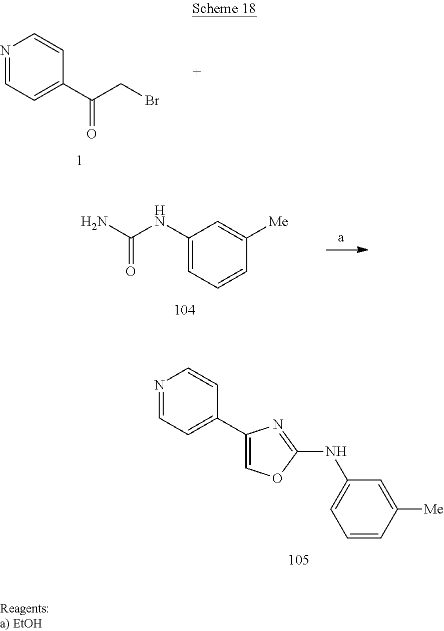

Similarly, reaction of bromide 1 with thioureas 106, 108, 110, 112, 114, 116, 118, 120, 122, 124, 126, 128, 130, 132, 134, 136 and 138 gives aminothiazoles 107, 109, 111, 113, 115, 117, 119, 121, 123, 125, 127, 129, 131, 133, 135, 137, and 139, respectively (Scheme 19).

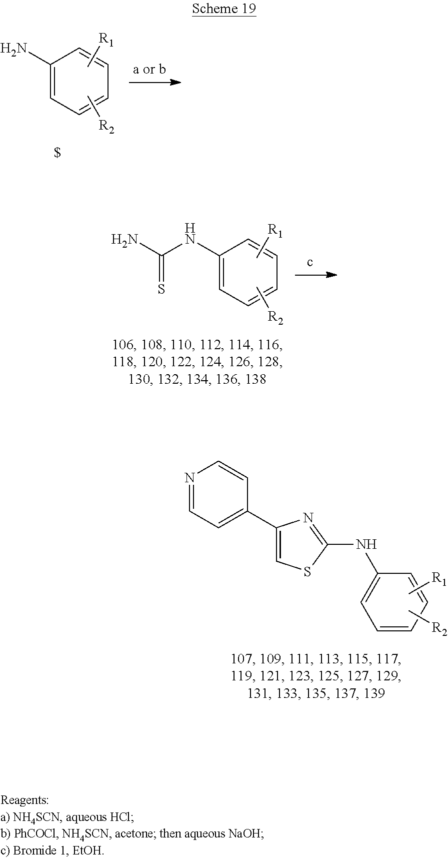

| R₁ | R₂ | Thiourea | Aminothiazole |
|---|---|---|---|
| 2-OH | H | 106 | 107 |
| 2-OMe | H | 108 | 109 |
| 2-Me | H | 110 | 111 |
| 2-F | H | 112 | 113 |
| 2-Cl | H | 114 | 115 |
| 2-CF₃ | H | 116 | 117 |
| 2-NO₂ | H | 118 | 119 |
| 3-F | H | 120 | 121 |
| 3-Br | H | 122 | 123 |
| 3-COCH₃ | H | 124 | 125 |
| 3-CN | H | 126 | 127 |
| 3-NO₂ | H | 128 | 129 |
| 4-COCH₃ | H | 130 | 131 |
| 4-CF₃ | H | 132 | 133 |
| 4-CN | H | 134 | 135 |
| 2-Me | 6-Me | 136 | 137 |
| 2-F | 6-F | 138 | 139 |

2-Methoxypyridine 4-carboxylic acid was converted to the acid chloride and treated with TMSCH₂N₂ and the intermediate diazoketone treated with HBr to give the bromoketone 140 (Scheme 20). Reaction of 140 with thiourea 4 gave amine 141. Reaction of 2-picoline N-oxide with Me₂SO₄ and KCN gave the nitrile 142, which was converted to the ketone 143 using MeMgBr. Bromination of 143 gave 144, which reacted with thiourea 4 to give amine 145. Formation of the Weinreb amide 146 and Grignard reaction gave ketone 147. Bromination of 147 afforded 148 which reacted with thiourea 4 to amine 149. Similarly, reaction of the 2-chloro bromoketone analogue gave amine 150. A similar sequence was used to furnish amines 154 and 158 from their corresponding pyridylcarboxylic acids.

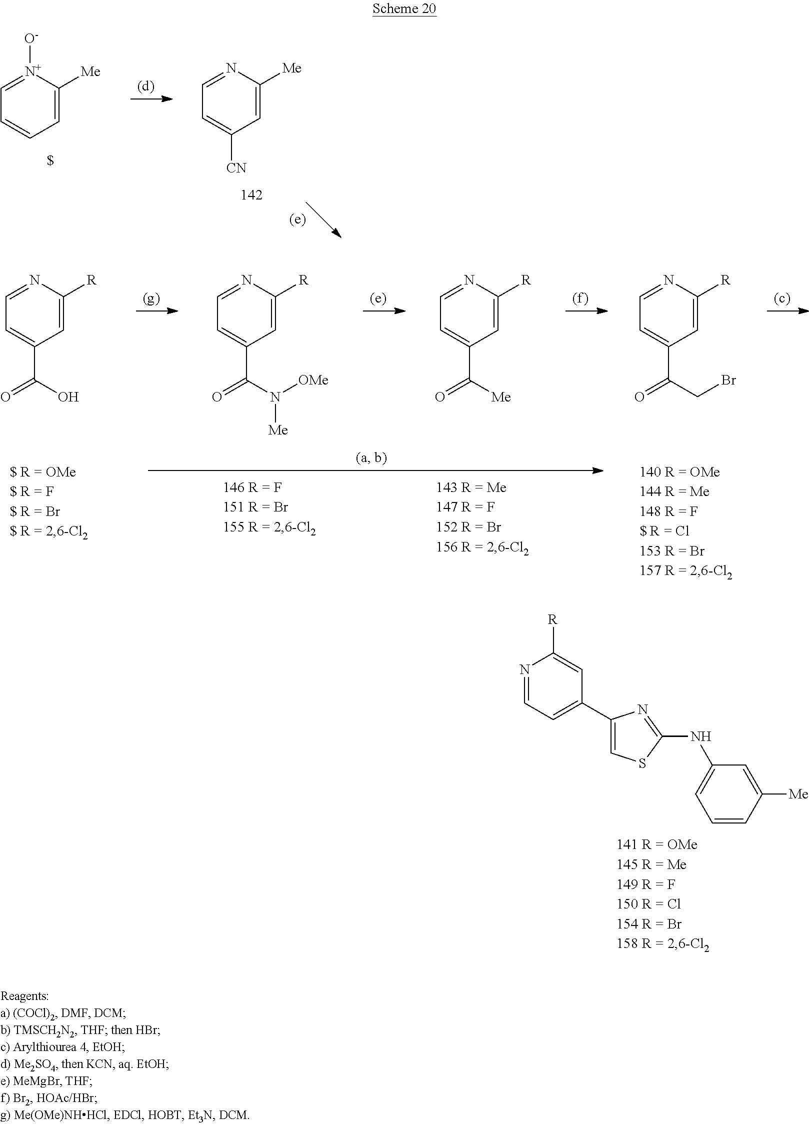

Sonogashira reaction of bromide 154 with TMS-acetylene and subsequent deprotection gave the alkyne 159, which was reduced to the alkane 160 (Scheme 21). Similarly, reaction of 154 with propargyl alcohol gave alkyne 161, which was reduced to alkane 162.

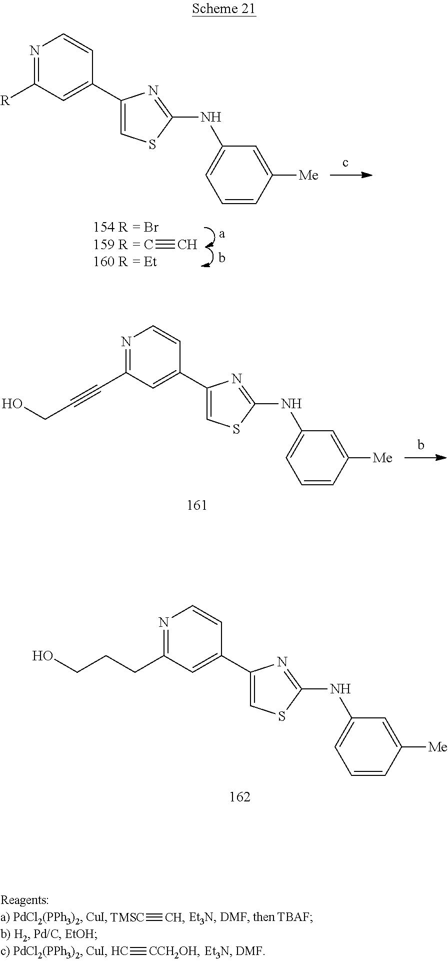

A series of 3-substituted pyridine carboxylic acids were elaborated to Weinreb amides 163, 167, 171 and 175, and then converted to the corresponding ketones 164, 168, 172, and 176 (Scheme 22). Bromination and condensation with thiourea 4 gave the amines 166, 170, 174, and 178. Nitration of 4-acetyl pyridine gave the 3-nitroketone 179, which was brominated to give 180 and then condensed with thiourea 4 to give amine 181. Reduction of 181 gave the 3-amino analogue 182, which was acetylated to give 183.

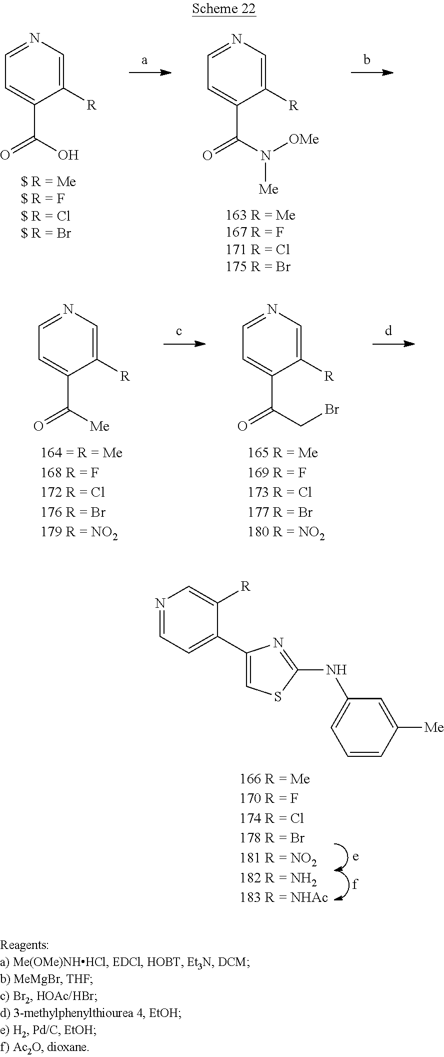

Sonogashira reaction of bromide 178 with TMS-acetylene and subsequent deprotection gave the alkyne 184, which was reduced to the alkane 185 (Scheme 23). Similarly, reaction of 178 with propargyl alcohol gave alkyne 186, which was reduced to alkane 187.

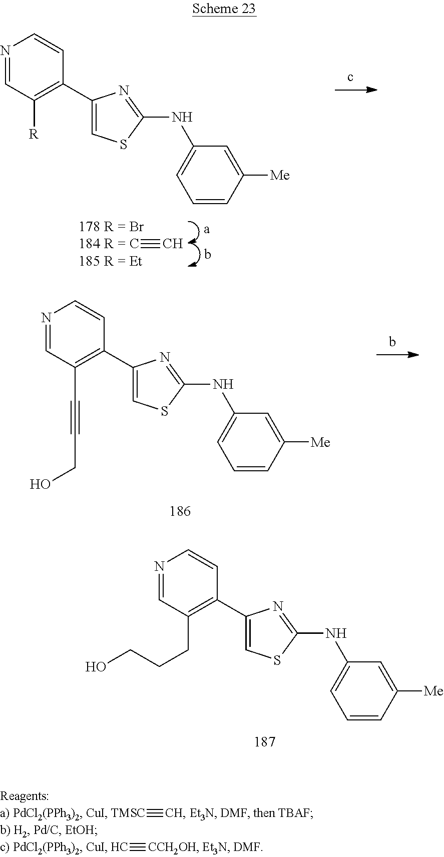

Oxidation of amine 5 with MCPBA gave the N-oxide 188 (Scheme 24).

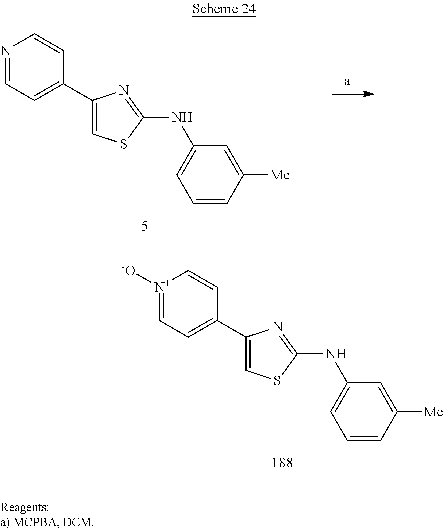

Reaction of 4-quinoline carboxylic acid gave the Weinreb amide 189 which underwent alkylation to give the ketone 190 (Scheme 25). Bromination of 190 gave the bromoketone 191 which was condensed with thiourea 4 to give amine 192. Alternatively, formation of the acid chloride and reaction with TMSCH₂N₂ and subsequent reaction with Br₂ gave the bromoketone 191. Similarly, reaction of 2-phenyl-4-quinoline carboxylic acid gave the bromoketone 193. Reaction with thiourea 4 gave amine 194.

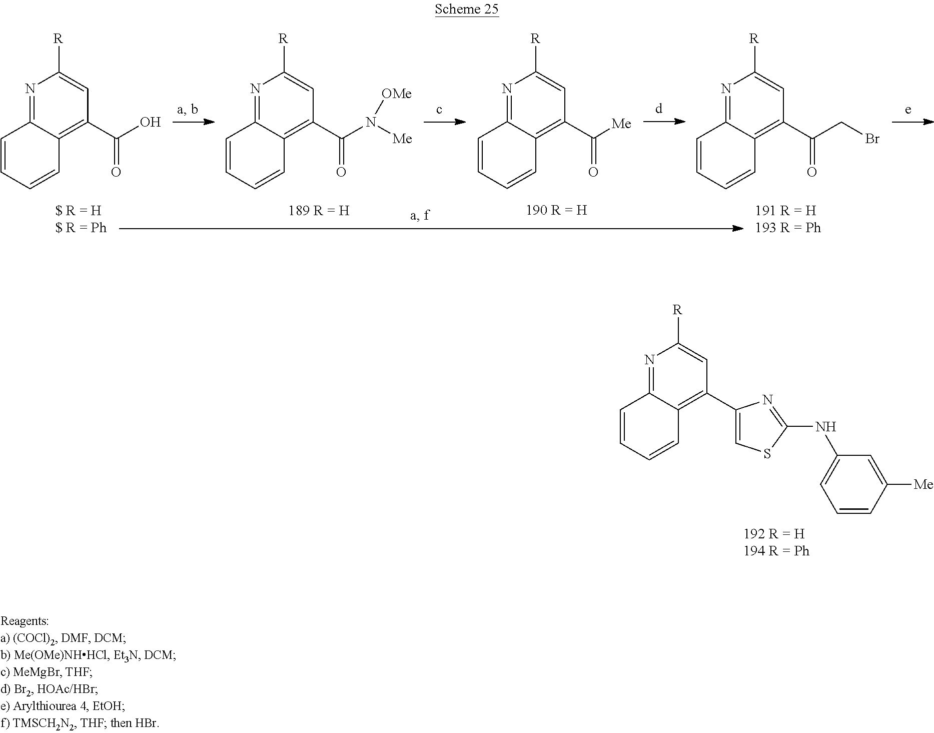

Condensation of 3-methylphenyl guanidine 204 with bromoketone 1 gave the imidazole 205 (Scheme 26).

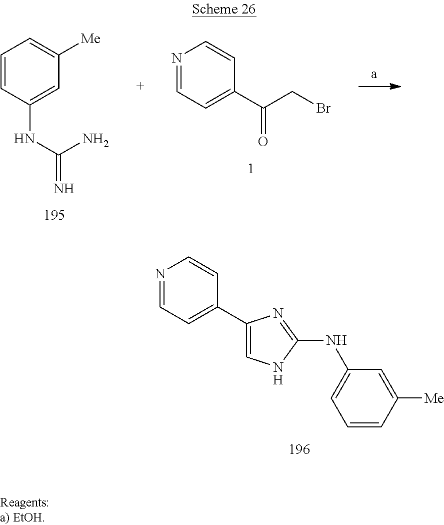

Reaction of bromide 154 with morpholine gave the amine 197 (Scheme 27). Similarly reaction of 154 with 1-methylpiperazine or aqueous dimethylamine gave the corresponding amines 198 and 199.

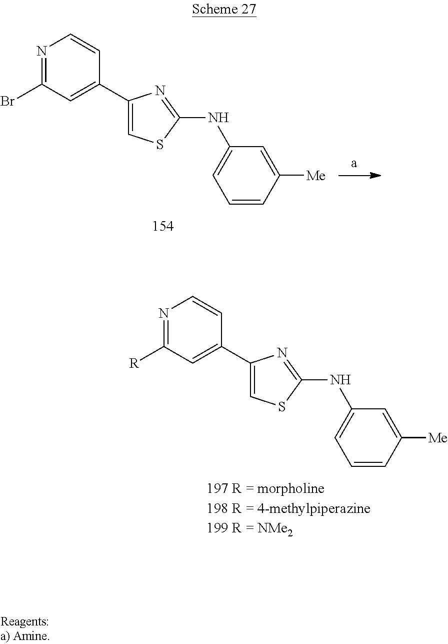

### Pharmaceutical Compositions

In another aspect, the compounds of the invention may be administered as a pharmaceutical composition containing, for example, any of the above-described compounds and a pharmaceutically acceptable carrier.

Pharmaceutically acceptable carriers are well known in the art and include, for example, aqueous solutions such as water or physiologically buffered saline or other solvents or vehicles such as glycols, glycerol, oils such as olive oil or injectable organic esters. In a specific embodiment, when such pharmaceutical compositions are for human administration, the aqueous solution is pyrogen free, or substantially pyrogen free. The excipients may be chosen, for example, to effect delayed release of an agent or to selectively target one or more cells, tissues or organs. The pharmaceutical composition may be in dosage unit form such as tablet, capsule, sprinkle capsule, granule, powder, syrup, suppository, injection or the like. The composition may also be present in a transdermal delivery system, e.g., a skin patch.

A pharmaceutically acceptable carrier may contain physiologically acceptable agents that act, for example, to stabilize or to increase the absorption of a compound of the instant invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The pharmaceutical composition also may comprise a liposome or other polymer matrix, which may have incorporated therein, for example, a compound of the invention. Liposomes, for example, which consist of phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition, or vehicle, such as a liquid or solid filler, diluent, excipient, solvent, or encapsulating material, involved in carrying or transporting the subject compounds from one organ, or portion of the body, to another organ, or portion of the body. Each carrier must be “acceptable” in the sense of being compatible with the other ingredients of the formulation and not injurious to the patient. Some examples of materials that can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. See Remington: The Science and Practice of Pharmacy, 20th ed. (Alfonso R. Gennaro ed.), 2000.

A pharmaceutical composition containing a compound of the instant invention may be administered to a host by any of a number of routes of administration including, for example, orally (for example, drenches as in aqueous or non-aqueous solutions or suspensions, tablets, boluses, powders, granules, pastes for application to the tongue); sublingually; anally, rectally, or vaginally (for example, as a pessary, cream, or foam); parenterally (including intramusclularly, intravenously, subcutaneously, or intrathecally as, for example, a sterile solution or suspension); nasally; intraperitoneally; subcutaneously; transdermally (for example as a patch applied to the skin); or topically (for example, as a cream, ointment or spray applied to the skin). The compound may also be formulated for inhalation. In certain embodiments, a compound of the instant invention may be simply dissolved or suspended in sterile water. Details of appropriate routes of administration and compositions suitable for same can be found in, for example, U.S. Pat. Nos. 6,110,973; 5,763,493; 5,731,000; 5,541,231; 5,427,798; 5,358,970; and 4,172,896, as well as in patents cited therein.

The formulations of the present invention may conveniently be presented in unit dosage form and may be prepared by any methods well known in the art of pharmacy. The amount of active ingredient that can be combined with a carrier material to produce a single dosage form will vary depending upon the host being treated and the particular mode of administration. The amount of active ingredient that can be combined with a carrier material to produce a single dosage form will generally be that amount of the compound that produces a therapeutic effect. Generally, out of one hundred percent, this amount will range from about 1 percent to about 99 percent of active ingredient, in some embodiments from about 5 percent to about 70 percent, and in more specific embodiments from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary, or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that may be used include polymeric substances and waxes. The active ingredient may also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions may also include adjuvants such as wetting agents, emulsifying and suspending agents, sweetening, flavoring, coloring, perfuming, and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Formulations of the pharmaceutical compositions of the invention for rectal, vaginal, or urethral administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Alternatively or additionally, compositions may be formulated for delivery via a catheter, stert, wire, or other intraluminal device. Delivery via such devices may be especially useful for delivery to the bladder, urethra, ureter, rectum, or intestine.

Formulations of the present invention which are suitable for vaginal administration also include pessaries, tampons, creams, gels, pastes, foams, or spray formulations containing such carriers as are known in the art to be appropriate.

Dosage forms for the topical or transdermal administration of a compound of this invention include powders, sprays, ointments, pastes, creams, lotions, gels, solutions, patches, and inhalants. The active compound may be mixed under sterile conditions with a pharmaceutically acceptable carrier, and with any preservatives, buffers, or propellants that may be required.

The ointments, pastes, creams, and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a compound of this invention, excipients such as lactose, talc, silicic acid, aluminum hydroxide, calcium silicates, and polyamide powder, or mixtures of these substances. Sprays may additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms may be made by dissolving or dispersing the compound in the proper medium. Absorption enhancers may also be used to increase the flux of the compound across the skin. The rate of such flux may be controlled by either providing a rate controlling membrane or dispersing the compound in a polymer matrix or gel.

Ophthalmic formulations, eye ointments, powders, solutions, and the like, are also contemplated as being within the scope of this invention.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, and intrasternal injection and infusion.

Pharmaceutical compositions of this invention suitable for parenteral administration comprise one or more compounds of the invention in combination with one or more pharmaceutically acceptable sterile isotonic aqueous or nonaqueous solutions, dispersions, suspensions, or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, chelators and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsuled matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

Methods of introduction may also be provided by rechargeable or biodegradable devices. Various slow release polymeric devices have been developed and tested in vivo in recent years for the controlled delivery of drugs. A variety of biocompatible polymers (including hydrogels), including both biodegradable and non-degradable polymers, may be used to form an implant for the sustained release of a compound at a particular target site.

In certain embodiments, the present invention provides compositions comprising at least one compound, as described above, or an analog, derivative, or functional equivalent thereof. As further detailed below, the inventive compositions may also comprise, or may be used in combination with, one or more known cytotoxic, vascular targeting agents or chemotherapeutic agents including, but not limited to, Xeloda™ (capecitabine), Paclitaxel™, FUDR (fluorouridine) Fludara™ (fludarabine phosphate), Gemzar™ (gemcitabine), methotrexate, cisplatin, carboplatin, adriamycin, avastin, tarceva, taxol, tamoxifen, Femora, temezolamide, cyclophosphamide, Erbitux, and Sutert.

### Methods of Use

As noted above, von Hippel-Lindau (VHL) disease is a hereditary condition that predisposes subjected individuals to the development of various malignant and benign tumors. Among the tumors resulting from this condition are renal cell carcinoma (RCC), in particular clear cell-RCC, pheochromocytomas, hemangioblastomas of the central nervous system and retina, endolymphatic sac tumors, renal cysts, pancreatic cysts, neuroendocrine tumors, endolymphatic sac tumors, and epididymal and broad ligament cystadenomas. Park, D. M., et al., PLoS Med. 2007, 4, E60. Accordingly, individuals affected by VHL disease may develop tumors of the retina, brain, spinal cord, ear, epidymis, broad ligament, adrenal gland, kidney, and pancreas. Accordingly, the invention further provides methods of using the compounds and compositions described herein in the treatment and prevention of such conditions.

In one aspect, the pharmaceutical compositions of the invention are used in methods for treating or preventing a disease. Accordingly, the methods comprise administering to the mammalian host in need thereof a therapeutically-effective amount of a compound of Formula II, or a pharmaceutically acceptable salt, derivative, or prodrug thereof:

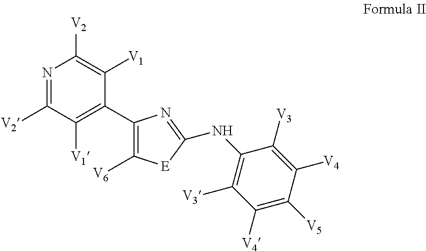

wherein:

E is S, O, or N—V₇;

V₁, V₁′, V₂, V₂′, V₃, V₃′, V₄, V₄′, V₅, V₆, and V₇ are independently hydrogen, halo, R, OH, OR, OC(O)H, OC(O)R, OC(O)NH₂, OC(O)NHR, OC(O)NRR, OP(O)(OH)₂, OP(O)(OR)₂, NO₂, NH₂, NHR, NRR, N⁺(—O⁻)RR, NHC(O)H, NHC(O)R, NRC(O)R, NHC(O)NH₂, NHC(O)NRR, NRC(O)NHR, N²C(O)NHR, SH, SR, S(O)H, S(O)R, SO₂R, SO₂NH₂, SO₂NHR, SO₂NRR, CF₃, CN, CO₂H, CO₂R, CHO, C(O)R, C(O)NH₂, C(O)NHR, C(O)NRR, CONHSO₂H, C(O)NHSO₂R, C(O)NRSO₂R, cyclic C₃-C₇ alkylamino, imidazolyl, piperazinyl, morpholinyl, piperidinyl, azepanyl, pyrrolidinyl or azetidinyl;

wherein each imidazolyl, piperazinyl, morpholinyl, piperidinyl, azepanyl, pyrrolidinyl and azetidinyl is optionally and independently substituted by one or more R¹, halo, OH, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N⁺(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, CONHSO₂H, C(O)NHSO₂R¹ or C(O)NR¹SO₂R¹;

and wherein groups V₁ and V₂, groups V₃ and V₄, and groups V₄ and V₅, taken together with the atoms to which they are attached, optionally and independently form a cyclic structure having from 4 to 8 atoms in the ring;

each R is independently C₁₋₆ alkyl, C₂₋₆ alkenyl, C₂₋₆ alkynyl, C₃₋₇ cyclic alkyl, aryl, aralkyl, heteroaryl, or heteroaralkyl, and is optionally and independently substituted with halo, OH, R¹, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N⁺(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, C(O)NHSO₂R¹, C(O)NR¹SO₂R¹, cyclic C₃-C₇ alkylamino, imidazolyl, piperazinyl, morpholinyl, piperidinyl, azepanyl, pyrrolidinyl, or azetidinyl;

wherein each imidazolyl, piperazinyl, morpholinyl, piperidinyl, azepanyl, pyrrolidinyl and azetidinyl is optionally and independently substituted with one or more R¹, halo, OH, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N⁺(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, CONHSO₂H, C(O)NHSO₂R¹ or C(O)NR¹SO₂R¹;

and wherein each heteroaryl group contains one or more heteroatoms in its ring system, independently selected from O, N or S;

each R¹ is independently C₁₋₆ alkyl or C₂₋₆ alkenyl, optionally and independently substituted with halo, OH, OR², OC(O)R², OC(O)NH₂, OC(O)NHR², OC(O)NR²R², OP(O)(OH)₂, OP(O)(OR²)₂, NO₂, NH₂, NHR², NR²R², N⁺(—O⁻)R²R², NHC(O)R², NR²C(O)R², NHC(O)NH₂, NHC(O)NR²R², NR²C(O)NHR², SH, SR², S(O)H, S(O)R², SO₂R², SO₂NH₂, SO₂NHR², SO₂NR²R², CF₃, CN, CO₂H, CO₂R², CHO, C(O)R², C(O)NH₂, C(O)NHR², C(O)NR²R², C(O)NHSO₂R², C(O)NR²SO₂R², cyclic C₃-C₇ alkylamino, imidazolyl, piperazinyl, morpholinyl, piperidinyl, azepanyl, pyrrolidinyl or azetidinyl;

wherein each imidazolyl, piperazinyl, morpholinyl, piperidinyl, azepanyl, pyrrolidinyl and azetidinyl is optionally and independently substituted with one or more R², halo, OH, OR², OC(O)R², OC(O)NH₂, OC(O)NHR², OC(O)NR²R², OP(O)(OH)₂, OP(O)(OR²)₂, NO₂, NH₂, NHR², NR²R², N⁺(—O⁻)R²R², NHC(O)H, NHC(O)R², NR²C(O)R², NHC(O)NH₂, NHC(O)NR²R², NR²C(O)NHR², SH, SR², S(O)H, S(O)R², SO₂R², SO₂NH₂, SO₂NHR², SO₂NR²R², CF₃, CN, CO₂H, CO₂R², CHO, C(O)R², C(O)NH₂, C(O)NHR², C(O)NR²R², CONHSO₂H, C(O)NHSO₂R² or C(O)NR²SO₂R²; and

each R² is independently C₁₋₆ alkyl, C₂₋₆ alkenyl, OH, OMe, NO₂, NH₂, CF₃, CN, CO₂H or SH.

In some embodiments, R is independently C₁₋₆ alkyl, C₂₋₄ alkenyl, C₃₋₇ cyclic alkyl, C₄₋₈ aryl, or C₄₋₈ heteroaryl, and is optionally and independently substituted with halo, OH, R¹, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N⁺(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, C(O)NHSO₂R¹, C(O)NR¹SO₂R¹, cyclic C₃-C₇ alkylamino, imidazolyl, piperazinyl, morpholinyl, piperidinyl, azepanyl, pyrrolidinyl, or azetidinyl.

In some embodiments, V₃, V₄, and V₅ are independently hydrogen; halo; R; OH; OR; CF₃; NO₂; NH₂; NHR; NRR; OP(O)(OH)₂; or OP(O)(OR)₂; and groups V₃ and V₄ and groups V₄ and V₅, taken together with the atoms to which they are attached, optionally and independently form a 5- or 6-membered ring structure.

In specific embodiments, V₃, V₄, and V₅ are independently hydrogen; halo; C₁₋₆ alkyl, optionally substituted with OH, NH₂, or N(CH₃)₂; OH; O—C₁₋₆ alkyl, optionally substituted with OH, NH₂, or N(CH₃)₂; CF₃; NO₂; NH₂; or OP(O)(OH)₂.

In some embodiments, V₄ is hydrogen; halo; R; OH; OR; CF₃; NO₂; NH₂; NHR; NRR; OP(O)(OH)₂; or OP(O)(OR)₂.

In more specific embodiments, V₄ is hydrogen; halo; C₁₋₆ alkyl, optionally substituted with OH, NH₂, or N(CH₃)₂; OH; O—C₁₋₆ alkyl, optionally substituted with OH, NH₂, or N(CH₃)₂; CF₃; NO₂; NH₂; or OP(O)(OH)₂.

In some embodiments, V₆ is hydrogen; R; CHO; CO₂R; C(O)NH₂; C(O)NHR; or C(O)NRR.

In more specific embodiments, V₆ is hydrogen; C₁₋₆ alkyl or C₂₋₄ alkenyl, optionally substituted with OH, OR¹, CO₂R¹, NH₂, NHR¹, or NR¹R¹; CHO; or CO₂R.

In even more specific embodiments, V₆ is hydrogen.

In some embodiments, V₁ and V₂ are independently hydrogen, halo, R, OH, OR, CF₃, NO₂, NH₂, NHR, NRR, or, taken together with the atoms to which they are attached, form a 5- or 6-membered ring structure.

In certain embodiments, the compound is a compound of Formula II, wherein V₁, V₁′, V₂, V₂′ are all hydrogen.

In some embodiments, the compound is a compound of Formula II, wherein E is S or O.

In more specific embodiments, E is S.

In even more specific embodiments, the compound is:
N-Phenyl-4-(4-pyridinyl)-1,3-thiazol-2-amine,
N-(3-Methylphenyl)-4-(4-pyridinyl)-1,3-thiazol-2-amine,
N-(3-Ethylphenyl)-4-(4-pyridinyl)-1,3-thiazol-2-amine,
N-(3-Isopropylphenyl)-4-(4-pyridinyl)-1,3-thiazol-2-amine,
N-(3-tert-Butylphenyl)-4-(4-pyridinyl)-1,3-thiazol-2-amine,
3-{[4-(4-Pyridinyl)-1,3-thiazol-2-yl]amino}phenol,
N-(3-Methoxyphenyl)-4-(4-pyridinyl)-1,3-thiazol-2-amine,
N-(3-Chlorophenyl)-4-(4-pyridinyl)-1,3-thiazol-2-amine,
4-(4-Pyridinyl)-N-[3-(trifluoromethyl)phenyl]-1,3-thiazol-2-amine,
N-(4-Methylphenyl)-4-(4-pyridinyl)-1,3-thiazol-2-amine,
2-(4-{[4-(4-Pyridinyl)-1,3-thiazol-2-yl]amino}phenyl)ethanol,
4-{[4-(4-Pyridinyl)-1,3-thiazol-2-yl]amino}phenol,
N-(4-Methoxyphenyl)-4-(4-pyridinyl)-1,3-thiazol-2-amine,
N-{4-[2-(Dimethylamino)ethoxy]phenyl}-4-(4-pyridinyl)-1,3-thiazol-2-amine,
N-(4-Chlorophenyl)-4-(4-pyridinyl)-1,3-thiazol-2-amine,
N-(4-Fluorophenyl)-4-(4-pyridinyl)-1,3-thiazol-2-amine,
N-(4-Nitrophenyl)-4-(4-pyridinyl)-1,3-thiazol-2-amine,
N-{4-[2-(Dimethylamino)ethyl]phenyl}-4-(4-pyridinyl)-1,3-thiazol-2-amine,
N¹-[4-(4-Pyridinyl)-1,3-thiazol-2-yl]-1,4-benzenediamine,
3-{[4-(4-Pyridinyl)-1,3-thiazol-2-yl]amino}phenyl Dihydrogen Phosphate, 4-{[4-(4-Pyridinyl)-1,3-thiazol-2-yl]amino}phenyl Dihydrogen Phosphate,
N-(3,4-Dimethylphenyl)-4-(4-pyridinyl)-1,3-thiazol-2-amine,
N-(3,5-Dimethylphenyl)-4-(4-pyridinyl)-1,3-thiazol-2-amine,
N-(2,3-Dihydro-1H-inden-5-yl)-4-(4-pyridinyl)-1,3-thiazol-2-amine,
2-Methyl-5-{[4-(4-pyridinyl)-1,3-thiazol-2-yl]amino}phenol,
2-Methoxy-5-{[4-(4-pyridinyl)-1,3-thiazol-2-yl]amino}phenol,
2-Methyl-4-{[4-(4-pyridinyl)-1,3-thiazol-2-yl]amino}phenol,
N-(3,4-Dimethoxyphenyl)-4-(4-pyridinyl)-1,3-thiazol-2-amine,
N-(3,5-Dimethoxyphenyl)-4-(4-pyridinyl)-1,3-thiazol-2-amine,
N-(1,3-Benzodioxol-5-yl)-4-(4-pyridinyl)-1,3-thiazol-2-amine,
N-(3,4-Dichlorophenyl)-4-(4-pyridinyl)-1,3-thiazol-2-amine,
4-(4-Pyridinyl)-N-(3,4,5-trimethoxyphenyl)-1,3-thiazol-2-amine,
N-(3-Methylphenyl)-4-(3-pyridinyl)-1,3-thiazol-2-amine,
4-{[4-(3-Pyridinyl)-1,3-thiazol-2-yl]amino}phenol,
N-(3-Methylphenyl)-4-(2-pyridinyl)-1,3-thiazol-2-amine,
4-{[4-(2-Pyridinyl)-1,3-thiazol-2-yl]amino}phenol,
5-Methyl-N-(3-methylphenyl)-4-(4-pyridinyl)-1,3-thiazol-2-amine,
N-(2,3-Dihydro-1H-inden-5-yl)-5-methyl-4-(4-pyridinyl)-1,3-thiazol-2-amine,
[2-(Phenylamino)-4-(4-pyridinyl)-1,3-thiazol-5-yl]methanol,
[2-[(3-Methylphenyl)amino]-4-(4-pyridinyl)-1,3-thiazol-5-yl]methanol,
[2-[(4-Methylphenyl)amino]-4-(4-pyridinyl)-1,3-thiazol-5-yl]methanol,
[2-(2,3-Dihydro-1H-inden-5-ylamino)-4-(4-pyridinyl)-1,3-thiazol-5-yl]methanol,
2-[(3-Methylphenyl)amino]-4-(4-pyridinyl)-1,3-thiazole-5-carbaldehyde,
Methyl (2E)-3-[2-[(3-Methylphenyl)amino]-4-(4-pyridinyl)-1,3-thiazol-5-yl]-2-propenoate,
Methyl 3-[2-[(3-Methylphenyl)amino]-4-(4-pyridinyl)-1,3-thiazol-5-yl]propanoate,
3-[2-[(3-Methylphenyl)amino]-4-(4-pyridinyl)-1,3-thiazol-5-yl]-1-propanol,
N-(3-Methylphenyl)-5-[3-(4-morpholinyl)propyl]-4-(4-pyridinyl)-1,3-thiazol-2-amine,
5-[(Dimethylamino)methyl]-N-(3-methylphenyl)-4-(4-pyridinyl)-1,3-thiazol-2-amine,
5-[(Diethylamino)methyl]-N-(3-methylphenyl)-4-(4-pyridinyl)-1,3-thiazol-2-amine,
N-(3-Methylphenyl)-5-(1-piperidinylmethyl)-4-(4-pyridinyl)-1,3-thiazol-2-amine,
N-(3-Methylphenyl)-5-(4-morpholinylmethyl)-4-(4-pyridinyl)-1,3-thiazol-2-amine,

Ethyl 2-[(3-methylphenyl)amino]-4-(4-pyridinyl)-1,3-thiazole-5-carboxylate,
Ethyl 2-(2,3-dihydro-1H-inden-5-ylamino)-4-(4-pyridinyl)-1,3-thiazole-5-carboxylate,
2-[(3-Methylphenyl)amino]-4-(4-pyridinyl)-1,3-thiazole-5-carboxamide,
N,N-Dimethyl-2-[(3-methylphenyl)amino]-4-(4-pyridinyl)-1,3-thiazole-5-carboxamide,
N-[2-(Dimethylamino)ethyl]-2-[(3-methylphenyl)amino]-4-(4-pyridinyl)-1,3-thiazole-5-carboxamide,
2-[(3-Methylphenyl)amino]-N-[3-(4-morpholinyl)propyl]-4-(4-pyridinyl)-1,3-thiazole-5-carboxamide,
N-Methyl-N-(3-methylphenyl)-4-(4-pyridinyl)-1,3-thiazol-2-amine,
N-Ethyl-N-(3-methylphenyl)-4-(4-pyridinyl)-1,3-thiazol-2-amine,
2-{3-Methyl[4-(4-pyridinyl)-1,3-thiazol-2-yl]anilino}ethanol,
N-[4-(4-Pyridinyl)-1,3-thiazol-2-yl]benzamide,
3-Methyl-N-[4-(4-pyridinyl)-1,3-thiazol-2-yl]benzamide,
4-Methyl-N-[4-(4-pyridinyl)-1,3-thiazol-2-yl]benzamide,
4-Methoxy-N-[4-(4-pyridinyl)-1,3-thiazol-2-yl]benzamide,
N-(3-Methylbenzyl)-4-(4-pyridinyl)-1,3-thiazol-2-amine,
N-(3-Methylphenyl)-4-(4-pyridinyl)-1,3-oxazol-2-amine,
2-{[4-(4-Pyridinyl)-1,3-thiazol-2-yl]amino}phenol,
N-(2-Methoxyphenyl)-4-(4-pyridinyl)-1,3-thiazol-2-amine,
N-(2-Methylphenyl)-4-(4-pyridinyl)-1,3-thiazol-2-amine,
N-(2-Fluorophenyl)-4-(4-pyridinyl)-1,3-thiazol-2-amine,
N-[2-Chlorophenyl]-4-(4-pyridinyl)-1,3-thiazol-2-amine,
4-(4-Pyridinyl)-N-[2-(trifluoromethyl)phenyl]-1,3-thiazol-2-amine,
N-[2-Nitrophenyl]-4-(4-pyridinyl)-1,3-thiazol-2-amine,
N-(3-Fluorophenyl)-4-(4-pyridinyl)-1,3-thiazol-2-amine,
N-(3-Bromophenyl)-4-(4-pyridinyl)-1,3-thiazol-2-amine,
1-(3-{[4-(4-Pyridinyl)-1,3-thiazol-2-yl]amino}phenyl)ethanone,
3-{[4-(4-Pyridinyl)-1,3-thiazol-2-yl]amino}benzonitrile,
N-(3-Nitrophenyl)-4-(4-pyridinyl)-1,3-thiazol-2-amine,
1-(4-{[4-(4-Pyridinyl)-1,3-thiazol-2-yl]amino}phenyl)ethanone,
4-(4-Pyridinyl)-N-[4-(trifluoromethyl)phenyl]-1,3-thiazol-2-amine,
4-{[4-(4-Pyridinyl)-1,3-thiazol-2-yl]amino}benzonitrile,
N-(2,6-Dimethylphenyl)-4-(4-pyridinyl)-1,3-thiazol-2-amine,
N-(2,6-Difluorophenyl)-4-(4-pyridinyl)-1,3-thiazol-2-amine,
N-{4-[2-(Methyloxy)-4-pyridinyl]-1,3-thiazol-2-yl}-N-(3-methylphenyl)amine,
N-(3-Methylphenyl)-4-(2-methyl-4-pyridinyl)-1,3-thiazol-2-amine,
4-(2-Fluoro-4-pyridinyl)-N-(3-methylphenyl)-1,3-thiazol-2-amine,
4-(2-Chloro-4-pyridinyl)-N-(3-methylphenyl)-1,3-thiazol-2-amine,
4-(2-Bromo-4-pyridinyl)-N-(3-methylphenyl)-1,3-thiazol-2-amine,
N-[4-(2,6-Dichloro-4-pyridinyl)-1,3-thiazol-2-yl]-N-(3-methylphenyl)amine,
4-(2-Ethynyl-4-pyridinyl)-N-(3-methylphenyl)-1,3-thiazol-2-amine,
4-(2-Ethyl-4-pyridinyl)-N-(3-methylphenyl)-1,3-thiazol-2-amine,
3-(4-{2-[(3-Methylphenyl)amino]-1,3-thiazol-4-yl}-2-pyridinyl)-2-propyn-1-ol,
3-(4-{2-[(3-Methylphenyl)amino]-1,3-thiazol-4-yl}-2-pyridinyl)-1-propanol,
N-(3-Methylphenyl)-4-(3-methyl-4-pyridinyl)-1,3-thiazol-2-amine,
4-(3-Fluoro-4-pyridinyl)-N-(3-methylphenyl)-1,3-thiazol-2-amine,
4-(3-Chloro-4-pyridinyl)-N-(3-methylphenyl)-1,3-thiazol-2-amine,
4-(3-Bromo-4-pyridinyl)-N-(3-methylphenyl)-1,3-thiazol-2-amine,
N-(3-Methylphenyl)-4-(2-nitro-4-pyridinyl)-1,3-thiazol-2-amine,
N-[4-(2-Amino-4-pyridinyl)-1,3-thiazol-2-yl]-N-(3-methylphenyl)amine,
N-(4-{2-[(3-Methylphenyl)amino]-1,3-thiazol-4-yl}-2-pyridinyl)acetamide,
4-(3-Ethynyl-4-pyridinyl)-N-(3-methylphenyl)-1,3-thiazol-2-amine,
4-(3-Ethyl-4-pyridinyl)-N-(3-methylphenyl)-1,3-thiazol-2-amine,
3-(4-{2-[(3-Methylphenyl)amino]-1,3-thiazol-4-yl}-3-pyridinyl)-2-propyn-1-ol,
3-(4-{2-[(3-Methylphenyl)amino]-1,3-thiazol-4-yl}-3-pyridinyl)-1-propanol,
N-(3-Methylphenyl)-4-(1-oxido-4-pyridinyl)-1,3-thiazol-2-amine,
N-(3-Methylphenyl)-4-(4-quinolinyl)-1,3-thiazol-2-amine,
N-(3-Methylphenyl)-4-(2-phenyl-4-quinolinyl)-1,3-thiazol-2-amine,
N-(3-Methylphenyl)-4-(4-pyridinyl)-1H-imidazol-2-amine,
N-(3-Methylphenyl)-4-[2-(4-morpholinyl)-4-pyridinyl]-1,3-thiazol-2-amine,
N-(3-Methylphenyl)-4-[2-(4-methyl-1-piperazinyl)-4-pyridinyl]-1,3-thiazol-2-amine, or
N,N-Dimethyl-4-{2-[(3-methylphenyl)amino]-1,3-thiazol-4-yl}-2-pyridinamine.

In certain embodiments, the disease treated or prevented according to the methods of the invention is a disease caused by a defect in the von Hippel-Lindau gene.

In some embodiments, the compounds of the invention target cells deficient in the von Hippel-Lindau gene.

In some embodiments, the compounds of the invention induce autophagic cell death.

In certain embodiments, the disease treated or prevented according to the methods of the invention is cancer.

In more specific embodiments, the cancer is a renal cell carcinoma, such as a clear cell-RCC, a pheochromocytoma, a hemangioblastoma of the central nervous system or retina, an endolymphatic sac tumor, a renal cyst, a pancreatic cyst, a neuroendocrine tumor, an endolymphatic sac tumor, or an epididymal or broad ligament cystadenoma.

In some embodiments, the tumors treated or prevented according to the methods of the invention are tumors of the retina, brain, spinal cord, ear, epidymis, broad ligament, adrenal gland, kidney, or pancreas.

The host receiving treatment according to the disclosed methods is any mammal in need of such treatment. Such mammals include, e.g., humans, ovines, bovines, equines, porcines, canines, felines, non-human primate, mice, and rats. In certain specific embodiments, the host is a human. In certain other specific embodiments, the host is a non-human mammal. In some embodiments, the host is a farm animal. In other embodiments, the host is a pet.

By “therapeutically-effective amount” is meant the concentration of a compound that is sufficient to elicit the desired therapeutic effect (e.g., treatment or prevention of a disease). It is generally understood that the effective amount of the compound will vary according to the weight, gender, age, and medical history of the host. Other factors that influence the effective amount may include, but are not limited to, the severity of the patient's condition, the disorder being treated, the stability of the compound, and, if desired, another type of therapeutic agent being administered with the compound of the invention. A larger total dose may be delivered by multiple administrations of the agent. Methods to determine efficacy and dosage are known to those skilled in the art. See, e.g., Roden, Harrison's Principles of Internal Medicine, Ch. 3, McGraw-Hill, 2004.

Actual dosage levels of the active ingredients in the pharmaceutical compositions of the invention may be varied so as to obtain an amount of the active ingredient that is effective to achieve the desired therapeutic response for a particular patient, composition, and mode of administration, without being toxic to the patient.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above.

If desired, the effective daily dose of the active compound may be administered as one, two, three, four, five, six, or more sub-doses administered separately at appropriate intervals throughout the day, optionally, in unit dosage forms. In certain embodiments of the present invention, the active compound may be administered two or three times daily. In specific embodiments, the active compound is administered once daily.

The preferred frequency of administration and effective dosage will vary from one individual to another and will depend upon the particular disease being treated and may be determined by one skilled in the art. However, it is contemplated that effective dosages of the inventive inhibitors may range from as low as about 1 mg per day to as high as about 1000 mg per day, including all intermediate dosages therebetween. More preferably, effective dosages may range from about 10 mg per day to about 100 mg per day, including all intermediate dosages therebetween. The inventive compositions may be administered in a single dosage, or in multiple, divided dosages.

As described above, the methods methods of the invention may in some embodiments be used for treating or preventing cancer. Such methods may, in certain embodiments, further comprise administration of a chemotherapeutic agent. Chemotherapeutic agents that may be coadministered with compounds and pharmaceutical compositions of the instant invention include: alemtuzumab, aminoglutethimide, amsacrine, anastrozole, asparaginase, bcg, bevacizumab, bicalutamide, bleomycin, bortezomib, buserelin, busulfan, campothecin, capecitabine, carboplatin, carmustine, CeaVac, cetuximab, chlorambucil, cisplatin, cladribine, clodronate, colchicine, cyclophosphamide, cyproterone, cytarabine, dacarbazine, daclizumab, dactinomycin, daunorubicin, dienestrol, diethylstilbestrol, docetaxel, doxorubicin, edrecolomab, epirubicin, epratuzumab, erlotinib, estradiol, estramustine, etoposide, everolimus, exemestane, filgrastim, fludarabine, fludrocortisone, fluorouracil, fluoxymesterone, flutamide, gemcitabine, gemtuzumab, genistein, goserelin, huJ591, hydroxyurea, ibritumomab, idarubicin, ifosfamide, IGN-101, imatinib, interferon, interleukin-2, irinotecan, ironotecan, letrozole, leucovorin, leuprolide, levamisole, lintuzumab, lomustine, MDX-210, mechlorethamine, medroxyprogesterone, megestrol, melphalan, mercaptopurine, mesna, methotrexate, mitomycin, mitotane, mitoxantrone, mitumomab, nilutamide, nocodazole, octreotide, oxaliplatin, paclitaxel, pamidronate, pentostatin, pertuzumab, plicamycin, porfimer, procarbazine, raltitrexed, rituximab, sorafinib, streptozocin, sunitinib, suramin, tamoxifen, temozolomide, temsirolimus, teniposide, testosterone, thalidomide, thioguanine, thiotepa, titanocene dichloride, topotecan, tositumomab, trastuzumab, tretinoin, vatalanib, vinblastine, vincristine, vindesine, and vinorelbine.

Other useful chemotherapeutic agents for combination with the compounds of the present invention include MDX-010; MAb, AME; ABX-EGF; EMD 72 000; apolizumab; labetuzumab; ior-t1; MDX-220; MRA; H-11 scFv; Oregovomab; huJ591 MAb, BZL; visilizumab; TriGem; TriAb; R3; MT-201; G-250, unconjugated; ACA-125; Onyvax-105; CDP-860; BrevaRex MAb; AR54; IMC-1C11; GlioMAb-H; ING-1; Anti-LCG MAbs; MT-103; KSB-303; Therex; KW-2871; Anti-HMI.24; Anti-PTHrP; 2C4 antibody; SGN-30; TRAIL-R1MAb, CAT; Prostate cancer antibody; H22xKi-4; ABX-MA1; Imuteran; and Monopharm-C.

These chemotherapeutic agents may be categorized by their mechanism of action into, for example, the following groups: anti-metabolites/anti-cancer agents, such as pyrimidine analogs (e.g., 5-fluorouracil, floxuridine, capecitabine, gemcitabine and cytarabine) and purine analogs, folate antagonists and related inhibitors (e.g., mercaptopurine, thioguanine, pentostatin and 2-chlorodeoxyadenosine (cladribine)); antiproliferative/antimitotic agents including natural products such as vinca alkaloids (e.g., vinblastine, vincristine, and vinorelbine), microtubule disruptors such as taxane (paclitaxel, docetaxel), vincristin, vinblastin, nocodazole, epothilones and navelbine, epidipodophyllotoxins (teniposide), DNA damaging agents (e.g., actinomycin, amsacrine, anthracyclines, bleomycin, busulfan, camptothecin, carboplatin, chlorambucil, cisplatin, cyclophosphamide, cytoxan, dactinomycin, daunorubicin, docetaxel, doxorubicin, epirubicin, hexamethylmelamineoxaliplatin, iphosphamide, melphalan, merchlorethamine, mitomycin, mitoxantrone, nitrosourea, paclitaxel, plicamycin, procarbazine, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (e.g., L-asparaginase, which systemically metabolizes L-asparagine and deprives cells which do not have the capacity to synthesize their own asparagine); antiplatelet agents; antiproliferative/antimitotic alkylating agents such as nitrogen mustards (e.g., mechlorethamine, cyclophosphamide and analogs, melphalan, chlorambucil), ethylenimines and methylmelamines (e.g., hexamethylmelamine and thiotepa), alkyl sulfonates-busulfan, nitrosoureas (e.g., carmustine (BCNU) and analogs, streptozocin), trazenes-dacarbazinine (DTIC); antiproliferative/antimitotic antimetabolites such as folic acid analogs (e.g., methotrexate); platinum coordination complexes (e.g., cisplatin, carboplatin), procarbazine, hydroxyurea, mitotane, aminoglutethimide; hormones, hormone analogs (e.g., estrogen, tamoxifen, goserelin, bicalutamide, nilutamide) and aromatase inhibitors (e.g., letrozole, anastrozole); anticoagulants (e.g., heparin, synthetic heparin salts and other inhibitors of thrombin); fibrinolytic agents (such as tissue plasminogen activator, streptokinase and urokinase), aspirin, COX-2 inhibitors, dipyridamole, ticlopidine, clopidogrel, abciximab; antimigratory agents; antisecretory agents (e.g., breveldin); immunosuppressives (e.g., cyclosporine, tacrolimus (FK-506), sirolimus (rapamycin), azathioprine, mycophenolate mofetil); anti-angiogenic compounds (e.g., TNP-470, genistein) and growth factor inhibitors (e.g., vascular endothelial growth factor (VEGF) inhibitors, fibroblast growth factor (FGF) inhibitors, epidermal growth factor (EGF) inhibitors); angiotensin receptor blocker; nitric oxide donors; anti-sense oligonucleotides; antibodies (e.g., trastuzumab and others listed above); cell cycle inhibitors and differentiation inducers (e.g., tretinoin); mTOR inhibitors, topoisomerase inhibitors (e.g., doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (e.g., cortisone, dexamethasone, hydrocortisone, methylpednisolone, prednisone, and prenisolone); growth factor signal transduction kinase inhibitors; mitochondrial dysfunction inducers and caspase activators; chromatin disruptors.

The pharmaceutical compositions of the instant invention may be coadministered with chemotherapeutic agents either singly or in combination.

Combination therapies comprising the inhibitors of the instant invention and a conventional chemotherapeutic agent may be advantageous over combination therapies known in the art because the combination allows the conventional chemotherapeutic agent to exert greater effect at lower dosage. In a specific embodiment, the effective dose (ED₅₀) for a chemotherapeutic agent, or combination of conventional chemotherapeutic agents, when used in combination with an epoxide inhibitor of the instant invention is at least 2 fold less than the ED₅₀ for the chemotherapeutic agent alone, and even more preferably at 5-fold, 10-fold, or even 25-fold less. Conversely, the therapeutic index (TI) for such chemotherapeutic agent or combination of such chemotherapeutic agent when used in combination with an epoxide inhibitor of the instant invention can be at least 2-fold greater than the TI for conventional chemotherapeutic regimen alone, and even more preferably at 5-fold, 10-fold, or even 25-fold greater.

In another aspect, the compounds and pharmaceutical compositions may be administered in combination with radiation therapy.

It will be readily apparent to one of ordinary skill in the relevant arts that other suitable modifications and adaptations to the methods and applications described herein may be made without departing from the scope of the invention or any embodiment thereof. Having now described the present invention in detail, the same will be more clearly understood by reference to the following Examples, which are included herewith for purposes of illustration only and are not intended to be limiting of the invention.

### EXAMPLES

### Synthetic Reactions

Analyses were carried out in the Campbell Microanalytical Laboratory, University of Otago, Dunedin, NZ. Melting points were determined on an Electrothermal 2300 Melting Point Apparatus. NMR spectra were obtained on a Bruker Avance 400 spectrometer at 400 MHz for ¹H and 100 MHz for ¹³C spectra. Spectra were obtained in [(CD₃)₂SO] unless otherwise specified, and were referenced to Me₄Si. Chemical shifts and coupling constants were recorded in units of ppm and Hz, respectively. Assignments were determined using COSY, HSQC, and HMBC two-dimensional experiments. Low resolution mass spectra were gathered by direct injection of methanolic solutions into a Surveyor MSQ mass spectrometer using an atmospheric pressure chemical ionization (APCI) mode with a corona voltage of 50 V and a source temperature of 400° C. Solutions in organic solvents were dried with anhydrous MgSO₄. Solvents were evaporated under reduced pressure on a rotary evaporator. Thin-layer chromatography was carried out on aluminium-backed silica gel plates (Merck 60 F₂₅₄) with visualization of components by UV light (254 nm) or exposure to I₂. Column chromatography was carried out on silica gel (Merck 230-400 mesh). DCM refers to dichloromethane; DME refers to dimethoxyethane, DMF refers to dry N,N-dimethylformamide; Et₂O refers to diethyl ether; EtOAc refers to ethyl acetate; EtOH refers to ethanol; MeOH refers to methanol; pet. ether refers to petroleum ether, boiling range 40-60° C.; THF refers to tetrahydrofuran dried over sodium benzophenone ketyl.

### Example 1-1

### N-Phenyl-4-(4-pyridinyl)-1,3-thiazol-2-amine (3)

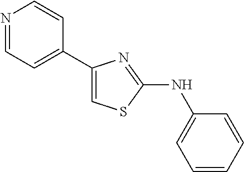

2-Bromo-1-(pyridin-4-yl)ethanone Hydrobromide (1). Br₂ (4.45 mL, 86.7 mmol) was added dropwise to a stirred a solution of 4-acetylpyridine (10.0 g, 82.5 mmol) in 30% HBr/HOAc (100 mL) at 0-5° C. The mixture was stirred at 40° C. for 1 h and then 75° C. for 1 h. The mixture was cooled to 20° C., diluted with Et₂O (400 mL) and stirred for 30 min. The precipitate was filtered, washed with Et₂O (25 mL) and dried under vacuum to give the hydrobromide salt 1 (22.39 g, 97%) as a cream powder: mp (HOAc/ether) 190-195° C. [lit. (Ogawa, K. J. Chem. Soc Perkin 1. 1985, 2417) mp 197-200° C.]; ¹H NMR δ 9.02 (br d, J=5.5 Hz, 2 H, H-2′, H-6′), 8.71 (br s, 1 H, N.HBr), 8.15 (br d, J=5.5 Hz, 2 H, H-3′, H-5′), 5.06 (s, 2 H, CH₂Br).

N-Phenyl-4-(4-pyridinyl)-1,3-thiazol-2-amine (3). A mixture of bromoketone hydrobromide 1 (0.91 g, 3.24 mmol) and phenylthiourea 2 (0.49 g, 3.24 mmol) in EtOH (20 mL) was stirred at reflux temperature for 2 h. The mixture was cooled to 20° C., diluted with water (50 mL), the pH adjusted to ca. 8 with aqueous NH₃ and the mixture stirred at 20° C. for 2 h. The precipitate was filtered, washed with water (5 mL) and dried to give amine 3 (0.77 g, 94%) as a white powder: mp (H₂O) 222-225° C. [lit. (Westphal, G. J. Prakt. Chem. 1976, 318, 875) mp (EtOH) 222° C.]; ¹H NMR δ 10.45 (br s, 1 H, NH), 8.75 (dd, J=5.1, 1.4 Hz, 2 H, H-2′, H-6′), 8.15 (dd, J=5.1, 1.4 Hz, 2 H, H-3′, H-5′), 8.01 (s, 1 H, H-5), 7.74 (dd, J=8.5, 0.9 Hz, 2 H, H-2″, H-6″) 7.37 (br dd, J=8.5, 7.4 Hz, 2 H, H-3″, H-5″), 6.99 (br t, J=7.4 Hz, 1 H, H-4″); ¹³C NMR δ 163.6, 146.4, 146.0, 144.8, 140.7 (2), 128.9 (2), 121.6, 121.0 (2), 117.0 (2), 111.5; MS m/z 254.4 (MH⁺, 100%).

### Example 1-2

### N-(3-Methylphenyl)-4-(4-pyridinyl)-1,3-thiazol-2-amine (5)

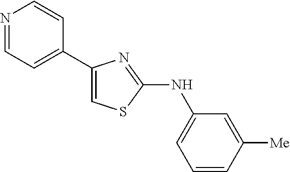

3-Methylphenylthiourea (4). NH₄SCN (3.55 g, 46.7 mmol) was added to a stirred solution of m-toluidine (5.00 g, 46.7 mmol) in 1 M HCl (47 mL) at 100° C. and the solution stirred at 100° C. for 16 h. The solution was diluted with water (60 mL) and stood at 5° C. for 2 h. The precipitate was filtered, washed with water (5 mL), washed with ether (5 mL), and dried to give thiourea 4 (3.21 g, 41%) as a white powder: mp (H₂O) 95-97° C. [lit. (Rasmussen, C. R. et al., Synthesis 1988, 456) mp (MeOH) 110-113° C.]; ¹H NMR δ 9.58 (s, 1 H, NH), 7.38 (br s, 2 H, NH₂), 7.15-7.23 (m, 3 H, H-2, H-4, H-5), 6.93 (br d, J=6.9 Hz, 1 H, H-6), 2.30 (s, 3 H, CH₃).

Alternative Preparation of 3-Methylphenylthiourea (4). Benzoyl chloride (7.5 mL, 64.7 mmol) was added dropwise to a solution of NH₄SCN (5.28 g, 69.3 mmol) in dry acetone (30 mL). The mixture was stirred at reflux temperature for 15 min. The heating was removed and m-toluidine (5.0 mL, 46.2 mmol) was added dropwise keeping the solution at reflux. The mixture was stirred at reflux temperature for a further 30 min, cooled to 20° C. and poured onto ice. The mixture was stirred for 30 min and then the precipitate was filtered and washed with water (15 mL) and dried. The solid was added to a solution of NaOH (10% w/v, 100 mL) at 80° C. and stirred at 80° C. for 30 min, then cooled to 20° C. and poured onto ice/HCl (6 M, 50 mL). The pH of the mixture was adjusted to 10 with cNH₃ solution and stirred for 30 min. The precipitate was filtered and washed with water (20 mL) and dried. The precipitate was purified by column chromatography, eluting with a gradient (30-100%) of EtOAc/pet. ether, to give thiourea 4 (6.62 g, 86%) as a white powder: mp (EtOAc) 109-111° C. [lit. (Rasmussen, C. R. et al., Synthesis 1988, 456) mp (MeOH) 110-113° C.]; ¹H NMR δ 9.58 (s, 1 H, NH), 7.38 (br s, 2 H, NH₂), 7.15-7.23 (m, 3 H, H-2, H-4, H-5), 6.93 (br d, J=6.9 Hz, 1 H, H-6), 2.30 (s, 3 H, CH₃).

N-(3-Methylphenyl)-4-(4-pyridinyl)-1,3-thiazol-2-amine (5) (“STF-62247”). A mixture of bromoketone hydrobromide 1 (1.13 g, 4.03 mmol) and 3-methylphenylthiourea 4 (0.67 g, 4.03 mmol) in EtOH (20 mL) was stirred at reflux temperature for 3 h. The mixture was cooled to 20° C., diluted with water (50 mL), the pH adjusted to ca. 8 with aqueous NH₃ and the mixture stirred at 20° C. for 2 h. The precipitate was filtered, washed with water (5 mL) and dried. The crude solid was purified by column chromatography, eluting with a gradient (50-100%) of EtOAc/pet. ether, to give amine 5 (0.94 g, 87%) as a cream powder: mp (EtOAc/pet. ether) 158-160° C.; ¹H NMR δ 10.27 (br s, 1 H, NH), 8.62 (dd, J=4.6, 1.6 Hz, 2 H, H-2′, H-6′), 7.84 (dd, J=4.6, 1.6 Hz, 2 H, H-3′, H-5′), 7.69 (s, 1 H, H-5), 7.57 (br d, J=7.9 Hz, 1 H, H-6″), 7.47 (br s, 1 H, H-2″), 7.24 (dd, J=7.8, 7.5 Hz, 1 H, H-5″), 6.81 (br d, J=7.5 Hz, 1 H, H-4″), 2.33 (s, 3 H, CH₃); ¹³C NMR δ 163.5, 150.1 (2), 147.6, 140.9, 140.8, 138.1, 128.8, 122.2, 119.8 (2), 117.5, 114.1, 107.2, 21.2; MS m/z 268.4 (MH⁺, 100%). Anal. calcd for C₁₅H₁₃N₃S.¼H₂O: C, 66.27; H, 5.01; N, 15.46. Found: C, 64.48; H, 5.08; N, 15.08%.

### Example 1-3

### N-(3-Ethylphenyl)-4-(4-pyridinyl)-1,3-thiazol-2-amine (7)

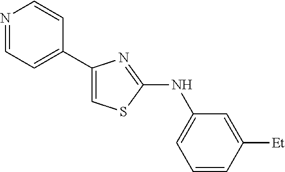

3-Ethylphenylthiourea (6). Benzoyl chloride (3.9 mL, 33.8 mmol) was added dropwise to a stirred solution of NH₄SCN (2.76 g, 36.2 mmol) in acetone (50 mL). The mixture was stirred at reflux temperature for 15 mins, the heating was removed and 3-ethylaniline (3.0 mL, 24.1 mmol) was added dropwise, keeping the solution at reflux temperature. The mixture was stirred at reflux temperature for 30 min, then poured onto ice (50 mL) and stirred for 15 min. The resulting mixture was filtered and washed with water (5 mL), and dried. The solid was added to a stirred solution of NaOH (10% w/v, 80 mL) at 80° C. The solution was stirred at 80° C. for 30 mins and then poured onto ice/HCl (6 M, 30 mL) and stirred for 10 mins. The pH was adjusted to 10 with cNH₃ solution, the resulting precipitate filtered and washed with water (10 mL) and dried. The crude solid was purified by column chromatography, eluting with a gradient (20-50%) of EtOAc/pet. ether, to give thiourea 6 (2.73 g, 63%) as an white powder: mp (EtOAc) 82-83° C.; ¹H NMR δ 9.60 (s, 1 H, NH), 7.42 (br s, 2 H, NH₂), 7.19-7.24 (m, 3 H, H-2, H-5, H-6), 6.95-6.99 (m, 1 H, H-4), 2.58 (q, J=7.4 Hz, 2 H, CH₂), 1.17 (t, J=7.4 Hz, 3 H, CH₃); MS m/z 181.5 (MH⁺, 100%). Anal. calcd for C₉H₁₂N₂S: C, 59.96; H, 6.71; N, 15.54. Found: C, 60.09; H, 6.97; N, 15.59%.

N-(3-Ethylphenyl)-4-(4-pyridinyl)-1,3-thiazol-2-amine (7). A mixture of bromoketone hydrobromide 1 (0.47 g, 1.7 mmol) and 3-ethylphenylthiourea (6) (0.30 g, 1.7 mmol) in EtOH (30 mL) was stirred at reflux temperature for 2 h. The mixture was cooled to 20° C., diluted with water (50 mL), the pH adjusted to ca. 8 with aqueous NH₃ and the mixture stirred at 0° C. for 2 h. The precipitate was filtered, washed with water (5 mL) and dried. The crude solid was purified by column chromatography, eluting with a gradient (50-80%) of EtOAc/pet. ether, to give amine 7 (0.45 g, 95%) as a cream powder: mp (EtOAc/pet. ether) 172-174° C.; ¹H NMR δ 10.28 (s, 1 H, NH), 8.62 (dd, J=4.5, 1.6 Hz, 2 H, H-2′, H-6′), 7.84 (dd, J=4.5, 1.6 Hz, 2 H, H-3′, H-5′), 7.68 (s, 1 H, H-5), 7.53-7.57 (m, 2 H, H-2″, H-6″), 7.26 (dd, J=8.0, 7.6 Hz, 1 H, H-5″), 6.85 (br d, J=7.6 Hz, 1 H, H-4″), 2.62 (q, J=7.6 Hz, 2 H, CH₂), 1.22 (t, J=7.6 Hz, 3 H, CH₃); ¹³C NMR δ 163.5, 150.0 (2), 147.6, 144.4, 141.0, 140.9, 128.8, 121.0, 119.7 (2), 116.3, 114.4, 107.2, 28.2, 15.2; MS m/z 282.5 (MH⁺, 100%). Anal. calcd for C₁₆H₁₅N₃S: C, 68.30; H, 5.37; N, 14.93. Found: C, 68.53; H, 5.52; N, 14.92%.

### Example 1-4

### N-(3-Isopropylphenyl)-4-(4-pyridinyl)-1,3-thiazol-2-amine (9)

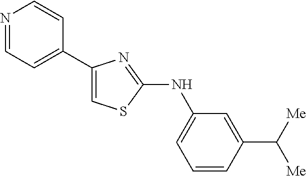

3-Isopropylphenylthiourea (8). NH₄SCN (2.40 g, 31.7 mmol) was added to a stirred solution of 3-isopropylaniline (4.28 g, 31.7 mmol) in 1 M HCl (32 mL) at 100° C. and the solution stirred at 100° C. for 16 h. The solution was diluted with water (60 mL) and stood at 5° C. for 2 h. The precipitate was filtered, washed with water (5 mL), washed with ether (5 mL), and dried. The crude solid was purified by column chromatography, eluting with EtOAc, to give thiourea 8 (1.38 g, 22%) as an oil: ¹H NMR δ 9.60 (s, 1 H, NH), 7.35 (br s, 2 H, NH₂), 7.19-7.26 (m, 3 H, H-2, H-4, H-5), 7.00 (dt, J=4.5, 1.6 Hz, 1 H, H-6), 2.86 (sept, J=6.9 Hz, 1 H, CH), 1.19 (d, J=6.9 Hz, 6 H, 2×CH₃); MS m/z 195.5 (MH⁺, 100%). Anal. calcd for C₁₀H₁₄N₂S.¼H₂O: C, 60.42; H, 7.35; N, 14.09. Found: C, 60.41; H, 7.22; N, 13.85%.

N-(3-Isopropylphenyl)-4-(4-pyridinyl)-1,3-thiazol-2-amine (9). A mixture of bromoketone hydrobromide 1 (0.92 g, 3.3 mmol) and 3-isopropylphenylthiourea (8) (0.64 g, 3.3 mmol) in EtOH (30 mL) was stirred at reflux temperature for 3 h. The mixture was cooled to 20° C., diluted with water (50 mL), the pH adjusted to ca. 8 with aqueous NH₃ and the mixture stirred at 0° C. for 2 h. The precipitate was filtered, washed with water (5 mL) and dried. The crude solid was purified by column chromatography, eluting with a gradient (50-100%) of EtOAc/pet. ether, to give amine 9 (0.81 g, 84%) as a cream powder: mp (EtOAc/pet. ether) 200-202° C.; ¹H NMR δ 10.29 (s, 1 H, NH), 8.61 (dd, J=4.5, 1.6 Hz, 2 H, H-2′, H-6′), 7.84 (dd, J=4.5, 1.6 Hz, 2 H, H-3′, H-5′), 7.69 (s, 1 H, H-5), 7.64 (t, J=1.8 Hz, 1 H, H-2″), 7.52 (ddd, J=7.7, 2.0, 1.4 Hz, 1 H, H-6″), 7.26 (t, J=7.8 Hz, 1 H, H-5″), 6.89 (br d, J=7.8 Hz, 1 H, H-4″), 2.90 (sept, J=6.9 Hz, 1 H, CH), 1.24 (d, J=6.9 Hz, 6 H, 2×CH₃); ¹³C NMR δ 163.5, 150.0 (2), 149.1, 147.5, 141.0, 140.9, 128.8, 119.7, 119.6 (2), 114.8, 114.5, 107.2, 33.4, 23.7 (2); MS m/z 296.5 (MH⁺, 100%). Anal. calcd for C₁₇H₁₇N₃S: C, 69.12; H, 5.80; N, 14.22. Found: C, 69.18; H, 5.92; N, 14.34%.

### Example 1-5

### N-(3-tert-Butylphenyl)-4-(4-pyridinyl)-1,3-thiazol-2-amine (11)

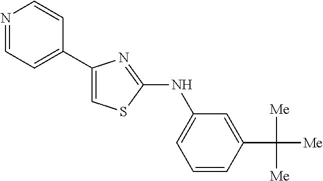

3-tert-Butylphenylthiourea (10). Benzoyl chloride (5.4 mL, 46.9 mmol) was added dropwise to a stirred solution of NH₄SCN (3.83 g, 50.3 mmol) in acetone (50 mL). The mixture was stirred at reflux temperature for 15 mins, the heating was removed and 3-t-butylaniline (5.0 g, 33.5 mmol) was added dropwise, keeping the solution at reflux temperature. The mixture was stirred at reflux temperature for 30 min, then poured onto ice (50 mL) and stirred for 15 min. The resulting mixture was filtered and washed with water (5 mL), and dried. The solid was added to a stirred solution of NaOH (10% w/v, 100 mL) at 80° C. The solution was stirred at 80° C. for 30 mins and then poured onto ice/HCl (6 M, 30 mL) and stirred for 10 mins. The pH was adjusted to 10 with cNH₃ solution, the resulting precipitate filtered and washed with water (10 mL) and dried. The crude solid was purified by column chromatography, eluting with 50% EtOAc/pet. ether, to give thiourea 10 (6.53 g, 94%) as a gum: ¹H NMR (CDCl₃) δ 7.95 (s, 1 H, NH), 7.35-7.38 (m, 2 H, H-4, H-5), 7.23-7.25 (m, 1 H, H-2), 7.04-7.07 (m, 1 H, H-6), 6.07 (br s, 2 H, NH₂), 1.31 [s, 9 H, C(CH₃)₃]. Anal. calcd for C₁₁H₁₆N₂S: C, 63.42; H, 7.74; N, 13.45. Found: C, 63.83; H, 7.64; N, 12.81%.

N-(3-tert-Butylphenyl)-4-(4-pyridinyl)-1,3-thiazol-2-amine (11). A mixture of bromoketone hydrobromide 1 (0.56 g, 2.0 mmol) and 3-tert-butylphenylthiourea (10) (0.41 g, 2.0 mmol) in EtOH (30 mL) was stirred at reflux temperature for 2 h. The mixture was cooled to 20° C., diluted with water (50 mL), the pH adjusted to ca. 8 with aqueous NH₃ and the mixture stirred at 0° C. for 2 h. The precipitate was filtered, washed with water (5 mL) and dried. The crude solid was purified by column chromatography, eluting with 50% EtOAc/pet. ether, to give amine 11 (0.50 g, 81%) as a cream powder: mp (EtOAc/pet. ether) 208-210° C.; ¹H NMR δ 10.30 (br s, 1 H, NH), 8.61 (dd, J=4.5, 1.6 Hz, 2 H, H-2′, H-6′), 7.83-7.87 (m, 3 H, H-3′, H-5′, H-2″), 7.69 (s, 1 H, H-5), 7.48 (ddd, J=8.0, 1.7, 0.8 Hz, 1 H, H-6″), 7.28 (dd, J=8.0, 7.8 Hz, 1 H, H-5″), 7.02 (ddd, J=7.8, 1.7, 0.8 Hz, 1 H, H-4″), 1.32 [s, 9 H, C(CH₃)₃]; ¹³C NMR δ 163.5, 151.4, 150.0 (2), 147.5, 141.0, 140.6, 128.5, 119.7 (2), 118.4, 114.2, 114.0, 107.2, 34.4, 31.0 (3); MS m/z 310.5 (MH⁺, 100%). Anal. calcd for C₁₈H₁₉N₃S: C, 69.87; H, 6.19; N, 13.58.

Found: C, 69.93; H, 6.07; N, 13.57%.

### Example 1-6

### 3-{[4-(4-Pyridinyl)-1,3-thiazol-2-yl]amino}phenol (13)

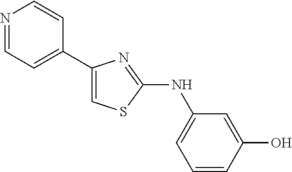

3-Hydroxyphenylthiourea (12). NH₄SCN (3.54 g, 46.6 mmol) was added to a stirred solution of 3-aminophenol (5.08 g, 46.6 mmol) in 1 M HCl (47 mL) at 100° C. and the solution stirred at 100° C. for 16 h. The solution was diluted with water (60 mL) and stood at 5° C. for 2 h. The precipitate was filtered, washed with water (5 mL), washed with ether (5 mL), and dried to give thiourea 12 (0.64 g, 8%) as a white powder: mp (H₂O) 172-175° C.; ¹H NMR δ 9.94 (s, 1 H, NH), 9.41 (br s, 1 H, OH), 7.34 (br s, 2 H, NH₂), 7.09 (t, J=8.0 Hz, 1 H, H-5), 6.88 (t, J=2.1 Hz, 1 H, H-2), 6.76 (ddd, J=8.0, 1.2 Hz, 1 H, H-4), 6.52 (ddd, J=8.1, 2.3, 0.8 Hz, 1 H, H-6).

3-{[4-(4-Pyridinyl)-1,3-thiazol-2-yl]amino}phenol (13). A mixture of bromoketone hydrobromide 1 (1.02 g, 3.63 mmol) and 3-hydroxyphenylthiourea (12) (0.61 g, 3.63 mmol) in EtOH (25 mL) was stirred at reflux temperature for 3 h. The mixture was cooled to 20° C., diluted with water (50 mL), the pH adjusted to ca. 8 with aqueous NH₃ and the mixture stirred at 5° C. for 1 h. The mixture was filtered, washed with water (5 mL) and Et₂O (5 mL) and dried to give phenol 13 (0.98 g, 99%) as a cream powder: mp (H₂O) 228-231° C.; ¹H NMR δ 10.26 (br s, 1 H, NH), 9.41 (br s, 1 H, OH), 8.67 (dd, J=4.7, 1.5 Hz, 2 H, H-2″, H-6″), 7.99 (dd, J=4.7, 1.5 Hz, 2 H, H-3″, H-5″), 7.80 (s, 1 H, H-5′), 7.35 (t, J=2.1 Hz, 1 H, H-2), 7.12 (t, J=8.0 Hz, 1 H, H-5), 7.13 (ddd, J=7.9, 2.2, 0.8 Hz, 1 H, H-6), 6.41 (ddd, J=7.9, 2.2, 0.8 Hz, 1 H, H-4); ¹³C NMR δ 163.4, 157.8, 148.4 (2), 147.1, 142.5, 141.8, 129.5, 120.3 (2), 108.8, 108.7, 107.9, 104.1; MS m/z 270.3 (MW, 100%). Anal. calcd for C₁₄H₁₁N₃OS.1¼H₂O: C, 57.62; H, 4.66; N, 14.39. Found: C, 57.47; H, 4.14; N, 14.21%.

### Example 1-7

### N-(3-Methoxyphenyl)-4-(4-pyridinyl)-1,3-thiazol-2-amine (15)

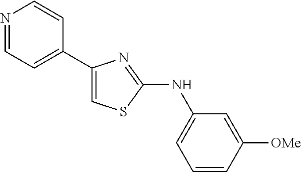

3-Methoxyphenylthiourea (14). NH₄SCN (3.09 g, 40.6 mmol) was added to a stirred solution of m-anisidine (5.00 g, 40.6 mmol) in 1 M HCl (41 mL) at 100° C. and the solution stirred at 100° C. for 16 h. The solution was diluted with water (60 mL) and stood at 5° C. for 2 h. The precipitate was filtered, washed with water (5 mL), washed with ether (5 mL), and dried to give thiourea 14 (1.34 g, 18%) as a white powder: mp (H₂O) 154-157° C. [lit. (Rasmussen et al., Synthesis 1988, 456) mp (MeOH) 157-159° C.]; ¹H NMR δ 9.65 (s, 1 H, NH), 7.42 (br s, 2 H, NH₂), 7.21 (dd, J=8.3, 7.9 Hz, 1 H, H-5), 7.12 (t, J=2.0 Hz, 1 H, H-2), 6.91 (dd, J=7.9, 1.3 Hz, 1 H, H-4), 6.69 (ddd, J=8.3, 2.4, 0.6 Hz, 1 H, H-6), 3.73 (s, 3 H, OCH₃).

N-(3-Methoxyphenyl)-4-(4-pyridinyl)-1,3-thiazol-2-amine (15). A mixture of bromoketone hydrobromide 1 (1.02 g, 3.62 mmol) and 3-methoxyphenylthiourea (14) (0.66 g, 3.62 mmol) in EtOH (20 mL) was stirred at reflux temperature for 3 h. The mixture was cooled to 20° C., diluted with water (50 mL), the pH adjusted to ca. 8 with aqueous NH₃ and the mixture stirred at 5° C. for 2 h. The precipitate was filtered, washed with water (5 mL) and dried. The crude solid was purified by column chromatography, eluting with a gradient (50-100%) of EtOAc/pet. ether, to give amine 15 (0.69 g, 68%) as a cream powder: mp (EtOAc/pet. ether) 176-178° C.; ¹H NMR δ 10.36 (br s, 1 H, NH), 8.62 (dd, J=4.5, 1.6 Hz, 2 H, H-2′, H-6′), 7.84 (dd, J=4.5, 1.6 Hz, 2 H, H-3′, H-5′), 7.71 (s, 1 H, H-5), 7.51 (t, J=2.2 Hz, 1 H, H-2″), 7.25 (t, J=8.1 Hz, 1 H, H-5″), 7.18 (br d, J=8.0 Hz, 1 H, H-4″), 6.56 (br d, J=8.0 Hz, 1 H, H-6″), 3.79 (s, 3 H, OCH₃); ¹³C NMR δ 163.3, 159.8, 150.1 (2), 147.6, 142.0, 140.9, 129.7, 119.7 (2), 109.4, 107.5, 106.8, 102.7, 54.8; MS m/z 284.5 (MH⁺, 100%). Anal. calcd for C₁₅H₁₃N₃OS: C, 63.58; H, 4.62; N, 14.83. Found: C, 63.79; H, 4.68; N, 14.87%.

### Example 1-8

### N-(3-Chlorophenyl)-4-(4-pyridinyl)-1,3-thiazol-2-amine (17)

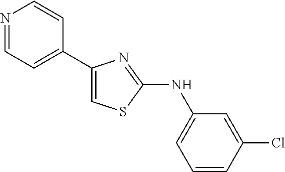

3-Chlorophenylthiourea (16). NH₄SCN (2.98 g, 39.2 mmol) was added to a stirred solution of 3-chloroaniline (5.00 g, 39.2 mmol) in 1 M HCl (40 mL) at 100° C. and the solution stirred at 100° C. for 16 h. The solution was diluted with water (60 mL) and stood at 5° C. for 2 h. The precipitate was filtered, washed with water (5 mL), washed with ether (5 mL), and dried to give thiourea 16 (3.63 g, 50%) as a white powder: mp (H₂O) 139-142° C. [lit. (Rasmussen, C. R. et al., Synthesis 1988, 456) mp (acetone/H₂O) 140-140.5° C.]; ¹H NMR δ 9.78 (s, 1 H, NH), 7.78 (br s, 2 H, NH₂), 7.70 (br s, 1 H, H-2), 7.30-7.35 (m, 2 H, H-4, H-5), 7.17-7.16 (m, 1 H, H-6).

N-(3-Chlorophenyl)-4-(4-pyridinyl)-1,3-thiazol-2-amine (17). A mixture of bromoketone hydrobromide 1 (1.13 g, 4.02 mmol) and 3-chlorophenylthiourea (16) (0.75 g, 4.02 mmol) in EtOH (20 mL) was stirred at reflux temperature for 3 h. The mixture was cooled to 20° C., diluted with water (50 mL), the pH adjusted to ca. 8 with aqueous NH₃ and the mixture stirred at 5° C. for 2 h. The precipitate was filtered, washed with water (5 mL) and dried. The crude solid was purified by column chromatography, eluting with a gradient (50-100%) of EtOAc/pet. ether, to give amine 17 (0.95 g, 82%) as a lemon powder: mp (EtOAc/pet. ether) 208-210° C.; ¹H NMR δ 10.57 (br s, 1 H, NH), 8.63 (dd, J=4.5, 1.6 Hz, 2 H, H-2′, H-6′), 7.93 (t, J=2.1 Hz, 1 H, H-2″), 7.83 (dd, J=4.5, 1.6 Hz, 2 H, H-3′, H-5′), 7.77 (s, 1 H, H-5), 7.61 (ddd, J=8.3, 2.0, 0.8 Hz, 1 H, H-6″), 7.38 (dd, J=8.3, 8.0 Hz, 1 H, H-4″), 7.03 (ddd, J=8.0, 2.0, 0.8 Hz, 1 H, H-4″); ¹³C NMR δ 162.9, 150.1 (2), 147.6, 142.1, 140.8, 133.2, 130.6, 120.8, 119.7 (2), 116.8, 115.3, 108.3. Anal. calcd for C₁₄H₁₀ClN₃S.¼EtOAc: C, 58.16; H, 3.90; N, 13.56. Found: C, 58.21; H, 3.88; N, 13.78%

### Example 1-9

### 4-(4-Pyridinyl)-N-[3-(trifluoromethyl)phenyl]-1,3-thiazol-2-amine (19)

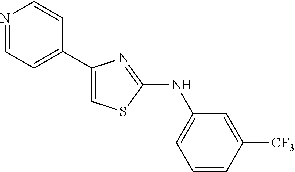

A mixture of bromoketone hydrobromide 1 (0.64 g, 2.27 mmol) and 3-trifluoromethylphenylthiourea (18) (0.50 g, 2.27 mmol) in EtOH (20 mL) was stirred at reflux temperature for 2 h. The mixture was cooled to 20° C., diluted with water (50 mL), the pH adjusted to ca. 8 with aqueous NH₃ and the mixture stirred at 20° C. for 1 h. The mixture was extracted with EtOAc (2×50 mL) and the combined organic fraction dried and the solvent evaporated. The residue was purified by column chromatography, eluting with a gradient (50-100%) of EtOAc/pet. ether, to give amine 19 (0.66 g, 90%) as a cream powder: mp (EtOAc/pet. ether) 239-241° C.; ¹H NMR δ 10.73 (br s, 1 H, NH), 8.63 (dd, J=4.5, 1.6 Hz, 2 H, H-2′, H-6′), 8.30 (br s, 1 H, H-2″), 7.90 (br dd, J=8.2, 1.8 Hz, 1 H, H-4″), 7.84 (dd, J=4.5, 1.6 Hz, 2 H, H-3′, H-5′), 7.80 (s, 1 H, H-5), 7.59 (dd, J=8.1, 7.9 Hz, 1 H, H-5″), 7.31 (d, J=7.9 Hz, 1 H, H-6″); ¹³C NMR δ 162.9, 150.1 (2), 147.5, 141.4, 140.8, 130.1, 129.6 (q, J=31 Hz), 124.4 (q, J=272 Hz), 120.3, 119.7 (2), 117.3 (q, J=4 Hz), 112.7 (d, J=4 Hz), 108.3; MS m/z 321.4 (MH⁺, 100%). Anal. calcd for C₁₅H₁₀F₃N₃S: C, 56.07; H, 3.14; N, 13.08. Found: C, 55.67; H, 3.07; N, 12.85%.

### Example 1-10

### N-(4-Methylphenyl)-4-(4-pyridinyl)-1,3-thiazol-2-amine (21)

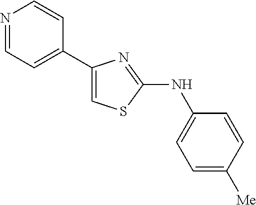

4-Methylphenylthiourea (20). NH₄SCN (3.55 g, 46.7 mmol) was added to a stirred solution of p-toluidine (5.00 g, 46.7 mmol) in 1 M HCl (50 mL) at 100° C. and the solution stirred at 100° C. for 16 h. The solution was diluted with water (60 mL) and stood at 5° C. for 2 h. The precipitate was filtered, washed with water (5 mL), washed with ether (5 mL), and dried to give thiourea 20 (1.88 g, 24%) as a white powder: mp (H₂O) 181-183° C. [lit. (Rasmussen et al., Synthesis 1988, 456) mp (MeOH) 194° C.]; ¹H NMR δ 9.45 (br s, 1 H, NH), 7.35 (br s, 2 H, NH₂), 7.24 (d, J=8.3 Hz, 2 H, H-3, H-5), 7.24 (d, J=8.36 Hz, 2 H, H-2, H-6), 2.27 (s, 3 H, CH₃).

N-(4-Methylphenyl)-4-(4-pyridinyl)-1,3-thiazol-2-amine (21). A mixture of bromoketone hydrobromide 1 (0.72 g, 2.56 mmol) and 4-methylphenylthiourea (20) (0.43 g, 2.56 mmol) in EtOH (20 mL) was stirred at reflux temperature for 3 h. The mixture was cooled to 20° C., diluted with water (50 mL), the pH adjusted to ca. 8 with aqueous NH₃ and the mixture stirred at 5° C. for 2 h. The precipitate was filtered, washed with water (5 mL) and dried. The crude solid was purified by column chromatography, eluting with a gradient (50-100%) of EtOAc/pet. ether, to give amine 21 (0.51 g, 74%) as a cream powder: mp (EtOAc) 230-233° C.; ¹H NMR δ 10.23 (br s, 1 H, NH), 8.61 (dd, J=4.5, 1.6 Hz, 2 H, H-2′, H-6′), 7.82 (dd, J=4.5, 1.6 Hz, 2 H, H-3′, H-5′), 7.66 (s, 1 H, H-5), 7.60 (br d, J=8.4 Hz, 2 H, H-2″, H-6″), 7.16 (br d, J=8.4 Hz, 2 H, H-3″, H-5″), 2.27 (s, 3 H, CH₃); ¹³C NMR δ 163.7, 150.0 (2), 147.6, 141.0, 138.5, 130.3, 129.3 (2), 119.8 (2), 117.1 (2), 107.0, 20.2; MS m/z 268.4 (MH⁺, 100%). Anal. calcd for C₁₅H₁₃N₃S: C, 67.39; H, 4.90; N, 15.72. Found: C, 67.53; H, 4.92; N, 15.50%.

### Example 1-11

### 2-(4-{[4-(4-Pyridinyl)-1,3-thiazol-2-yl]amino}phenyl)ethanol (23)

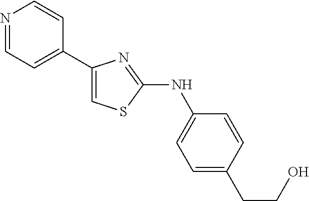

1-(4-(2-Hydroxyethyl)phenyl)thiourea (22). NH₄SCN (2.91 g, 38.2 mmol) was added to a stirred solution of 2-(4-aminophenyl)ethanol (5.24 g, 38.2 mmol) in 1 M HCl (38 mL) at 100° C. and the mixture stirred at 100° C. for 48 h. The mixture was cooled to 20° C., diluted with water (60 mL) and cooled at 0° C. for 1 h. The mixture was extracted with EtOAc (3×50 mL) and the combined organic fraction dried and the solvent evaporated. The residue was purified by column chromatography, eluting with EtOAc, to give thiourea 22 (0.41 g, 5%) as a tan gum: ¹H NMR δ 9.56 (br s, 1 H, NH), 7.30 (br s, 2 H, NH₂), 7.26 (br d, J=8.4 Hz, 2 H, H-3, H-5), 7.16 (br d, J=8.4 Hz, 2 H, H-2, H-6), 4.58 (t, J=5.2 Hz, 1 H, OH), 3.55-3.61 (m, 2 H, H-1), 2.68 (t, J=7.0 Hz, 2 H, H-2); MS m/z 197.3 (MH⁺, 100%).

2-(4-{[4-(4-Pyridinyl)-1,3-thiazol-2-yl]amino}phenyl)ethanol (23). A mixture of bromoketone hydrobromide 1 (0.58 g, 2.06 mmol) and thiourea 22 (0.41 g, 2.06 mmol) in EtOH (30 mL) was stirred at reflux temperature for 4 h. The mixture was cooled to 20° C., diluted with water (50 mL), the pH adjusted to ca. 8 with aqueous NH₃ and the mixture stirred at 0° C. for 1 h. The precipitate was filtered, washed with water (5 mL), and dried. The crude solid was purified by column chromatography, eluting with EtOAc, to give alcohol 23 (0.33 g, 54%) as a white powder: mp (EtOAc) 179-181° C.; ¹H NMR δ 10.24 (br s, 1 H, NH), 8.61 (dd, J=4.5, 1.6 Hz, 2 H, H-2″, H-6″), 7.84 (dd, J=4.5, 1.6 Hz, 2 H, H-3″, H-5″′), 7.66 (s, 1 H, H-5″), 7.60 (br d, J=8.5 Hz, 2 H, H-3′, H-5′), 7.20 (br d, J=8.5 Hz, 2 H, H-2′, H-6′), 4.58 (t, J=5.2 Hz, 1 H, OH), 3.55-3.61 (m, 2 H, H-1), 2.69 (t, J=7.1 Hz, 2 H, H-2); ¹³C NMR δ 163.6, 150.0 (2), 147.6, 141.0, 138.9, 132.5 (2), 119.8 (2), 117.0 (2), 107.0, 62.2, 38.3; MS m/z 298.4 (MH⁺, 100%). Anal. calcd for C₁₆H₁₅N₃OS.¼CH₃OH: C, 63.91; H, 5.28; N, 13.76.Found: C, 63.94; H, 5.26; N, 14.03%.

### Example 1-12

### 4-{[4-(4-Pyridinyl)-1,3-thiazol-2-yl]amino}phenol (25)

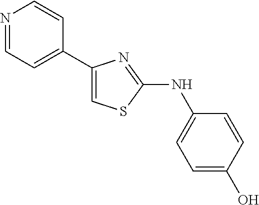

A mixture of bromoketone hydrobromide 1 (1.05 g, 3.74 mmol) and 4-hydroxyphenylthiourea (24) (0.63 g, 3.74 mmol) in EtOH (30 mL) was stirred at reflux temperature for 3 h. The mixture was cooled to 20° C., diluted with water (50 mL), the pH adjusted to ca. 8 with aqueous NH₃ and the mixture stirred at 0° C. for 1 h. The precipitate was filtered, washed with water (5 mL), washed with Et₂O (5 mL), and dried to give phenol 25 (0.82 g, 81%) as a cream powder: mp (H₂O) 269-272° C.; ¹H NMR δ 9.97 (br s, 1 H, NH), 9.11 (s, 1 H, OH), 8.59 (dd, J=4.5, 1.6

Hz, 2 H, H-2″, H-6″), 7.81 (dd, J=4.5, 1.6 Hz, 2 H, H-3″, H-5″), 7.57 (s, 1 H, H-5′), 7.48 (ddd, J=8.9, 3.4, 2.2 Hz, 2 H, H-3, H-5), 6.78 (ddd, J=8.9, 3.4, 2.2 Hz, 2 H, H-2, H-6); ¹³C NMR δ 164.6, 152.4, 150.0 (2), 147.6, 141.1, 132.9, 119.8 (2), 119.4 (2), 115.4 (2), 106.3; MS m/z 270.3 (MH⁺, 100%). Anal. calcd for C₁₄H₁₁N₃OS: C, 62.43; H, 4.12; N, 15.60.Found: C, 62.19; H, 4.24; N, 15.48%.

### Example 1-13

### N-(4-Methoxyphenyl)-4-(4-pyridinyl)-1,3-thiazol-2-amine (27)

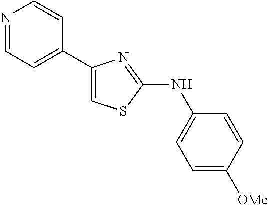

4-Methoxyphenylthiourea (26). NH₄SCN (3.09 g, 40.6 mmol) was added to a stirred solution of p-anisidine (5.00 g, 40.6 mmol) in 1 M HCl (41 mL) at 100° C. and the solution stirred at 100° C. for 16 h. The solution was diluted with water (60 mL) and stood at 5° C. for 2 h. The precipitate was filtered, washed with water (5 mL), washed with ether (5 mL), and dried to give thiourea 26 (0.85 g, 11%) as a white powder: mp (H₂O) 206-209° C. [lit. (Rasmussen et al., Synthesis 1988, 456) mp (MeOH) 212-214° C.]; ¹H NMR δ 9.44 (s, 1 H, NH), 7.15-7.30 (m, 4 H, NH₂, H-3, H-5), 6.89 (ddd, J=9.0, 3.4, 2.2 Hz, 2 H, H-2, H-6), 3.74 (s, 3 H, OCH₃).

N-(4-Methoxyphenyl)-4-(4-pyridinyl)-1,3-thiazol-2-amine (27). A mixture of bromoketone hydrobromide 1 (1.26 g, 4.50 mmol) and 4-methoxyphenylthiourea (26) (0.82 g, 4.50 mmol) in EtOH (20 mL) was stirred at reflux temperature for 1 h. The mixture was cooled to 20° C., diluted with water (50 mL), the pH adjusted to ca. 8 with aqueous NH₃ and the mixture stirred at 5° C. for 2 h. The precipitate was filtered, washed with water (5 mL) and dried. The crude solid was purified by column chromatography, eluting with EtOAc, to give amine

27 (0.76 g, 60%) as a cream powder: mp (EtOAc) 178-180° C.; ¹H NMR δ 10.13 (br s, 1 H, NH), 8.61 (dd, J=4.5, 1.6 Hz, 2 H, H-2′, H-6′), 7.83 (dd, J=4.5, 1.6

Hz, 2 H, H-3′, H-5′), 7.60 (m, 3 H, H-5, H-3″, H-5″), 6.95 (ddd, J=6.8, 3.5, 2.2 Hz, 2 H, H-2″, H-6″), 3.74 (s, 3 H, OCH₃); ¹³C NMR δ 164.1, 154.2, 150.0 (2), 147.6, 141.0, 134.4, 119.8 (2), 118.7 (2), 114.2 (2), 106.6, 55.1; MS m/z 284.5 (MH⁺, 100%). Anal. calcd for C₁₅H₁₃N₃OS: C, 63.58; H, 4.62; N, 14.83.Found: C, 63.45; H, 4.65; N, 14.82%.

### Example 1-14

### N-{4-[2-(Dimethylamino)ethoxy]phenyl}-4-(4-pyridinyl)-1,3-thiazol-2-amine (29)

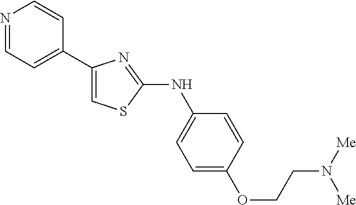

1-(4-(2-(Dimethylamino)ethoxy)phenyl)thiourea (28). Thiophosgene (0.95 mL, 12.45 mmol) was added dropwise to a stirred solution of 4-(2-(dimethylamino)ethoxy)aniline (2.04 g, 11.32 mmol) and iPr₂NEt (2.37 mL, 13.58 mmol) in dry THF (50 mL) at 0° C. and the solution stirred at 20° C. for 2 h. The solution was partitioned between DCM (50 mL) and water (50 mL), the organic fraction dried and the solvent evaporated. The residue was dissolved in DME (80 mL) and dry NH₃ gas bubbled through the solution for 10 min. The solution was stirred at 20° C. for 16 h and the solvent evaporated to give thiourea 28 (1.78 g, 66%) as a gum: ¹H NMR δ 9.63 (br s, 1 H, NH), 7.34 (br s, 2 H, NH₂), 7.26 (br d, J=8.9 Hz, 2 H, H-3, H-5), 6.90 (br d, J=8.9 Hz, 2 H, H-2, H-6), 4.08 (t, J=5.7 Hz, 2 H, CH₂O), 2.76 (t, J=5.7 Hz, 2 H, CH₂N), 2.32 [s, 6 H, N(CH₃)₂]; MS m/z 240.4 (MH⁺, 100%).

N-{4-[2-(Dimethylamino)ethoxy]phenyl}-4-(4-pyridinyl)-1,3-thiazol-2-amine (29). A mixture of bromoketone hydrobromide 1 (0.63 g, 2.25 mmol) and 1-(4-(2-(dimethylamino)ethoxy)phenyl)thiourea (28) (0.54 g, 2.25 mmol) in EtOH (20 mL) was stirred at reflux temperature for 2 h. The mixture was cooled to 20° C., diluted with water (50 mL), the pH adjusted to ca. 8 with aqueous NH₃ and the mixture stirred at 0° C. for 1 h. The precipitate was filtered, washed with water (5 mL), and dried. The crude solid was purified by column chromatography, eluting with a gradient (0-20%) of MeOH/EtOAc, followed by 1% aqueous NH₃/MeOH/EtOAc, to give amine 29 (447 mg, 58%) as a an oil: ¹H NMR δ 10.13 (s, 1 H, NH), 8.60 (dd, J=4.5, 1.6 Hz, 2 H, H-2′, H-6′), 7.83 (dd, J=4.5, 1.6 Hz, 2 H, H-3′, H-5′), 7.59-7.64 (m, 3 H, H-5, H-2″, H-6″), 6.95 (ddd, J=9.0, 3.5, 2.2 Hz, 2 H, H-3″, H-5″), 4.02 (br t, J=5.9 Hz, 2 H, CH₂O), 2.61 (br t, J=5.9 Hz, 2 H, CH₂N), 2.22 [s, 6 H, N(CH₃)₃]; ¹³C NMR δ 164.0, 153.4, 150.0 (2), 147.6, 141.0, 134.4, 119.8 (2), 118.7 (2), 114.8 (2), 106.6, 66.0, 57.6, 45.4 (2); MS m/z 341.5 (MH⁺, 100%). The dihydrochloride salt was crystallized from MeOH/EtOAc: mp (MeOH/EtOAc) 160-164° C. Anal. calcd for C₁₈H₂₂Cl₂N₄OS.1¼H₂O: C, 49.60; H, 5.66; N, 12.85.Found: C, 49.49; H, 5.75; N, 12.67%.

### Example 1-15

### N-(4-Chlorophenyl)-4-(4-pyridinyl)-1,3-thiazol-2-amine (31)

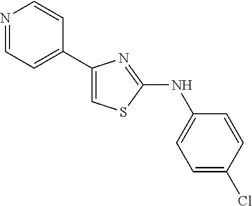

4-Chlorophenylthiourea (30). NH₄SCN (3.04 g, 40.0 mmol) was added to a stirred solution of 4-chloroaniline (5.10 g, 40.0 mmol) in 1 M HCl (40 mL) at 100° C. and the solution stirred at 100° C. for 16 h. The solution was diluted with water (60 mL) and stood at 5° C. for 2 h. The precipitate was filtered, washed with water (5 mL), washed with ether (5 mL), and dried. The crude solid was purified by column chromatography, eluting with a gradient (50-100%) of EtOAc/pet. ether, to give thiourea 30 (3.28 g, 44%) as a white powder: mp (EtOAc/pet. ether) 172-172° C. [lit. (Rasmussen et al., Synthesis 1988, 456) mp (iPrOH) 176-178° C.]; ¹H NMR δ 9.92 (s, 1 H, NH), 7.55 (br s, 2 H, NH₂), 6.47 (ddd, J=8.9, 3.0, 2.2 Hz, 2 H, H-2, H-6), 6.35 (ddd, J=8.9, 3.0, 2.2 Hz, 2 H, H-3, H-5).

N-(4-Chlorophenyl)-4-(4-pyridinyl)-1,3-thiazol-2-amine (31). A mixture of bromoketone hydrobromide 1 (1.24 g, 4.41 mmol) and 4-chlorophenylthiourea (30) (0.82 g, 4.41 mmol) in EtOH (20 mL) was stirred at reflux temperature for 3 h. The mixture was cooled to 20° C., diluted with water (50 mL), the pH adjusted to ca. 8 with aqueous NH₃ and the mixture stirred at 5° C. for 2 h. The precipitate was filtered, washed with water (5 mL) and dried. The crude solid was purified by column chromatography, eluting with a gradient (50-100%) of EtOAc/pet. ether, to give amine 31 (0.49 g, 39%) as a cream powder: mp (EtOAc/pet. ether) 259-261° C.; ¹H NMR δ 10.49 (br s, 1 H, NH), 8.61 (dd, J=4.5, 1.6 Hz, 2 H, H-2′, H-6′), 7.86 (dd, J=4.5, 1.6 Hz, 2 H, H-3′, H-5′), 7.76 (ddd, J=9.0, 3.2, 2.1 Hz, 2 H, H-2″, H-6″), 7.74 (s, 1 H, H-5), 7.40 (ddd, J=8.9, 3.2, 2.1 Hz, 2 H, H-3″, H-5″); ¹³C NMR δ 163.1, 150.0 (2), 147.6, 140.8, 139.8, 128.7 (2), 124.6, 119.8 (2), 118.3 (2), 107.8. Anal. calcd for C₁₄H₁₀ClN₃S: C, 58.43; H, 3.50; N, 14.60.Found: C, 58.46; H, 3.60; N, 14.57%.

### Example 1-16

### N-(4-Fluorophenyl)-4-(4-pyridinyl)-1,3-thiazol-2-amine (33)

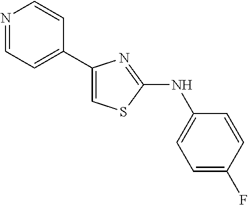

A mixture of bromoketone hydrobromide 1 (0.83 g, 2.94 mmol) and 4-fluorophenylthiourea (32) (0.50 g, 2.94 mmol) in EtOH (20 mL) was stirred at reflux temperature for 2 h. The mixture was cooled to 20° C., diluted with water (50 mL), the pH adjusted to ca. 8 with aqueous NH₃ and the mixture stirred at 5° C. for 2 h. The precipitate was filtered, washed with water (5 mL) and dried. The crude solid was purified by column chromatography, eluting with a gradient (50-100%) of EtOAc/pet. ether, to give amine 33 (0.76 g, 96%) as a cream powder: mp (EtOAc/pet. ether) 211-213° C.; ¹H NMR δ 10.37 (br s, 1 H, NH), 8.61 (dd, J=4.5, 1.6 Hz, 2 H, H-2′, H-6′), 7.84 (dd, J=4.5, 1.6 Hz, 2 H, H-3′, H-5′), 7.73-7.78 (m, 2 H, H-3′, H-5′), 7.70 (s, 1 H, H-5), 7.17-7.23 (m, 2 H, H-2″, H-6″); ¹³C NMR δ 163.5, 156.9 (d, J=241 Hz), 150.0 (2), 147.6, 140.9, 137.4, 119.8 (2), 118.4 (2, d, J=7 Hz), 115.4 (2, d, J=22 Hz), 107.3. Anal. calcd for C₁₄H₁₀FN₃S: C, 61.98; H, 3.72; N, 15.49.Found: C, 62.14; H, 3.79; N, 15.49%.

### Example 1-17

### N-(4-Nitrophenyl)-4-(4-pyridinyl)-1,3-thiazol-2-amine (35)

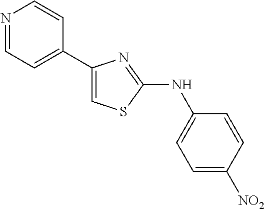

4-Nitrophenylthiourea (34). NH₄SCN (2.75 g, 36.2 mmol) was added to a stirred solution of 4-nitroaniline (5.00 g, 36.2 mmol) in 1 M HCl (40 mL) at 100° C. and the solution stirred at 100° C. for 16 h. The solution was diluted with water (60 mL) and stood at 5° C. for 2 h. The precipitate was filtered, washed with water (5 mL), washed with ether (5 mL), and dried. The crude solid was purified by column chromatography, eluting with a gradient (50-100%) of EtOAc/pet. ether, to give thiourea 34 (2.16 g, 30%) as a tan powder: mp (EtOAc/pet. ether) 213-215° C. [lit. (Rasmussen et al., Synthesis 1988, 456) mp (acetone) 220-242° C.]; ¹H NMR δ 10.24 (s, 1 H, NH), 8.18 (ddd, J=9.2, 3.1, 2.2 Hz, 2 H, H-3, H-5), 8.00 (br s, 2 H, NH₂), 7.85 (ddd, J=9.2, 3.1, 2.2 Hz, 2 H, H-2, H-6).

N-(4-Nitrophenyl)-4-(4-pyridinyl)-1,3-thiazol-2-amine (35). A mixture of bromoketone hydrobromide 1 (1.91 g, 6.80 mmol) and 4-nitrophenylthiourea

(34) (1.34 g, 6.80 mmol) in EtOH (50 mL) was stirred at reflux temperature for 2 h. The mixture was cooled to 20° C., diluted with water (50 mL), the pH adjusted to ca. 8 with aqueous NH₃ and the mixture extracted with EtOAc (3×50 mL). The organic fraction was dried and the solvent evaporated. The crude solid was purified by column chromatography, eluting with a gradient (0-10%) of MeOH/EtOAc, to give amine 35 (1.93 g, 95%) as a cream powder: mp (MeOH/EtOAc)>290° C.; ¹H NMR δ 11.16 (br s, 1 H, NH), 8.63 (dd, J=4.5, 1.6 Hz, 2 H, H-2′, H-6′), 8.27 (ddd, J=9.3, 3.1, 2.0 Hz, 2 H, H-3″, H-5″), 7.96 (ddd, J=9.3, 3.1, 2.0 Hz, 2 H, H-2″, H-6″), 7.90-7.93 (m, 3 H, H-5, H-3′, H-5′); ¹³C NMR δ 162.2, 150.0 (2), 147.9, 146.6, 140.6, 140.3, 125.5 (2), 120.0 (2), 116.4 (2), 109.7. Anal. calcd for C₁₄H₁₀N₄O₂S.¼EtOAc: C, 56.24; H, 3.78; N, 17.49.Found: C, 56.49; H, 4.08; N, 17.07%.

### Example 1-18

### N-{4-[2-(Dimethylamino)ethyl]phenyl}-4-(4-pyridinyl)-1,3-thiazol-2-amine (36)

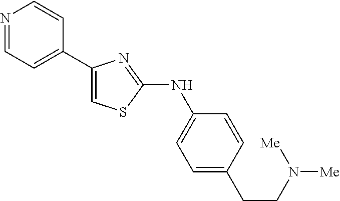

Methanesulfonyl chloride (77 μL, 1.00 mmol) was added dropwise to a stirred solution of alcohol 23 (270 mg, 0.91 mmol) and iPr₂NEt (190 μL, 1.09 mmol) in dry DCM (30 mL) and the solution was stirred at 20° C. for 4 h. The solution was diluted with DCM (50 mL) and washed with 1 M HCl (10 mL), saturated aqueous KHCO₃ solution (20 mL), brine (10 mL), dried, and the solvent evaporated. The residue was dissolved in DMF (5 mL) and anhydrous Me₂NH gas was bubbled through the solution for 5 min and the solution stirred at 20° C. for 16 h. The solvent was evaporated and the residue was purified by column chromatography, eluting with a gradient (0-20%) of MeOH/EtOAc, followed by 1% aqueous NH₃/20% MeOH/EtOAc, to give amine 36 (165 mg, 56%) as a oil: ¹H NMR δ 10.25 (br s, 1 H, NH), 8.61 (dd, J=4.5, 1.6 Hz, 2 H, H-2′, H-6′), 7.84 (dd, J=4.5, 1.6 Hz, 2 H, H-3′, H-5′), 7.66 (s, 1 H, H-5), 7.61 (br d, J=8.5 Hz, 2 H, H-3″, H-5″), 7.20 (br d, J=8.5 Hz, 2 H, H-2″, H-6″), 2.66 (br dd, J=8.2, 7.1 Hz, 2 H, CH₂), 2.44 (br dd, J=8.2, 7.1 Hz, 2 H, CH₂N), 2.19 [s, 6 H, N(CH₃)₃]; ¹³C NMR δ 163.6, 150.0 (2), 147.6, 141.0, 138.8, 133.5, 129.0 (2), 119.8 (2), 117.0 (2), 107.0, 60.8, 44.9 (2), 33.5; MS m/z 325.5 (MH⁺, 100%). The hydrochloride salt was crystallized from MeOH/EtOAc: mp (MeOH/EtOAc) 234-237° C. Anal. calcd for C₁₈H₂₁ClN₄S.½CH₃OH: C, 58.95; H, 6.15; N, 14.86.Found: C, 58.96; H, 6.04; N, 15.06%.

### Example 1-19

### N¹-[4-(4-Pyridinyl)-1,3-thiazol-2-yl]-1,4-benzenediamine (37)

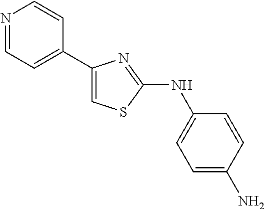

A mixture of amine 35 (0.20 g, 0.67 mmol) and Pd/C (100 mg) in EtOH (150 mL) was stirred under H₂ (60 psi) for 16 h. The mixture was cooled, filtered through Celite, washed with EtOH (3×20 mL) and the solvent evaporated to give diamine 37 (0.16 g, 90%) as a tan powder: mp (MeOH/EtOAc) 209-211° C.; ¹H NMR δ 9.79 (br s, 1 H, NH), 8.58 (dd, J=4.5, 1.6 Hz, 2 H, H-2″, H-6″), 7.80 (dd, J=4.5, 1.6 Hz, 2 H, H-3″, H-5″), 7.54 (s, 1 H, H-5′), 7.28 (ddd, J=8.7, 3.1, 2.0 Hz, 2 H, H-2, H-6), 6.57 (ddd, J=8.7, 3.1, 2.0 Hz, 2 H, H-3, H-5), 4.88 (br s, 2 H, NH₂); ¹³C NMR δ 165.6, 150.0 (2), 147.7, 144.4, 141.2, 120.2 (2), 119.8 (2), 115.3, 114.3 (2), 105.9. Anal. calcd for C₁₄H₁₂N₄S: C, 62.66; H, 4.51.Found: C, 62.64; H, 5.00%.

### Example 1-20

### 3-{[4-(4-Pyridinyl)-1,3-thiazol-2-yl]amino}phenyl Dihydrogen Phosphate (39)

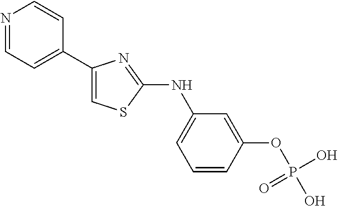

Di(tert-butyl) 3-{[4-(4-Pyridinyl)-1,3-thiazol-2-yl]amino}phenyl Phosphate (38). A solution of tetrazole (3% w/w, 14.4 mL, 4.9 mmol) in MeCN was added to a stirred solution of phenol 13 (450 mg, 1.7 mmol) and di-tert-butyl diisopropylphosphoramidite (0.68 mL, 2.2 mmol) in dry THF (15 mL) and the solution stirred at 20° C. under N₂ for 16 h. The solution was cooled to −40° C. and a dried solution of MCPBA (50%, 0.82 g, 2.4 mmol) in DCM (10 mL) was added and the solution stirred at −40° C. for 15 minutes. A solution of NaHSO₃ (10%, 2 mL) was added and the mixture partitioned between water and DCM. The organic fraction was washed with dilute aqueous ammonia, dried, and the solvent evaporated. The residue was purified by chromatography, eluting with a gradient (50-100%) of EtOAc/pet. ether, to give diester 38 (0.36 g, 46%) as a soft solid: ¹H NMR δ 8.63 (dd, J=4.7, 1.4 Hz, 2 H, H-2″, H-6″), 7.72 (dd, J=4.7, 1.4 Hz, 2 H, H-3″, H-5″), 7.53 (br s, 1 H, H-2), 7.24 (dd, J=8.1, 7.8 Hz, 1 H, H-5), 7.18 (br d, J=7.8 Hz, 1 H, H-6), 7.03 (s, 1 H, H-5′), 6.92 (dd, J=8.1, 1.0 Hz, 1 H, H-4), 1.54 (s, 18 H, 2×OtBu), NH not observed.

3-{[4-(4-Pyridinyl)-1,3-thiazol-2-yl]amino}phenyl Dihydrogen Phosphate (39). Trifluoroacetic acid (0.59 mL, 7.7 mmol) was added to a stirred solution of phosphate diester 38 (355 mg, 0.77 mmol) in DCM (20 mL) and the solution stirred at 20° C. for 16 h. The solvent was evaporated and the residue triturated with MeOH to give the phosphate as the trifluoroacetate salt 39 (127 mg, 36%) as a white solid, mp (MeOH) 258-261° C.; ¹H NMR δ 10.45 (br s, 1 H, NH), 8.61 (br d, J=5.0 Hz, 2 H, H-2″, H-6″), 7.94 (br d, J=5.0 Hz, 2 H, H-3″, H-5″), 7.84 (br s, 1 H, H-2), 7.77 (s, 1 H, H-5′), 7.38 (br d, J=8.0 Hz, 1 H, H-6), 7.29 (br t, J=8.0 Hz, 1 H, H-5), 6.56 (br d, J=8.0 Hz, 1 H, H-4), 2×OH not observed.

### Example 1-21

### 4-{[4-(4-Pyridinyl)-1,3-thiazol-2-yl]amino}phenyl Dihydrogen Phosphate (41)

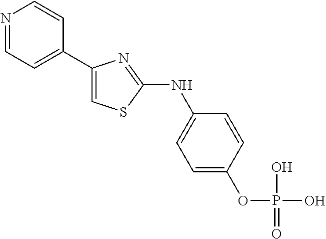

Di(tert-butyl) 4-{[4-(4-Pyridinyl)-1,3-thiazol-2-yl]amino}phenyl Phosphate (40). A solution of tetrazole (3% w/w, 34.5 mL, 14.8 mmol) in MeCN was added to a stirred solution of alcohol 25 (1.08 g, 4.0 mmol) and di-tert-butyl diisopropylphosphoramidite (1.64 mL, 5.2 mmol) in dry THF (50 mL) and the solution stirred at 20° C. under N₂ for 16 h. The solution was cooled to −40° C. and a dried solution of MCPBA (50%, 1.66 g, 4.8 mmol) in DCM (10 mL) was added and the solution stirred at −40° C. for 15 minutes. A solution of NaHSO₃ (10%, 2 mL) was added and the mixture partitioned between water and DCM. The organic fraction was washed with dilute aqueous ammonia, dried, and the solvent evaporated. The residue was purified by chromatography, eluting with a gradient (50-100%) of EtOAc/pet. ether, to give diester 40 (550 mg, 30%) as a gum: ¹H NMR (CD₃OD) δ 8.50 (dd, J=4.7, 1.6 Hz, 2 H, H-2″, H-6″), 7.86 (dd, J=4.7, 1.6 Hz, 2 H, H-3″, H-5″), 7.69 (ddd, J=8.9, 3.0, 1.9 Hz, 2 H, H-3, H-5), 7.38 (s, 1 H, H-5′), 7.16 (ddd, J=8.9, 3.0, 1.9 Hz, 2 H, H-2, H-6), 1.50 (s, 18 H, 2×OtBu); MS m/z 462.9 (MH⁺, 100%).

4-{[4-(4-Pyridinyl)-1,3-thiazol-2-yl]amino}phenyl Dihydrogen Phosphate (41). Trifluoroacetic acid (1.2 mL, 15.6 mmol) was added to a stirred solution of phosphate diester 40 (476 mg, 1.05 mmol) in DCM (20 mL) and the solution stirred at 20° C. for 3 h. The solvent was evaporated and the residue triturated with ether to give phosphate 41 (430 mg, 88%) as a tan gum: ¹H NMR δ 11.50 (br s, 2 H, 2×OH), 10.30 (br s, 1 H, NH), 8.62 (d, J=6.0 Hz, 2 H, H-2″, H-6″), 7.88 (d, J=6.0 Hz, 2 H, H-3″, H-5″), 7.88 (d, J=8.9 Hz, 2 H, H-3, H-5), 7.71 (s, 1 H, H-5′), 7.17 (d, J=8.9, 3.0, 1.9 Hz, 2 H, H-2, H-6).

### Example 1-22

### N-(3,4-Dimethylphenyl)-4-(4-pyridinyl)-1,3-thiazol-2-amine (43)

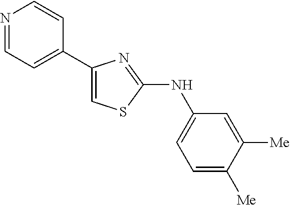

3,4-Dimethylphenylthiourea (42). NH₄SCN (6.3 g, 83.3 mmol) was added to a stirred solution of 3,4-dimethylaniline (10.1 g, 83.3 mmol) in 1 M HCl (84 mL) at 100° C. and the solution stirred at 100° C. for 16 h. The solution was diluted with water (60 mL) and stood at 5° C. for 2 h. The precipitate was filtered, washed with water (5 mL), washed with ether (5 mL), and dried to give thiourea 42 (2.04 g, 14%) as a white powder: mp (H₂O) 181-182° C. [lit. (Rasmussen, C. R. et al., Synthesis 1988, 456) mp (DME/Et₂O) 184-186° C.]; ¹H NMR δ 9.48 (s, 1 H, NH), 7.05-7.25 (m, 5 H, H-2, H-5, H-6, NH₂), 2.18 (s, 6 H, 2×CH₃).

N-(3,4-Dimethylphenyl)-4-(4-pyridinyl)-1,3-thiazol-2-amine (43). A mixture of bromoketone hydrobromide 1 (0.70 g, 2.5 mmol) and 3,4-dimethylphenylthiourea 42 (0.45 g, 2.5 mmol) in EtOH (20 mL) was stirred at reflux temperature for 3 h. The mixture was cooled to 20° C., diluted with water (50 mL), the pH adjusted to ca. 8 with aqueous NH₃ and the mixture stirred at 0° C. for 1 h. The precipitate was filtered, washed with water (5 mL) and dried. The crude solid was purified by column chromatography, eluting with 50% EtOAc/pet. ether, to give amine 43 (0.59 g, 85%) as a cream powder: mp (MeOH/EtOAc) 177-179° C.; ¹H NMR δ 10.15 (br s, 1 H, NH), 8.61 (dd, J=4.5, 1.6 Hz, 2 H, H-2′, H-6′), 7.83 (dd, J=4.5, 1.6 Hz, 2 H, H-3′, H-5′), 7.64 (s, 1 H, H-5), 7.47 (dd, J=8.1, 2.2 Hz, 1 H, H-6″), 7.42 (d, J=2.2 Hz, 1 H, H-2″), 7.10 (d, J=8.1 Hz, 1 H, H-5″), 2.34 (s, 3 H, CH₃), 2.18 (s, 3 H, CH₃); ¹³C NMR δ 163.8, 150.0 (2), 147.6, 141.0, 138.7, 136.5, 129.8, 129.2, 119.7 (2), 118.4, 114.6, 106.8, 19.7, 18.5; MS m/z 282.5 (MH⁺, 100%). Anal. calcd for C₁₆H₁₅N₃S.¼CH₃OH: C, 67.45; H, 5.57; N, 14.52.Found: C, 67.54; H, 5.61; N, 14.58%.

### Example 1-23

### N-(3,5-Dimethylphenyl)-4-(4-pyridinyl)-1,3-thiazol-2-amine (45)

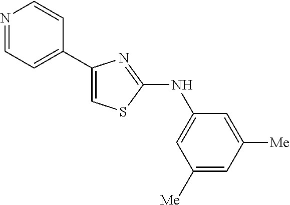

3,5-Dimethylphenylthiourea (44). NH₄SCN (2.51 g, 33.0 mmol) was added to a stirred solution of m-toluidine (4.00 g, 33.0 mmol) in 1 M HCl (35 mL) at 100° C. and the solution stirred at 100° C. for 16 h. The solution was diluted with water (60 mL) and stood at 5° C. for 2 h. The precipitate was filtered, washed with water (5 mL), washed with ether (5 mL), and dried to give thiourea 44 (1.61 g, 17%) as a white powder: mp (H₂O) 174-176° C. [lit. (Rasmussen, C. R. et al., Synthesis 1988, 456) mp (iPrOH) 172-173° C.]; ¹H NMR δ 9.52 (s, 1 H, NH), 7.36 (br s, 2 H, NH₂), 6.96 (s, 2 H, H-2, H-6), 6.76 (s, 1 H, H-4), 2.24 (s, 6 H, 2×CH₃); 13C NMR δ 180.7, 138.6, 137.7 (2), 126.0, 120.7 (2), 20.8 (2); MS m/z 181.4 (MH⁺, 100%).

N-(3,5-Dimethylphenyl)-4-(4-pyridinyl)-1,3-thiazol-2-amine (45). A mixture of bromoketone hydrobromide 1 (842 mg, 3.0 mmol) and thiourea 44 (540 mg, 3.0 mmol) in EtOH (30 mL) was stirred at reflux temperature for 3 h. The mixture was cooled to 20° C., diluted with water (50 mL), the pH adjusted to ca. 8 with aqueous NH₃ and the mixture stirred at 20° C. for 2 h. The precipitate was filtered, washed with water (5 mL) and dried. The crude solid was purified by column chromatography, eluting with a gradient (50-100%) of EtOAc/pet. ether, to give amine 45 (767 mg, 91%) as a cream powder: mp (EtOAc/pet. ether) 218-220° C.; ¹H NMR δ 10.21 (br s, 1 H, NH), 8.63 (dd, J=4.6, 1.6 Hz, 2 H, H-2′, H-6′), 7.86 (dd, J=4.6, 1.6 Hz, 2 H, H-3′, H-5′), 7.71 (s, 1 H, H-5), 7.33 (s, 2 H, H-2″, H-6″), 6.64 (s, 1 H, H-4″), 2.28 (s, 6 H, 2×CH₃); ¹³C NMR δ 163.6, 149.6 (2), 147.4, 140.7, 137.9 (2), 123.1, 119.9 (2), 114.8 (2), 107.6, 21.2 (2); MS m/z 282.4 (MH⁺, 100%). Anal. calcd for C₁₆H₁₅N₃S: C, 68.30; H, 5.37; N, 14.93.Found: C,67.91; H, 5.20; N, 14.92%.

### Example 1-24

### N-(2,3-Dihydro-1H-inden-5-yl)-4-(4-pyridinyl)-1,3-thiazol-2-amine (47)

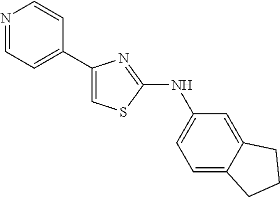

N-(2,3-Dihydro-1H-inden-5-yl)thiourea (46). NH₄SCN (2.87 g, 37.8 mmol) was added to a stirred solution of 5-indanamine (5.03 g, 37.8 mmol) in 1 M HCl (40 mL) at 100° C. and the solution stirred at 100° C. for 16 h. The solution was diluted with water (60 mL) and stood at 5° C. for 2 h. The precipitate was filtered, washed with water (5 mL), washed with ether (5 mL), and dried to give thiourea 46 (2.21 g, 30%) as a tan powder: mp (H₂O) 163-166° C. [lit. (Rasmussen et al., Synthesis 1988, 456) mp (acetone) 176-179° C.]; ¹H NMR δ 9.52 (s, 1 H, NH), 7.27 (br s, 2 H, NH₂), 7.21 (br s, 1 H, H-4), 7.16 (d, J=8.0 Hz, 1 H, H-7), 7.04 (br dd, J=8.0, 1.6 Hz, 1 H, H-6), 2.79-2.87 (m, 4 H, H-1, H-3), 1.96-2.05 (m, 2 H, H-2).

Alternative Preparation of N-(2,3-Dihydro-1H-inden-5-yl)thiourea (46). Benzoyl chloride (6.3 mL, 54.5 mmol) was added dropwise to a solution of NH₄SCN (4.44 g, 58.4 mmol) in dry acetone (50 mL). The mixture was stirred at reflux temperature for 15 min. The heating was removed and a solution of 5-indanamine (5.18 g, 38.9 mmol) in acetone (20 mL) was added dropwise keeping the solution at reflux. The mixture was stirred at reflux temperature for a further 30 min, cooled to 20° C. and poured onto ice. The mixture was stirred for 30 min and the precipitate was filtered and washed with water (15 mL) and dried. The solid was added to a solution of NaOH (10% w/v, 100 mL) at 80° C. and stirred at 80° C. for 30 min, then cooled to 20° C. and poured onto ice/HCl (6 M, 50 mL). The pH of the mixture was adjusted to 10 with cNH₃ solution and stirred for 30 min. The precipitate was filtered and washed with water (20 mL) and dried. The precipitate was purified by column chromatography, eluting with a gradient (30-100%) of EtOAc/pet. ether, to give thiourea 46 (5.91 g, 79%) as cream needles: mp (EtOAc/pet ether) 175-177° C. [lit. (Rasmussen et al., Synthesis 1988, 456) mp (acetone) 176-179° C.]; ¹H NMR δ 9.52 (s, 1 H, NH), 7.27 (br s, 2 H, NH₂), 7.21 (br s, 1 H, H-4), 7.16 (d, J=8.0 Hz, 1 H, H-7), 7.04 (br dd, J=8.0, 1.6 Hz, 1 H, H-6), 2.79-2.87 (m, 4 H, H-1, H-3), 1.96-2.05 (m, 2 H, H-2).

N-(2,3-Dihydro-1H-inden-5-yl)-4-(4-pyridinyl)-1,3-thiazol-2-amine (47). A mixture of bromoketone hydrobromide 1 (0.73 g, 2.60 mmol) and 1-(2,3-dihydro-1H-inden-5-yl)thiourea (46) (0.50 g, 2.60 mmol) in EtOH (25 mL) was stirred at reflux temperature for 3 h. The mixture was cooled to 20° C., diluted with water (50 mL), the pH adjusted to ca. 8 with aqueous NH₃ and the mixture stirred at 5° C. for 1 h. The mixture was filtered, washed with water (5 mL) and Et₂O (5 mL) and dried. The residue was purified by column chromatography, eluting with a gradient (0-10%) of MeOH/EtOAc, to give amine 47 (0.66 g, 87%) as a tan powder: mp (MeOH/EtOAc) 190-192° C.; ¹H NMR δ 10.18 (s, 1 H, NH), 8.61 (dd, J=4.5, 1.6 Hz, 2 H, H-2′, H-6′), 7.83 (dd, J=4.5, 1.6 Hz, 2 H, H-3′, H-5′), 7.65 (s, 1 H, H-5), 7.56 (br s, 1 H, H-4″), 7.45 (dd, J=8.1, 2.0 Hz, 1 H, H-6″), 7.19 (d, J=8.1 Hz, 1 H, H-7″), 2.89 (br t, J=7.4 Hz, 2 H, H-1″), 2.82 (br t, J=7.3 Hz, 2 H, H-3″), 2.03 (pent, J=7.4 Hz, 2 H, H-2″); ¹³C NMR δ 163.9, 150.0 (2), 147.6, 144.4, 141.0, 139.3, 136.8, 124.3, 119.8 (2), 115.4, 113.3, 106.8, 32.5, 31.6, 25.1; MS m/z 294.4 (MH⁺, 100%). Anal. calcd for C₁₇H₁₅N₃S.½CH₃OH: C, 67.93; H, 5.53; N, 13.58.Found: C, 67.58; H, 5.21; N, 13.53%.

### Example 1-25

### 2-Methyl-5-{[4-(4-pyridinyl)-1,3-thiazol-2-yl]amino}phenol (49)

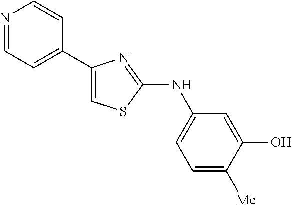

3-Hydroxy-4-methylphenylthiourea (48). NH₄SCN (3.78 g, 49.6 mmol) was added to a stirred solution of 3-hydroxy-4-methoxyaniline (6.11 g, 49.6 mmol) in 1 M HCl (50 mL) at 100° C. and the solution stirred at 100° C. for 16 h. The solution was diluted with water (60 mL) and stood at 5° C. for 2 h. The precipitate was filtered, washed with water (5 mL), washed with ether (5 mL), and dried to give thiourea 48 (3.34 g, 37%) as a tan powder: ¹H NMR δ 9.46 (s, 1 H, NH), 9.34 (s, 1 H, OH), 7.25 (br s, 2 H, NH₂), 6.98 (d, J=8.0 Hz, 1 H, H-5), 6.74 (d, J=2.0 Hz, 1 H, H-2), 6.63 (dd, J=8.0, 2.0 Hz, 1 H, H-6), 2.07 (s, 3 H, CH₃).

2-Methyl-5-{[4-(4-pyridinyl)-1,3-thiazol-2-yl]amino}phenol (49). A mixture of bromoketone hydrobromide 1 (1.23 g, 4.39 mmol) and 3-hydroxy-4-methylphenylthiourea (48) (0.80 g, 4.39 mmol) in EtOH (25 mL) was stirred at reflux temperature for 3 h. The mixture was cooled to 20° C., diluted with water (50 mL), the pH adjusted to ca. 8 with aqueous NH₃ and the mixture stirred at 5° C. for 1 h. The mixture was filtered, washed with water (5 mL) and Et₂O (5 mL) and dried. The residue was purified by column chromatography, eluting with a gradient (50-80%) of EtOAc/pet. ether, to give phenol 49 (0.94 g, 74%) as a white powder: mp (EtOAc/pet. ether) 213-215° C.; ¹H NMR δ 10.10 (s, 1 H, NH), 9.35 (br s, 1 H, OH), 8.60 (dd, J=4.5, 1.6 Hz, 2 H, H-2″, H-6″), 7.89 (dd, J=4.5, 1.6 Hz, 2 H, H-3″, H-5″), 7.64 (s, 1 H, H-5′), 7.36 (d, J=2.1 Hz, 1 H, H-2), 6.99 (d, J=8.4 Hz, 1 H, H-6), 6.91 (dd, J=8.4, 2.1 Hz, 1 H, H-5), 2.08 (s, 3 H, CH₃); ¹³C NMR δ 163.6, 155.5, 149.9 (2), 147.6, 141.0, 139.5, 130.4, 119.9 (2), 117.0, 107.9, 106.8, 104.1, 15.3; MS m/z 284.9 (MH⁺, 100%). Anal. calcd for C₁₅H₁₃N₃OS: C, 63.58; H, 4.62; N, 14.83.Found: C, 63.48; H, 4.71; N, 14.89%.

### Example 1-26

### 2-Methoxy-5-{[4-(4-pyridinyl)-1,3-thiazol-2-yl]amino}phenol (51)

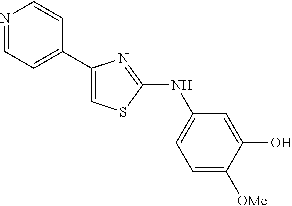

3-Hydroxy-4-methoxyphenylthiourea (50). NH₄SCN (2.65 g, 34.8 mmol) was added to a stirred solution of 3-hydroxy-4-methoxyaniline (4.84 g, 34.8 mmol) in 1 M HCl (40 mL) at 100° C. and the solution stirred at 100° C. for 16 h. The solution was diluted with water (60 mL) and stood at 5° C. for 2 h. The precipitate was filtered, washed with water (5 mL), washed with ether (5 mL), and dried to give thiourea 50 (3.10 g, 45%) as a tan powder: ¹H NMR δ 9.37 (s, 1 H, NH), 9.07 (s, 1 H, OH), 7.20 (br s, 2 H, NH₂), 6.85 (d, J=8.6 Hz, 1 H, H-5), 6.78 (d, J=2.5 Hz, 1 H, H-2), 6.66 (dd, J=8.6, 2.5 Hz, 1 H, H-6), 3.74 (s, 3 H, OCH₃).

2-Methoxy-5-{[4-(4-pyridinyl)-1,3-thiazol-2-yl]amino}phenol (51). A mixture of bromoketone hydrobromide 1 (1.40 g, 4.99 mmol) and 3-hydroxy-4-methoxyphenylthiourea (50) (0.96 g, 4.99 mmol) in EtOH (30 mL) was stirred at reflux temperature for 3 h. The mixture was cooled to 20° C., diluted with water (50 mL), the pH adjusted to ca. 8 with aqueous NH₃ and the mixture stirred at 5° C. for 1 h. The mixture was filtered, washed with water (5 mL) and Et₂O (5 mL) and dried. The residue was purified by column chromatography, eluting with a gradient (50-100%) of EtOAc/pet. ether, to give phenol 51 (1.06 g, 71%) as a white powder: mp (EtOAc) 230-232° C.; ¹H NMR δ 10.02 (s, 1 H, NH), 9.10 (br s, 1 H, OH), 8.60 (dd, J=4.6, 1.6 Hz, 2 H, H-2″, H-6″), 7.85 (dd, J=4.6, 1.6 Hz, 2 H, H-3″, H-5″), 7.61 (s, 1 H, H-5′), 7.29 (d, J=2.6 Hz, 1 H, H-2), 7.03 (dd, J=8.7, 2.6 Hz, 1 H, H-6), 6.89 (d, J=8.7 Hz, 1 H, H-5), 3.74 (s, 3 H, OCH₃); ¹³C NMR δ 163.9, 149.9 (2), 147.6, 146.8, 142.8, 141.0, 134.9, 119.8 (2), 113.2, 107.9, 106.5, 106.0, 56.0; MS m/z 300.4 (MH⁺, 100%). Anal. calcd for C₁₅H₁₃N₃O₂S.¼CH₃OH: C, 59.60; H, 4.59; N, 13.67.Found: C, 59.67; H, 4.51; N, 13.83%.

### Example 1-27

### 2-Methyl-4-{[4-(4-pyridinyl)-1,3-thiazol-2-yl]amino}phenol (53)

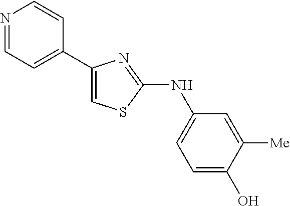

4-Hydroxy-3-methylphenylthiourea (52). NH₄SCN (2.56 g, 33.6 mmol) was added to a stirred solution of 4-hydroxy-3-methylaniline (4.14 g, 33.6 mmol) in 1 M HCl (35 mL) at 100° C. and the solution stirred at 100° C. for 16 h. The solution was diluted with water (60 mL) and stood at 5° C. for 2 h. The mixture was extracted with EtOAc (3×50 mL), the combined organic fraction dried and the solvent evaporated. The residue was purified by column chromatography, eluting with a gradient (50-60%) of EtOAc/pet. ether, to give thiourea 52 (0.77 g, 13%) as a tan powder: mp (EtOAc/pet. ether) 166-170° C.; ¹H NMR δ 9.27 (s, 1 H, NH), 9.23 (s, 1 H, OH), 7.11 (br s, 2 H, NH₂), 6.94 (br s, 1 H, H-2), 6.87 (br d, J=8.4 Hz, 1 H, H-5), 6.72 (d, J=8.4 Hz, 1 H, H-6), 2.09 (s, 3 H, CH₃). Anal. calcd for C₈H₁₀N₂OS: C, 52.72; H, 5.53; N, 15.37.Found: C, 52.71; H, 5.62; N, 15.19%.

2-Methyl-4-{[4-(4-pyridinyl)-1,3-thiazol-2-yl]amino}phenol (53). A mixture of bromoketone hydrobromide 1 (0.79 g, 2.81 mmol) and 1-(4-hydroxy-3-methylphenyl)thiourea (52) (0.51 g, 2.81 mmol) in EtOH (30 mL) was stirred at reflux temperature for 3 h. The mixture was cooled to 20° C., diluted with water (50 mL), the pH adjusted to ca. 8 with aqueous NH₃ and the mixture stirred at 5° C. for 1 h. The mixture was filtered, washed with water (5 mL) and Et₂O (5 mL) and dried. The residue was purified by column chromatography, eluting with a gradient (50-100%) of EtOAc/pet. ether, to give phenol 53 (0.64 g, 80%) as a white powder: mp (EtOAc/pet. ether) 212-215° C.; ¹H NMR δ 9.89 (s, 1 H, NH), 9.00 (br s, 1 H, OH), 8.59 (dd, J=4.5, 1.6 Hz, 2 H, H-2″, H-6″), 7.81 (dd, J=4.5, 1.6 Hz, 2 H, H-3″, H-5″), 7.56 (s, 1 H, H-5′), 6.35 (dd, J=8.5, 2.7 Hz, 1 H, H-5), 7.29 (d, J=2.7 Hz, 1 H, H-3), 6.77 (d, J=8.5 Hz, 1 H, H-6), 2.15 (s, 3 H, CH₃); ¹³C NMR δ 164.8, 150.6, 150.0 (2), 147.6, 141.0, 132.7, 124.1, 120.9, 119.7 (2), 116.7, 114.8, 106.1, 16.2; MS m/z 284.3 (MH⁺, 100%). Anal. calcd for C₁₅H₁₃N₃OS: C, 63.58; H, 4.62; N, 14.83.Found: C, 63.88; H, 4.69; N, 14.85%.

### Example 1-28

### N-(3,4-Dimethoxyphenyl)-4-(4-pyridinyl)-1,3-thiazol-2-amine (55)

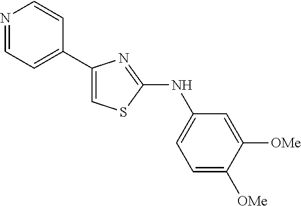

3,4-Dimethoxyphenylthiourea (54). NH₄SCN (2.48 g, 32.6 mmol) was added to a stirred solution of 3,4-dimethoxymethylaniline (5.00 g, 32.6 mmol) in 1 M HCl (35 mL) at 100° C. and the solution stirred at 100° C. for 16 h. The solution was diluted with water (60 mL) and stood at 5° C. for 2 h. The precipitate was filtered, washed with water (5 mL), washed with ether (5 mL), and dried to give thiourea 54 (2.33 g, 34%) as a tan powder: ¹H NMR δ 9.48 (s, 1 H, NH), 7.25 (br s, 2 H, NH₂), 7.00 (br s, 1 H, H-2), 6.90 (d, J=8.6 Hz, 1 H, H-5), 6.79 (dd, J=8.6, 2.3 Hz, 1 H, H-6), 3.73 (s, 6 H, 2×OCH₃).

N-(3,4-Dimethoxyphenyl)-4-(4-pyridinyl)-1,3-thiazol-2-amine (55). A mixture of bromoketone hydrobromide 1 (1.08 g, 3.84 mmol) and 3,4-methoxyphenylthiourea (54) (0.82 g, 3.84 mmol) in EtOH (25 mL) was stirred at reflux temperature for 3 h. The mixture was cooled to 20° C., diluted with water (50 mL), the pH adjusted to ca. 8 with aqueous NH₃ and the mixture stirred at 5° C. for 1 h. The mixture was filtered, washed with water (5 mL) and Et₂O (5 mL) and dried. The residue was purified by column chromatography, eluting with a gradient (50-100%) of EtOAc/pet. ether, to give amine 55 (1.04 g, 87%) as a cream powder: mp (EtOAc/pet. ether) 192-194° C.; ¹H NMR δ 10.16 (s, 1 H, NH), 8.60 (dd, J=4.5, 1.6 Hz, 2 H, H-2′, H-6′), 7.82 (dd, J=4.5, 1.6 Hz, 2 H, H-3′, H-5′), 7.63 (s, 1 H, H-5), 7.49 (d, J=2.5 Hz, 1 H, H-2″), 7.17 (dd, J=8.7, 2.5 Hz, 1 H, H-6″), 6.95 (d, J=8.7 Hz, 1 H, H-5″), 3.81 (s, 3 H, OCH₃), 3.74 (s, 3 H, OCH₃); ¹³C NMR δ 163.9, 150.2 (2), 148.9, 147.5, 143.7, 141.0, 134.9, 119.7 (2), 112.8, 108.9, 106.7, 102.8, 55.8, 55.2; MS m/z 314.4 (MH⁺, 100%). Anal. calcd for C₁₆H₁₅N₃O₂S: C, 61.32; H, 4.82; N, 13.41.Found: C, 61.24; H, 4.79; N, 13.45%.

### Example 1-29

### N-(3,5-Dimethoxyphenyl)-4-(4-pyridinyl)-1,3-thiazol-2-amine (57)

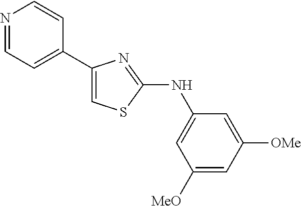

3,5-Dimethoxyphenylthiourea (56). NH₄SCN (2.51 g, 33.0 mmol) was added to a stirred solution of 3,5-dimethoxyaniline (5.06 g, 33.0 mmol) in 1 M HCl (35 mL) at 100° C. and the solution stirred at 100° C. for 16 h. The solution was diluted with water (60 mL) and stood at 5° C. for 2 h. The precipitate was filtered, washed with water (5 mL), washed with ether (5 mL), and dried to give thiourea 56 (2.92 g, 42%) as a white powder: mp (EtOAc/pet. ether) 173-175° C. ¹H NMR δ 9.61 (s, 1 H, NH), 7.44 (br s, 2 H, NH₂), 6.63 (d, J=2.2 Hz, 2 H, H-2, H-6), 6.26 (t, J=2.2 Hz, 1 H, H-4), 3.72 (s, 6 H, 2×OCH₃); ¹³C NMR δ 180.7, 160.3 (2), 140.5, 100.7 (2), 96.3, 55.0 (2); MS m/z 213.4 (MH⁺, 100%). Anal. calcd for C₉H₁₂N₂O₂S: C, 50.92; H, 5.70; N, 13.20.Found: C, 51.02; H, 5.76; N, 13.24%.

N-(3,5-Dimethoxyphenyl)-4-(4-pyridinyl)-1,3-thiazol-2-amine (57). A mixture of bromoketone hydrobromide 1 (842 mg, 3.0 mmol) and thiourea 56 (636 mg, 3.0 mmol) in EtOH (30 mL) was stirred at reflux temperature for 3 h. The mixture was cooled to 20° C., diluted with water (50 mL), the pH adjusted to ca. 8 with aqueous NH₃ and the mixture stirred at 5° C. for 2 h. The precipitate was filtered, washed with water (5 mL) and dried. The crude solid was purified by column chromatography, eluting with a gradient (50-100%) of EtOAc/pet. ether, to give amine 57 (860 mg, 91%) as a cream powder: mp (EtOAc/pet. ether) 229-231° C.; ¹H NMR δ 10.34 (br s, 1 H, NH), 8.62 (dd, J=4.5, 1.6 Hz, 2 H, H-2′, H-6′), 7.82 (dd, J=4.5, 1.6 Hz, 2 H, H-3′, H-5′), 7.72 (s, 1 H, H-5), 6.96 (d, J=2.2 Hz, 2 H, H-2″, H-6″), 6.17 (t, J=2.2 Hz,1 H, H-4″), 3.77 (s, 6 H, 2×OCH₃); ¹³C NMR δ 163.6, 160.7 (2), 150.2 (2), 147.6, 142.4, 140.9, 119.7 (2), 107.6, 95.5 (2), 93.6, 59.4 (2); MS m/z 314.5 (MH⁺, 100%). Anal. calcd for C₁₆H₁₅N₃O₂S.¼H₂O: C, 60.46; H, 4.92; N, 13.22.Found: C, 60.65; H, 4.75; N, 13.51%.

### Example 1-30

### N-(1,3-Benzodioxol-5-yl)-4-(4-pyridinyl)-1,3-thiazol-2-amine (59)

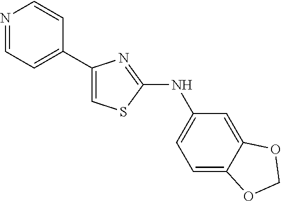

N-(1,3-Benzodioxol-5-yl)thiourea (58). NH₄SCN (2.78 g, 36.5 mmol) was added to a stirred solution of benzo[d][1,3]dioxol-5-amine (5.00 g, 36.5 mmol) in 1 M HCl (40 mL) at 100° C. and the solution stirred at 100° C. for 16 h. The solution was diluted with water (60 mL) and stood at 5° C. for 2 h. The precipitate was filtered, washed with water (5 mL), washed with ether (5 mL), and dried to give thiourea 58 (2.50 g, 35%) as a tan powder: mp (H₂O) 190-193° C. [lit. (Rasmussen et al., Synthesis 1988, 456) mp (MeOH) 209-211° C.]; ¹H NMR δ 9.46 (s, 1 H, NH), 7.47 (br s, 2 H, NH₂), 7.00 (br d, J=2.1 Hz, 1 H, H-4), 6.85 (d, J=8.3 Hz, 1 H, H-7), 6.69 (dd, J=8.3, 2.1 Hz, 1 H, H-6), 6.00 (s, 2 H, H-2).

N-(1,3-Benzodioxol-5-yl)-4-(4-pyridinyl)-1,3-thiazol-2-amine (59). A mixture of bromoketone hydrobromide 1 (0.73 g, 2.61 mmol) and 1-(benzo[d][1,3]dioxol-5-yl)thiourea (58) (0.51 g, 2.61 mmol) in EtOH (25 mL) was stirred at reflux temperature for 3 h. The mixture was cooled to 20° C., diluted with water (50 mL), the pH adjusted to ca. 8 with aqueous NH₃ and the mixture stirred at 5° C. for 1 h. The mixture was filtered, washed with water (5 mL) and Et₂O (5 mL) and dried. The residue was purified by column chromatography, eluting with 5% MeOH/DCM, to give amine 59 (0.71 g, 92%) as a tan powder: mp (MeOH/EtOAc) 201-203° C.; ¹H NMR δ 10.21 (s, 1 H, NH), 8.60 (dd, J=4.5, 1.6 Hz, 2 H, H-2′, H-6′), 7.81 (dd, J=4.5, 1.6 Hz, 2 H, H-3′, H-5′), 7.64 (s, 1 H, H-5), 7.44 (d, J=2.2 Hz, 1 H, H-4″), 7.08 (dd, J=8.4, 2.2 Hz, 1 H, H-6″), 6.90 (d, J=8.4 Hz, 1 H, H-5″), 6.00 (s, 2 H, H-2″); ¹³C NMR δ 163.8, 150.0 (2), 147.5, 147.3, 141.7, 140.9, 135.6, 119.7 (2), 109.7, 108.2, 106.9, 100.8, 99.6; MS m/z 298.3 (MH⁺, 100%). Anal. calcd for C₁₅H₁₁N₃O₂S.¾H₂O: C, 57.96; H, 4.05; N, 13.51.

Found: C, 58.03; H, 3.76; N, 13.16%.

### Example 1-31

### N-(3,4-Dichlorophenyl)-4-(4-pyridinyl)-1,3-thiazol-2-amine (61)

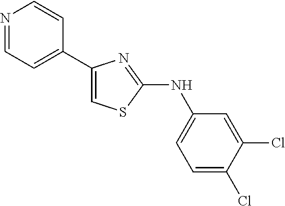

A mixture of bromoketone hydrobromide 1 (0.69 g, 2.44 mmol) and 3,4-dichlorophenylthiourea (60) (0.54 g, 2.44 mmol) in EtOH (25 mL) was stirred at reflux temperature for 2 h. The mixture was cooled to 20° C., diluted with water (50 mL), the pH adjusted to ca. 8 with aqueous NH₃ and the mixture stirred at 5° C. for 1 h. The mixture was extracted with EtOAc (3×50 mL) and the combined organic fraction dried and the solvent evaporated. The residue was purified by column chromatography, eluting with a gradient (50-100%) of EtOAc/pet. ether, to give amine 61 (0.77 g, 97%) as a cream powder: mp (EtOAc) 242-244° C.; ¹H NMR δ 10.68 (br s, 1 H, NH), 8.63 (dd, J=4.5, 1.6 Hz, 2 H, H-2′, H-6′), 8.13 (d, J=2.5 Hz, 1 H, H-2″), 7.83 (dd, J=4.5, 1.6 Hz, 2 H, H-3′, H-5′), 7.80 (s, 1 H, H-5), 7.64 (dd, J=8.8, 2.5 Hz, 1 H, H-6″), 7.57 (d, J=8.8 Hz, 1 H, H-5″); ¹³C NMR δ 162.7, 150.1 (2), 147.6, 140.7, 140.6, 131.0, 130.7, 122.3, 119.7 (2), 117.8, 117.0, 108.4. Anal. calcd for C₁₄H₉Cl₂N₃S.¼EtOAc: C, 52.38; H, 3.27; N, 12.21.Found: C, 52.51; H, 3.51,N, 11.96%.

### Example 1-32

### 4-(4-Pyridinyl)-N-(3,4,5-trimethoxyphenyl)-1,3-thiazol-2-amine (63)

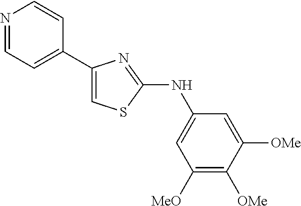

3,4,5-Trimethoxyphenylthiourea (62). NH₄SCN (2.51 g, 33.0 mmol) was added to a stirred solution of 3,4,5-trimethoxyaniline (6.04 g, 33.0 mmol) in 1 M HCl (35 mL) at 100° C. and the solution stirred at 100° C. for 16 h. The solution was diluted with water (60 mL) and stood at 5° C. for 2 h. The precipitate was filtered, washed with water (5 mL), washed with ether (5 mL), and dried to give thiourea 62 (1.18 g, 15%) as a white powder: mp (EtOAc/pet. ether) 201-203° C. [lit. (Protiva, M., et. al., Coll. Czech. Chem. Commun. 1975, 40, 3904.) mp 203-205° C.]; ¹H NMR δ 9.55 (s, 1 H, NH), 7.39 (br s, 2 H, NH₂), 6.70 (s, 2 H, H-2, H-6), 3.73 (s, 6 H, 2×OCH₃), 3.64 (s, 3 H, OCH₃); ¹³C NMR δ 180.6, 156.2 (2), 134.5, 134.4, 101.0 (2), 59.9, 55.7 (2); MS m/z 242 (MH⁺, 100%). Anal. calcd for C₁₀H₁₄N₂O₃S: C, 49.57; H, 5.82; N, 11.56.Found: C, 49.84; H, 5.80; N, 11.57%.

4-(4-Pyridinyl)-N-(3,4,5-trimethoxyphenyl)-1,3-thiazol-2-amine (63). A mixture of bromoketone hydrobromide 1 (842 mg, 3.0 mmol) and 3,4,5-trimethoxyphenylthiourea (62) (726 mg, 3.0 mmol) in EtOH (30 mL) was stirred at reflux temperature for 3 h. The mixture was cooled to 20° C., diluted with water (50 mL), the pH adjusted to ca. 8 with aqueous NH₃ and the mixture stirred at 5° C. for 2 h. The precipitate was filtered, washed with water (5 mL) and dried. The crude solid was purified by column chromatography, eluting with a gradient (50-100%) of EtOAc/pet. ether, to give amine 63 (215 mg, 21%) as a cream powder: mp (MeOH/EtOAc) 207-209° C.; ¹H NMR δ 10.34 (br s, 1 H, NH), 8.62 (dd, J=4.5, 1.6 Hz, 2 H, H-2″, H-6″), 7.82 (dd, J=4.5, 1.6 Hz, 2 H, H-3″, H-5″), 7.72 (s, 1 H, H-5), 6.96 (d, J=2.2 Hz, 2 H, H-2′, H-6′), 3.77 (s, 6 H, 2×OCH₃), 3.64 (s, 3 H, OCH₃); ¹³C NMR δ 163.5, 152.9, 150.0 (2), 147.4, 141.0, 137.0 (2), 132.1, 119.7 (2), 107.3, 94.9 (2), 60.0, 55.7 (2); MS m/z 314.5 (MH⁺, 100%). Anal. calcd for C₁₇H₁₇N₃O₃S.¼CH₃OH: C, 58.96; H, 5.16; N, 11.96.Found: C, 58.79; H, 5.08; N, 11.95%.

### Example 1-33

### N-(3-Methylphenyl)-4-(3-pyridinyl)-1,3-thiazol-2-amine (65)

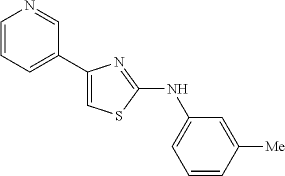

2-Bromo-1-(pyridin-3-yl)ethanone hydrobromide (64). Br₂ (2.0 mL, 38.6 mmol) was added dropwise to a stirred a solution of 3-acetylpyridine (4.45 g, 36.7 mmol) in 30% HBr/HOAc (75 mL) at 10-15° C. The mixture was stirred at 40° C. for 1 h and then 75° C. for 1 h. The mixture was cooled to 20° C., diluted with Et₂O (200 mL) and stirred for 30 min. The precipitate was filtered, washed with Et₂O (20 mL) and dried under vacuum to give bromoketone 64 as the hydrobromide salt (9.79 g, 95%) as a cream powder: mp 195-198° C. [lit. (McKennis, H. et al., J. Org. Chem. 1963, 28, 383.) mp (MeOH/ether) 185-188° C.]; ¹H NMR δ 9.23 (dd, J=2.2, 0.7 Hz, 1 H, H-2′), 8.91 (dd, J=5.1, 1.6 Hz, 1 H, H-6′), 8.53 (dd, J=8.1, 2.0 Hz, 1 H, H-4′), 7.78 (dd, J=8.1, 5.1 Hz, 1 H, H-5′), 7.70 (br s, 1 H, N.HBr), 5.02 (s, 2 H, H-2).

N-(3-Methylphenyl)-4-(3-pyridinyl)-1,3-thiazol-2-amine (65). A mixture of bromoketone hydrobromide 64 (1.17 g, 4.2 mmol) and 3-methylphenylthiourea (4) (0.64 g, 4.2 mmol) in EtOH (25 mL) was stirred at reflux temperature for 3 h. The mixture was cooled to 20° C., diluted with water (50 mL), the pH adjusted to ca. 8 with aqueous NH₃ and the mixture stirred at 0° C. for 1 h. The precipitate was filtered, washed with water (5 mL) and dried. The crude solid was purified by column chromatography, eluting with EtOAc, to give amine 65 (0.86 g, 77%) as a cream powder: mp (EtOAc) 171-173° C.; ¹H NMR δ 10.24 (br s, 1 H, NH), 9.13 (dd, J=2.2, 0.7 Hz, 1 H, H-2′), 8.51 (dd, J=4.7, 1.6 Hz, 1 H, H-6′), 8.24 (ddd, J=8.0, 2.1, 1.7 Hz, 1 H, H-4′), 7.57 (dd, J=8.1, 1.9 Hz, 1 H, H-6″), 7.50 (s, 1 H, H-5), 7.48 (d, J=1.9 Hz, 1 H, H-2″), 7.45 (ddd, J=8.0, 4.7, 0.7 Hz, 1 H, H-5′), 7.24 (dd, J=7.9, 7.5 Hz, 1 H, H-5″), 6.80 (br d, J=7.5 Hz, 1 H, H-4″), 2.32 (s, 3 H, CH₃); ¹³C NMR δ 163.6, 148.3, 147.1, 146.8, 140.9, 138.0, 132.6, 130.0, 128.8, 123.6, 122.1, 117.4, 114.9, 104.3, 21.2; MS m/z 268.4 (MH⁺, 100%). Anal. calcd for C₁₅H₁₃N₃: C, 67.39; H, 4.90; N, 15.72.Found: C, 67.20; H, 5.07; N, 15.77%.

### Example 1-34

### 4-{[4-(3-Pyridinyl)-1,3-thiazol-2-yl]amino}phenol (66)

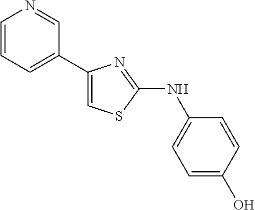

4-{[4-(3-Pyridinyl)-1,3-thiazol-2-yl]amino}phenol (66). A mixture of bromoketone hydrobromide 64 (0.84 g, 3.0 mmol) and 1-(4-hydroxyphenyl)thiourea (24) (0.50 g, 3.0 mmol) in EtOH (20 mL) was stirred at reflux temperature for 3 h. The mixture was cooled to 20° C., diluted with water (50 mL), the pH adjusted to ca. 8 with aqueous NH₃ and the mixture stirred at 0° C. for 1 h. The precipitate was filtered, washed with water (5 mL) and dried to give phenol 66 (0.77 g, 95%) as a cream powder: mp (H₂O) 275-278° C.; ¹H NMR δ 9.95 (br s, 1 H, NH), 9.10 (m, 2 H, H-2″, OH), 8.49 (dd, J=4.7, 1.6 Hz, 1 H, H-6″), 8.21 (ddd, J=8.3, 2.1, 1.6 Hz, 1 H, H-4″), 7.47 (ddd, J=8.9, 3.4, 2.2 Hz, 2 H, H-3, H-5), 7.44 (ddd, J=8.3, 4.7, 0.7 Hz, 1 H, H-5″), 7.38 (s, 1 H, H-5′), 6.77 (ddd, J=8.9, 3.4, 2.2 Hz, 2 H, H-2, H-6); ¹³C NMR δ 164.7, 152.4, 148.2, 147.1, 146.8, 133.0, 132.6, 130.2, 123.5, 119.3 (2), 115.4 (2), 103.3; MS m/z 270.4 (MH⁺, 100%). Anal. calcd for C₁₄H₁₁N₃OS.¼H₂O: C, 61.41; H, 4.23; N, 15.34.Found: C, 61.66; H, 4.45; N, 15.46%.

### Example 1-35

### N-(3-Methylphenyl)-4-(2-pyridinyl)-1,3-thiazol-2-amine (68)

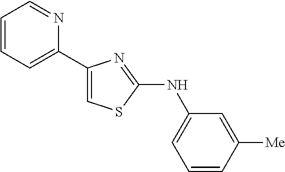

2-Bromo-1-(pyridin-2-yl)ethanone hydrobromide (67). Br₂ (4.1 mL, 80.6 mmol) was added dropwise to a stirred a solution of 2-acetylpyridine (9.30 g, 76.8 mmol) in 30% HBr/HOAc (150 mL) at 10-15° C. The mixture was stirred at 40° C. for 1 h and then 75° C. for 1 h. The mixture was cooled to 20° C., diluted with Et₂O (200 mL) and stirred for 30 min. The precipitate was filtered, washed with Et₂O (30 mL) and dried under vacuum to give the bromoketone hydrobromide salt 67 (19.46 g, 90%) as a cream powder: mp 200-205° C. (dec) [lit. (Ogawa, K. J. Chem. Soc. Perkin 1 1985, 2417.) mp 203-205° C.]; ¹H NMR δ 8.74 (ddd, J=4.5, 1.5, 1.0 Hz, 1 H, H-6′), 8.00-8.08 (m, 2 H, H-3′, H-5′), 7.71 (ddd, J=6.9, 4.5, 2.0 Hz, 1 H, H-4′), 7.70 (br s, 1 H, N.HBr), 5.01 (s, 2 H, H-2).

N-(3-Methylphenyl)-4-(2-pyridinyl)-1,3-thiazol-2-amine (68). A mixture of bromoketone hydrobromide 67 (0.96 g, 3.4 mmol) and 3-methylphenylthiourea (4) (0.57 g, 3.4 mmol) in EtOH (25 mL) was stirred at reflux temperature for 3 h. The mixture was cooled to 20° C., diluted with water (40 mL), the pH adjusted to ca. 8 with aqueous NH₃ and the mixture stirred at 0° C. for 1 h. The precipitate was filtered, washed with water (5 mL) and dried. The crude solid was purified by column chromatography, eluting with EtOAc, to give amine 68 (0.74 g, 81%) as a cream powder: mp (EtOAc) 164-166° C.; ¹H NMR δ 10.20 (br s, 1 H, NH), 8.58 (ddd, J=4.7, 1.7, 0.8 Hz, 1 H, H-6′), 7.99 (dt, J=7.9, 0.9 Hz, 1 H, H-3′), 7.99 (ddd, J=7.9, 7.5, 1.8 Hz, 1 H, H-4′), 7.58 (br d, J=8.1 Hz, 1 H, H-6′), 7.52 (s, 1 H, H-5), 7.47 (br s, 1 H, H-2″), 7.31 (ddd, J=7.5, 4.7, 1.2 Hz, 1 H, H-5′), 7.24 (t, J=7.8 Hz, 1 H, H-5″), 6.80 (br d, J=7.5 Hz, 1 H, H-4″), 2.33 (s, 3 H, CH₃); ¹³C NMR δ 163.3, 152.0, 150.2, 149.3, 141.0, 138.0, 137.1, 128.8, 122.4, 122.0, 120.2, 117.4, 114.0, 106.5, 21.2; MS m/z 268.4 (MH⁺, 100%). Anal. calcd for C₁₅H₁₃N₃: C, 67.39; H, 4.90; N, 15.72.Found: C, 67.13; H, 5.10; N, 15.67%.

### Example 1-36

### 4-{[4-(2-Pyridinyl)-1,3-thiazol-2-yl]amino}phenol (69)

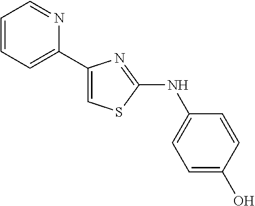

4-{[4-(2-Pyridinyl)-1,3-thiazol-2-yl]amino}phenol (69). A mixture of bromoketone hydrobromide 67 (0.78 g, 2.8 mmol) and 1-(4-hydroxyphenyl)thiourea (24) (0.46 g, 2.8 mmol) in EtOH (20 mL) was stirred at reflux temperature for 3 h. The mixture was cooled to 20° C., diluted with water (50 mL), the pH adjusted to ca. 8 with aqueous NH₃ and the mixture stirred at 0° C. for 1 h. The precipitate was filtered, washed with water (5 mL) and dried. The crude solid was purified by column chromatography, eluting with EtOAc, to give phenol 69 (0.67 g, 90%) as a cream powder: mp (EtOAc) 185-186° C.; ¹H NMR δ 9.91 (br s, 1 H, NH), 9.09 (s, 1 H, OH), 8.56 (ddd, J=4.7, 1.7, 0.8 Hz, 1 H, H-6″), 7.95 (br d, J=7.9 Hz, 1 H, H-3″), 7.87 (ddd, J=7.9, 7.5 Hz, 1 H, H-4″), 7.49 (ddd, J=8.9, 3.4, 2.2 Hz, 2 H, H-3, H-5), 7.43 (s, 1 H, H-5′), 7.30 (ddd, J=7.5, 4.7, 1.2 Hz, 1 H, H-5″), 6.77 (ddd, J=8.9, 3.4, 2.2 Hz, 2 H, H-2, H-6); ¹³C NMR δ 164.4, 152.3, 152.2, 150.2, 149.2, 137.0, 133.1, 122.3, 120.2, 119.2 (2), 115.4 (2), 105.5; MS m/z 270.4 (MH⁺, 100%). Anal. calcd for C₁₄H₁₁N₃OS: C, 62.43; H, 4.12; N, 15.60.

Found: C, 62.30; H, 4.37; N, 15.69%.

### Example 1-37

### 5-Methyl-N-(3-methylphenyl)-4-(4-pyridinyl)-1,3-thiazol-2-amine (71)

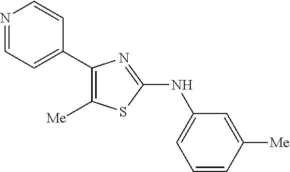

2-Bromo-1-(4-pyridinyl)-1-propanone Hydrobromide (70). Br₂ (4.0 mL, 78.6 mmol) was added dropwise to a stirred a solution of 4-propionylpyridine (10.1 g, 74.9 mmol) in 30% HBr/HOAc (80 mL) at 15° C. The mixture was stirred at 40° C. for 1 h and then at 80° C. for 1 h. The mixture was cooled to 20° C., diluted with Et₂O (400 mL) and stirred at 0° C. for 30 min. The precipitate was filtered, washed with Et₂O (25 mL) and dried under vacuum to give the bromoketone hydrobromide salt 70 (21.21 g, 96%) as a white powder: mp (Et₂O/H₂O) 195-197° C.; ¹H NMR δ 8.98 (dd, J=4.8, 1.6 Hz, 2 H, H-2′, H-6′), 8.15 (dd, J=4.8, 1.6 Hz, 2 H, H-3′, H-5′), 6.48 (br s, 1 H, N.HBr), 5.87 (q, J=6.5 Hz, 1 H, H-2), 1.81 (d, J=6.5 Hz, 3 H, H-3).

5-Methyl-N-(3-methylphenyl)-4-(4-pyridinyl)-1,3-thiazol-2-amine (71). A mixture of bromide 70 (0.92 g, 3.1 mmol) and 3-methylphenylthiourea (4) (0.52 g, 3.1 mmol) in EtOH (30 mL) was stirred at reflux temperature for 3 h. The mixture was cooled to 20° C., diluted with water (50 mL), the pH adjusted to ca. 9 with aqueous NH₃ and the mixture stirred at 5° C. for 2 h. The precipitate was filtered, washed with water (5 mL) and dried. The crude solid was purified by column chromatography, eluting with EtOAc, to give amine 71 (0.82 g, 94%) as a cream powder: mp (EtOAc) 193-195° C.; ¹H NMR δ 10.03 (s, 1 H, NH), 8.62 (dd, J=4.5, 1.6 Hz, 2 H, H-2′, H-6′), 7.67 (dd, J=4.5, 1.6 Hz, 2 H, H-3′, H-5′), 7.50 (br dd, J=8.0, 1.9 Hz, 1 H, H-6″), 7.38 (br s, 1 H, H-2″), 7.19 (dd, J=8.0, 7.5 Hz, 1 H, H-5″), 6.77 (br d, J=7.5 Hz, 1 H, H-4″), 2.30 (s, 6 H, 2×CH₃); ¹³C NMR δ 159.5, 149.7 (2), 142.3, 141.8, 140.9, 138.0, 128.7, 121.9 (2), 121.8, 120.5, 117.3, 113.9, 21.2, 11.9; MS m/z 282.5 (MH⁺, 100%). Anal. calcd for C₁₆H₁₅N₃S: C, 68.30; H, 5.37; N, 14.93.Found: C, 68.33; H, 5.51; N, 14.90%.

### Example 1-38

### N-(2,3-Dihydro-1H-inden-5-yl)-5-methyl-4-(4-pyridinyl)-1,3-thiazol-2-amine (72)

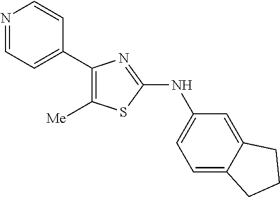

N-(2,3-Dihydro-1H-inden-5-yl)-5-methyl-4-(4-pyridinyl)-1,3-thiazol-2-amine (72). A mixture of bromide 70 (0.50 g, 1.7 mmol) and thiourea 46 (0.32 g, 1.7 mmol) in EtOH (30 mL) was stirred at reflux temperature for 2 h. The mixture was cooled to 20° C., diluted with water (60 mL), the pH adjusted to ca. 9 with aqueous NH₃ and the mixture stirred at 0° C. for 1 h. The precipitate was filtered, washed with water (5 mL) and dried. The crude solid was purified by column chromatography, eluting with 50% EtOAc/pet ether, to give amine 72 (0.47 g, 90%) as a tan powder: mp (EtOAc/Et₂O) 180-181° C.; ¹H NMR δ 9.95 (s, 1 H, NH), 8.63 (dd, J=4.5, 1.6 Hz, 2 H, H-2′, H-6′), 7.65 (dd, J=4.5, 1.6 Hz, 2 H, H-3′, H-5′), 7.48 (br s, 1 H, H-4″), 7.38 (dd, J=8.1, 2.0 H, 1 H, H-6″), 7.15 (d, J=8.1 Hz, 1 H, H-7″), 2.84 (br t, J=7.4 Hz, 2 H, H-3″), 2.79 (br t, J=7.4 Hz, 2 H, H-1″), 2.49 (s, 3 H, H-1′), 2.01 (br sept, J=7.4 Hz, 2 H, H-2″); ¹³C NMR δ 159.9, 149.7 (2), 144.3, 142.4, 141.9, 139.4, 136.4, 124.6, 121.9 (2), 120.0, 115.2, 113.1, 32.5, 31.6, 25.1, 11.9; MS m/z 308.5 (MH⁺, 100%). Anal. calcd for C₁₈H₁₇N₃S.Et₂O: C, 70.02; H, 6.03; N, 12.89.Found: C, 70.47; H, 6.02; N, 12.88%.

### Example 1-39

### [2-(Phenylamino)-4-(4-pyridinyl)-1,3-thiazol-5-yl]methanol (73)

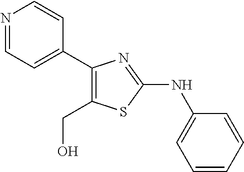

[2-(Phenylamino)-4-(4-pyridinyl)-1,3-thiazol-5-yl]methanol (73). A mixture of amine 3 (0.37 g, 1.46 mmol), 40% aqueous formaldehyde solution (5 mL) and Et₃N (1 mL) in THF (5 mL) was stirred in a glass pressure vessel at 130° C. for 1 h. The mixture was cooled to 20° C., the reaction quenched with cNH₃ solution (2 mL) and the mixture diluted with water (50 mL). The mixture was extracted with EtOAc (3×20 mL), the combined organic fraction was dried and the solvent evaporated. The crude solid was purified by column chromatography, eluting with a gradient (0-5%) of MeOH/DCM, to give (i) starting material 3 (71 mg, 19%) and (ii) alcohol 73 (258 mg, 62%) as a white powder: mp (MeOH/DCM) 170-172° C.; ¹H NMR δ 10.19 (br s, 1 H, NH), 8.64 (dd, J=4.5, 1.6 Hz, 2 H, H-2″, H-6″), 7.65-7.69 (m, 4 H, H-3″, H-5″, H-2′, H-6″), 7.33 (tt, J=7.4, 1.9 Hz, 2 H, H-3″, H-5″), 6.96 (br t, J=7.4 Hz, 1 H, H-4″), 5.72 (t, J=5.4 Hz, 1 H, OH), 4.70 (d, J=5.4 Hz, 2 H, H-1); ¹³C NMR δ 161.1, 149.8 (2), 142.7, 141.5, 140.9, 128.9 (2), 127.7, 122.1 (2), 121.2, 116.8 (2), 55.4; MS m/z 284.4 (MH⁺, 100%). Anal. calcd for C₁₅H₁₃N₃OS.H₂O: C, 59.78; H, 5.01; N, 13.94.Found: C, 60.12; H, 4.57; N, 13.92%.

### Example 1-40

### [2-[(3-Methylphenyl)amino]-4-(4-pyridinyl)-1,3-thiazol-5-yl]methanol (74)

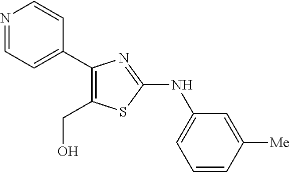

[2-[(3-Methylphenyl)amino]-4-(4-pyridinyl)-1,3-thiazol-5-yl]methanol (74). A mixture of amine 5 (1.02 g, 1.87 mmol), 40% aqueous formaldehyde solution (10 mL) and Et₃N (3 mL) in THF (10 mL) was stirred in a glass pressure vessel at 130° C. for 1 h. The mixture was cooled to 20° C., the reaction quenched with cNH₃ solution (6 mL) and the mixture diluted with water (100 mL). The mixture was extracted with EtOAc (3×50 mL), the combined organic fraction was dried and the solvent evaporated. The crude solid was purified by column chromatography, eluting with a gradient (0-5%) of MeOH/DCM, to give (i) starting material 5 (93 mg, 9%) and (ii) alcohol 74 (874 mg, 77%) as a white powder: mp (MeOH/DCM) 161-163° C.; ¹H NMR δ 10.12 (br s, 1 H, NH), 8.64 (dd, J=4.5, 1.6 Hz, 2 H, H-2″, H-6″), 7.65 (dd, J=4.5, 1.6 Hz, 2 H, H-3″, H-5″), 7.52 (dd, J=8.0, 1.8 Hz, 1 H, H-6″), 7.40 (br s, 1 H, H-2″), 7.21 (dd, J=8.0, 7.5 Hz, 1 H, H-5″), 6.79 (br d, J=7.5 Hz, 1 H, H-4″), 5.71 (t, J=5.5 Hz, 1 H, OH), 4.70 (d, J=5.5 Hz, 2 H, H-1), 2.30 (s, 3 H, CH₃); ¹³C NMR δ 161.2, 149.8 (2), 142.7, 141.5, 140.9, 138.0, 128.7, 127.6, 122.1 (2), 122.0, 117.4, 114.1, 55.4, 21.2; MS m/z 298.5 (MH⁺, 100%). Anal. calcd for C₁₆H₁₅N₃OS: C, 64.62; H, 5.08; N, 14.13.Found: C, 64.70; H, 5.18; N, 14.08%.

### Example 1-41

### [2-[(4-Methylphenyl)amino]-4-(4-pyridinyl)-1,3-thiazol-5-yl]methanol (75)

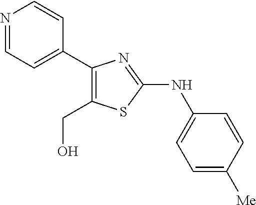

[2-[(4-Methylphenyl)amino]-4-(4-pyridinyl)-1,3-thiazol-5-yl]methanol (75). A mixture of amine 21 (0.25 g, 0.94 mmol), 40% aqueous formaldehyde solution (5 mL) and Et₃N (1 mL) in THF (5 mL) was stirred in a glass pressure vessel at 130° C. for 2 h. The mixture was cooled to 20° C., the reaction quenched with cNH₃ solution (2 mL) and the mixture diluted with water (50 mL). The mixture was extracted with EtOAc (3×20 mL), the combined organic fraction was dried and the solvent evaporated. The crude solid was purified by column chromatography, eluting with a gradient (0-5%) of MeOH/DCM, to give (i) starting material 21 (95 mg, 38%) and (ii) alcohol 75 (162 mg, 58%) as a white powder: mp (MeOH/DCM) 208-212° C. (dec.); ¹H NMR δ 10.08 (br s, 1 H, NH), 8.64 (dd, J=4.5, 1.6 Hz, 2 H, H-2″, H-6″), 7.64 (dd, J=4.5, 1.6 Hz, 2 H, H-3″, H-5″), 7.53 (br d, J=8.5 Hz, 2 H, H-2″, H-6″), 7.13 (br d, J=8.5 Hz, 2 H, H-3″, H-5″), 5.70 (t, J=5.4 Hz, 1 H, OH), 4.68 (d, J=5.4 Hz, 2 H, H-1), 2.25 (s, 3 H, CH₃); ¹³C NMR δ 161.4, 149.8 (2), 142.8, 141.5, 138.5, 130.1, 129.3 (2), 127.3, 122.1 (2), 117.1 (2), 55.4, 20.2; MS m/z 298.5 (MH⁺, 100%). Anal. calcd for C₁₆H₁₅N₃OS.⅓CH₂Cl₂: C, 60.24; H, 4.85; N, 12.90.Found: C, 60.05; H, 4.94; N, 12.82%.

### Example 1-42

### [2-(2,3-Dihydro-1H-inden-5-ylamino)-4-(4-pyridinyl)-1,3-thiazol-5-yl]methanol (76)

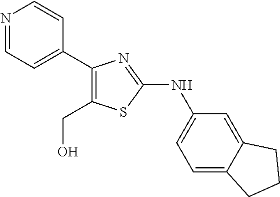

[2-(2,3-Dihydro-1H-inden-5-ylamino)-4-(4-pyridinyl)-1,3-thiazol-5-yl]methanol (76). A mixture of amine 47 (0.25 g, 0.77 mmol), 40% aqueous formaldehyde solution (5 mL) and Et₃N (1 mL) in THF (5 mL) was stirred in a glass pressure vessel at 130° C. for 2 h. The mixture was cooled to 20° C., the reaction quenched with cNH₃ solution (2 mL) and the mixture diluted with water (50 mL). The mixture was extracted with EtOAc (3×20 mL), the combined organic fraction was dried and the solvent evaporated. The crude solid was purified by column chromatography, eluting with a gradient (0-5%) of MeOH/DCM, to give alcohol 76 (172 mg, 69%) as a white powder: mp (MeOH/EtOAc) 185-187° C.; ¹H NMR δ 10.05 (br s, 1 H, NH), 8.64 (dd, J=4.5, 1.6 Hz, 2 H, H-2″, H-6″), 7.64 (dd, J=4.5, 1.6 Hz, 2 H, H-3″, H-5″), 7.49 (d, J=2.0 Hz, 1 H, H-4″), 7.39 (dd, J=8.1, 2.0 Hz, 1 H, H-6″), 7.16 (d, J=8.1 Hz, 1 H, H-5″), 5.68 (t, J=5.4 Hz, 1 H, OH), 4.68 (d, J=5.4 Hz, 2 H, H-1), 2.85 (t, J=7.4 Hz, 2 H, H-3″), 2.80 (t, J=7.4 Hz, 2 H, H-1″), 2.00 (sept, J=7.4 Hz, 2 H, H-2″); ¹³C NMR δ 161.6, 149.7 (2), 144.3, 142.8, 141.5, 139.3, 136.6, 127.1, 124.3, 122.1 (2), 115.3, 113.2, 55.4, 32.5, 31.6, 25.1; MS m/z 324.6 (MH⁺, 100%). Anal. calcd for C₁₈H₁₇N₃OS: C, 66.85; H, 5.30; N, 12.99.Found: C, 66.80; H, 5.30; N, 12.98%.

### Example 1-43

### 2-[(3-Methylphenyl)amino]-4-(4-pyridinyl)-1,3-thiazole-5-carbaldehyde (77)

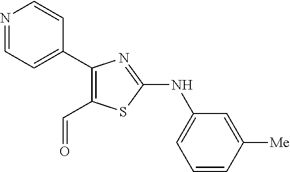

2-[(3-Methylphenyl)amino]-4-(4-pyridinyl)-1,3-thiazole-5-carbaldehyde (77). Mn0₂ (1.23 g, 14.1 mmol) was added to a stirred solution of alcohol 74 (0.84 g, 2.8 mmol) in benzene (40 mL) at 80° C. and the mixture stirred at 80° C. for 2 h. The mixture was cooled to 20° C., filtered through Celite, which was washed with benzene (2×40 mL). The solvent of combined organic fraction was evaporated. The crude solid was purified by column chromatography, eluting with a gradient (0-5%) of MeOH/DCM, to give aldehyde 77 (346 mg, 42%) as a white powder: mp (MeOH/EtOAc) 220-223° C.; ¹H NMR δ 11.12 (s, 1 H, CHO), 9.81 (br s, 1 H, NH), 8.74 (dd, J=4.4, 1.6 Hz, 2 H, H-2′, H-6′), 7.80 (dd, J=4.4, 1.6 Hz, 2 H, H-3′, H-5′), 7.50 (br dd, J=8.0, 1.7 Hz, 1 H, H-6″), 7.40 (br s, 1 H, H-2″), 7.28 (dd, J=8.0, 7.6 Hz, 1 H, H-5″), 6.94 (br d, J=7.6 Hz, 1 H, H-4″), 2.32 (s, 3 H, CH₃); ¹³C NMR δ 181.9, 167.6, 151.4, 150.1 (2), 140.0, 139.3, 138.5, 129.0, 124.8, 124.3, 123.6 (2), 119.2, 115.9, 21.0; MS m/z 296.4 (MH⁺, 100%). Anal. calcd for C₁₆H₁₃N₃OS: C, 65.06; H, 4.44; N, 14.23.Found: C, 64.75; H, 4.56; N, 14.08%.

### Example 1-44

### Methyl (2E)-3-[2-[(3-Methylphenyl)amino]-4-(4-pyridinyl)-1,3-thiazol-5-yl]-2-propenoate (78)

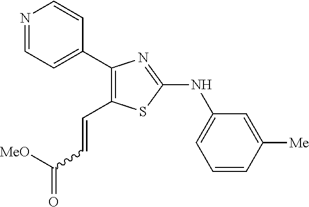

Methyl (2E)-3-[2-[(3-Methylphenyl)amino]-4-(4-pyridinyl)-1,3-thiazol-5-yl]-2-propenoate (78). A solution of aldehyde 77 (123 mg, 0.42 mmol) and methyl (triphenylphosphoranylidene)acetate (139 mg, 0.42 mmol) in DCM (10 mL) was stirred at 20° C. for 48 h. The solvent was evaporated and the crude solid was purified by column chromatography, eluting with 50% EtOAc/pet ether, to give ester 78 (80 mg, 54%) as a white powder: mp (EtOAc/pet. ether) 205-207° C.; ¹H NMR δ 10.78 (br s, 1 H, NH), 8.74 (dd, J=4.4, 1.6 Hz, 2 H, H-2″, H-6″), 7.67 (d, J=15.3 Hz, 1 H, H-3), 7.60 (dd, J=4.4, 1.6 Hz, 2 H, H-3″, H-5″), 7.51 (br dd, J=8.0, 2.0 Hz, 1 H, H-6″), 7.40 (br s, 1 H, H-2″), 7.25 (dd, J=8.0, 7.5 Hz, 1 H, H-5″′), 6.88 (br d, J=7.5 Hz, 1 H, H-4″), 6.02 (d, J=15.3 Hz, 1 H, H-2), 3.68 (s, 3 H, OCH₃), 2.31 (s, 3 H, CH₃); ¹³C NMR δ 166.2, 163.4, 152.4, 150.1 (2), 140.8, 139.8, 138.3, 133.9, 128.9, 123.4, 123.2 (2), 119.7, 118.6, 116.2, 115.2, 51.3, 21.1; MS m/z 352.7 (MH⁺, 100%). Anal. calcd for C₁₉H₁₇N₃O₂S: C, 64.94; H, 4.88; N, 11.96.Found: C, 64.67; H, 5.12; N, 11.74%.

### Example 1-45

### Methyl 3-[2-[(3-Methylphenyl)amino]-4-(4-pyridinyl)-1,3-thiazol-5-yl]propanoate (79)

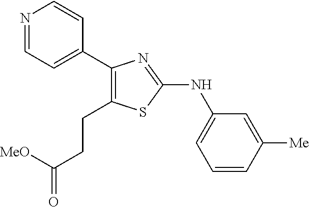

Methyl 3-[2-[(3-Methylphenyl)amino]-4-(4-pyridinyl)-1,3-thiazol-5-yl]propanoate (79). A suspension of alkene 78 (1.06 g, 3.0 mmol) and Pd/C (100 mg) in EtOH/EtOAc (1:1, 100 mL) under H₂ (60 psi) was stirred at 20° C. for 16 h. The mixture was filtered through Celite, washed with EtOH (30 mL) and the solvent was evaporated. The crude solid was purified by column chromatography, eluting with a gradient (50-100%) of EtOAc/pet ether, to give ester 79 (791 mg, 74%) as a yellow powder: mp (EtOAc/pet. ether) 121-123° C.; ¹H NMR δ 10.08 (br s, 1 H, NH), 8.65 (dd, J=4.6, 1.5 Hz, 2 H, H-2″, H-6″), 7.62 (dd, J=4.6, 1.5 Hz, 2 H, H-3″, H-5″), 7.49 (br d, J=8.0 Hz, 1 H, H-6″), 7.37 (br s, 1 H, H-2″), 7.19 (br t, J=7.8 Hz, 1 H, H-5″), 6.77 (br d, J=7.4 Hz, 1 H, H-4″), 3.59 (s, 3 H, OCH₃), 3.25 (br t, J=7.3 Hz, 2 H, H-3), 2.70 (br t, J=7.3 Hz, 2 H, H-2), 2.29 (s, 3 H, CH₃); ¹³C NMR δ 171.9, 160.4, 149.8 (2), 143.2, 141.9, 140.9, 138.0, 128.7, 124.1, 122.4 (2), 122.0, 117.4, 114.0, 51.3, 34.9, 21.7, 21.2; MS m/z 354.6 (MH⁺, 100%). Anal. calcd for C₁₉H₁₉N₃O₂S: C, 64.57; H, 5.42; N, 11.89.Found: C, 64.55; H, 5.35; N, 11.97%.

### Example 1-46

### 3-[2-[(3-Methylphenyl)amino]-4-(4-pyridinyl)-1,3-thiazol-5-yl]-1-propanol (81)

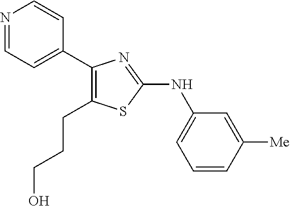

3-[2-[(3-Methylphenyl)amino]-4-(4-pyridinyl)-1,3-thiazol-5-yl]propanoic Acid (80). A solution of NaOH (2 M, 20 mL, 40 mM) was added to a stirred solution of ester 79 (703 mg) in MeOH (50 mL) at 20° C. and the solution was stirred at 20° C. for 6 h. The MeOH was evaporated and the pH of the residual solution adjusted to 6 with 6 M HCl. The suspension was chilled at 5° C. for 16 h, the precipitate filtered and washed with water (2 mL) and dried. The crude solid was triturated with DCM and dried to give acid 80 (674 mg, 99%) as a white powder: mp (H₂O) 235-239° C.; ¹H NMR δ 10.10 (br s, 1 H, NH), 8.62 (dd, J=4.5, 1.6 Hz, 2 H, H-2″, H-6″), 7.64 (dd, J=4.5, 1.6 Hz, 2 H, H-3″, H-5″), 7.50 (br d, J=8.1 Hz, 1 H, H-6″), 7.37 (br s, 1 H, H-2″), 7.18 (br t, J=7.7 Hz, 1 H, H-5″), 6.76 (br d, J=7.5 Hz, 1 H, H-4″), 3.08 (br t, J=7.4 Hz, 2 H, H-3), 2.45 (br t, J=7.4 Hz, 2 H, H-2), 2.28 (s, 3 H, CH₃), OH not observed; MS m/z 340.6 (MH⁺, 100%). Anal. calcd for C₁₈H₁₇N₃O₂S.11/4H₂O: C, 59.50; H, 5.45; N, 11.56.Found: C, 59.18; H, 4.94; N, 11.35%.

3-[2-[(3-Methylphenyl)amino]-4-(4-pyridinyl)-1,3-thiazol-5-yl]-1-propanol (81). LiAlH₄ (60 mg, 1.7 mmol) was added to a stirred solution of acid 80 (270 mg, 0.8 mmol) in THF (40 mL) at 0° C. and the mixture stirred at 20° C. for 4 h. Water (5 mL) was added dropwise to the stirred solution and the mixture stirred for 5 min, then 6 M HCl (5 mL) was added and the mixture stirred at 20° C. for 1 h. The organic solvent was evaporated and the residue was partitioned between EtOAc (50 mL) and dilute aqueous NH₃ solution (50 mL). The organic fraction was dried and the solvent evaporated. The crude solid was purified by column chromatography, eluting with EtOAc, to give propanol 81 (131 mg, 50%) as a white powder: mp (EtOAc) 150-152° C.; ¹H NMR δ 10.05 (br s, 1 H, NH), 8.63 (dd, J=4.5, 1.6 Hz, 2 H, H-2″, H-6″), 7.65 (dd, J=4.5, 1.6 Hz, 2 H, H-3″, H-5″), 7.50 (br d, J=8.0 Hz, 1 H, H-6″), 7.38 (br s, 1 H, H-2″), 7.20 (br t, J=7.8 Hz, 1 H, H-5″), 6.78 (br d, J=7.5 Hz, 1 H, H-4″), 4.56 (t, J=5.0 Hz, 1 H, OH), 3.48 (dt, J=5.9, 5.0 Hz, 2 H, H-1), 2.95 (br t, J=7.9 Hz, 2 H, H-3), 2.29 (s, 3 H, CH₃), 1.75-1.82 (m, 2 H, H-2); ¹³C NMR δ 160.0, 149.8 (2), 142.3, 142.1, 141.0, 138.0, 128.7, 126.5, 122.2 (2), 121.9, 117.3, 114.0, 59.6, 34.4, 22.9, 21.2; MS m/z 326.6 (MH⁺, 100%).

### Example 1-47

### N-(3-Methylphenyl)-5-[3-(4-morpholinyl)propyl]-4-(4-pyridinyl)-1,3-thiazol-2-amine (82)

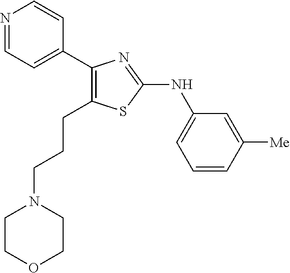

N-(3-Methylphenyl)-5-[3-(4-morpholinyl)propyl]-4-(4-pyridinyl)-1,3-thiazol-2-amine (82). MsCl (23 μL, 0.30 mmol) was added to a stirred solution of alcohol 81 (74 mg, 0.23 mmol) and iPr₂NEt (60 μL, 0.34 mmol) in DCM (10 mL) at 0° C. and the mixture stirred at 20° C. for 4 h. The solvent was evaporated and the residue dissolved in morpholine (4 mL) and the mixture was stirred at 60° C. for 6 h. The solvent was evaporated and the residue was partitioned between aqueous NH₃ solution (20 mL) and EtOAc (50 mL). The organic fraction was washed with water (2×5 mL), washed with brine (5 mL) and the solution was dried and the solvent was evaporated. The crude solid was purified by column chromatography, eluting with a gradient (0-5%) of MeOH/DCM, to give morpholide 82 (64 mg, 70%) as a white powder: mp (MeOH/EtOAc) 128-131° C.; ¹H NMR δ 10.06 (br s, 1 H, NH), 8.63 (dd, J=4.6, 1.5 Hz, 2 H, H-2′, H-6′), 7.65 (dd, J=4.6, 1.5 Hz, 2 H, H-3′, H-5′), 7.50 (br d, J=7.8 Hz, 1 H, H-6″), 7.37 (br s, 1 H, H-2″), 7.20 (br t, J=7.8 Hz, 1 H, H-5″), 6.77 (br d, J=7.5 Hz, 1 H, H-4″), 3.55 (bt t, J=4.5 Hz, 4 H, 2×CH₂O), 2.93 (br t, J=7.3 Hz, 2 H, H-1″), 2.27-2.33 (m, 9 H, 3×CH₂N, CH₃), 1.75-1.81 (m, 2 H, H-2″); ¹³C NMR δ 160.1, 149.7 (2), 142.6, 142.1, 140.9, 138.0, 128.7, 126.1, 122.3 (2), 121.9, 117.3, 114.0, 66.1 (2), 56.5, 53.1 (2), 27.7, 23.5, 21.2; MS m/z 367.7 (MH⁺, 100%).

### Example 1-48

### 5-[(Dimethylamino)methyl]-N-(3-m ethylphenyl)-4-(4-pyridinyl)-1,3-thiazol-2-amine (83)

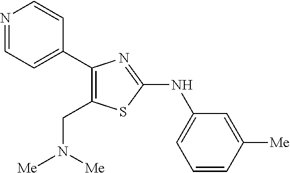

5-[(Dimethylamino)methyl]-N-(3-methylphenyl)-4-(4-pyridinyl)-1,3-thiazol-2-amine (83). MsCl (120 μL, 1.5 mmol) was added to a stirred solution of alcohol 74 (340 mg, 1.1 mmol) and iPr₂NEt (300 μL, 1.7 mmol) in DCM (30 mL) at 0° C. and the mixture stirred at 20° C. for 3 h. The solvent was evaporated and the residue dissolved in DMF (3 mL) and 40% aqueous MeNH₂ was added (2.9 mL, 23 mmol) and the mixture was stirred at 80° C. for 4 h. The solvent was evaporated and the residue partitioned between aqueous Na₂CO₃ solution (5% w/v, 20 mL) and EtOAc (50 mL). The organic fraction was washed with water (2×5 mL), washed with brine (5 mL) and the solution dried, and the solvent was evaporated. The crude solid was purified by column chromatography, eluting with a gradient (0-5%) of MeOH/DCM, to give amine 83 (124 mg, 33%) as a white powder: mp (MeOH/DCM) 157-159° C.; ¹H NMR δ 10.12 (br s, 1 H, NH), 8.64 (dd, J=4.5, 1.6 Hz, 2 H, H-2′, H-6′), 7.66 (dd, J=4.5, 1.6 Hz, 2 H, H-3′, H-5′), 7.51 (br dd, J=7.9, 1.9 Hz, 1 H, H-6″), 7.39 (br s, 1 H, H-2″), 7.20 (dd, J=7.9, 7.5 Hz, 1 H, H-5″), 6.79 (d, J=7.5 Hz, 1 H, H-4″), 3.63 (s, 2 H, H-1″), 2.33 (s, 3 H, CH₃), 2.23 [s, 6 H, N(CH₃)₂]; ¹³C NMR δ 161.5, 149.7 (2), 143.6, 141.8, 140.9, 138.0, 128.7, 125.1, 122.5 (2), 122.0, 117.4, 114.1, 54.8, 44.9 (2), 21.2; MS m/z 325.6 (MH⁺, 100%). Anal. calcd for C₁₈H₂₀N₄S: C, 66.63; H, 6.21; N, 17.27.Found: C, 66.51; H, 6.29; N, 17.04%.

### Example 1-49

### 5-[(Diethylamino)methyl]-N-(3-methylphenyl)-4-(4-pyridinyl)-1,3-thiazol-2-amine (84)

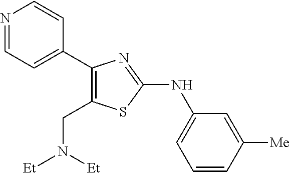

5-[(Diethylamino)methyl]-N-(3-methylphenyl)-4-(4-pyridinyl)-1,3-thiazol-2-amine (84). MsCl (105 μL, 1.3 mmol) was added to a stirred solution of alcohol 74 (306 mg, 1.0 mmol) and iPr₂NEt (270 μL, 1.6 mmol) in DCM (20 mL) at 0° C. and the mixture stirred at 20° C. for 3 h. The solvent was evaporated and the residue dissolved in DMF (3 mL) and Et₂NH (2.1 mL, 21 mmol) was added and the mixture was stirred at 50° C. for 4 h. The solvent was evaporated and the residue partitioned between aqueous Na₂CO₃ solution (5% w/v, 20 mL) and EtOAc (50 mL). The organic fraction was washed with water (2×5 mL), washed with brine (5 mL) and the solution dried, and the solvent was evaporated. The crude solid was purified by column chromatography, eluting with a gradient (0-5%) of MeOH/DCM, to give amine 84 (168 mg, 46%) as a white powder: mp (MeOH/EtOAc) 123-125° C.; ¹H NMR δ 10.02 (br s, 1 H, NH), 8.62 (dd, J=4.5, 1.6 Hz, 2 H, H-2′, H-6′), 7.64 (dd, J=4.5, 1.6 Hz, 2 H, H-3′, H-5′), 7.50 (br dd, J=8.5, 2.3 Hz, 1 H, H-6″), 7.39 (br s, 1 H, H-2″), 7.20 (dd, J=8.5, 7.5 Hz, 1 H, H-5″), 6.77 (d, J=7.5 Hz, 1 H, H-4″), 3.80 (s, 2 H, H-1″), 2.52 (q, J=7.1 Hz, 4 H, 2×CH₂N), 2.29 (s, 3 H, CH₃), 0.98 (t, J=7.1 Hz, 6 H, 2×CH₃); ¹³C NMR δ 161.4, 149.7 (2), 142.7, 142.0, 141.0, 138.0, 128.7, 128.0, 122.4 (2), 121.9, 117.4, 114.0, 49.4, 46.4 (2), 21.2, 11.6 (2); MS m/z 353.6 (MH⁺, 100%). Anal. calcd for C₂₀H₂₄N₄S.½CH₃OH: C, 66.82; H, 7.11; N, 15.20.Found: C, 66.41; H, 7.09; N, 14.94%.

### Example 1-50

### N-(3-Methylphenyl)-5-(1-piperidinylmethyl)-4-(4-pyridinyl)-1,3-thiazol-2-amine (85)

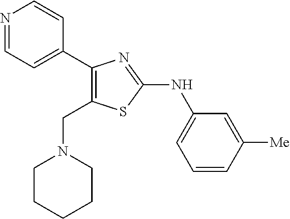

N-(3-Methylphenyl)-5-(1-piperidinylmethyl)-4-(4-pyridinyl)-1,3-thiazol-2-amine (85). MsCl (107 μL, 1.4 mmol) was added to a stirred solution of alcohol 74 (311 mg, 1.0 mmol) and iPr₂NEt (273 μL, 1.6 mmol) in DCM (20 mL) at 0° C. and the mixture stirred at 20° C. for 3 h. The solvent was evaporated and the residue dissolved in DMF (3 mL) and piperidine (2.1 mL, 21 mmol) was added and the mixture was stirred at 50° C. for 4 h. The solvent was evaporated and the residue partitioned between aqueous Na₂CO₃ solution (5% w/v, 20 mL) and EtOAc (50 mL). The organic fraction was washed with water (2×5 mL), washed with brine (5 mL) and the solution dried, and the solvent was evaporated. The crude solid was purified by column chromatography, eluting with a gradient (0-5%) of MeOH/DCM, to give amine 85 (208 mg, 54%) as a white powder: mp (MeOH/DCM) 150-151° C.; ¹H NMR δ 10.05 (br s, 1 H, NH), 8.63 (dd, J=4.5, 1.6 Hz, 2 H, H-2′, H-6′), 7.64 (dd, J=4.5, 1.6 Hz, 2 H, H-3′, H-5′), 7.51 (br d, J=7.7 Hz, 1 H, H-6″), 7.38 (br s, 1 H, H-2″), 7.20 (br t, J=7.7 Hz, 1 H, H-5″), 6.78 (d, J=7.5 Hz, 1 H, H-4″), 3.67 (s, 2 H, H-1″), 2.39-2.44 (m, 4 H, 2×CH₂N), 2.29 (s, 3 H, CH₃), 1.47-1.53 (m, 4 H, 2×CH₂), 1.36-1.42 (m, 2 H, CH₂); ¹³C NMR δ 161.6, 149.7 (2), 143.4, 141.9, 140.9, 138.0, 128.8, 125.6, 122.5 (2), 122.0, 117.5, 114.1, 54.3, 53.9 (2), 25.4 (2), 23.7, 21.2; MS m/z 365.8 (MH⁺, 100%). Anal. calcd for C₂₁H₂₄N₄S.½CH₃OH: C, 67.86; H, 6.89; N, 14.72.Found: C, 67.74; H, 6.72; N, 14.90%.

### Example 1-51

### N-(3-Methylphenyl)-5-(4-morpholinylmethyl)-4-(4-pyridinyl)-1,3-thiazol-2-amine (86)

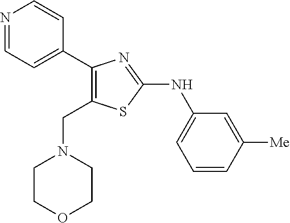

N-(3-Methylphenyl)-5-(4-morpholinylmethyl)-4-(4-pyridinyl)-1,3-thiazol-2-amine (86). MsCl (58 μL, 0.74 mmol) was added to a stirred solution of alcohol 74 (200 mg, 0.67 mmol) and iPr₂NEt (234 μL, 1.4 mmol) in DCM (20 mL) at 0° C. and the mixture stirred at 20° C. for 1 h. The solvent was evaporated and the residue dissolved in DMF (3 mL) and morpholine (1.2 mL, 13.5 mmol) was added and the mixture was stirred at 80° C. for 4 h. The solvent was evaporated and the residue partitioned between aqueous Na₂CO₃ solution (5% w/v, 20 mL) and EtOAc (50 mL). The organic fraction was washed with water (2×5 mL), washed with brine (5 mL) and the solution dried, and the solvent was evaporated. The crude solid was purified by column chromatography, eluting with a gradient (0-5%) of MeOH/DCM, to give morpholide 86 (153 mg, 62%) as a white powder: mp (MeOH/DCM) 160-161° C.; ¹H NMR δ 10.09 (br s, 1 H, NH), 8.64 (dd, J=4.5, 1.6 Hz, 2 H, H-2′, H-6′), 7.66 (dd, J=4.5, 1.6 Hz, 2 H, H-3′, H-5′), 7.50 (br dd, J=8.1, 1.9 Hz, 1 H, H-6″), 7.38 (br s, 1 H, H-2″), 7.20 (dd, J=8.1, 7.5 Hz, 1 H, H-5″), 6.78 (d, J=7.5 Hz, 1 H, H-4″), 3.72 (s, 2 H, H-1″), 3.58 (br t, J=4.5 Hz, 4 H, 2×CH₂N), 2.47 (br t, J=4.5 Hz, 4 H, 2×CH₂O), 2.30 (s, 3 H, CH₃); ¹³C NMR δ 161.7, 149.7 (2), 143.9, 141.8, 140.9, 138.0, 128.8, 124.0, 122.5 (2), 122.0, 117.5, 114.1, 66.1 (2), 53.8, 53.0 (2), 21.2; MS m/z 367.7 (MH⁺, 100%). Anal. calcd for C₂₀H₂₂N₄OS: C, 65.55; H, 6.05; N, 15.29.Found: C, 65.42; H, 6.02; N, 15.25%.

### Example 1-52

### Ethyl 2-[(3-Methylphenyl)amino]-4-(4-pyridinyl)-1,3-thiazole-5-carboxylate (88)

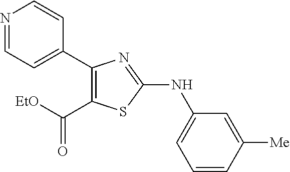

Ethyl 2-Bromo-3-oxo-3-(4-pyridinyl)propanoate Hydrobromide (87). Br₂ (1.4 mL, 27.7 mmol) was added dropwise to a stirred a solution of ethyl isonicotinoyl acetate (5.09 g, 26.3 mmol) in CHCl₃ (100 mL) at 5° C. The mixture was stirred at 40° C. for 1 h and then at 70° C. for 1 h. The mixture was cooled to 20° C., diluted with Et₂O (200 mL) and stirred at 5° C. for 30 min. The precipitate was filtered, washed with Et₂O (25 mL) and dried under vacuum to give the bromoketone hydrobromide salt 87 (8.19 g, 88%) as a white powder: mp (Et₂O/CHCl₃) 141-143° C.; ¹H NMR δ 8.93 (dd, J=4.6, 1.7 Hz, 2 H, H-2′, H-6′), 7.97 (dd, J=4.6, 1.7 Hz, 2 H, H-3′, H-5′), 6.48 (br s, 1 H, N.HBr), 6.70 (s, 1 H, CHBr), 4.21 (q, J=7.1 Hz, 2 H, CH₂O), 1.15 (t, J=7.1 Hz, 3 H, CH₃).

Ethyl 2-[(3-Methylphenyl)amino]-4-(4-pyridinyl)-1,3-thiazole-5-carboxylate (88). A mixture of bromide 87 (0.51 g, 1.4 mmol) and 3-methylphenylthiourea 4 (0.24 g, 1.4 mmol) in EtOH (20 mL) was stirred at reflux temperature for 2 h. The mixture was cooled to 0° C. for 2 h and the precipitate was filtered, washed with EtOH (2 mL) and dried to give ester 88 (0.25 g, 51%) as a yellow powder: mp (EtOH) 233-235° C.; ¹H NMR δ 10.89 (s, 1 H, NH), 8.91 (dd, J=5.2, 1.4 Hz, 2 H, H-2′, H-6′), 7.67 (dd, J=5.2, 1.4 Hz, 2 H, H-3′, H-5′), 7.48 (br dd, J=8.0, 1.9 Hz, 1 H, H-6″), 7.37 (br s, 1 H, H-2″), 7.26 (br dd, J=8.0, 7.5 Hz, 1 H, H-5″), 6.92 (br d, J=7.5 Hz, 1 H, H-4″), 4.20 (q, J=7.1 H, 2 H, CH₂O), 2.31 (s, 3 H, CH₃), 1.21 (t, J=7.1 Hz, 3 H, CH₃); ¹³C NMR δ 165.3, 160.3, 151.5, 148.9, 142.6 (2), 139.5, 138.4, 129.0, 126.8 (2), 123.9, 118.8, 115.4, 114.5, 61.2, 21.1, 13.8; MS m/z 340.6 (MH⁺, 100%).

### Example 1-53

### Ethyl 2-(2,3-Dihydro-1H-inden-5-ylamino)-4-(4-pyridinyl)-1,3-thiazole-5-carboxylate (89)

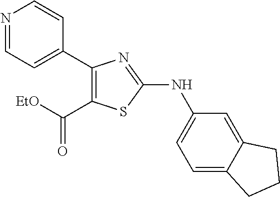

Ethyl 2-(2,3-Dihydro-1H-inden-5-ylamino)-4-(4-pyridinyl)-1,3-thiazole-5-carboxylate (89). A mixture of bromide 87 (1.80 g, 5.1 mmol) and N-(2,3-dihydro-1H-inden-5-yl)thiourea (46) (0.98 g, 5.1 mmol) in EtOH (40 mL) was stirred at reflux temperature for 2 h. The mixture was cooled to 20° C., diluted with water (100 mL), the pH adjusted to ca. 9 with aqueous NH₃ and the mixture stirred at 0° C. for 1 h. The precipitate was filtered, washed with water (15 mL) and dried. The crude solid was purified by column chromatography, eluting with a gradient (0-5%) of MeOH/DCM, to give ester 89 (0.50 g, 76%) as a yellow powder: mp (MeOH/DCM) 203-205° C.; ¹H NMR δ 10.72 (s, 1 H, NH), 8.65 (dd, J=4.4, 1.6 Hz, 2 H, H-2′, H-6′), 7.68 (dd, J=4.4, 1.6 Hz, 2 H, H-3′, H-5′), 7.42 (br s, 1 H, H-4″), 7.32 (dd, J=8.1, 2.0 Hz, 1 H, H-6″), 7.21 (d, J=8.1 Hz, 1 H, H-7″), 4.15 (q, J=7.1 H, 2 H, CH₂O), 2.86 (br t, J=7.4 Hz, 2 H, H-3″), 2.81 (br t, J=7.4 Hz, 2 H, H-1″), 2.01 (sept, J=7.4 Hz, 2 H, H-2″), 1.17 (t, J=7.1 Hz, 3 H, CH₃); ¹³C NMR δ 165.7, 160.6, 154.9, 149.2 (2), 144.7, 141.6, 138.6, 138.1, 124.6, 123.9 (2), 116.7, 114.7, 111.1, 60.6, 32.4, 31.6, 25.0, 13.8; MS m/z 366.7 (MW, 100%). Anal. calcd for C₂₀H₁₉N₃O₂S: C, 65.73; H, 5.24; N, 11.50.Found: C, 65.44; H, 5.27; N, 11.75%.

### Example 1-54

### 2-[(3-Methylphenyl)amino]-4-(4-pyridinyl)-1,3-thiazole-5-carboxamide (91)

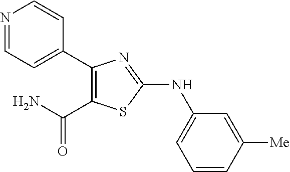

2-[(3-Methylphenyl)amino]-4-(4-pyridinyl)-1,3-thiazole-5-carboxylic acid (90). A mixture of ester 88 (480 mg, 1.4 mmol) and aqueous NaOH (1 M, 20 mL, 20 mmol) in MeOH (20 mL) was stirred at 60° C. for 2 h. The organic solvent was evaporated and the pH adjusted to 6 with 6 M HCl and the mixture was stood at 5° C. for 16 h. The precipitate was filtered and dried and the solid was triturated with DCM to give carboxylic acid 90 (245 mg, 56%) as a yellow powder: mp (H₂O) 160-162° C.; ¹H NMR δ 13.0 (br s, 1 H, CO₂H), 10.67 (s, 1 H, NH), 8.64 (dd, J=4.5, 1.6 Hz, 2 H, H-2′, H-6′), 7.74 (dd, J=4.5, 1.6 Hz, 2 H, H-3′, H-5′), 7.46 (br d, J=8.0 Hz, 1 H, H-6″), 7.36 (br s, 1 H, H-2″), 7.25 (br t, J=7.8 Hz, 1 H, H-5″), 6.87 (br d, J=7.5 Hz, 1 H, H-4″), 2.30 (s, 3 H, CH₃); ¹³C NMR δ 164.5, 162.1, 153.5, 149.0 (2), 141.7, 139.9, 138.3, 128.9, 124.0 (2), 123.4, 118.4, 115.1, 114.0, 21.1; MS m/z 312.5 (MH⁺, 100%). Anal. calcd for C₁₆H₁₃N₃O₂S.H₂O: C, 58.35; H, 4.59; N, 12.76.Found: C, 58.67; H, 4.32; N, 12.79%.

2-[(3-Methylphenyl)amino]-4-(4-pyridinyl)-1,3-thiazole-5-carboxamide (91). Oxalyl chloride (34 μL, 0.40 mmol) was added dropwise to a stirred solution of acid 90 (83 mg, 0.27 mmol) and DMF (1 drop) in DCM (5 mL) and the mixture was stirred at 20° C. for 2 h. The solvent as evaporated and the residue suspended in DCM (5 mL) and NH₃ gas was bubbled through the mixture for 5 min the mixture stirred at 20° C. for 16 h. The solvent was evaporated and the crude solid was purified by column chromatography, eluting with a gradient (0-10%) of MeOH/DCM, to give carboxamide 91 (6 mg, 7%) as a tan gum: ¹H NMR δ 10.45 (s, 1 H, NH), 8.63 (dd, J=4.5, 1.6 Hz, 2 H, H-2′, H-6′), 7.70 (dd, J=4.5, 1.6 Hz, 2 H, H-3′, H-5′), 7.48-7.54 (m, 3 H, H-6″, CONH₂), 7.38 (br s, 1 H, H-2″), 7.23 (br t, J=7.8 Hz, 1 H, H-5″), 6.85 (br d, J=7.5 Hz, 1 H, H-4″), 2.30 (s, 3 H, CH₃); MS m/z 311.5 (MH⁺, 100%).

### Example 1-55

### N,N-Dimethyl-2-[(3-methylphenyl)amino]-4-(4-pyridinyl)-1,3-thiazole-5-carboxamide (92)

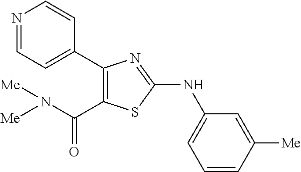

N,N-Dimethyl-2-[(3-methylphenyl)amino]-4-(4-pyridinyl)-1,3-thiazole-5-carboxamide (92). Oxalyl chloride (34 μL, 0.40 mmol) was added dropwise to a stirred solution of acid 90 (83 mg, 0.27 mmol) and DMF (1 drop) in DCM (5 mL) and the mixture was stirred at 20° C. for 2 h. The solvent as evaporated and the residue suspended in DCM (5 mL) and dimethylamine (40% aqueous solution, 5 mL) was added and the mixture stirred at 20° C. for 16 h. The solvent was evaporated and the mixture was partitioned between EtOAc (50 mL) and dilute aqueous NH₃ (20 mL). The organic fraction was dried and the solvent evaporated. The crude solid was purified by column chromatography, eluting with a gradient (0-10%) of MeOH/DCM, to give carboxamide 92 (27 mg, 30%) as a tan solid: mp (MeOH/EtOAc) 193-196° C.; ¹H NMR δ 10.42 (s, 1 H, NH), 8.65 (dd, J=4.5, 1.6 Hz, 2 H, H-2′, H-6′), 7.57 (dd, J=4.5, 1.6 Hz, 2 H, H-3′, H-5′), 7.53 (br d, J=8.1 Hz, 1 H, H-6″), 7.43 (br s, 1 H, H-2″), 7.25 (br t, J=7.8 Hz, 1 H, H-5″), 6.85 (br d, J=7.5 Hz, 1 H, H-4″), 2.89 [br s, 6 H, CON(CH₃)₂], 2.32 (s, 3 H, CH₃); MS m/z 339.6 (MH⁺, 100%).

### Example 1-56

N-[2-(Dimethylamino)ethyl]-2-[(3-methylphenyl)amino]-4-(4-pyridinyl)-1,3-thiazole-5-carboxamide (93)

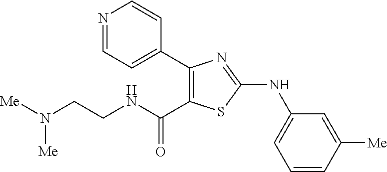

N-[2-(Dimethylamino)ethyl]-2-[(3-methylphenyl)amino]-4-(4-pyridinyl)-1,3-thiazole-5-carboxamide (93). Oxalyl chloride (53 μL, 0.63 mmol) was added dropwise to a stirred solution of acid 90 (130 mg, 0.42 mmol) and DMF (1 drop) in DCM (5 mL) and the mixture was stirred at 20° C. for 2 h. The solvent as evaporated and the residue suspended in DCM (5 mL) and N,N-dimethylethylenediamine (140 μL, 1.25 mmol) was added and the mixture stirred at 20° C. for 16 h. The mixture was partitioned between EtOAc (50 mL) and dilute aqueous NH₃ (20 mL). The organic fraction was dried and the solvent evaporated. The crude solid was purified by column chromatography, eluting with a gradient (0-10%) of MeOH/DCM, to give carboxamide 93 (39 mg, 24%) as a tan powder: mp (MeOH/DCM) 189-191° C.; ¹H NMR δ 10.46 (s, 1 H, NH), 8.63 (dd, J=4.5, 1.6 Hz, 2 H, H-2′, H-6′), 8.02 (t, J=5.5 Hz, 1 H, CONH), 7.70 (dd, J=4.5, 1.6 Hz, 2 H, H-3′, H-5′), 7.49 (br d, J=8.1 Hz, 1 H, H-6″), 7.39 (br s, 1 H, H-2″), 7.25 (br t, J=7.8 Hz, 1 H, H-5″), 6.85 (br d, J=7.5 Hz, 1 H, H-4″), 3.22-3.24 (m, 2 H, CH₂N), 2.40 (br t, J=6.4 Hz, 2 H, CH₂N), 2.32 (s, 3 H, CH₃), 2.20 [s, 6 H, N(CH₃)₂]; ¹³C NMR δ 162.2, 160.7, 149.5 (2), 146.8, 141.2, 140.3, 138.2, 128.9, 122.8, 122.7 (2), 118.4, 118.0, 114.6, 57.3, 44.7 (2), 37.0, 21.1; MS m/z 382.8 (MH⁺, 100%). Anal. calcd for C₂₀H₂₃N₅OS.¼CH₂Cl₂: C, 60.39; H, 5.88; N, 17.39.

Found: C, 60.04; H, 5.95; N, 17.06%.

### Example 1-57

### 2-[(3-Methylphenyl)amino]-N-[3-(4-morpholinyl)propyl]-4-(4-pyridinyl)-1,3-thiazole-5-carboxamide (94)

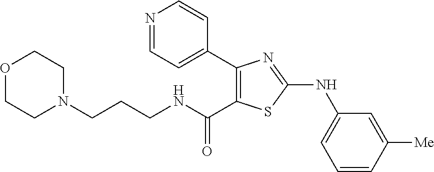

2-[(3-Methylphenyl)amino]-N-[3-(4-morpholinyl)propyl]-4-(4-pyridinyl)-1,3-thiazole-5-carboxamide (94). Oxalyl chloride (41 μL, 0.48 mmol) was added dropwise to a stirred solution of acid 90 (100 mg, 0.32 mmol) and DMF (1 drop) in DCM (5 mL) and the mixture was stirred at 20° C. for 2 h. The solvent as evaporated and the residue suspended in DCM (5 mL) and 3-(4-morpholinyl)propylamine (140 μL, 0.96 mmol) was added and the mixture stirred at 20° C. for 16 h. The mixture was partitioned between EtOAc (50 mL) and dilute aqueous NH₃ (20 mL). The organic fraction was dried and the solvent evaporated. The crude solid was purified by column chromatography, eluting with a gradient (0-10%) of MeOH/DCM, to give carboxamide 94 (30 mg, 21%) as a tan powder: mp (MeOH/EtOAc) 190-192° C.; ¹H NMR δ 10.46 (s, 1 H, NH), 8.62 (dd, J=4.5, 1.6 Hz, 2 H, H-2′, H-6′), 8.24 (t, J=5.5 Hz, 1 H, CONH), 7.66 (dd, J=4.5, 1.6 Hz, 2 H, H-3′, H-5′), 7.50 (br d, J=8.1 Hz, 1 H, H-6″), 7.39 (br s, 1 H, H-2″), 7.23 (br t, J=7.8 Hz, 1 H, H-5″), 6.85 (br d, J=7.5 Hz, 1 H, H-4″), 3.52-3.58 (m, 4 H, 2×CH₂O), 3.20 (dt, J=6.7, 5.9 Hz, 2 H, CH₂N), 2.25 (m, 9 H, 3×CH₂N, CH₃), 1.58-1.65 (m, 2 H, CH₂); ¹³C NMR δ 162.0, 160.8, 149.5 (2), 146.7, 141.3, 140.3, 138.2, 128.9, 122.8, 122.7 (2), 118.3, 117.9, 114.6, 66.0 (2), 55.8, 53.1 (2), 37.8, 25.2, 21.1; MS m/z 439.0 (MH⁺, 100%). Anal. calcd for C₂₃H₂₇N₅O₂S.H₂O: C, 60.64; H, 6.42; N, 15.37.Found: C, 60.70; H, 6.27; N, 15.18%.

### Example 1-58

### N-Methyl-N-(3-methylphenyl)-4-(4-pyridinyl)-1,3-thiazol-2-amine (95)

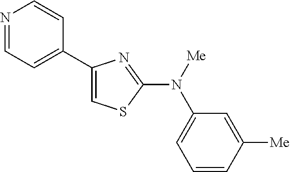

N-Methyl-N-(3-methylphenyl)-4-(4-pyridinyl)-1,3-thiazol-2-amine (95). NaH (216 mg, 5.4 mmol) was added to a stirred solution of amine 5 (760 mg, 2.8 mmol) in DMF (5 mL) and the mixture stirred at 20° C. for 2 min. MeI (0.18 mL, 2.8 mmol) was added dropwise and the mixture stirred at 20° C. for 1 h. The reaction was quenched with saturated aqueous NH₄Cl solution (15 mL) and the mixture extracted with EtOAc (3×15 mL). The combined organic fraction was dried and the solvent evaporated. The crude solid was purified by column chromatography, eluting with a gradient (50-100%) of EtOAc/pet. ether, to give amine 95 (0.69 g, 86%) as a colourless oil: ¹H NMR (CDCl₃) δ 8.60 (dd, J=4.6, 1.5 Hz, 2 H, H-2′, H-6′), 7.71 (dd, J=4.6, 1.6 Hz, 2 H, H-3′, H-5′), 7.33 (dd, J=7.8, 7.6 Hz, 1 H, H-5″), 7.20-7.24 (m, 2 H, H-2″, H-6″), 7.11 (br d, J=7.6 Hz, 1 H, H-4″), 6.90 (s, 1 H, H-5), 3.60 (s, 3 H, NCH₃), 2.39 (s, 3 H, CH₃); ¹³C NMR (CDCl₃) δ 170.1, 150.1 (2), 149.0, 146.1, 141.9, 139.9, 129.6, 127.5, 125.7, 122.1, 120.2 (2), 105.4, 40.4, 21.4; MS m/z 282.5 (MH⁺, 100%). Anal. calcd for C₁₆H₁₅N₃S: C, 68.30; H, 5.37; N, 14.93.Found: C, 67.97; H, 5.45; N, 14.86%.

### Example 1-59

### N-Ethyl-N-(3-methylphenyl)-4-(4-pyridinyl)-1,3-thiazol-2-amine (96)

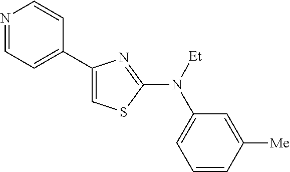

N-Ethyl-N-(3-methylphenyl)-4-(4-pyridinyl)-1,3-thiazol-2-amine (96). NaH (151 mg, 3.8 mmol) was added to a stirred solution of amine 5 (533 mg, 2.0 mmol) in DMF (8 mL) and the mixture stirred at 20° C. for 2 min. EtI (0.16 mL, 2.0 mmol) was added dropwise and the mixture stirred at 20° C. for 1 h. The reaction was quenched with saturated aqueous NH₄Cl solution (10 mL) and the mixture extracted with EtOAc (3×15 mL). The combined organic fraction was dried and the solvent evaporated. The crude solid was purified by column chromatography, eluting with 50% EtOAc/pet. ether, to give amine 96 (0.55 g, 93%) as a colourless oil: ¹H NMR (CDCl₃) δ 8.60 (dd, J=4.6, 1.5 Hz, 2 H, H-2′, H-6′), 7.71 (dd, J=4.6, 1.6 Hz, 2 H, H-3′, H-5′), 7.34 (br dd, J=8.6, 7.4 Hz, 1 H, H-5″), 7.14-7.20 (m, 3 H, H-2″, H-4″, H-6″), 6.87 (s, 1 H, H-5), 4.08 (q, J=7.1 Hz, 2 H, CH₂N), 2.39 (s, 3 H, CH₃), 1.32 (t, J=7.1 Hz, 3 H, CH₃); ¹³C NMR (CDCl₃) δ 170.0, 150.1 (2), 149.0, 144.6, 142.0, 140.1, 129.8, 128.2, 127.6, 124.1, 120.2 (2), 105.0, 47.7, 21.4, 13.1; MS m/z 296.5 (MH⁺, 100%). Anal. calcd for C₁₇H₁₇N₃S: C, 69.12; H, 5.80; N, 14.22.Found: C, 68.90; H, 5.89; N, 14.28%.

### Example 1-60

### 2-{3-Methyl[4-(4-pyridinyl)-1,3-thiazol-2-yl]anilino}ethanol (97)

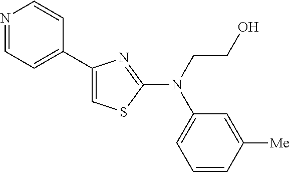

2-{3-Methyl[4-(4-pyridinyl)-1,3-thiazol-2-yl]anilino}ethanol (97). NaH (107 mg, 2.7 mmol) was added to a stirred solution of amine 5 (649 mg, 2.4 mmol) in DMF (10 mL) and the mixture stirred at 20° C. for 2 min. Iodoethanol (0.19 mL, 2.4 mmol) was added dropwise and the mixture stirred at 20° C. for 48 h. The reaction was quenched with saturated aqueous NH₄Cl solution (10 mL) and the mixture extracted with EtOAc (3×15 mL). The combined organic fraction was dried and the solvent evaporated. The crude solid was purified by column chromatography, eluting with a gradient (50-100%) of EtOAc/pet. ether, to give (i) starting material 5 (0.22 g, 34%) and (ii) amine 97 (0.15 g, 19%) as a white powder: mp (EtOAc/pet. ether) 149-151° C.; ¹H NMR δ 8.58 (dd, J=4.5, 1.6 Hz, 2 H, H-2″, H-6″), 7.78 (dd, J=4.5, 1.6 Hz, 2 H, H-3″, H-5″′), 7.47 (s, 1 H, H-5″), 7.30-7.40 (m, 3 H, H-2′, H-4′, H-5′), 7.34 (br dd, J=7.4 Hz, 1 H, H-6′), 4.80 (t, J=5.5 Hz, 1 H, OH), 4.03 (t, J=6.2 Hz, 2 H, H-2), 3.71 (dt, J=6.2, 5.5 Hz, 2 H, H-1), 2.35 (s, 3 H, CH₃); ¹³C NMR δ 169.4, 149.9 (2), 147.7, 144.8, 141.1, 139.4, 129.6, 127.9, 127.0, 123.6, 119.8 (2), 106.6, 57.7, 54.9, 20.8; MS m/z 312.6 (MH⁺, 100%). Anal. calcd for C₁₇H₁₇N₃OS: C, 65.57; H, 5.50; N, 13.49.Found: C, 65.87; H, 5.43; N, 13.65%.

### Example 1-61

### N-[4-(4-Pyridinyl)-1,3-thiazol-2-yl]benzamide (99)

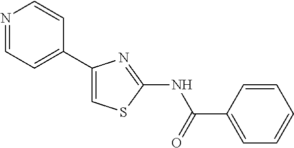

4-(4-Pyridinyl)-1,3-thiazol-2-amine (98). A mixture of bromoketone hydrobromide 1 (5.0 g, 17.8 mmol) and thiourea (1.36 g, 17.8 mmol) in EtOH (100 mL) was stirred at reflux temperature for 2 h. The mixture was cooled to 20° C., diluted with water (50 mL), the pH adjusted to ca. 8 with aqueous NH₃ and the mixture stirred at 0° C. for 1 h. The precipitate was filtered, washed with water (5 mL) and ether (5 mL) and dried to give amine 98 (2.70 g, 86%) as a pink powder: mp (EtOH/H₂O) 278-282° C. [lit. (Westphal, G. et al., J. Prakt. Chemie (Leipzig) 1976, 318, 875) mp (EtOH) 276-277° C.]; ¹H NMR δ 8.53 (dd, J=4.6, 1.6 Hz, 2 H, H-2′, H-6′), 7.91 (dd, J=4.6, 1.6 Hz, 2 H, H-3′, H-5′), 7.38 (s, 1 H, H-5), 7.16 (br s, 2 H, NH₂); MS m/z 178.3 (MH⁺, 100%).

N-[4-(4-Pyridinyl)-1,3-thiazol-2-yl]benzamide (99). Benzoyl chloride (0.44 mL, 3.8 mmol) was added to a stirred solution of amine 98 (0.44 g, 2.5 mmol) and DMAP (30 mg, 0.25 mmol) in pyridine (30 mL) and the solution stirred at 20° C. for 16 h. The solvent was evaporated and the residue partitioned between water (70 mL) and EtOAc (70 mL). The organic fraction was washed with water (30 mL), dried and the solvent evaporated. The crude solid was purified by column chromatography, eluting with a gradient (0-10%) of MeOH/DCM, to give benzamide 99 (448 mg, 61%) as a white powder: mp (MeOH/DCM) 241-243° C.; ¹H NMR δ 12.84 (s, 1 H, NHCO), 8.64 (dd, J=4.5, 1.6 Hz, 2 H, H-2″, H-6″), 8.14 (br d, J=7.1 Hz, 2 H, H-2, H-6), 8.04 (s, 1 H, H-5′), 7.90 (dd, J=4.5, 1.6 Hz, 2 H, H-3″, H-5″), 7.56 (tt, J=7.4, 1.3 Hz, 1 H, H-4), 7.35-7.39 (m, 2 H, H-3, H-5); ¹³C NMR δ 165.3, 159.0, 150.1 (2), 146.6, 140.9, 132.6, 131.7, 128.5 (2), 128.1 (2), 119.9 (2), 112.6; MS m/z 282.4 (MH⁺, 100%). Anal. calcd for C₁₅H₁₁N₃OS: C, 64.04; H, 3.94; N, 14.94.Found: C, 63.75; H, 4.12; N, 14.93%.

### Example 1-62

### 3-Methyl-N-[4-(4-pyridinyl)-1,3-thiazol-2-yl]benzamide (100)

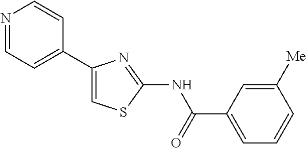

3-Methyl-N-[4-(4-pyridinyl)-1,3-thiazol-2-yl]benzamide (100). Oxalyl chloride (0.26 mL, 3.0 mmol) was added to a stirred solution of m-toluic acid (0.41 g, 3.0 mmol) and DMF (3 drops) in DCM (30 mL) and the solution stirred at 20° C. for 1 h. A solution of amine 98 (354 mg, 2.0 mmol) was added and the mixture stirred at 20° C. for 16 h. The solvent was evaporated and the residue partitioned between water (70 mL) and EtOAc (70 mL). The organic fraction was washed with water (30 mL), dried and the solvent evapporated. The crude solid was purified by column chromatography, eluting with a gradient (0-10%) of MeOH/EtOAc, to give benzamide 100 (110 mg, 19%) as a cream powder: mp (MeOH/EtOAc) 201-202° C.; ¹H NMR δ 12.77 (br s, 1 H, CONH), 8.64 (dd, J=4.6, 1.6 Hz, 2 H, H-2″, H-6″), 8.05 (s, 1 H, H-5), 7.98 (br s, 1 H, H-2), 7.92 (br d, J=6.9 Hz, 1 H, H-6), 7.88 (dd, J=4.6, 1.6 Hz, 2 H, H-3″, H-5″), 7.42-7.48 (m, 2 H, H-4, H-5), 2.41 (s, 3 H, CH₃); ¹³C NMR δ 165.4, 159.0, 150.1 (2), 146.5, 140.9, 137.8, 133.1, 131.7, 128.6, 128.3, 125.2, 119.2 (2), 112.5, 20.7; MS m/z 296.4 (WI', 100%). Anal. calcd for C₁₆H₁₃N₃OS: C, 65.06; H, 4.44; N, 14.23.Found: C, 65.19 H, 4.56; N, 14.31%.

### Example 1-63

### 4-Methyl-N-[4-(4-pyridinyl)-1,3-thiazol-2-yl]benzamide (101)

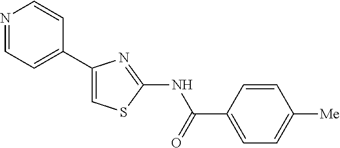

4-Methyl-N-[4-(4-pyridinyl)-1,3-thiazol-2-yl]benzamide (101). Oxalyl chloride (0.26 mL, 3.0 mmol) was added to a stirred solution of p-toluic acid (0.41 g, 3.0 mmol) and DMF (3 drops) in DCM (30 mL) and the solution stirred at 20° C. for 1 h. A solution of amine 98 (354 mg, 2.0 mmol) was added and the mixture stirred at 20° C. for 16 h. The solvent was evaporated and the residue partitioned between water (70 mL) and EtOAc (70 mL). The organic fraction was washed with water (30 mL), dried and the solvent evaporated. The crude solid was purified by column chromatography, eluting with a gradient (0-10%) of MeOH/EtOAc, to give benzamide 101 (328 mg, 56%) as a cream powder: mp (MeOH/EtOAc) 238-241° C.; ¹H NMR δ 12.74 (s, 1 H, CONH), 8.63 (dd, J=4.5, 1.6 Hz, 2 H, H-2″, H-6″), 8.04 (br d, J=8.0 Hz, 2 H, H-2, H-6), 8.02 (s, 1 H, H-5), 7.89 (dd, J=4.6, 1.6 Hz, 2 H, H-3″, H-5″), 7.35 (br d, J=8.0 Hz, 2 H, H-3, H-5), 2.39 (s, 3 H, CH₃); ¹³C NMR δ 165.2, 159.0, 150.1 (2), 146.5, 142.9, 140.9, 129.2 (2), 128.9, 128.1 (2), 119.2 (2), 112.5, 20.9; MS m/z 296.4 (WI', 100%). Anal. calcd for C₁₆H₁₃N₃OS: C, 65.06; H, 4.44; N, 14.23.Found: C, 64.96 H, 4.62; N, 14.24%.

### Example 1-64

### 4-Methoxy-N-[4-(4-pyridinyl)-1,3-thiazol-2-yl]benzamide (102)

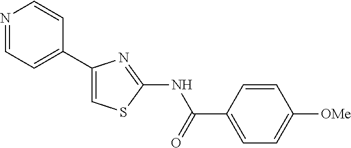

4-Methoxy-N-[4-(4-pyridinyl)-1,3-thiazol-2-yl]benzamide (102). 4-Methoxybenzoyl chloride (0.77 mL, 5.5 mmol) was added to a stirred solution of amine 98 (0.89 g, 5.0 mmol) and DMAP (60 mg, 0.5 mmol) in pyridine (30 mL) and the solution stirred at 20° C. for 16 h. The solvent was evaporated and the residue partitioned between water (70 mL) and EtOAc (70 mL). The organic fraction was washed with water (30 mL), dried and the solvent evaporated. The crude solid was purified by column chromatography, eluting with a gradient (0-10%) of MeOH/DCM, to give benzamide 102 (448 mg, 61%) as a white powder: mp (MeOH/DCM) 271-274° C.; ¹H NMR δ 12.66 (s, 1 H, NHCO), 8.63 (dd, J=4.5, 1.6 Hz, 2 H, H-2″, H-6″), 8.15 (ddd, J=8.9, 2.9, 2.0 Hz, 2 H, H-2, H-6), 8.01 (s, 1 H, H-5′), 7.88 (dd, J=4.5, 1.6 Hz, 2 H, H-3″, H-5″), 7.09 (ddd, J=8.9, 2.9, 2.0 Hz, 2 H, H-3, H-5), 3.86 (s, 3 H, OCH₃); ¹³C NMR δ 164.6, 162.7, 159.2, 150.1 (2), 146.5, 141.0, 130.2 (2), 123.8, 119.9 (2), 113.8 (2), 112.4, 55.4; MS m/z 312.5 (MH⁺, 100%). Anal. calcd for C₁₆H₁₃N₃O₂S: C, 61.72; H, 4.21; N, 13.50.

Found: C, 61.38; H, 4.48; N, 13.41%.

### Example 1-65

### N-(3-Methylbenzyl)-4-(4-pyridinyl)-1,3-thiazol-2-amine (103)

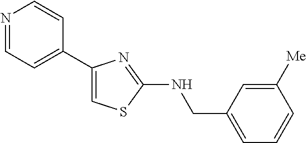

N-(3-Methylbenzyl)-4-(4-pyridinyl)-1,3-thiazol-2-amine (103). A mixture of 2-aminothiazole 98 (0.31 g, 1.8 mmol) and 3-methylbenzaldehyde (0.25 mL, 2.1 mmol) in THF (20 mL) was stirred at reflux temperature for 4 h. The mixture was cooled to 20° C. and a solution of NaBH₄ (265 mg, 7.0 mmol) in water (4 mL) was added dropwise and the mixture was stirred at 20° C. for 30 min. The mixture was diluted with water (30 mL) and extracted with EtOAc (3×25 mL). The combined organic fraction was dried and the solvent evaporated. The crude solid was purified by column chromatography, eluting with a gradient (50-100%) of EtOAc/pet. ether, to give amine 103 (0.11 g, 22%) as a cream powder: mp (MeOH/EtOAc) 192-194° C.; ¹H NMR δ 8.54 (dd, J=4.5, 1.6 Hz, 2 H, H-2′, H-6′), 8.30 (t, J=5.8 Hz, 1 H, NH), 7.75 (dd, J=4.5, 1.6 Hz, 2 H, H-3′, H-5′), 7.42 (s, 1 H, H-5), 7.17-7.24 (m, 3 H, H-2″, H-4″, H-5″), 7.07 (br d, J=6.9 Hz, 1 H, H-6″), 4.48 (d, J=5.8 Hz, 2 H, CH₂N), 2.30 (s, 3 H, CH₃); MS m/z 282.5 (MH⁺, 100%). Anal. calcd for C₁₆H₁₅N₃S.½CH₃OH: C, 67.87; H, 5.48; N, 14.72.Found: C, 67.78; H, 5.42; N, 14.92%.

### Example 1-66

### N-(3-Methylphenyl)-4-(4-pyridinyl)-1,3-oxazol-2-amine (105)

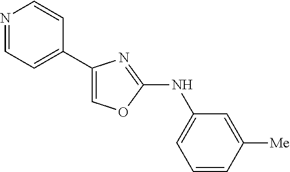

N-(3-Methylphenyl)-4-(4-pyridinyl)-1,3-oxazol-2-amine (105). A mixture of bromoketone hydrobromide 1 (0.32 g, 1.15 mmol) and 3-methylphenylurea 104 (0.17 g, 1.15 mmol) in EtOH (20 mL) was stirred at reflux temperature for 16 h. The mixture was cooled to 20° C., diluted with water (50 mL), the pH adjusted to ca. 8 with aqueous NH₃ and the mixture stirred at 20° C. for 2 h. The precipitate was filtered, washed with water (5 mL) and dried. The crude solid was purified by column chromatography, eluting with a gradient (0-5%) of MeOH/DCM, to give amine 105 (49 mg, 17%) as a cream powder: mp (EtOAc/DCM) 201-204° C.; ¹H NMR δ 10.18 (br s, 1 H, NH), 8.60 (dd, J=4.5, 1.6 Hz, 2 H, H-2′, H-6′), 8.40 (s, 1 H, H-5), 7.71 (dd, J=4.5, 1.6 Hz, 2 H, H-3′, H-5′), 7.54 (dd, J=8.1, 1.9 Hz, 1 H, H-6″), 7.46 (br s, 1 H, H-2″), 7.23 (dd, J=8.1, 7.5 Hz, 1 H, H-5″), 6.80 (br d, J=7.5 Hz, 1 H, H-4″), 2.32 (s, 3 H, CH₃); ¹³C NMR δ 157.1, 150.0 (2), 139.1, 138.5, 138.0, 136.9, 130.8, 128.7, 122.1, 119.1 (2), 117.1, 113.7, 21.2; MS m/z 252.4 (MH⁺, 100%). Anal. calcd for C₁₅H₁₃N₃O.¼CH₃OH: C, 70.64; H, 5.44; N, 16.21.Found: C, 70.64; H, 5.44; N, 16.51%.

### Example 1-67

### 2-{[4-(4-Pyridinyl)-1,3-thiazol-2-yl]amino}phenol (107)

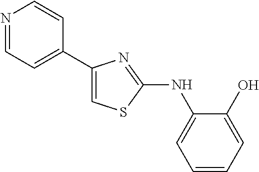

2-{[4-(4-Pyridinyl)-1,3-thiazol-2-yl]amino}phenol (107). A mixture of bromoketone hydrobromide 1 (0.34 g, 1.2 mmol) and N-(2-hydroxyphenyl)thiourea (106) (0.20 g, 1.2 mmol) in EtOH (15 mL) was stirred at reflux temperature for 1 h. The mixture was cooled to 20° C., diluted with water (50 mL), the pH adjusted to ca. 8 with aqueous NH₃ and the mixture stirred at 0° C. for 1 h. The precipitate was filtered, washed with water (5 mL) and dried. The crude solid was purified by column chromatography, eluting with a gradient (50-100%) of EtOAc/pet. ether, to give amine 107 (0.28 g, 86%) as a cream powder: mp (EtOAc/pet. ether) 221-223° C.; ¹H NMR δ 9.86 (br s, 1 H, OH), 9.56 (br s, 1 H, NH), 8.60 (dd, J=4.5, 1.6 Hz, 2 H, H-2″, H-6″), 8.26-8.31 (m, 1 H, H-4), 7.82 (dd, J=4.5, 1.6 Hz, 2 H, H-3″, H-5″), 7.63 (s, 1 H, H-5′), 6.83-6.91 (m, 3 H, H-3, H-5, H-6); ¹³C NMR δ 164.5, 150.0 (2), 147.1, 146.4, 141.1, 128.9, 122.4, 119.7 (2), 119.1, 119.0, 114.9, 107.6; MS m/z 270.5 (MH⁺, 100%). Anal. calcd for C₁₄H₁₁N₃OS: C, 62.43; H, 4.12; N, 15.60.Found: C, 62.59; H, 4.25; N, 15.42%.

### Example 1-68

### N-(2-Methoxyphenyl)-4-(4-pyridinyl)-1,3-thiazol-2-amine (109)

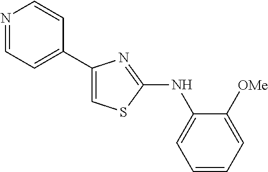

2-Methoxyphenylthiourea (108). NH₄SCN (3.37 g, 44.3 mmol) was added to a stirred solution of o-anisidine (5.00 g, 44.3 mmol) in 1 M HCl (45 mL) at 100° C. and the solution stirred at 100° C. for 16 h. The solution was diluted with water (60 mL) and the pH adjusted to 8 with aqueous NH₃ and the mixture at 5° C. for 2 h. The precipitate was filtered, washed with water (5 mL), washed with ether (5 mL), and dried. The crude solid was purified by column chromatography, eluting with 40% EtOAc/pet. ether, to give thiourea 108 (1.93 g, 24%) as a white powder: mp (EtOAc/pet. ether) 151-153° C. [lit. (Rasmussen, C. R. et al., Synthesis 1988, 456) mp (MeOH) 153-155° C.]; ¹H NMR δ 8.99 (s, 1 H, NH), 7.81 (br d, J=7.7 Hz, 1 H, H-3), 7.40 br s, 2 H, NH₂), 7.13 (ddd, J=8.2, 7.6, 1.6 Hz, 1 H, H-5), 7.04 (dd, J=8.2, 1.3 Hz, 1 H, H-6), 6.90 (ddd, J=7.7, 7.6, 1.3 Hz, 1 H, H-4), 3.81 (s, 3 H, OCH₃); MS m/z 183.3 (MH⁺, 100%).

N-(2-Methoxyphenyl]-4-(4-pyridinyl)-1,3-thiazol-2-amine (109). A mixture of bromoketone hydrobromide 1 (0.29 g, 1.1 mmol) and 2-methoxyphenylthiourea (108) (0.19 g, 1.1 mmol) in EtOH (15 mL) was stirred at reflux temperature for 1 h. The mixture was cooled to 20° C., diluted with water (50 mL), the pH adjusted to ca. 8 with aqueous NH₃ and the mixture stirred at 0° C. for 1 h. The precipitate was filtered, washed with water (5 mL) and dried. The crude solid was purified by column chromatography, eluting with a gradient (50-100%) of EtOAc/pet. ether, to give amine 109 (0.28 g, 93%) as a cream powder: mp (EtOAc/pet. ether) 190-191° C.; ¹H NMR δ 9.67 (br s, 1 H, NH), 8.60 (dd, J=4.5, 1.6 Hz, 2 H, H-2′, H-6′), 8.48 (br d, J=7.7 Hz, 1 H, H-3″), 7.83 (dd, J=4.5, 1.6 Hz, 2 H, H-3′, H-5′), 7.66 (s, 1 H, H-5), 6.97-7.00 (m, 3 H, H-4″, H-5″, H-6″), 3.87 (s, 3 H, OCH₃); ¹³C NMR δ 164.0, 150.0 (2), 148.0, 147.2, 141.0, 129.0, 122.1, 120.6, 119.7 (2), 118.2, 110.9, 108.0, 55.6; MS m/z 284.5 (MW, 100%). Anal. calcd for C₁₅H₁₃N₃OS: C, 63.58; H, 4.62; N, 14.83.Found: C, 63.63; H, 4.64; N, 14.77%.

### Example 1-69

### N-(2-Methylphenyl)-4-(4-pyridinyl)-1,3-thiazol-2-amine (111)

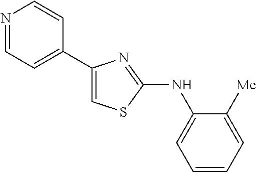

2-Methylphenylthiourea (110). NH₄SCN (3.55 g, 46.7 mmol) was added to a stirred solution of o-toluidine (5.00 g, 46.7 mmol) in 1 M HCl (47 mL) at 100° C. and the solution stirred at 100° C. for 16 h. The solution was diluted with water (60 mL) and the pH adjusted to 8 with aqueous NH₃ and the mixture at 5° C. for 2 h. The precipitate was filtered, washed with water (5 mL), washed with ether (5 mL), and dried. The crude solid was purified by column chromatography, eluting with 40% EtOAc/pet. ether, to give thiourea 110 (0.74 g, 10%) as a white powder: mp (EtOAc/pet. ether) 149-151° C. [lit. (Rasmussen, C. R. et al., Synthesis 1988, 456) mp (MeOH) 160.5-162.5° C.]; ¹H NMR δ 9.18 (s, 1 H, NH), 7.11 (m, 6 H, H-2, H-4, H-5, H-6, NH₂), 2.18 (s, 3 H, CH₃); MS m/z 167.3 (MW, 100%).

N-(2-Methylphenyl)-4-(4-pyridinyl)-1,3-thiazol-2-amine (111). A mixture of bromoketone hydrobromide 1 (291 mg, 1.0 mmol) and 2-methylphenylthiourea (110) (172 mg, 1.0 mmol) in EtOH (10 mL) was stirred at reflux temperature for 3 h. The mixture was cooled to 20° C., diluted with water (50 mL), the pH adjusted to ca. 8 with aqueous NH₃ and the mixture stirred at 20° C. for 2 h. The precipitate was filtered, washed with water (5 mL) and dried. The crude solid was purified by column chromatography, eluting with a gradient (50-100%) of EtOAc/pet. ether, to give amine 111 (0.24 g, 87%) as a cream powder: mp (EtOAc/pet. ether) 158-160° C.; ¹H NMR δ 9.44 (br s, 1 H, NH), 8.58 (dd, J=4.6, 1.5 Hz, 2 H, H-2′, H-6′), 7.96 (br d, J=8.0 Hz, 1 H, H-3″), 7.78 (dd, J=4.6, 1.5 Hz, 2 H, H-3′, H-5′), 7.61 (s, 1 H, H-5), 7.21-7.26 (m, 2 H, H-5″, H-6″), 7.04 (dt, J=7.4, 0.9 Hz, 1 H, H-4″), 2.29 (s, 3 H, CH₃); ¹³C NMR δ 165.8, 150.0 (2), 147.4, 141.1, 139.1, 130.6, 129.1, 126.5, 123.6, 121.2, 119.7 (2), 107.2, 17.9; MS m/z 268.5 (MW, 100%). Anal. calcd for C₁₅H₁₃N₃S: C, 67.39; H, 4.90; N, 15.72.

Found: C, 67.67; H, 4.97; N, 15.61%.

### Example 1-70

### N-(2-Fluorophenyl)-4-(4-pyridinyl)-1,3-thiazol-2-amine (113)

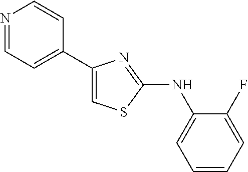

N-(2-Fluorophenyl)-4-(4-pyridinyl)-1,3-thiazol-2-amine (113). A mixture of bromoketone hydrobromide 1 (0.37 g, 1.3 mmol) and N-(2-fluorophenyl)thiourea (112) (0.22 g, 1.3 mmol) in EtOH (15 mL) was stirred at reflux temperature for 1 h. The mixture was cooled to 20° C., diluted with water (50 mL), the pH adjusted to ca. 8 with aqueous NH₃ and the mixture stirred at 0° C. for 1 h. The precipitate was filtered, washed with water (5 mL) and dried. The crude solid was purified by column chromatography, eluting with a gradient (50-100%) of EtOAc/pet. ether, to give amine 113 (0.31 g, 87%) as a cream powder: mp (EtOAc/pet. ether) 190-192° C.; ¹H NMR δ 10.16 (br s, 1 H, NH), 8.63 (dd, J=4.5, 1.6 Hz, 2 H, H-2′, H-6′), 8.56 (dt, J=8.4, 1.5 Hz, 1 H, H-3″), 7.84 (dd, J=4.5, 1.6 Hz, 2 H, H-3′, H-5′), 7.73 (s, 1 H, H-5), 7.22-7.30 (m, 2 H, H-4″, H-6″), 7.01-7.06 (m, 1 H, H-5″); ¹³C NMR δ 163.5, 151.6 (d, J=243 Hz), 150.4 (2), 147.3, 140.9, 128.7 (d, J=11 Hz), 124.6 (d, J=3 Hz), 122.3 (d, J=7 Hz), 119.8, 119.8 (2), 115.0 (d, J=19 Hz), 108.5; MS m/z 272.5 (MH⁺, 100%). Anal. calcd for C₁₄H₁₀H₃S: C, 61.98; H, 3.72; N, 15.49.Found: C, 61.89; H, 3.37; N, 15.68%.

### Example 1-71

### N-[2-Chlorophenyl]-4-(4-pyridinyl)-1,3-thiazol-2-amine (115)

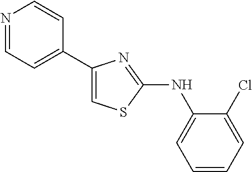

2-Chlorophenylthiourea (114). NH₄SCN (3.12 g, 47.5 mmol) was added to a stirred solution of 2-chloroaniline (6.07 g, 47.5 mmol) in 1 M HCl (48 mL) at 100° C. and the solution stirred at 100° C. for 16 h. The solution was diluted with water (60 mL) and the pH adjusted to 8 with aqueous NH₃ and the mixture was stirred at 5° C. for 2 h. The precipitate was filtered, washed with water (5 mL), washed with ether (5 mL), and dried. The crude solid was purified by column chromatography, eluting with 40% EtOAc/pet. ether, to give thiourea 114 (1.23 g, 14%) as a white powder: mp (EtOAc/pet. ether) 141-143° C. [lit. (Rasmussen, C. R. et al., Synthesis 1988, 456) mp (acetone) 145-146.5° C.]; ¹H NMR δ 9.28 (s, 1 H, NH), 7.65 (dd, J=8.0, 1.4 Hz, 1 H, H-3), 7.48 (dd, J=8.0, 1.4 Hz, 1 H, H-6), 7.38 (br s, 2 H, NH₂), 7.32 (ddd, J=8.0, 7.6, 1.4 Hz, 1 H, H-5), 7.23 (ddd, J=8.0, 7.6, 1.4 Hz, 1 H, H-4); MS m/z 187.5/189.4 (MH⁺, 100%).

N-[2-Chlorophenyl]-4-(4-pyridinyl)-1,3-thiazol-2-amine (115). A mixture of bromoketone hydrobromide 1 (0.30 g, 1.1 mmol) and 2-chlorophenylthiourea (114) (0.20 g, 1.1 mmol) in EtOH (15 mL) was stirred at reflux temperature for 1 h. The mixture was cooled to 20° C., diluted with water (50 mL), the pH adjusted to ca. 8 with aqueous NH₃ and the mixture stirred at 0° C. for 1 h. The precipitate was filtered, washed with water (5 mL) and dried. The crude solid was purified by column chromatography, eluting with a gradient (50-100%) of EtOAc/pet. ether, to give amine 115 (0.27 g, 90%) as a cream powder: mp (EtOAc/pet. ether) 152-154° C.; ¹H NMR δ 9.79 (br s, 1 H, NH), 8.58 (dd, J=4.5, 1.6 Hz, 2 H, H-2′, H-6′), 8.44 (dd, J=8.3, 1.4 Hz, 1 H, H-3″), 7.82 (dd, J=4.5, 1.6 Hz, 2 H, H-3′, H-5′), 7.73 (s, 1 H, H-5), 7.50 (dd, J=8.0, 1.4 Hz, 1 H, H-6″), 7.40 (ddd, J=8.0, 7.5, 1.5 Hz, 1 H, H-5″), 7.09 (ddd, J=8.3, 7.5, 1.5 Hz, 1 H, H-4″); MS m/z 288.5/290.5 (MH⁺, 100%). Anal. calcd for C₁₄H₁₀ClN₃S: C, 58.43; H, 3.50; N, 14.60.Found: C, 58.34; H, 3.62; N, 14.63%.

### Example 1-72

### 4-(4-Pyridinyl)-N-[2-(trifluoromethyl)phenyl]-1,3-thiazol-2-amine (117)

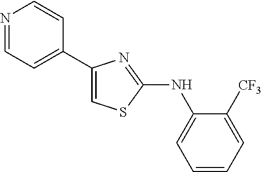

4-(4-Pyridinyl)-N-[2-(trifluoromethyl)phenyl]-1,3-thiazol-2-amine (117). A mixture of bromoketone hydrobromide 1 (0.30 g, 1.1 mmol) and 2-trifluorophenylthiourea (116) (0.23 g, 1.1 mmol) in EtOH (15 mL) was stirred at reflux temperature for 1 h. The mixture was cooled to 20° C., diluted with water (50 mL), the pH adjusted to ca. 8 with aqueous NH₃ and the mixture stirred at 0° C. for 1 h. The precipitate was filtered, washed with water (5 mL) and dried. The crude solid was purified by column chromatography, eluting with a gradient (50-100%) of EtOAc/pet. ether, to give amine 117 (0.31 g, 93%) as a cream powder: mp (EtOAc/pet. ether) 170-172° C.; ¹H NMR δ 9.61 (br s, 1 H, NH), 8.57 (dd, J=4.5, 1.6 Hz, 2 H, H-2″, H-6″), 8.11 (br d, J=8.1 Hz, 1 H, H-3′), 7.67-7.76 (m, 5 H, H-5′, H-6′, H-3″, H-5″), 7.37 (br t, J=7.6 Hz, 1 H, H-4′); ¹³C NMR δ 165.7, 150.0 (2), 147.3, 140.9, 138.3, 133.3, 126.4 (q, J=5 Hz), 125.8, 124.5, 123.7 (q, J=273 Hz), 121.2 (q, J=29 Hz), 119.7 (2), 108.6; MS m/z 322.6 (MH⁺, 100%). Anal. calcd for C₁₅H₁₀F₃N₃S: C, 56.07; H, 3.14; N, 17.74.Found: C, 56.07; H, 3.27; N, 13.08%.

### Example 1-73

### N-[2-Nitrophenyl]-4-(4-pyridinyl)-1,3-thiazol-2-amine (119)

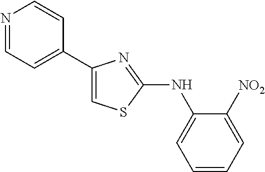

N-[2-Nitrophenyl]-4-(4-pyridinyl)-1,3-thiazol-2-amine (119). A mixture of bromoketone hydrobromide 1 (0.32 g, 1.1 mmol) and 2-nitrophenylthiourea (118) (0.22 g, 1.1 mmol) in EtOH (15 mL) was stirred at reflux temperature for 1 h. The mixture was cooled to 20° C., diluted with water (50 mL), the pH adjusted to ca. 8 with aqueous NH₃ and the mixture stirred at 0° C. for 1 h. The precipitate was filtered, washed with water (5 mL) and dried. The crude solid was purified by column chromatography, eluting with a gradient (50-100%) of EtOAc/pet. ether, to give amine 119 (0.32 g, 93%) as a cream powder: mp (EtOAc/pet. ether) 127-130° C.; ¹H NMR δ 10.55 (br s, 1 H, NH), 8.60 (dd, J=4.5, 1.6 Hz, 2 H, H-2′, H-6′), 8.27 (dd, J=8.4, 1.0 Hz, 1 H, H-3″), 8.07 (dd, J=8.3, 1.0 Hz, 1 H, H-6″), 7.85 (s, 1 H, H-5), 7.72-7.76 (m, 3 H, H-3′, H-5′, H-5″), 7.24 (ddd, J=8.4, 7.4, 1.2 Hz, 1 H, H-4″); ¹³C NMR δ 162.7, 150.0 (2), 147.3, 140.6, 138.3, 134.9, 134.8, 125.4, 122.3, 121.6, 119.8 (2), 110.3; MS m/z 299.5 (MH⁺, 100%). Anal. calcd for C₁₄H₁₀N₄O₂S: C, 56.37; H, 3.38; N, 18.78.Found: C, 56.29; H, 3.39; N, 18.84%.

### Example 1-74

### N-(3-Fluorophenyl)-4-(4-pyridinyl)-1,3-thiazol-2-amine (121)

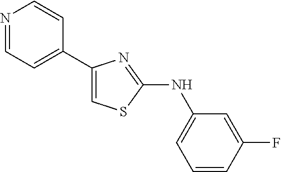

3-Fluorophenylthiourea (120). Reaction of NH₄SCN (0.53 g, 7.0 mmol) and benzoylchloride (0.82 mL, 7.2 mmol) with 3-chloroaniline (0.45 mL, 4.6 mmol) gave thiourea 120 (0.49 g, 62%) as a white powder: mp (H₂O) 100-102° C. [lit. (Schnur, R. C.; et.al. J. Med. Chem. 1991, 34, 914-918.) mp 113-114° C.]; ¹H NMR δ 9.81 (s, 1 H, NH), 7.60 (br s, 2 H, NH₂), 7.55 (dt, J=11.5, 2.4 Hz, 1 H, H-2), 7.33 (dt, J=9.2, 6.8 Hz, 1 H, H-5), 7.16 (ddd, J=6.8, 1.9, 1.0 Hz, 1 H, H-6), 6.85-6.93 (m 1 H, H-4).

N-(3-Fluorophenyl)-4-(4-pyridinyl)-1,3-thiazol-2-amine (121). Reaction of bromoketone hydrobromide 1 (0.65 g, 2.3 mmol) and 3-fluorophenylthiourea (120) (0.39 g, 2.3 mmol) gave amine 121 (0.45 g, 71%) as a white powder: mp (EtOAc/pet. ether) 229-230° C.; ¹H NMR δ 10.59 (br s, 1 H, NH), 8.63 (dd, J=4.5, 1.6 Hz, 2 H, H-2′, H-6′), 7.35 (dd, J=4.5, 1.6 Hz, 2 H, H-3′, H-5′), 7.28-7.31 (m, 1 H, H-2″), 7.76 (s, 1 H, H-5), 7.34-7.41 (m, 2 H, H-5″, H-6″), 6.77-6.84 (m, 1 H, H-4″); ¹³C NMR δ 163.0, 162.4 (d, J=240 Hz), 150.1 (2), 147.6, 142.4 (d, J=11 Hz), 140.8, 130.5 (d, J=10 Hz), 119.8 (2), 112.7 (d, J=2 Hz), 108.0, 107.5 (d, J=21 Hz), 103.5 (d, J=26 Hz). Anal. calcd for C₁₄H₁₀FN₃S: C, 61.98; H, 3.72; N, 15.49.Found: C, 61.91; H, 3.79; N, 15.20%.

### Example 1-75

### N-(3-Bromophenyl)-4-(4-pyridinyl)-1,3-thiazol-2-amine (123)

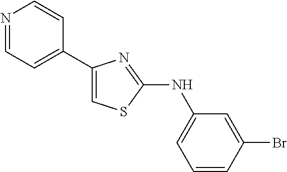

3-Bromophenylthiourea (122). Benzoyl chloride (4.5 mL, 38.6 mmol) was added dropwise to a stirred solution of NH₄SCN (3.14 g, 41.3 mmol) in acetone (50 mL). The mixture was stirred at reflux temperature for 15 min, the heating was removed and 3-bromoaniline (3.0 mL, 27.5 mmol) was added dropwise, keeping the solution at reflux temperature. The mixture was stirred at reflux temperature for 30 min, then poured onto ice (100 mL) and stirred for 15 min. The resulting mixture was filtered and washed with water (5 mL), and dried. The solid was added to a stirred solution of NaOH (10% w/v, 100 mL) at 80° C. The solution was stirred at 80° C. for 30 min and then poured onto ice/HCl (6 M, 30 mL) and stirred for 10 mins. The pH was adjusted to 10 with cNH₃ solution, the resulting precipitate filtered and washed with water (10 mL) and dried. The crude solid was purified by column chromatography, eluting with a gradient (20-50%) of EtOAc/pet. ether, to give thiourea 122 (5.18 g, 82%) as a tan powder: mp (EtOAc/pet. ether) 151-152° C. [lit (Mahapatra, G. N.J. Indian Chem. Soc. 1956, 33, 923-924) mp (EtOH) 138° C.]; ¹H NMR δ 9.78 (s, 1 H, NH), 7.83 (br s, 1 H, H-2,), 7.58 (br s, 2 H, NH₂), 7.35-7.38 (m, 1 H, H-5), 7.25-7.29 (m, 2 H, H-4, H-6).

N-(3-Bromophenyl)-4-(4-pyridinyl)-1,3-thiazol-2-amine (123). A mixture of bromoketone hydrobromide 1 (0.72 g, 2.6 mmol) and 3-bromophenylthiourea (122) (0.59 g, 2.6 mmol) in EtOH (40 mL) was stirred at reflux temperature for 2 h. The mixture was cooled to 20° C., diluted with water (50 mL), the pH adjusted to ca. 8 with aqueous NH₃ and the mixture stirred at 0° C. for 2 h. The precipitate was filtered, washed with water (5 mL) and dried. The crude solid was purified by column chromatography, eluting with EtOAc, to give amine 123 (0.62 g, 72%) as a cream powder: mp (EtOAc/pet. ether) 219-221° C.; ¹H NMR δ 10.55 (br s, 1 H, NH), 8.63 (dd, J=4.5, 1.6 Hz, 2 H, H-2′, H-6′), 8.07 (t, J=2.0 Hz, 1 H, H-2″), 7.85 (dd, J=4.5, 1.6 Hz, 2 H, H-3′, H-5′), 7.78 (s, 1 H, H-5), 7.67 (ddd, J=8.2, 2.0, 0.8 Hz, 1 H, H-6″), 7.31 (br t, J=8.1 Hz, 1 H, H-5″), 7.16 (ddd, J=7.9, 2.0, 0.8 Hz, 1 H, H-4″); ¹³C NMR δ 162.9, 150.1 (2), 147.6, 142.3, 140.8, 130.8, 127.3, 121.7, 119.7 (2), 119.1, 115.6, 108.1; MS m/z 332.5/334.5 (MH⁺, 100%). Anal. calcd for C₁₄H₁₀BrN₃S.¼CH₃CO₂CH₂CH₃: C, 50.86; H, 3.41; N, 11.86.Found: C, 51.02; H, 3.41; N, 11.86%.

### Example 1-76

### 1-(3-{[4-(4-Pyridinyl)-1,3-thiazol-2-yl]amino}phenyl)ethanone (125)

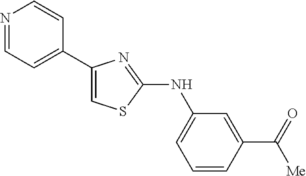

N-(3-Acetylphenyl)thiourea (124). NH₄SCN (2.88 g, 37.8 mmol) was added to a stirred solution of 3-aminoacetophenone (5.11 g, 37.8 mmol) in 1 M HCl (38 mL) at 100° C. and the solution stirred at 100° C. for 16 h. The solution was diluted with water (60 mL) and stood at 5° C. for 1 h. The precipitate was filtered, washed with water (5 mL), washed with ether (5 mL), and dried. The crude solid was purified by column chromatography, eluting with a gradient (50-100%) of EtOAc/pet. ether, to give to give thiourea 124 (0.81 g, 11%) as a white powder: mp (EtOAc/pet. ether) 153-156° C.; ¹H NMR δ 9.83 (s, 1 H, NH), 7.50 (br s, 2 H, NH₂), 8.02 (t, J=1.8 Hz, 1 H, H-2′), 7.67-7.73 (m, 2 H, H-4′, H-6′), 7.46 (t, J=7.9 Hz, 1 H, H-5′), 2.56 (s, 3 H, COCH₃).

1-(3-{[4-(4-Pyridinyl)-1,3-thiazol-2-yl]amino}phenyl)ethanone (125). A mixture of bromoketone hydrobromide 1 (0.87 g, 3.1 mmol) and N-(3-acetylphenyl)thiourea (124) (0.60 g, 3.1 mmol) in EtOH (20 mL) was stirred at reflux temperature for 3 h. The mixture was cooled to 20° C., diluted with water (50 mL), the pH adjusted to ca. 8 with aqueous NH₃ and the mixture stirred at 20° C. for 2 h. The precipitate was filtered, washed with water (5 mL) and dried. The crude solid was purified by column chromatography, eluting with a gradient (50-100%) of EtOAc/pet. ether, to give amine 125 (0.85 g, 93%) as a white powder: mp (EtOAc/pet. ether) 203-206° C.; ¹H NMR δ 10.58 (br s, 1 H, NH), 8.62 (dd, J=4.5, 1.6 Hz, 2 H, H-2″, H-6″), 8.45 (t, J=1.9 Hz, 1 H, H-2′), 7.95 (ddd, J=8.0, 2.3, 1.0 Hz, 1 H, H-4′), 7.88 (dd, J=4.5, 1.6 Hz, 2 H, H-3″, H-5″), 7.76 (s, 1 H, H-5″), 7.59 (dt, J=8.0, 1.5 Hz, 1 H, H-5′), 7.51 (br t, J=7.9 Hz, 1 H, H-5′), 2.61 (s, 3 H, H-2); ¹³C NMR δ 197.5, 163.1, 150.0 (2), 147.6, 141.1, 140.8, 137.5, 129.3, 121.2 (2), 119.8 (2), 116.0, 107.9, 26.6; MS m/z 296.6 (MH⁺, 100%). Anal. calcd for C₁₆H₁₃N₃OS: C, 65.06; H, 4.44; N, 14.23.Found: C, 64.76; H, 4.29; N, 14.36%.

### Example 1-77

### 3-{[4-(4-Pyridinyl)-1,3-thiazol-2-yl]amino}benzonitrile (127)

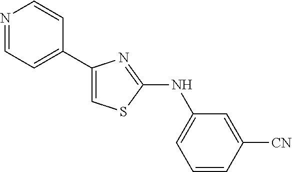

3-Cyanophenylthiourea (126). A mixture of 3-aminobenzonitrile (1.0 g, 8.5 mmol) and NH₄SCN (0.64 g, 8.5 mmol) in dilute HCl (1 M, 9 mL) was stirred at reflux temperature for 16 h. The mixture was cooled and water (50 ml) added and the pH was adjusted to 10 with cNH₃ solution, the resulting precipitate was filtered and washed with water (10 mL) and dried. The crude solid was purified by column chromatography, eluting with 40% EtOAc/DCM, to give thiourea 126 (0.22 g, 15%) as a tan powder: mp (EtOAc/DCM) 158-160° C.; ¹H NMR δ 9.89 (s, 1 H, NH), 8.03 (br s, 1 H, H-2), 7.70 (dt, J=7.6, 2.0 Hz, 1 H, H-4), 7.60 (br s, 2 H, NH₂), 7.48-7.55 (m, 2 H, H-5, H-6).

3-{[4-(4-Pyridinyl)-1,3-thiazol-2-yl]amino}benzonitrile (127). A mixture of bromoketone 1 (0.17 g, 0.62 mmol) and 3-cyanophenylhiourea (126) (0.11 g, 0.62 mmol) in EtOH (10 mL) was stirred at reflux temperature for 2 h. The mixture was cooled to 20° C., diluted with water (50 mL), the pH adjusted to ca. 8 with aqueous NH₃ and the mixture stirred at 20° C. for 1 h. The precipitate was filtered, washed with water (5 mL) and dried. The crude solid was purified by column chromatography, eluting with 5% MeOH/DCM, to give amine 127 (0.15 g, 90%) as a white powder: mp (MeOH/DCM) 252-253° C.; ¹H NMR δ 10.73 (s, 1 H, NH), 8.64 (dd, J=4.5, 1.6 Hz, 2 H, H-2″, H-6″), 8.18 (t, J=1.8 Hz, 1 H, H-2), 8.01 (ddd, J=8.4, 2.3, 0.9 Hz, 1 H, H-6), 7.86 (dd, J=4.5, 1.6 Hz, 2 H, H-3″, H-5″), 7.81 (s, 1 H, H-5′), 7.58 (br t, J=8.0 Hz, 1 H, H-5), 7.43 (dt, J=7.7, 1.1 Hz, 1 H, H-4); MS m/z 279.5 (MH⁺, 100%). Anal. calcd for C₁₅H₁₀N₄S.1/2H₂O: C, 63.70; H, 3.74; N, 19.81.Found: C, 63.63; H, 3.92; N, 19.72%.

### Example 1-78

### N-(3-Nitrophenyl)-4-(4-pyridinyl)-1,3-thiazol-2-amine (129)

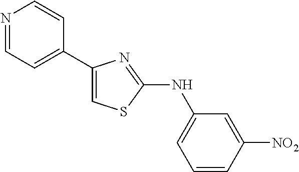

3-Nitrophenylthiourea (128). NH₄SCN (2.89 g, 38.0 mmol) was added to a stirred solution of m-nitroaniline (5.00 g, 36.2 mmol) in 1 M HCl (37 mL) at 100° C. and the solution stirred at 100° C. for 16 h. The solution was diluted with water (60 mL) and stood at 5° C. for 2 h. The precipitate was filtered, washed with water (5 mL), washed with ether (5 mL). The precipitate was purified by column chromatography, eluting with a gradient (50-80%) of EtOAc/pet. ether, to give thiourea 128 (0.45 g, 6%) as an orange powder: mp (EtOAc) 152-154° C.; ¹H NMR δ 10.07 (s, 1 H, NH), 8.61 (t, J=2.2 Hz, 1 H, H-2), 7.93 (ddd, J=8.2, 2.2, 0.9 Hz, 1 H, H-6), 7.83 (ddd, J=8.2, 2.2, 0.9 Hz, 1 H, H-4), 7.60 (br s, 2 H, NH₂), 7.58 (t, J=8.2 Hz, 1 H, H-5).

N-(3-Nitrophenyl)-4-(4-pyridinyl)-1,3-thiazol-2-amine (129). A mixture of bromoketone hydrobromide 1 (0.58 g, 2.1 mmol) and 3-nitrophenylthiourea (128) (0.41 g, 2.1 mmol) in EtOH (40 mL) was stirred at reflux temperature for 2 h. The mixture was cooled to 20° C., diluted with water (50 mL), the pH adjusted to ca. 8 with aqueous NH₃ and the mixture stirred at 0° C. for 2 h. The precipitate was filtered, washed with water (5 mL) and dried. The crude solid was purified by column chromatography, eluting with EtOAc, to give amine 129 (0.43 g, 69%) as a orange powder: mp (EtOAc/pet. ether) 244-247° C.; ¹H NMR δ 10.89 (br s, 1 H, NH), 8.95 (t, J=2.2 Hz, 1 H, H-2″), 8.64 (dd, J=4.5, 1.6 Hz, 2 H, H-2′, H-6′), 7.97 (ddd, J=8.2, 2.1, 0.7 Hz, 1 H, H-4″), 7.88 (dd, J=4.5, 1.6 Hz, 2 H, H-3′, H-5′), 7.80-7.84 (m, 2 H, H-5, H-6″), 7.63 (t, J=8.2 Hz, 1 H, H-5″); ¹³C NMR δ 162.6, 150.1 (2), 148.2, 147.6, 141.8, 140.7, 130.2, 122.8, 119.7 (2), 115.5, 110.7, 108.6; MS m/z 299.5 (MH⁺, 100%). Anal. calcd for C₁₄H₁₀N₄O₂S.¼CH₃OH: C, 55.87; H, 3.62; N, 18.29.Found: C, 55.85; H, 3.56; N, 18.21%.

### Example 1-79

### 1-(4-{[4-(4-Pyridinyl)-1,3-thiazol-2-yl]amino}phenyl)ethanone (131)

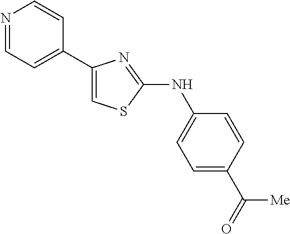

4-Acetylphenylthiourea (130). NH₄SCN (2.85 g, 37.4 mmol) was added to a stirred solution of 4-acetylaniline (5.05 g, 37.4 mmol) in 1 M HCl (40 mL) at 100° C. and the solution stirred at 100° C. for 16 h. The solution was diluted with water (60 mL) and stood at 5° C. for 2 h. The precipitate was filtered, washed with water (5 mL), washed with ether (5 mL), and dried. The crude solid was suspended in hot acetone/MeOH, filtered, and the solvent evaporated to give thiourea 130 (1.70 g, 23%) as a white powder: mp (acetone/MeOH) 202-205° C.; ¹H NMR δ 9.90 (s, 1 H, NH), 7.90 (ddd, J=8.8, 2.3, 2.0 Hz, 2 H, H-3′, H-5′), 7.68 (ddd, J=8.8, 2.3, 2.0 Hz, 2 H, H-2′, H-6′), 7.60 (br s, 2 H, NH₂), 2.53 (s, 3 H, COCH₃).

1-(4-{[4-(4-Pyridinyl)-1,3-thiazol-2-yl]amino}phenyl)ethanone (131). A mixture of bromoketone hydrobromide 1 (0.60 g, 2.14 mmol) and 4-acetylphenylthiourea (130) (0.41 g, 2.14 mmol) in EtOH (20 mL) was stirred at reflux temperature for 3 h. The mixture was cooled to 20° C., diluted with water (50 mL), the pH adjusted to ca. 8 with aqueous NH₃ and the mixture stirred at 20° C. for 2 h. The precipitate was filtered, washed with water (5 mL) and dried. The crude solid was purified by column chromatography, eluting with 50% EtOAc/pet. ether, to give amine 131 (0.40 g, 64%) as a cream powder: mp (EtOAc/pet. ether) 229-231° C.; ¹H NMR δ 10.80 (br s, 1 H, NH), 8.63 (dd, J=4.5, 1.6 Hz, 2 H, H-2″, H-6″), 8.01 (br d, J=8.8 Hz, 2 H, H-2′, H-6′), 7.89 (dd, J=4.5, 1.6 Hz, 2 H, H-3″, H-5″′), 7.85 (br d, J=8.8 Hz, 2 H, H-3′, H-5′), 7.82 (s, 1 H, H-5″), 2.53 (s, 3 H, H-2); ¹³C NMR δ 195.9, 162.5, 150.1 (2), 147.7, 144.8, 140.7, 129.8 (2), 119.8 (2), 116.0 (2), 108.7, 26.1, one carbon not observed. Anal. calcd for C₁₆H₁₃N₃OS: C, 65.06; H, 4.44; N, 14.23.Found: C, 65.11; H, 4.40; N, 14.13%.

### Example 1-80

### 4-(4-Pyridinyl)-N-[4-(trifluoromethyl)phenyl]-1,3-thiazol-2-amine (133)

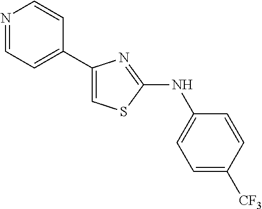

N-[4-(Trifluoromethyl)phenyl]thiourea (132). NH₄SCN (2.74 g, 36.0 mmol) was added to a stirred solution of 4-trifluoromethylaniline (5.80 g, 36.0 mmol) in 1 M HCl (40 mL) at 100° C. and the solution stirred at 100° C. for 16 h. The solution was diluted with water (60 mL) and stood at 5° C. for 2 h. The precipitate was filtered, washed with water (5 mL), washed with ether (5 mL), and dried. The crude solid was purified by column chromatography, eluting with a gradient (50-100%) of EtOAc/pet. ether, to give thiourea 132 (0.98 g, 12%) as a white powder: mp (EtOAc/pet. ether) 122-125° C. [lit. (Rasmussen et al., Synthesis 1988, 456) mp (toluene) 143.5-146° C.]; ¹H NMR δ 9.97 (s, 1 H, NH), 9.80 (br s, 2 H, NH₂), 7.73 (d, J=8.6 Hz, 2 H, H-3′, H-5′), 7.65 (br d, J=8.6 Hz, 2 H, H-2′, H-6′), MS m/z 221.4 (MH⁺, 100%).

4-(4-Pyridinyl)-N-[4-(trifluoromethyl)phenyl]-1,3-thiazol-2-amine (133). A mixture of bromoketone hydrobromide 1 (0.27 g, 1.0 mmol) and 4-trifluoromethylphenylthiourea (132) (0.22 g, 1.0 mmol) in EtOH (15 mL) was stirred at reflux temperature for 3 h. The mixture was cooled to 20° C., diluted with water (50 mL), the pH adjusted to ca. 8 with aqueous NH₃ and the mixture stirred at 20° C. for 2 h. The precipitate was filtered, washed with water (5 mL) and dried. The crude solid was purified by column chromatography, eluting with EtOAc/pet. ether, to give amine 133 (0.17 g, 54%) as a cream powder: mp (EtOAc/pet. ether) 238-241° C.; ¹H NMR δ 10.77 (br s, 1 H, NH), 8.63 (dd, J=4.5, 1.6 Hz, 2 H, H-2″, H-6″), 7.94 (br d, J=8.6 Hz, 2 H, H-3′, H-5′), 7.89 (dd, J=4.5, 1.6 Hz, 2 H, H-3″, H-5″), 7.81 (s, 1 H, H-5″), 7.71 (br d, J=8.6 Hz, 2 H, H-2′, H-6′); ¹³C NMR δ 162.7, 150.1 (2), 147.7, 144.1, 140.7, 126.2 (q, J=4 Hz, 2), 124.5 (q, J=271 Hz), 121.0 (q, J=32 Hz), 119.8 (2), 116.6 (2), 108.6; MS m/z 322.5 (MW, 100%). Anal. calcd for C₁₅H₁₀F₃N₃S: C, 56.07; H, 3.14; N, 13.08.Found: C, 56.47; H, 3.20; N, 13.15%.

### Example 1-81

### 4-{[4-(4-Pyridinyl)-1,3-thiazol-2-yl]amino}benzonitrile (135)

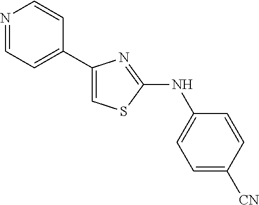

N-(4-Cyanophenyl)thiourea (134). NH₄SCN (3.83 g, 50.4 mmol) was added to a stirred solution of 4-aminobenzonitrile (5.95 g, 50.4 mmol) in 1 M HCl (50 mL) at 100° C. and the solution stirred at 100° C. for 16 h. The solution was diluted with water (60 mL) and stood at 5° C. for 2 h. The precipitate was filtered, washed with water (5 mL), washed with ether (5 mL), and dried. The crude solid was purified by column chromatography, eluting with 40% EtOAc/DCM, to give thiourea 134 (3.58 g, 40%) as a white powder: mp (EtOAc/DCM)>310° C. [lit. (Cohen, V. I. et. al. Bull. Soc. Chim. France 1965, 8, 2314-2317) mp 239° C.]; ¹H NMR δ 10.06 (s, 1 H, NH), 7.70-7.80 (m, 6 H, NH₂, H-2′, H-3′, H-5′, H-6′); MS m/z 178.4 (MH⁺, 100%).

4-{[4-(4-Pyridinyl)-1,3-thiazol-2-yl]amino}benzonitrile (135). A mixture of bromoketone hydrobromide 1 (0.31 g, 1.1 mmol) and N-(4-cyanophenyl)thiourea (134) (0.19 g, 1.1 mmol) in EtOH (15 mL) was stirred at reflux temperature for 1 h. The mixture was cooled to 20° C., diluted with water (50 mL), the pH adjusted to ca. 8 with aqueous NH₃ and the mixture stirred at 20° C. for 2 h. The precipitate was filtered, washed with water (5 mL) and dried. The crude solid was purified by column chromatography, eluting with a gradient (50-100%) of EtOAc/pet. ether, to give amine 135 (0.27 g, 90%) as a cream powder: mp (EtOAc/pet. ether)>300° C.; ¹H NMR δ 10.89 (br s, 1 H, NH), 8.64 (dd, J=4.6, 1.5 Hz, 2 H, H-2″, H-6″), 7.86 (m, 4 H, H-2, H-6, H-3″, H-5″), 7.84 (s, 1 H, H-5′), 7.78 (br d, J=8.8 Hz, 2 H, H-3, H-5); ¹³C NMR δ 162.4, 150.1 (2), 147.8, 144.5, 140.7, 133.4 (2), 119.9 (2), 119.3, 116.8 (2), 109.1, 102.4; MS m/z 279.5 (MH⁺, 100%). Anal. calcd for C₁₅H₁₀N4S: C, 64.73; H, 3.62; N, 20.13.Found: C, 64.50; H, 3.80; N, 20.17%.

### Example 1-82

### N-(2,6-Dimethylphenyl)-4-(4-pyridinyl)-1,3-thiazol-2-amine (137)

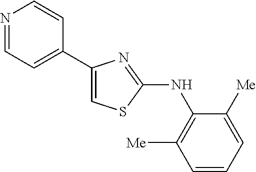

N-(2,6-dimethylphenyl)-4-(4-pyridinyl)-1,3-thiazol-2-amine (137). A mixture of bromoketone hydrobromide 1 (0.30 g, 1.1 mmol) and N-(2,6-difluorophenyl)thiourea (136) (0.20 g, 1.1 mmol) in EtOH (15 mL) was stirred at reflux temperature for 1 h. The mixture was cooled to 20° C., diluted with water (50 mL), the pH adjusted to ca. 8 with aqueous NH₃ and the mixture stirred at 0° C. for 1 h. The precipitate was filtered, washed with water (5 mL) and dried. The crude solid was purified by column chromatography, eluting with EtOAc, to give amine 137 (0.28 g, 91%) as a white powder: mp (EtOAc/pet. ether) 208-210° C.; ¹H NMR δ 9.34 (s, 1 H, NH), 8.55 (dd, J=4.6, 1.6 Hz, 2 H, H-2′, H-6′), 7.72 (dd, J=4.6, 1.6 Hz, 2 H, H-3′, H-5′), 7.46 (s, 1 H, H-5), 7.12-7.19 (m, 3 H, H-3″, H-4″, H-5″), 2.23 (s, 6 H, 2×CH₃); ¹³C NMR δ 168.5, 149.9 (2), 147.9, 141.4, 137.7, 135.7 (2), 128.4 (2), 127.0, 119.8 (2), 106.1, 17.8 (2); MS m/z 282.6 (MH⁺, 100%). Anal. calcd for C₁₆H₁₅N₃S: C, 68.30; H, 5.37; N, 14.93.Found: C, 68.52; H, 5.47; N, 15.14%.

### Example 1-83

### N-(2,6-Difluorophenyl)-4-(4-pyridinyl)-1,3-thiazol-2-amine (139)

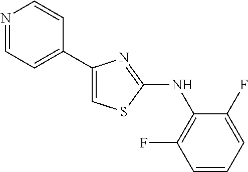

N-(2,6-Difluorophenyl)-4-(4-pyridinyl)-1,3-thiazol-2-amine (139). A mixture of bromoketone hydrobromide 1 (0.30 g, 1.1 mmol) and N-(2-fluorophenyl)thiourea (138) (0.20 g, 1.1 mmol) in EtOH (15 mL) was stirred at reflux temperature for 1 h. The mixture was cooled to 20° C., diluted with water (50 mL), the pH adjusted to ca. 8 with aqueous NH₃ and the mixture stirred at 0° C. for 1 h. The precipitate was filtered, washed with water (5 mL) and dried. The crude solid was purified by column chromatography, eluting with EtOAc, to give amine 139 (0.29 g, 91%) as a white powder: mp (EtOAc/pet. ether) 209-211° C.; ¹H NMR δ 9.75 (s, 1 H, NH), 8.55 (dd, J=4.5, 1.6 Hz, 2 H, H-2′, H-6′), 7.69 (dd, J=4.5, 1.6 Hz, 2 H, H-3′, H-5′), 7.65 (s, 1 H, H-5), 7.32-7.40 (m, 1 H, H-4″), 7.20-7.26 (m, 2 H, H-3″, H-5″); ¹³C NMR δ 165.9, 157.4 (dd, J=49, 5 Hz, 2), 150.0 (2), 147.5, 140.8, 127.1 (t, J=10 Hz), 119.7 (2), 117.2 (t, J=16 Hz), 112.6 (dd, J=18, 5 Hz, 2), 108.2; MS m/z 290.5 (MH⁺, 100%). Anal. calcd for C₁₄H₉F₂N₃S: C, 58.12; H, 3.14; N, 14.52.Found: C, 58.26; H, 3.42; N, 14.40%.

### Example 1-84

### N-{4-[2-(Methyloxy)-4-pyridinyl]-1,3-thiazol-2-yl}-N-(3-methylphenyl)amine (141)

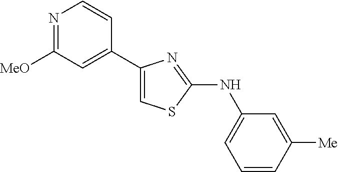

2-Bromo-1-[2-(methyloxy)-4-pyridinyl]ethanone (140). Oxalyl chloride (3.6 mL, 42.4 mmol) was added to a stirred suspension of 2-methoxy-4-pyridinecarboxylic acid (1.30 g, 8.5 mmol) and DMF (3 drops) in DCM (30 mL) and the mixture was stirred at 20° C. for 3 days. The solvent was evaporated and the residue dissolved in THF (20 mL) and added to a stirred solution of TMSCH₂N₂ (2 M in pet. ether) (10.6 mL, 21.2 mmol) at 0° C. The mixture was stirred at 0° C. for 5 h and then 48% HBr (9 mL) was added dropwise and the mixture stirred at 20° C. for 16 h. The mixture was neutralised with saturated aqueous KHCO₃ and extracted with EtOAc (3×50 mL). The organic fraction was washed with water (30 mL), washed with brine (30 mL), dried and the solvent evaporated. The residue was purified by column chromatography, eluting with a gradient (10-20%) of EtOAc/pet. ether, to give bromide 140 (1.08 g, 55%) as a cream powder: ¹H NMR δ 8.35 (dd, J=5.3, 0.7 Hz, 1 H, H-6′), 7.40 (dd, J=5.3, 0.7 Hz, 1 H, H-5′), 7.31 (dd, J=1.4, 0.7 Hz, 1 H, H-3′), 6.60 (br s, 1 H, NH.HBr), 4.97 (s, 2 H, H-2), 3.91 (s, 3 H, OCH₃); MS m/z 230.4/232.4 (MH⁺, 100%). The HBr salt was a cream solid: mp 116° C. (dec.). Anal. calcd for C₈H₉BrNO₂: C, 30.90; H, 2.92; N, 4.50.Found: C, 31.16; H, 3.05; N, 4.43%.

N-{4-[2-(Methyloxy)-4-pyridinyl]-1,3-thiazol-2-yl}-N-(3-methylphenyl)amine (141). A mixture of bromoketone hydrobromide 140 (0.21 g, 0.9 mmol) and 3-methylphenylthiourea (4) (0.15 g, 0.9 mmol) in EtOH (20 mL) was stirred at reflux temperature for 3 h. The mixture was cooled to 20° C., diluted with water (30 mL), the pH adjusted to ca. 8 with aqueous NH₃ and the mixture stirred at 5° C. for 2 h. The precipitate was filtered, washed with water (5 mL) and dried. The crude solid was purified by column chromatography, eluting with 20% EtOAc/pet. ether, to give amine 141 (0.20 g, 87%) as a cream powder: mp (EtOAc/pet. ether) 131-133° C.; ¹H NMR δ 10.25 (br s, 1 H, NH), 8.18 (dd, J=5.5, 0.4 Hz, 1 H, H-6″), 7.67 (s, 1 H, H-5), 7.56 (br d, J=8.1 Hz, 1 H, H-6′), 7.45-7.48 (m, 2 H, H-2′, H-5″), 7.27 (br s, 1 H, H-3″), 7.23 (br t, J=7.8 Hz, 1 H, H-5′), 6.81 (d, J=7.5 Hz, 1 H, H-4′), 3.89 (s, 3 H, OCH₃), 2.32 (s, 3 H, CH₃); ¹³C NMR δ 164.3, 163.3, 147.5, 147.2, 144.1, 140.8, 138.0, 128.8, 122.1, 117.5, 114.1, 113.8, 107.3, 106.0, 53.0, 21.2; MS m/z 298.5 (MH⁺, 100%). Anal. calcd for C₁₆H₁₅N₃OS: C, 64.62; H, 5.08; N, 14.13.Found: C, 64.71; H, 5.13; N, 14.22%.

### Example 1-85

### N-(3-Methylphenyl)-4-(2-methyl-4-pyridinyl)-1,3-thiazol-2-amine (145)

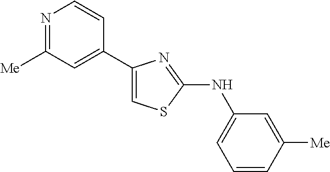

2-Methyl-4-pyridinecarbonitrile (142). Me₂SO₄ (6.2 mL, 65.6 mmol) was added to 2-methylpyridine 1-oxide at 20° C. and the mixture stirred at 20° C. for 16 h. A solution of KCN (47 g, 72 mmol) in water/EtOH (1:1, 150 mL) was added and the mixture stirred at 20° C. for 16 h. The solvent was reduced to half volume and the mixture extracted with CHCl₃ (3×50 mL), the combined organic fraction washed with brine (50 mL) and dried. The solvent as evaporated and the residue purified by column chromatography, eluting with 20% EtOAc/pet. ether, to give nitrile 142 as an oil: ¹H NMR (CDCl₃) δ 8.67 (d, J=5.1 Hz, 1 H, H-6), 7.39 (br s, 1 H, H-3), 7.33 (dd, J=5.1, 0.7 Hz, 1 H, H-5), 2.63 (s, 3 H, CH₃); MS m/z 119.3 (MH⁺, 100%).

1-(2-Methyl-4-pyridinyl)ethanone (143). A solution of MeMgBr (3 M in hexanes, 6.0 mL, 17.9 mmol) was added slowly to a stirred solution of nitrile 142 (1.76 g, 14.9 mmol) in dry THF (100 mL) at 0° C. and the mixture was stirred at reflux temperature for 16 h. The mixture was cooled to 20° C. and the reaction quenched with 6 M HCl (30 mL). The aqueous fraction was separated and the organic fraction extracted with 1 M HCl (30 mL). The combined acidic fraction was stirred at 80° C. for 1 h, cooled to 0° C. and made basic with cNH₃ solution. The mixture was extracted with CHCl₃ (3×50 mL), the combined organic fraction was washed with brine (50 mL), dried and the solvent evaporated. The residue was purified by column chromatography, eluting with 50% EtOAc/pet. ether, to give ketone 143 (0.50 g, 25%) as an oil: ¹H NMR (CDCl₃) δ 8.68 (d, J=5.1 Hz, 1 H, H-6′), 7.59 (br s, 1 H, H-3′), 7.52 (d, J=5.1 Hz, 1 H, H-5′), 2.65 (s, 3 H, H-2), 2.61 (s, 3 H, CH₃); MS m/z 136.5 (MH⁺, 100%).

2-Bromo-1-(2-methyl-4-pyridinyl)ethanone (144). Br₂ (0.2 mL, 3.9 mmol) was added dropwise to a stirred a solution of ketone 143 (0.50 g, 3.3 mmol) in 30% HBr/HOAc (10 mL) at 15° C. The mixture was stirred at 40° C. for 1 h and then at 80° C. for 1 h. The mixture was cooled to 20° C., diluted with Et₂O (40 mL) and stirred at 0° C. for 30 min. The precipitate was filtered, washed with Et₂O (5 mL) and dried under vacuum to give the hydrobromide salt 144 (0.98 g, 90%) as a white powder: mp (Et₂O/H₂O) 183-186° C.; ¹H NMR δ 8.90 (d, J=5.4 Hz, 1 H, H-6′), 8.14 (s, 1 H, H-3′), 8.03 (d, J=5.4 Hz, 1 H, H-5′), 5.80 (br s, 1 H, N.HBr), 5.03 (s, 2 H, H-2), 2.72 (s, 3 H, CH₃). Anal. calcd for C₈H₉BrNO.1¼HBr: C, 30.49; H, 2.96; N, 4.44.Found: C, 30.65; H, 3.39; N, 4.34%.

N-(3-Methylphenyl)-4-(2-methyl-4-pyridinyl)-1,3-thiazol-2-amine (145). A mixture of bromide 144 (0.45 g, 1.5 mmol) and 3-methylphenylthiourea (4) (0.25 g, 1.5 mmol) in EtOH (20 mL) was stirred at reflux temperature for 2 h. The mixture was cooled to 20° C., diluted with water (40 mL), the pH adjusted to ca. 9 with aqueous NH₃ and the mixture stirred at 0° C. for 1 h. The precipitate was filtered, washed with water (5 mL) and dried. The crude solid was purified by column chromatography, eluting with a gradient (50-100%) of EtOAc/pet. ether, to give amine 145 (0.37 g, 87%) as a cream powder: mp (EtOAc/pet. ether) 198-200° C.; ¹H NMR δ 10.25 (s, 1 H, NH), 8.48 (d, J=5.1 Hz, 1 H, H-6′), 7.71 (br s, 1 H, H-3′), 7.62-7.66 (m, 2 H, H-5, H-5′), 7.56 (br d, J=8.1 Hz, 1 H, H-6″), 7.48 (br s, 1 H, H-2″), 7.24 (dd, J=8.1, 7.5 Hz, 1 H, H-5″), 6.80 (br d, J=7.5 Hz, 1 H, H-4″), 2.54 (s, 3 H, CH₃), 2.33 (s, 3 H, CH₃); ¹³C NMR δ 163.4, 158.2, 149.3, 147.8, 141.2, 140.8, 138.0, 128.8, 122.1, 118.9, 117.5, 117.1, 114.1, 106.8, 24.1, 21.2; MS m/z 282.5 (MH⁺, 100%). Anal. calcd for C₁₆H₁₅N₃S: C, 68.30; H, 5.37; N, 14.93.Found: C, 67.99; H, 5.25; N, 14.95%.

### Example 1-86

### 4-(2-Fluoro-4-pyridinyl)-N-(3-methylphenyl)-1,3-thiazol-2-amine (149)

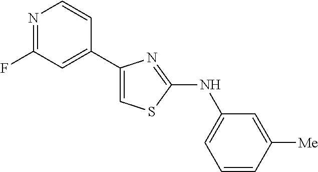

2-Fluoro-N-methyl-N-(methyloxy)-4-pyridinecarboxamide (146). Et₃N (7.9 mL, 57 mmol) was added to a stirred suspension of 2-fluoro-4-pyridinecarboxylic acid (2.0 g, 14.2 mmol), EDCI (3.0 g, 15.6 mmol), HOBT (2.1 g, 15.6 mmol) and MeNHOMe.HCl (2.1 g, 21.3 mmol) in dry DCM (55 mL), and the mixture was stirred at 20° C. for 20 h. The resulting solution was diluted with DCM (110 mL) and washed with water (2×50 mL), washed with brine (50 mL), dried and the solvent evaporated. The residue was purified by column chromatography, eluting with 50% EtOAc/pet. ether, to give amide 146 (1.68 g, 64%) a colourless oil: ¹H NMR (CDCl₃) δ 8.30 (d, J=5.1 Hz, 1 H, H-6), 7.41 (dt, J=5.1, 1.7 Hz, 1 H, H-5), 7.2 (br s, 1 H, H-3), 0.56 (s, 3 H, OCH₃), 3.38 (s, 3 H, NCH₃); MS m/z 185.5 (MH⁺, 100%).

1-(2-Fluoro-4-pyridinyl)ethanone (147). A solution of MeMgBr (3 M in Et₂O, 3.8 mL, 11.4 mmol) was added slowly to a stirred solution of amide 146 (1.4 g, 7.6 mmol) in dry THF (40 mL) at 0° C. and the mixture was stirred at 0° C. for 1 h. The reaction mixture was quenched with saturated aqueous NH₄Cl solution (15 mL) and the mixture extracted with EtOAc (3×50 mL). The combined organic fraction was washed with water (50 mL) washed with brine (50 mL), dried and the solvent evaporated. The residue was purified by column chromatography, eluting with 30% EtOAc/pet. ether, to give ketone 147 (1.01 g, 96%) as a colourless liquid that crystallized upon standing: mp (EtOAc/pet. ether) 38-39° C.; ¹H NMR (CDCl₃) δ 8.41 (d, J=5.1 Hz, 1 H, H-6′), 7.63 (ddd, J=5.2, 3.2, 1.2 Hz, 1 H, H-5′), 7.37 (br s, 1 H, H-3′), 2.63 (s, 3 H, H-2); MS m/z 140.5 (MH¹), 172.5 (MH⁺+MeOH, 100%).

2-Fluoro-1-(2-bromo-4-pyridinyl)ethanone Hydrobromide (148). 1-(2-Fluoro-4-pyridinyl)ethanone (147) (938 mg, 6.74 mmol) was dissolved in glacial acetic acid and treated with bromine (0.39 mL, 7.5 mmol) followed by HBr/AcOH (30% w/v, 1.5 mL, 7.5 mmol) at 5-10° C. The reaction mixture was stirred at 20° C. for 3 h, and then diluted with Et₂O (100 mL) and the formed precipitate was filtered off. The solid was washed with Et₂O and dried under high vacuum to afford the crude bromoketone 148 (941 mg) as white solid, which was used in the next step without further purification.

4-(2-Fluoro-4-pyridinyl)-N-(3-methylphenyl)-1,3-thiazol-2-amine (149). A mixture of bromoketone hydrobromide 148 (941 mg, 3.15 mmol) and 3-methylphenylthiourea (4) (524 mg, 3.15 mmol) in EtOH (25 mL) was stirred at reflux temperature for 3 h. The mixture was cooled to 20° C., diluted with water (80 mL), the pH adjusted to ca. 8 with aqueous NH₃ and the mixture stirred at 20° C. for 2 h. The precipitate was filtered, washed with water (20 mL) and dried. The crude solid was purified by column chromatography, eluting with a gradient (10-20%) of EtOAc/pet. ether, to give a crude solid, which was further purified by preparative HPLC to give amine 149 (25 mg, 3%) as a white solid: mp (CH₃CN/H₂O) 162-164° C.; ¹H NMR (CD₃OD) δ 8.19 (d, J=5.4 Hz, 1 H, H-6″), 7.78 (dt, J=5.4, 1.8 Hz, 1 H, H-5″), 7.52-7.50 (m, 3 H, H-3″, H-5, H-6′), 7.44 (br s, 1 H, H-2′), 7.23 (t, J=7.8 Hz, 1 H, H-5′), 6.85 (br d, J=7.5, 1 H, H-4′), 2.34 (s, 3 H, CH₃), NH not observed; ¹³C NMR (CD₃OD) δ 164.3, 164.1 (d, J=235.7 Hz), 147.6 (d, J=9 Hz), 146.7, 146.6 (d, J=14.5 Hz), 140.4, 138.1, 128.1, 122.1, 117.6 (d, J=3.7 Hz), 117.4, 114.0, 107.2, 104.7 (d, J=38.3 Hz), 19.8; MS m/z 286.5 (MH⁺, 100%). Anal. calcd for C₁₅H₁₂FN₃S.½H₂O: C, 61.21; H, 4.45; N, 14.28.Found: C, 60.89; H, 4.25; N, 14.07%.

### Example 1-87

### 4-(2-Chloro-4-pyridinyl)-N-(3-methylphenyl)-1,3-thiazol-2-amine (150)

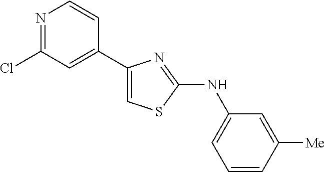

4-(2-Chloro-4-pyridinyl)-N-(3-methylphenyl)-1,3-thiazol-2-amine (150). A mixture of 2-bromo-1-(2-chloro-4-pyridinyl)ethanone (0.51 g, 2.2 mmol) and 3-methylphenylthiourea (4) (0.36 g, 2.2 mmol) in EtOH (35 mL) was stirred at reflux temperature for 1 h. The mixture was cooled to 20° C., diluted with water (80 mL), the pH adjusted to ca. 8 with aqueous NH₃ and the mixture stirred at 0° C. for 1 h. The precipitate was filtered, washed with water (5 mL) and dried. The crude solid was purified by column chromatography, eluting with EtOAc, to give amine 150 (0.62 g, 94%) as a white powder: mp (EtOAc/pet. ether) 195-197° C.; ¹H NMR δ 10.30 (s, 1 H, NH), 8.45 (br d, J=5.1 Hz, 1 H, H-6′), 7.93 (br d, J=1.2 Hz, 1 H, H-3′), 7.87 (dd, J=5.2, 1.4 Hz, 1 H, H-5′), 7.84 (s, 1 H, H-5), 7.55 (br d, J=8.0 Hz, 1 H, H-6″), 7.47 (br s, 1 H, H-2″), 7.25 (br t, J=7.8 Hz, 1 H, H-5″), 6.82 (br d, J=7.4 Hz, 1 H, H-4″), 2.33 (s, 3 H, CH₃); ¹³C NMR δ 163.6, 151.0 (2), 150.3, 146.2, 144.4, 140.6, 138.1, 128.8, 122.3, 119.7, 117.6, 114.2, 109.1, 21.9; MS m/z 302.5 (MH⁺, 100%). Anal. calcd for C₁₅H₁₂ClN₃S: C, 59.70; H, 4.01; N, 13.92.Found: C, 59.75; H, 4.07; N, 13.82%.

### Example 1-88

### 4-(2-Bromo-4-pyridinyl)-N-(3-methylphenyl)-1,3-thiazol-2-amine (154)

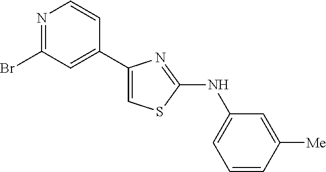

2-Bromo-N-methyl-N-(methyloxy)-4-pyridinecarboxamide (151). Et₃N (14.9 mL, 107 mmol) was added to a stirred suspension of 2-bromo-4-pyridinecarboxylic acid (5.40 g, 26.7 mmol), EDCI (5.64 g, 29.4 mmol), HOBT (3.97 g, 29.4 mmol) and MeNHOMe.HC1 (3.91 g, 40.0 mmol) in dry DCM (100 mL), and the mixture was stirred at 20° C. for 16 h. The resulting solution was diluted with DCM (200 mL) and washed with water (2×50 mL), washed with brine (50 mL), dried and the solvent evaporated. The residue was purified by column chromatography, eluting with 50% EtOAc/pet. ether, to give amide 151 (4.56 g, 70%) as an oil: ¹H NMR (CDCl₃) δ 8.45 (d, J=5.0 Hz, 1 H, H-6), 7.72 (br s, 1 H, H-3), 7.48 (dd, J=5.0, 1.3 Hz, 1 H, H-5), 3.56 (s, 3 H, OCH₃), 3.67 (s, 3 H, NCH₃); MS m/z 245.3/247.3 (MH⁺, 100%).

1-(2-Bromo-4-pyridinyl)ethanone (152). A solution of MeMgBr (3 M in hexanes, 9.3 mL, 27.9 mmol) was added slowly to a stirred solution of amide 151 (4.56 g, 18.6 mmol) in dry THF (100 mL) at 0° C. and the mixture was stirred at 0° C. for 3 h. The reaction mixture was quenched with saturated aqueous NH₄Cl solution (30 mL) and the mixture extracted with EtOAc (3×50 mL). The combined organic fraction was washed with water (50 mL) washed with brine (50 mL), dried and the solvent evaporated. The residue was purified by column chromatography, eluting with 30% EtOAc/pet. ether, to give ketone 152 (3.51 g, 94%) as an oil: ¹H NMR (CDCl₃) δ 8.57 (dd, J=5.1, 0.4 Hz, 1 H, H-6′), 7.91 (dd, J=1.4, 0.4 Hz, 1 H, H-3′), 7.68 (dd, J=5.1, 1.4 Hz, 1 H, H-5′), 2.61 (s, 3 H, H-2); MS m/z 200.3/204.3 (MH⁺, 100%). Anal. calcd for C₇H₆BrNO.¼CH₃CO₂CH₂CH₃: C, 42.68; H, 3.34; N, 6.63.Found: C, 42.62; H, 3.06; N, 6.97%.

2-Bromo-1-(2-bromo-4-pyridinyl)ethanone Hydrobromide (153). Br₂ (0.81 mL, 15.9 mmol) was added dropwise to a stirred a solution of ketone 152 (3.02 g, 15.1 mmol) in 30% HBr/HOAc (20 mL) at 15° C. The mixture was stirred at 20° C. for 1 h, then at 40° C. for 1 h and then at 75° C. for 1 h. The mixture was cooled to 20° C., diluted with Et₂O (50 mL) and stirred for 30 min at 0° C. The precipitate was filtered, washed with Et₂O (5 mL) and dried under vacuum to give the hydrobromide salt 153 (5.28 g, 97%) as a white powder: mp (HOAc/ether)>290° C.; ¹H NMR δ 8.71 (br s, 1 H, N.HBr), 8.63 (dd, J=5.1, 0.5 Hz, 1 H, H-6′), 8.10 (br s, 1 H, H-3′), 7.88 (dd, J=5.1, 1.5 Hz, 1 H, H-5′), 5.00 (s, 2 H, H-2); MS m/z 310.4/312.4/314.4 (MH+MeOH', 60, 100, 60%).

4-(2-Bromo-4-pyridinyl)-N-(3-methylphenyl)-1,3-thiazol-2-amine (154). A mixture of bromoketone hydrobromide 153 (0.62 g, 1.7 mmol) and 3-methylphenylthiourea (4) (0.25 g, 1.7 mmol) in EtOH (20 mL) was stirred at reflux temperature for 2 h. The mixture was cooled to 20° C., diluted with water (50 mL), the pH adjusted to ca. 8 with aqueous NH₃ and the mixture stirred at 20° C. for 2 h. The precipitate was filtered, washed with water (5 mL) and dried. The crude solid was purified by column chromatography, eluting with 40% EtOAc/pet. ether, to give amine 154 (0.50 g, 84%) as a cream powder: mp (EtOAc/pet. ether) 202-204° C.; ¹H NMR δ 10.31 (br s, 1 H, NH), 8.42 (d, J=5.2 Hz, 1 H, H-6″), 8.06 (br s, 1 H, H-3″), 7.90 (dd, J=5.2, 1.4 Hz, 1 H, H-5″), 7.85 (s, 1 H, H-5), 7.52 (br d, J=8.0 Hz, 1 H, H-6′), 7.47 (br s, 1 H, H-2′), 7.25 (br t, J=7.8 Hz, 1 H, H-5′), 6.83 (br d, J=7.5 Hz, 1 H, H-4′), 2.32 (s, 3 H, CH₃); ¹³C NMR δ 163.6, 150.8, 146.0, 144.1, 142.1, 140.7, 138.1, 128.8, 123.4, 122.3, 119.5, 117.6, 114.2, 109.1, 21.2; MS m/z 346.5/348.5 (MH⁺, 100%). Anal. calcd for C₁₅H₁₂BrN₃S: C, 52.03; H, 3.49; N, 12.14.Found: C, 52.21; H, 3.51; N, 12.13%.

### Example 1-89

### N-[4-(2,6-Dichloro-4-pyridinyl)-1,3-thiazol-2-yl]-N-(3-methylphenyl)amine (158)

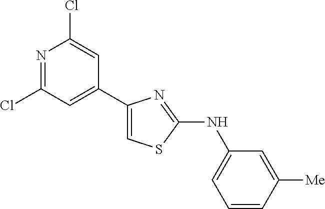

2,6-Dichloro-N-methyl-N-(methyloxy)-4-pyridinecarboxamide (155). Et₃N (5.8 mL, 41.6 mmol) was added to a stirred suspension of 2,6-dichloro-4-pyridinecarboxylic acid (2.0 g, 10.4 mmol), EDCI (2.2 g, 11.4 mmol), HOBT (1.54 g, 11.4 mmol) and MeNHOMe.HC1 (1.52 g, 15.6 mmol) in dry DCM (50 mL), and the mixture was stirred at 20° C. for 20 h. The resulting solution was diluted with DCM (100 mL) and washed with water (2×50 mL), washed with brine (50 mL), dried and the solvent evaporated. The residue was purified by column chromatography, eluting with 20% EtOAc/pet. ether, to give amide 155 (2.01 g, 83%) as a colourless oil: ¹H NMR (CDCl₃) δ 7.51 (s, 2 H, H-2′, H-6′), 3.59 (s, 3 H, OCH₃), 3.38 (s, 3 H, NCH₃); MS m/z 235.4, 237.4 (MH⁺, 100, 70%).

1-(2,6-Dichloro-4-pyridinyl)ethanone (156). A solution of MeMgBr (3 M in Et₂O, 4.3 mL, 12.8 mmol) was added slowly to a stirred solution of amide 155 (2.0 g, 8.5 mmol) in dry THF (40 mL) at 0° C. and the mixture was stirred at 0° C. for 3 h. The reaction mixture was quenched with aq. NH₄Cl solution (15 mL) and the mixture extracted with EtOAc (3×50 mL). The combined organic fraction was washed with water (50 mL), washed with brine (50 mL), dried and the solvent evaporated. The crude product was recrystallized to give the ketone 156 as white crystals (1.61 g, 99%): mp (EtOAc) 50-51° C.; ¹H NMR (CDCl₃) δ 7.68 (s, 2 H, H-3′, H-5′), 2.61 (s, 3 H, H-2); MS m/z 190.4, 192.4 (MH⁺, 100, 70%).

2,6-Dichloro-1-(2-bromo-4-pyridinyl)ethanone Hydrobromide (157). 1-(3-Chloro-4-pyridinyl)ethanone 156 (653 mg, 4.2 mmol) was dissolved in HOAc and treated with Br₂ (0.24 mL, 4.66 mmol) followed by HBr/AcOH (30% w/v, 0.93 mL, 4.66 mmol) at 5-10° C. The reaction mixture was stirred at 20° C. for 3 h and then diluted with Et₂O (100 mL), washed with sat. aq. Na₂S₂O₃ solution (30 mL), washed with water (30 mL), washed with brine (30 mL) and dried. The solvent was evaporated and the crude product was dried under high vacuum to afford the bromoketone 157 (1.32 g) as a pale yellow solid which was used without further purification.

4-(2,6-Dichloro-4-pyridinyl)-N-(3-methylphenyl)-1,3-thiazol-2-amine (158). A mixture of bromoketone hydrobromide 157 (1.32 g, 3.8 mmol) and 3-methylphenylthiourea (4) (632 mg, 3.8 mmol) in EtOH (25 mL) was stirred at reflux temperature for 3 h. The mixture was cooled to 20° C., diluted with water (80 mL), the pH adjusted to ca. 8 with aq. NH₃ and the mixture stirred at 20° C. for 2 h. The precipitate was filtered, washed with water (20 mL) and dried. The crude solid was purified by column chromatography, eluting with 20% EtOAc/pet. ether, to give amine 158 (904 mg, 71%) as a beige solid: mp (EtOAc/pet. ether) 204-206° C.; ¹H NMR δ 10.34 (s, 1 H, NH), 7.99 (s, 1 H, H-5), 7.97 (s, 2 H, H-3″, H-5″), 7.52 (br d, J=8.0 Hz, 1 H, H-6′), 7.45 (br s, 1 H, H-2′), 7.26 (t, J=7.8 Hz, 1 H, H-5′), 6.83 (d, J=7.5 Hz, 1 H, H-4′), 2.32 (s, 3 H, CH₃); ¹³C NMR δ 163.8, 149.9 (2), 147.2, 145.2, 140.6, 138.2, 129.0, 122.6, 119.2 (2), 117.8, 114.4, 111.0, 21.3; MS m/z 336.6, 338.6 (MH⁺, 100, 70%). Anal. calcd for C₁₅H₁₁Cl₂N₃S: C, 53.58; H, 3.30; N, 12.50.Found: C, 53.55; H, 3.25; N, 12.28%.

### Example 1-90

### 4-(2-Ethynyl-4-pyridinyl)-N-(3-methylphenyl)-1,3-thiazol-2-amine (159)

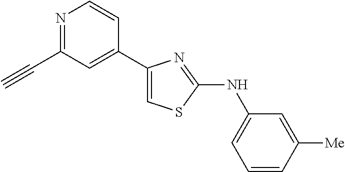

4-(2-Ethynyl-4-pyridinyl)-N-(3-methylphenyl)-1,3-thiazol-2-amine (159). PdCl₂(PPh₃)₂ (35 mg, 0.05 mmol) was added to a purged mixture of bromide (154) (346 mg, 1.0 mmol), trimethylsilylacetylene (0.15 mL, 1.1 mmol), CuI (10 mg, 0.05 mmol) in Et₃N (3 mL) and DMF (3 mL) and the mixture was stirred at 50° C. for 16 h in a sealed tube. The mixture was cooled to 20° C., diluted with water (100 mL), and extracted with EtOAc (3×50 mL). The combined organic fraction was washed with water (2×40 mL), washed with brine (50 mL) and dried. The solvent was evaporated and the crude solid was dissolved in THF (20 mL) and a solution of TBAF (1.0 M, 1.5 mL, 1.5 mmol) was added. The mixture was stirred at 20° C. for 16 h and the solvent was evaporated. The residue was suspended in water (50 mL), and extracted with EtOAc (3×30 mL). The combined organic fraction was washed with water (40 mL), washed with brine (40 mL) and dried. The solvent was evaporated and the crude solid was purified by column chromatography, eluting with 50% EtOAc/pet. ether, to give amine 159 (0.18 g, 62%) as a tan powder: mp (EtOAc/pet. ether) 192-194° C.; ¹H NMR 6 10.29 (s, 1 H, NH), 8.60 (dd, J=5.2, 1.7 Hz, 1 H, H-6″), 8.02 (d, J=1.6, 0.7 Hz, 1 H, H-3″), 7.87 (dd, J=5.2, 1.7 Hz, 1 H, H-5″), 7.81 (s, 1 H, H-5), 7.56 (br d, J=8.0 Hz, 1 H, H-6′), 7.47 (br s, 1 H, H-2′), 7.25 (br t, J=7.8 Hz, 1 H, H-5′), 6.82 (br d, J=7.5 Hz, 1 H, H-4′), 4.34 (s, 1 H, CH), 2.33 (s, 3 H, CH₃); ¹³C NMR δ 163.6, 150.7, 146.7, 142.2, 141.6, 140.8, 138.2, 128.9, 123.2, 122.3, 119.8, 117.5, 114.1, 108.5, 83.2, 80.1, 21.3; MS m/z 292.6 (MH⁺, 100%). Anal. calcd for C₁₇H₁₃N₃S.¼CH₃OH: C, 69.21; H, 4.71; N, 14.04.Found: C, 69.44; H, 4.57; N, 13.86%.

### Example 1-91

### 4-(2-Ethyl-4-pyridinyl)-N-(3-methylphenyl)-1,3-thiazol-2-amine (160)

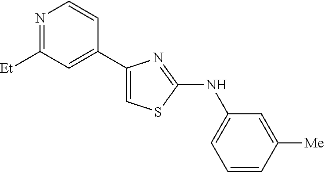

4-(2-Ethyl-4-pyridinyl)-N-(3-methylphenyl)-1,3-thiazol-2-amine (160). A mixture of amine 159 (120 mg, 0.41 mmol) and Pd/C (25 mg) in EtOH (20 mL) was stirred under H₂ (60 psi) for 16 h. The mixture was filtered through Celite, washed with EtOH. The solvent was evaporated and the crude solid was purified by column chromatography, eluting with 50% EtOAc/pet. ether, to give amine 160 (45 mg, 37%) as a white powder: mp (EtOAc/pet. ether) 206-209° C.; ¹H NMR 6 10.30 (s, 1 H, NH), 8.50 (d, J=5.2 Hz, 1 H, H-6″), 7.13 (br s, 1 H, H-3″), 7.65-7.68 (m, 2 H, H-5, H-5″), 7.50-7.54 (m, 2 H, H-2′, H-6′), 7.24 (br t, J=7.7 Hz, 1 H, H-5′), 6.81 (br d, J=7.5 Hz, 1 H, H-4′), 2.80 (q, J=7.6 Hz, 2 H, CH₂), 2.32 (s, 3 H, CH₃), 1.27 (t, J=7.6 Hz, 3 H, CH₃); ¹³C NMR δ 163.4, 163.2, 149.4, 147.9, 141.4, 140.8, 138.1, 128.8, 122.1, 117.8, 117.5, 117.3, 114.1, 106.8, 30.6, 21.2, 13.5; MS m/z 296.6 (MH⁺, 100%). Anal. calcd for C₁₇H₁₇N₃S: C, 69.12; H, 5.80; N, 14.22.Found: C, 69.14; H, 5.87; N, 14.22%.

### Example 1-92

### 3-(4-{2-[(3-Methylphenyl)amino]-1,3-thiazol-4-yl}-2-pyridinyl)-2-propyn-1-ol (161)

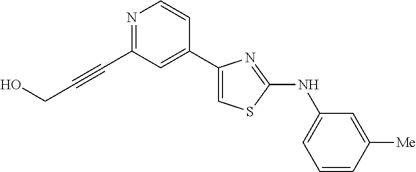

3-(4-{2-[(3-Methylphenyl)amino]-1,3-thiazol-4-yl}-2-pyridinyl)-2-propyn-1-ol (161). PdCl₂(PPh₃)₂ (108 mg, 0.15 mmol) was added to a purged mixture of bromide 154 (1.07 g, 3.1 mmol), propargyl alcohol (0.20 mL, 3.4 mmol), CuI (29 mg, 0.15 mmol) in Et₃N (4 mL) and DMF (5 mL) and the mixture was stirred at 50° C. for 16 h in a sealed tube. The mixture was cooled to 20° C., diluted with water (100 mL), and extracted with EtOAc (3×50 mL). The combined organic fraction was washed with water (2×40 mL), washed with brine (50 mL) and dried. The solvent was evaporated and the crude solid was purified by column chromatography, eluting with EtOAc, to give amine 161 (0.81 g, 81%) as a tan powder: mp (EtOAc/pet. ether) 143-145° C.; ¹H NMR δ 10.28 (s, 1 H, NH), 8.59 (d, J=5.2 Hz, 1 H, H-6′), 7.95 (d, J=1.6 Hz, 1 H, H-3′), 7.82 (dd, J=5.2, 1.7 Hz, 1 H, H-5′), 7.79 (s, 1 H, H-5″), 7.57 (br d, J=8.1 Hz, 1 H, H-6″), 7.43 (br s, 1 H, H-2″), 7.24 (br t, J=7.8 Hz, 1 H, H-5″′), 6.81 (br d, J=7.4 Hz, 1 H, H-4″), 5.42 (t, J=6.0 Hz, 1 H, OH), 4.36 (d, J=6.0 Hz, 2 H, H-1), 2.32 (s, 3 H, CH₃); ¹³C NMR δ 163.5, 150.6, 146.8, 142.7, 141.5, 140.7, 138.1, 128.8, 122.6, 122.3, 119.2, 117.5, 114.1, 108.1, 89.2, 83.6, 49.2, 21.2; MS m/z 322.6 (MH⁺, 100%). Anal. calcd for C₁₈H₁₅N₃OS: C, 67.27; H, 4.70; N, 13.07.Found: C, 67.05; H, 4.81; N, 12.62%.

### Example 1-93

### 3-(4-{2-[(3-Methylphenyl)amino]-1,3-thiazol-4-yl}-2-pyridinyl)-1-propanol (162)

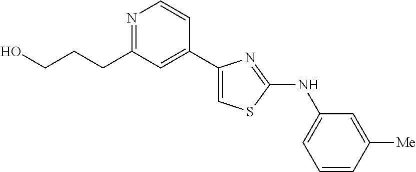

3-(4-{2-[(3-Methylphenyl)amino]-1,3-thiazol-4-yl}-2-pyridinyl)-1-propanol (162). A mixture of amine 161 (700 mg, 2.17 mmol) and Pd/C (100 mg) in EtOH (50 mL) was stirred under H₂ (60 psi) for 16 h. The mixture was filtered through Celite, washed with EtOH. The solvent was evaporated and the crude solid was purified by column chromatography, eluting with 50% EtOAc/pet. ether, to give amine 162 (470 mg, 67%) as a yellow foam: ¹H NMR δ 10.25 (s, 1 H, NH), 8.50 (d, J=5.2 Hz, 1 H, H-6′), 7.72 (br s, 1 H, H-3′), 7.63-7.67 (m, 2 H, H-5′, H-5″), 7.49-7.54 (m, 2 H, H-2″, H-6″), 7.25 (br t, J=7.7 Hz, 1 H, H-5′″), 6.81 (br d, J=7.5 Hz, 1 H, H-4″), 4.49 (t, J=5.1 Hz, 1 H, OH), 3.45-3.50 (m, 2 H, H-1), 2.81 (dd, J=7.9, 7.5 Hz, 2 H, H-3), 2.33 (s, 3 H, CH₃), 1.92 (m, 2 H, H-2); ¹³C NMR δ 163.4, 162.0, 149.4, 147.9, 141.3, 140.8, 138.1, 128.8, 122.1, 118.5, 117.5, 117.3, 114.1, 106.8, 60.2, 34.1, 32.3, 21.2; MS m/z 326.6 (MH⁺, 100%). Anal. calcd for C₁₈H₁₉N₃OS.¼CH₃OH: C, 65.74; H, 6.05; N, 12.60.Found: C, 65.72; H, 6.20; N, 12.58%.

### Example 1-94

### N-(3-Methylphenyl)-4-(3-methyl-4-pyridinyl)-1,3-thiazol-2-amine (166)

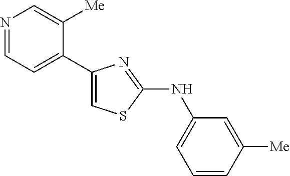

N,3-Dimethyl-N-(methyloxy)-4-pyridinecarboxamide (163). Et₃N (4.1 mL, 29.4 mmol) was added to a stirred suspension of 3-methyl-4-pyridinecarboxylic acid (1.00 g, 7.4 mmol), EDCI (1.55 g, 8.1 mmol), HOBT (1.09 g, 8.1 mmol) and MeNHOMe.HCl (1.08 g, 11.0 mmol) in dry DCM (80 mL), and the mixture was stirred at 20° C. for 16 h. The resulting solution was diluted with DCM (100 mL) and washed with water (2×30 mL), washed with brine (30 mL), dried and the solvent evaporated. The residue was purified by column chromatography, eluting with a gradient of (50-100%) of EtOAc/pet. ether, to give amide 163 (0.84 g, 63%) as an oil: ¹H NMR (CDCl₃) δ 8.50 (s, 1 H, H-2), 8.48 (d, J=4.9 Hz, 1 H, H-6), 7.17 (d, J=4.9 Hz, 1 H, H-5), 3.45 (s, 3 H, OCH₃), 3.35 (s, 3 H, NCH₃), 2.32 (s, 3 H, CH₃); MS m/z 181.4 (MH⁺, 100%).

1-(3-Methyl-4-pyridinyl)ethanone (164). A solution of MeMgBr (3 M in hexanes, 1.7 mL, 5.1 mmol) was added slowly to a stirred solution of amide 163 (0.84 g, 4.6 mmol) in dry THF (30 mL) at 0° C. and the mixture was stirred at 0° C. for 3 h. The reaction mixture was quenched with saturated aqueous NH₄Cl solution (20 mL) and the mixture extracted with EtOAc (3×50 mL). The combined organic fraction was washed with water (40 mL) washed with brine (40 mL), dried and the solvent evaporated. The residue was purified by column chromatography, eluting with 50% EtOAc/pet. ether, to give ketone 164 (0.49 g, 78%) as an oil: ¹H NMR (CDCl₃) δ 8.58 (d, J=5.0 Hz, 1 H, H-6′), 8.56 (s, 1 H, H-2′), 7.42 (d, J=5.0 Hz, 1 H, H-5′), 2.58 (s, 3 H, H-2), 2.47 (s, 3 H, CH₃); MS m/z 136.5 (MH⁺, 100%).

2-Bromo-1-(3-methyl-4-pyridinyl)ethanone Hydrobromide (165). Br₂ (0.21 mL, 4.0 mmol) was added dropwise to a stirred a solution of ketone 164 (0.49 g, 3.7 mmol) in 30% HBr/HOAc (10 mL) at 20° C. The mixture was stirred at 20° C. for 1 h, then at 40° C. for 1 h and then at 75° C. for 1 h. The mixture was cooled to 20° C., diluted with Et₂O (40 mL) and stirred for 30 min at 0° C. The precipitate was filtered, washed with Et₂O (5 mL) and dried under vacuum to give the crude hydrobromide salt 165 (0.75 g, 69%) as an tan powder: ¹H NMR δ 8.80-8.84 (m, 2 H, H-2′, H-6′), 8.04 (d, J=5.5 Hz, 1 H, H-5′), 5.15 (br s, 1 H, N.HBr), 4.63 (s, 2 H, H-2); MS m/z 216.3/218.3 (MH⁺, 100%).

N-(3-Methylphenyl)-4-(3-methyl-4-pyridinyl)-1,3-thiazol-2-amine (166). A mixture of bromoketone 165 (0.53 g, 1.8 mmol) and 3-methylphenylthiourea (4) (0.30 g, 1.8 mmol) in EtOH (20 mL) was stirred at reflux temperature for 2 h. The mixture was cooled to 20° C., diluted with water (50 mL), the pH adjusted to ca. 8 with aqueous NH₃ and the mixture stirred at 20° C. for 2 h. The precipitate was filtered, washed with water (5 mL) and dried. The crude solid was purified by column chromatography, eluting with 50% EtOAc/pet. ether, to give amine 166 (0.42 g, 83%) as a tan powder: mp (EtOAc/pet. ether) 170-172° C.; ¹H NMR δ 10.22 (br s, 1 H, NH), 8.48 (s, 1 H, H-2′), 8.45 (d, J=5.1 Hz, 1 H, H-6′), 7.68 (d, J=5.1 Hz, 1 H, H-5′), 7.52 (br s, 1 H, H-2″), 7.49 (br d, J=8.1 Hz, 1 H, H-6″), 7.30 (s, 1 H, H-5), 7.20 (br t, J=7.8 Hz, 1 H, H-5″), 6.79 (br d, J=7.5 Hz, 1 H, H-4″), 2.53 (s, 3 H, CH₃), 2.30 (s, 3 H, CH₃); ¹³C NMR δ 162.6, 151.7, 147.7, 147.3, 140.9, 140.7, 138.0, 129.7, 128.7, 122.3, 122.0, 117.4, 114.0, 108.9, 21.2, 18.3; MS m/z 282.5 (MH⁺, 100%). Anal. calcd for C₁₆H₁₅N₃S: C, 68.30; H, 5.37; N, 14.93.Found: C, 68.09; H, 5.43; N, 14.67%.

### Example 1-95

### 4-(3-Fluoro-4-pyridinyl)-N-(3-methylphenyl)-1,3-thiazol-2-amine (170)

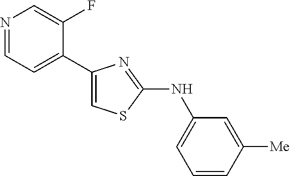

3-Fluoro-N-methyl-N-(methyloxy)-4-pyridinecarboxamide (167). Et₃N (7.9 mL, 57 mmol) was added to a stirred suspension of 3-fluoro-4-pyridinecarboxylic acid (2.0 g, 14.2 mmol), EDCI (3.0 g, 15.6 mmol), HOBt (2.1 g, 15.6 mmol) and MeNHOMe.HC1 (2.1 g, 21.3 mmol) in dry DCM (55 mL), and the mixture was stirred at 20° C. for 20 h. The resulting solution was diluted with DCM (110 mL) and washed with water (2×50 mL), washed with brine (50 mL), dried and the solvent evaporated. The residue was purified by column chromatography, eluting with 50% EtOAc/pet. ether, to give amide 167 (1.60 g, 62%) as a colourless oil: ¹H NMR (CDCl₃) δ 8.55 (s, 1 H, H-2), 8.50 (dd, J=4.8, 1.1 Hz, 1 H, H-6), 7.35 (t, J=5.1 Hz, 1 H, H-5); 3.54 (br s, 3 H, OCH₃), 3.38 (br s, 3 H, NCH₃); MS m/z 185.5 (MH⁺, 100%).

1-(3-Fluoro-4-pyridinyl)ethanone (168). A solution of MeMgBr (3 M in Et₂O, 4.1 mL, 12.2 mmol) was added slowly to a stirred solution of amide 167 (1.5 g, 8.14 mmol) in dry THF (40 mL) at 0° C. and the mixture was stirred at 0° C. for 3 h. The reaction mixture was quenched with saturated aqueous NH₄Cl solution (15 mL) and the mixture extracted with EtOAc (3×50 mL). The combined organic fraction was washed with water (50 mL) washed with brine (50 mL), dried and the solvent evaporated. The residue was purified by column chromatography, eluting with 30% EtOAc/pet. ether, to give ketone 168 (1.00 g, 88%) as a colourless liquid: ¹H NMR (CDCl₃) δ (2 rotamers) δ 8.64 (s, 0.5 H, H-2′), 8.63 (s, 0.5 H, H-2′), 8.57 (d, J=4.9 Hz, 0.5 H, H-6′), 8.56 (d, J=4.9 Hz, 0.5 H, H-6′), 7.68 (dd, J=4.9, 0.4 Hz, 0.5 H, H-5′), 7.66 (dd, J=4.9, 0.4 Hz, 0.5 H, H-5′), 2.69 (s, 1.5 H, H-2), 2.68 (s, 1.5 H, H-2); MS m/z 140.5 (MH⁺, 100%), 172.5 (MH⁺+MeOH).

3-Fluoro-1-(2-bromo-4-pyridinyl)ethanone Hydrobromide (169). 1-(3-Fluoro-4-pyridinyl)ethanone (168) (880 mg, 6.33 mmol) was dissolved in glacial acetic acid (20 mL) and treated with bromine (0.36 mL, 7.0 mmol) followed by HBr/AcOH (30% w/v, 1.4 mL, 7.0 mmol) at 5-10° C. The reaction mixture was stirred at 20° C. for 3 h, it was then diluted with Et₂O (100 mL) and the precipitate was filtered off, washed with Et₂O and dried under high vacuum to afford the crude bromoketone 169 (1.65 g) as a pale yellow solid that was used without further purification.

4-(3-Fluoro-4-pyridinyl)-N-(3-methylphenyl)-1,3-thiazol-2-amine (170). A mixture of bromoketone hydrobromide 169 (1.5 g, 5.0 mmol) and 3-methylphenylthiourea (4) (831 mg, 5.0 mmol) in EtOH (20 mL) was stirred at reflux temperature for 3 h. The mixture was cooled to 20° C., diluted with water (80 mL), the pH adjusted to ca. 8 with aqueous NH₃ and the mixture stirred at 20° C. for 2 h. The precipitate was filtered, washed with water (20 mL) and dried. The crude solid was purified by column chromatography, eluting with 20% EtOAc/pet. ether, to give amine 170 (1.02 g, 72%) as a beige solid: mp (EtOAc/pet. ether) 159-161° C. ¹H NMR δ (2 rotamers) δ 10.31 (s, 1 H, NH), 8.64 (s, 0.5 H, H-2′), 8.63 (s, 0.5 H, H-2′), 8.54 (d, J=5.0 Hz, 0.5 H, H-6′), 8.53 (d, J=5.0 Hz, 0.5 H, H-6′), 8.03 (d, J=5 Hz, 0.5 H, H-5′), 8.01 (d, J=5 Hz, 0.5 H, H-5′), 7.60-7.54 (m, 2 H, H-5, H-6″), 7.47 (br s, 1 H, H-2″), 7.25 (t, J=7.8 Hz, 1 H, H-5″), 6.82 (d, J=7.5 Hz, 1 H, H-4″), 2.33 (s, 3 H, CH₃); ¹³C NMR δ 163.0, 156.0 (d, J=256.4 Hz), 146.5 (d, J=4.8 Hz), 141.5 (d, J=2.4 Hz), 140.8, 138.4 (d, J=24.6 Hz), 138.2, 128.9, 128.4 (d, J=8.7 Hz), 122.5 (d, J=11.0 Hz), 117.7, 114.3, 112.2 (d, J=14.2 Hz), 21.3, 1 resonance not observed; MS m/z 286.6 (MH⁺, 100%); HRMS calcd for C₁₅H₁₃FN₃S (MH¹) m/z 286.0814, found 286.0813.

### Example 1-96

### 4-(3-Chloro-4-pyridinyl)-N-(3-methylphenyl)-1,3-thiazol-2-amine (174)

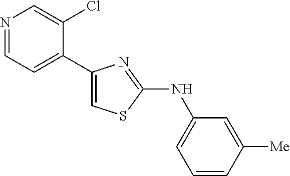

3-Chloro-N-methyl-N-(methyloxy)-4-pyridinecarboxamide (171). Et₃N (7.1 mL, 50.8 mmol) was added to a stirred suspension of 3-chloro-4-pyridinecarboxylic acid (2.0 g, 12.7 mmol), EDCI (2.68 g, 14 mmol), HOBT (1.9 g, 14.0 mmol) and MeNHOMe.HCl (1.86 g, 19.0 mmol) in dry DCM (50 mL), and the mixture was stirred at 20° C. for 20 h. The resulting solution was diluted with DCM (110 mL) and washed with water (2×50 mL), washed with brine (50 mL), dried and the solvent evaporated. The residue was purified by column chromatography, eluting with 50% EtOAc/pet. ether, to give amide 171 (1.46 g, 58%) as a white solid: mp (EtOAc/pet. ether) 54-56° C.; ¹H NMR (CDCl₃) δ 8.65 (s, 1 H, H-2), 8.55 (d, J=4.8 Hz, 1 H, H-6), 7.25 (d, J=4.8 Hz, 1 H, H-5), 3.49 (s, 3 H, OCH₃), 3.39 (s, 3 H, NCH₃); MS m/z 201.4/203.4 (MH⁺, 100%).

1-(3-Chloro-4-pyridinyl)ethanone (172). A solution of MeMgBr (3 M in Et₂O, 3.2 mL, 9.6 mmol) was added slowly to a stirred solution of amide 171 (1.28 g, 6.4 mmol) in dry THF (40 mL) at 0° C. and the mixture was stirred at 0° C. for 3 h then 1 h at 20° C. The reaction mixture was quenched with saturated aqueous NH₄Cl solution (15 mL) and the mixture extracted with EtOAc (3×50 mL). The combined organic fraction was washed with water (50 mL), washed with brine (50 mL), dried and the solvent evaporated. The residue was purified by column chromatography, eluting with 30% EtOAc/pet. ether, to give ketone 172 (883 mg, 89%) as a colourless liquid: ¹H NMR (CDCl₃) δ 8.69 (s, 1 H, H-2′), 8.59 (d, J=4.9 Hz, 1 H, H-6′), 7.37 (dd, J=4.9, 0.6 Hz, H-5′), 2.65 (s, 3 H, CH₃); MS m/z 156.4 (MH⁺, 100%), 188.4 (MH⁺+MeOH).

3-Chloro-1-(2-bromo-4-pyridinyl)ethanone Hydrobromide (173). 1-(3-Chloro-4-pyridinyl)ethanone (172) (653 mg, 4.2 mmol) was dissolved in glacial acetic acid and treated with Br₂ (0.24 mL, 4.66 mmol) followed by HBr/AcOH (30% w/v, 0.93 mL, 4.66 mmol) at 5-10° C. The reaction mixture was stirred at 20° C. for 3 h, it was then diluted with Et₂O (80 mL) and the formed precipitate was filtered off and washed with Et₂O and dried under high vacuum to afford the crude bromoketone hydrobromide 173 (1.27 g) as a pale yellow solid which was used in the next step without further purification.

4-(3-Chloro-4-pyridinyl)-N-(3-methylphenyl)-1,3-thiazol-2-amine (174). A mixture of bromoketone hydrobromide 173 (1.23 g, 4 mmol) and 3-methylphenylthiourea (4) (651 mg, 4 mmol) in EtOH (25 mL) was stirred at reflux temperature for 3 h. The mixture was cooled to 20° C., diluted with water (80 mL), the pH adjusted to ca. 8 with aqueous NH₃ and the mixture stirred at 20° C. for 2 h. The precipitate was filtered, washed with water (20 mL) and dried. The crude solid was purified by column chromatography, eluting with 20% EtOAc/pet. ether, to give amine 174 (340 mg, 28%) as beige crystals: mp (EtOAc) 161-163° C.; ¹H NMR (CDCl₃) δ 8.64 (s, 1 H, H-2″), 8.52 (d, J=5.1 Hz, 1 H, H-6″), 7.96 (d, J=5.1 Hz, 1 H, H-5″), 7.55 (s, 1 H, H-5), 7.29-7.24 (m, 2 H, H-5′, H-6′), 7.18 (br s, 1 H, H-2′), 6.94 (br d, J=7.2 Hz, 1 H, H-4′), 2.38 (s, 3 H, CH₃); NH not observed; ¹³C NMR (CDCl₃) δ 163.8, 150.7, 148.0, 145.2, 139.9, 139.6, 139.4, 129.4, 128.6, 124.5, 124.4, 119.3, 115.6, 110.7, 21.5; MS m/z 302.6/304.6 (MH⁺, 100, 35%); HRMS calcd for C₁₅H₁₃³⁵ClN₃S (MH⁺) m/z 302.05187, found 302.05141.

### Example 1-97

### 4-(3-Bromo-4-pyridinyl)-N-(3-methylphenyl)-1,3-thiazol-2-amine (178)

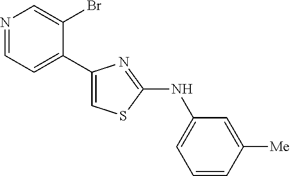

3-Bromo-N-methyl-N-(methyloxy)-4-pyridinecarboxamide (175). Et₃N (5.9 mL, 42.4 mmol) was added to a stirred suspension of 3-bromo-4-pyridinecarboxylic acid (2.14 g, 10.6 mmol), EDCI (2.23 g, 11.7 mmol), HOBT (1.57 g, 11.7 mmol) and MeNHOMe.HC1 (1.55 g, 15.9 mmol) in dry DCM (100 mL), and the mixture was stirred at 20° C. for 16 h. The resulting solution was diluted with DCM (100 mL) and washed with water (2×30 mL), washed with brine (30 mL), dried and the solvent evaporated. The residue was purified by column chromatography, eluting with 50% EtOAc/pet. ether, to give amide 175 (1.74 g, 67%) as an oil: ¹H NMR (CDCl₃) δ 8.81 (br s, 1 H, H-2), 8.64 (d, J=4.8 Hz, 1 H, H-6), 7.53 (d, J=4.8 Hz, 1 H, H-5), 3.47 (s, 3 H, OCH₃), 3.28 (s, 3 H, NCH₃); MS m/z 245.3/247.3 (MH⁺, 100%).

1-(3-Bromo-4-pyridinyl)ethanone (176). A solution of MeMgBr (3 M in hexanes, 3.5 mL, 10.5 mmol) was added slowly to a stirred solution of amide 175 (1.72 g, 7.0 mmol) in dry THF (50 mL) at 0° C. and the mixture was stirred at 0° C. for 3 h. The reaction mixture was quenched with saturated aqueous NH₄Cl solution (20 mL) and the mixture extracted with EtOAc (3×50 mL). The combined organic fraction was washed with water (40 mL) washed with brine (40 mL), dried and the solvent evaporated. The residue was purified by column chromatography, eluting with 30% EtOAc/pet. ether, to give ketone 176 (0.91 g, 65%) as an oil: ¹H NMR (CDCl₃) δ 8.81 (s, 1 H, H-2′), 8.64 (d, J=4.9 Hz, 1 H, H-6′), 7.30 (d, J=4.8 Hz, 1 H, H-5′), 2.63 (s, 3 H, H-2); MS m/z 200.3/204.3 (MH⁺, 100%). Also recovered: starting material (0.33 g, 19%).

2-Bromo-1-(3-bromo-4-pyridinyl)ethanone Hydrobromide (177). Br₂ (0.26 mL, 5.0 mmol) was added dropwise to a stirred a solution of ketone 176 (0.90 g, 4.5 mmol) in 30% HBr/HOAc (10 mL) at 15° C. The mixture was stirred at 20° C. for 1 h, then at 40° C. for 1 h and then at 75° C. for 1 h. The mixture was cooled to 20° C., diluted with Et₂O (40 mL) and stirred for 30 min at 0° C. The precipitate was filtered, washed with Et₂O (5 mL) and dried under vacuum to give the hydrobromide salt. The salt was dissolved in water, the solution neutralised with cNH₃ and extracted with EtOAc. The combined organic extract was dried and the solvent evaporated. The residue was purified by column chromatography, eluting with 50% EtOAc/pet. ether, to give the crude bromoketone 177 (0.31 g, 25%) as an oil: ¹H NMR (CDCl₃) δ 8.81 (s, 1 H, H-2′), 8.62 (d, J=4.9 Hz, 1 H, H-6′), 7.30 (d, J=4.9 Hz, 1 H, H-5′), 2.63 (s, 3 H, H-2); which was used directly.

4-(3-Bromo-4-pyridinyl)-N-(3-methylphenyl)-1,3-thiazol-2-amine (178). A mixture of bromoketone 177 (0.30 g, 1.1 mmol) and 3-methylphenylthiourea (4) (0.18 g, 1.1 mmol) in EtOH (20 mL) was stirred at reflux temperature for 2 h. The mixture was cooled to 20° C., diluted with water (50 mL), the pH adjusted to ca. 8 with aqueous NH₃ and the mixture stirred at 20° C. for 2 h. The precipitate was filtered, washed with water (5 mL) and dried. The crude solid was purified by column chromatography, eluting with 60% EtOAc/pet. ether, to give amine 178 (0.29 g, 77%) as a tan powder: mp (EtOAc/pet. ether) 180-182° C.; ¹H NMR δ 10.27 (br s, 1 H, NH), 8.82 (s, 1 H, H-2″), 8.61 (d, J=5.0 Hz, 1 H, H-6″), 7.87 (d, J=5.0 Hz, 1 H, H-5″), 7.68 (s, 1 H, H-5), 7.58-7.64 (m, 2 H, H-2′, H-6′), 7.22 (br dd, J=8.1, 7.7 Hz, 1 H, H-5′), 6.80 (br d, J=7.7 Hz, 1 H, H-4′), 2.30 (s, 3 H, CH₃); ¹³C NMR δ 162.7, 152.6, 148.5, 145.5, 141.3, 140.7, 138.0, 128.7, 124.9, 122.2, 118.1, 117.5, 114.1, 110.6, 21.7; MS m/z 346.5/348.5 (MH⁺, 100%). Anal. calcd for C₁₅H₁₂BrN₃S: C, 52.03; H, 3.49; N, 12.14.Found: C, 51.85; H, 3.50; N, 12.04%.

### Example 1-98

### N-(3-Methylphenyl)-4-(2-nitro-4-pyridinyl)-1,3-thiazol-2-amine (181)

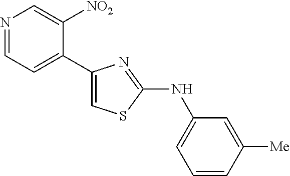

1-(2-Nitro-4-pyridinyl)ethanone (179). 4-Acetylpyridine (2.0 mL, 18.2 mmol) was added slowly to TFAA (10.6 mL, 76.4 mmol) at 0° C. and the mixture was stirred at 0° C. for 1 h. cHNO₃ (2.4 mL, 38.2 mmol) was added to the mixture dropwise and the mixture stirred at 0° C. for 8 h. The reaction mixture was added dropwise to a stirred solution of Na₂S₂O₅ (2.54 g, 18.2 mmol) in water (20 mL) at 0° C. and the mixture stirred at 0° C. for 16 h. The pH of the solution was adjusted to 6-7 with 1 M NaOH solution and the mixture was extracted with DCM (3×50 mL). The combined organic fraction was washed with water (50 mL), washed with brine (50 mL), dried and the solvent evaporated. The residue was purified by column chromatography, eluting with 50% EtOAc/pet. ether, to give ketone 179 (Katritzky, A. R. et. al. Org. Biomolec. Chem. 2005, 3, 538-541) (1.66 g, 55%) as an oil: ¹H NMR (CDCl₃) δ 9.37 (s, 1 H, H-2′), 8.96 (d, J=4.9 Hz, 1 H, H-6′), 7.33 (dd, J=4.9, 0.4 Hz, 1 H, H-5′), 2.59 (s, 3 H, H-2); MS m/z 167.4 (MH⁺, 100%).

2-Bromo-1-(2-nitro-4-pyridinyl)ethanone Hydrobromide (180). Br₂ (0.53 mL, 10.4 mmol) was added dropwise to a stirred a solution of ketone 179 (1.64 g, 9.9 mmol) in 30% HBr/HOAc (20 mL) at 15° C. The mixture was stirred at 20° C. for 16 h. The mixture diluted with Et₂O (80 mL) and stirred for 30 min at 0° C. The precipitate was filtered, washed with Et₂O (5 mL) and dried under vacuum to give the crude hydrobromide salt 180 (2.76 g, 86%) as a white powder: ¹H NMR δ 9.48 (s, 1 H, H-2′), 9.10 (d, J=4.9 Hz, 1 H, H-6′), 7.81 (dd, J=4.9, 0.4 Hz, 1 H, H-5′), 4.86 (s, 2 H, H-2) N.HBr not observed; MS m/z 277.4/279.4 (MH⁺, 100%).

N-(3-Methylphenyl)-4-(2-nitro-4-pyridinyl)-1,3-thiazol-2-amine (181). A mixture of bromoketone hydrobromide 180 (2.00, 6.1 mmol) and 3-methylphenylthiourea (4) (1.02 g, 6.1 mmol) in EtOH (50 mL) was stirred at reflux temperature for 2 h. The mixture was cooled to 20° C., diluted with water (100 mL), the pH adjusted to ca. 8 with aqueous NH₃ and the mixture stirred at 20° C. for 2 h. The precipitate was filtered, washed with water (5 mL) and dried. The crude solid was purified by column chromatography, eluting with 60% EtOAc/pet. ether, to give amine 181 (1.74 g, 91%) as an orange powder: mp (EtOAc/pet. ether) 198-200° C.; ¹H NMR δ 10.29 (br s, 1 H, NH), 9.01 (br s, 1 H, H-2′) 8.09, (d, J=5.2 Hz, 1 H, H-6′), 7.96 (d, J=5.2 Hz, 1 H, H-5′), 7.79 (s, 1 H, H-5), 7.54 (br s, 1 H, H-2″), 7.15-7.19 (m, 2 H, H-5″, H-6″), 6.76-7.81 (m, 1 H, H-4″), 2.342 (s, 3 H, CH₃); ¹³C NMR δ 163.4, 152.1, 144.7, 144.3, 143.1, 140.5, 138.3, 133.8, 128.6, 122.8, 122.3, 117.3, 114.1, 111.5, 21.1; MS m/z 313.6 (MH⁺, 100%). Anal. calcd for C₁₅H₁₂N₄O₂S: C, 57.68; H, 3.87; N, 17.94.Found: C, 57.81; H, 3.92; N, 18.00%.

### Example 1-99

### N-[4-(2-Amino-4-pyridinyl)-1,3-thiazol-2-yl]-N-(3-methylphenyl)amine (182)

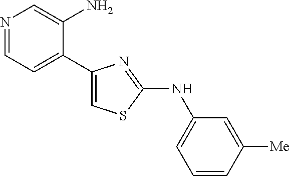

N-[4-(2-Amino-4-pyridinyl)-1,3-thiazol-2-yl]-N-(3-methylphenyl)amine (182). A mixture of nitropyridine 181 (511 mg, 1.64 mmol) and Pd/C (50 mg) in EtOH (100 mL) was stirred under H₂ (60 psi) for 16 h. The mixture was filtered through Celite, washed with EtOH. The solvent was evaporated and the crude solid was purified by column chromatography, eluting with a gradient (50-100%) of EtOAc/pet. ether, to give amine 182 (338 mg, 73%) as a white powder: mp (EtOAc/pet. ether) 159-161° C.; ¹H NMR δ 10.26 (s, 1 H, NH), 8.10 (br s, 1 H, H-2″), 7.77 (d, J=5.1 Hz, 1 H, H-6″), 7.44 (d, J=5.1 Hz, 1 H, H-5″), 7.41 (br s, 1 H, H-2), 7.38 (s, 1 H, H-5′), 7.30 (br d, J=8.1 Hz, 1 H, H-6), 7.22 (br t, J=7.8 Hz, 1 H, H-5), 6.82 (br d, J=7.4 Hz, 1 H, H-4′), 6.21 (br s, 2 H, NH₂), 2.33 (s, 3 H, CH₃); ¹³C NMR δ 163.3, 147.8, 141.3, 140.8, 139.4, 138.4, 136.8, 129.0, 122.4, 122.2, 120.9, 117.8, 114.4, 105.7, 21.3; MS m/z 283.6 (MH⁺, 100%). Anal. calcd for C₁₅H₁₄N₄S: C, 63.80; H, 5.00; N, 19.84.Found: C, 64.09; H, 5.21; N, 19.54%.

### Example 1-100

### N-(4-{2-[(3-Methylphenyl)amino]-1,3-thiazol-4-yl}-2-pyridinyl)acetamide (183)

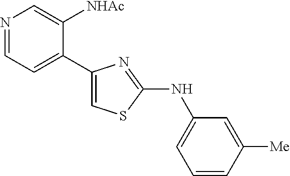

N-(4-{2-[(3-Methylphenyl)amino]-1,3-thiazol-4-yl}-2-pyridinyl)acetamide (183). Ac₂O (0.23 mL, 2.4 mmol) was added to a stirred solution of amine 182 (230 mg, 0.8 mmol) in dioxane (10 mL) and the mixture was stirred at 20° C. for 3 days. The mixture was diluted with water (30 mL), and extracted with EtOAc (3×50 mL). The combined organic fraction was dried and the solvent evaporated. The crude solid was purified by column chromatography, eluting with a gradient (50-100%) of EtOAc/pet. ether, to give amine 183 (148 mg, 56%) as a tan powder: mp (EtOAc/pet. ether) 189-191° C.; ¹H NMR δ 10.79 (s, 1 H, NH), 10.38 (s, 1 H, NH), 9.29 (br s, 1 H, H-2′), 8.33 (d, J=5.1 Hz, 1 H, H-6′), 7.76 (d, J=5.1 Hz, 1 H, H-5′), 7.44 (br d, J=8.1 Hz, 1 H, H-6″), 7.34 (br s, 1

H, H-2″), 7.60 (s, 1 H, H-5″), 7.27 (br t, J=7.8 Hz, 1 H, H-5″), 6.86 (br d, J=7.5 Hz, 1 H, H-4″), 2.32 (s, 3 H, CH₃), 1.99 (s, 3 H, CH₃); ¹³C NMR δ 168.2, 164.1, 145.7, 144.7 (2), 140.5, 138.5, 131.4, 130.2, 129.1, 123.0, 121.5, 118.3, 114.9, 109.1, 24.0, 21.2; MS m/z 325.7 (MH⁺, 100%). Anal. calcd for C₁₇H₁₆N₄OS: C, 62.94; H, 4.97; N, 17.27.Found: C, 62.98; H, 4.90; N, 17.03%.

### Example 1-101

### 4-(3-Ethynyl-4-pyridinyl)-N-(3-methylphenyl)-1,3-thiazol-2-amine (184)

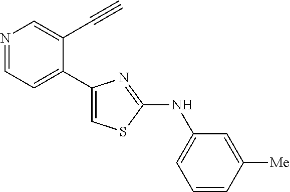

4-(3-Ethynyl-4-pyridinyl)-N-(3-methylphenyl)-1,3-thiazol-2-amine (184). PdCl₂(PPh₃)₂ (52 mg, 0.07 mmol) was added to a purged mixture of bromide (178) (517 mg, 1.5 mmol), trimethylsilylacetylene (0.23 mL, 1.6 mmol), CuI (14 mg, 0.07 mmol) in Et₃N (3 mL) and DMF (3 mL) and the mixture was stirred at 50° C. for 16 h in a sealed tube. The mixture was cooled to 20° C., diluted with water (100 mL), and extracted with EtOAc (3×50 mL). The combined organic fraction was washed with water (2×40 mL), washed with brine (50 mL) and dried. The solvent was evaporated and the crude solid was dissolved in THF (20 mL) and a solution of TBAF (1.0 M, 2.2 mL, 2.2 mmol) was added. The mixture was stirred at 20° C. for 16 h and the solvent was evaporated. The residue was suspended in water (50 mL), and extracted with EtOAc (3×30 mL). The combined organic fraction was washed with water (40 mL), washed with brine (40 mL) and dried. The solvent was evaporated and the crude solid was purified by column chromatography, eluting with 50% EtOAc/pet. ether, to give amine 184 (0.39 g, 89%) as a tan powder: mp (EtOAc/pet. ether) 158-160° C.; ¹H NMR 6 10.27 (s, 1 H, NH), 8.73 (s, 1 H, H-2′), 8.64 (d, J=5.5 Hz, 1 H, H-6′), 8.00 (s, 1 H, H-5), 7.98 (dd, J=5.5, 0.4 Hz, 1 H, H-5′), 7.58 (br d, J=8.1 Hz, 1 H, H-6″), 7.48 (br s, 1 H, H-2″), 7.24 (br t, J=7.8 Hz, 1 H, H-5″), 6.81 (br d, J=7.5 Hz, 1 H, H-4″), 4.72 (s, 1 H, CH), 2.32 (s, 3 H, CH₃); ¹³C NMR δ 162.6, 154.4, 149.4, 145.7, 141.5, 140.7, 138.0, 128.8, 122.2, 121.6, 117.5, 114.3, 114.1, 110.7, 88.1, 80.9, 21.2; MS m/z 296.6 (MH⁺, 100%). Anal. calcd for C₁₇H₁₃N₃S: C, 69.12; H, 5.80;

N, 14.22.Found: C, 68.70; H, 5.83; N, 14.13%.

### Example 1-102

### 4-(3-Ethyl-4-pyridinyl)-N-(3-methylphenyl)-1,3-thiazol-2-amine (185)

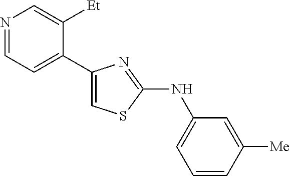

4-(3-Ethyl-4-pyridinyl)-N-(3-methylphenyl)-1,3-thiazol-2-amine (185). A mixture of amine 184 (233 mg, 0.80 mmol) and Pd/C (25 mg) in EtOH (20 mL) was stirred under H₂ (60 psi) for 16 h. The mixture was filtered through Celite, washed with EtOH. The solvent was evaporated and the crude solid was purified by column chromatography, eluting with 50% EtOAc/pet. ether, to give amine 185 (47 mg, 20%) as a cream powder: mp (EtOAc/pet. ether) 151-153° C.; ¹H NMR 6 10.20 (s, 1 H, NH), 8.51 (br s, 1 H, H-2″), 8.43 (d, J=5.0 Hz, 1 H, H-6″), 7.54 (d, J=5.0 Hz, 1 H, H-5″), 7.50 (br s, 1 H, H-2′), 7.44 (br d, J=8.1 Hz, 1 H, H-6′), 7.24 (s, 1 H, H-5), 7.19 (br t, J=7.8 Hz, 1 H, H-5′), 6.78 (br d, J=7.5 Hz, 1 H, H-4′), 2.98 (q, J=7.5 Hz, 2 H, CH₂), 2.29 (s, 3 H, CH₃), 1.18 (t, J=7.5 Hz, 3 H, CH₃); ¹³C NMR δ 162.9, 150.9, 148.1, 147.1, 140.8, 140.5, 137.8, 135.9, 128.6, 122.9, 122.0, 117.2, 114.0, 108.1, 23.7, 21.1, 15.3; MS m/z 296.6 (MH⁺, 100%). Anal. calcd for C₁₇H₁₇N₃S: C, 69.12; H, 5.80; N, 14.22.Found: C, 68.70; H, 5.83; N, 14.13%.

### Example 1-103

### 3-(4-{2-[(3-Methylphenyl)amino]-1,3-thiazol-4-yl}-3-pyridinyl)-2-propyn-1-ol (186)

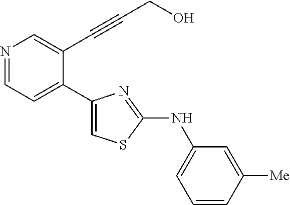

3-(4-{2-[(3-Methylphenyl)amino]-1,3-thiazol-4-yl}-3-pyridinyl)-2-propyn-1-ol (186). PdCl₂(PPh₃)₂ (101 mg, 0.15 mmol) was added to a purged mixture of bromide 178 (1.00 g, 2.9 mmol), propargyl alcohol (0.19 mL, 3.2 mmol), CuI (28 mg, 0.15 mmol) in Et₃N (4 mL) and DMF (5 mL) and the mixture was stirred at 50° C. for 16 h in a sealed tube. The mixture was cooled to 20° C., diluted with water (100 mL), and extracted with EtOAc (3×50 mL). The combined organic fraction was washed with water (2×40 mL), washed with brine (50 mL) and dried. The solvent was evaporated and the crude solid was purified by column chromatography, eluting with EtOAc, to give amine 186 (0.34 g, 34%) as a tan powder: mp (EtOAc/pet. ether) 155-158° C.; ¹H NMR δ 10.27 (s, 1 H, NH), 8.66 (s, 1 H, H-2′), 8.60 (d, J=5.1 Hz, 1 H, H-6′), 8.12 (s, 1 H, H-5″), 8.02 (d, J=5.1 Hz, 1 H, H-5′), 7.60 (br d, J=8.0 Hz, 1 H, H-6″′), 7.45 (br s, 1 H, H-2″′), 7.25 (br t, J=7.8 Hz, 1 H, H-5″′), 6.81 (br d, J=7.5 Hz, 1 H, H-4″′), 5.45 (br s, 1 H, OH), 4.45 (d, J=3.7 Hz, 2 H, H-1), 2.32 (s, 3 H, CH₃); ¹³C NMR δ 162.3, 153.6, 149.0, 145.7, 140.9, 140.8, 138.0, 128.8, 122.2, 121.6, 117.5, 114.7, 114.1, 110.8, 97.3, 81.2, 49.6, 21.2; MS m/z 322.6 (MH⁺, 100%). Anal. calcd for C₁₈H₁₅N₃OS.¼CH₃OH: C, 66.54; H, 4.90; N, 12.76.Found: C, 66.87; H, 5.02; N, 12.42%.

### Example 1-104

### 3-(4-{2-[(3-Methylphenyl)amino]-1,3-thiazol-4-yl}-3-pyridinyl)-1-propanol (187)

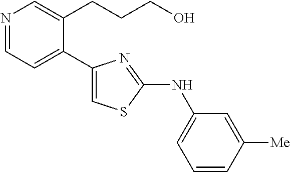

3-(4-{2-[(3-Methylphenyl)amino]-1,3-thiazol-4-yl}-3-pyridinyl)-1-propanol (187). A mixture of amine 186 (205 mg, 0.64 mmol) and Pd/C (40 mg) in EtOH (50 mL) was stirred under H₂ (60 psi) for 16 h. The mixture was filtered through Celite, washed with EtOH. The solvent was evaporated and the crude solid was purified by column chromatography, eluting with EtOAc, to give amine 187 (78 mg, 37%) as a tan gum: ¹H NMR δ 10.20 (s, 1 H, NH), 8.48 (s, 1 H, H-2′), 8.43 (d, J=5.1 Hz, 1 H, H-6′), 7.57 (d, J=5.1 Hz, 1 H, H-5′), 7.44-7.48 (m, 2 H, H-2″′, H-6″′), 7.26 (s, 1 H, H-5″), 7.20 (br t, J=7.8 Hz, 1 H, H-5″′), 6.78 (br d, J=7.5 Hz, 1 H, H-4″), 4.45 (t, J=5.1 Hz, 1 H, OH), 3.35-3.42 (m, 2 H, H-1), 3.00 (br t, J=7.8 Hz, 2 H, H-3), 2.30 (s, 3 H, CH₃), 1.67-1.72 (m, 2 H, H-2); ¹³C NMR δ 162.9, 151.3, 148.0, 147.2, 140.8, 140.7, 138.0, 134.3, 128.7, 123.0, 122.0, 117.3, 114.0, 108.2, 60.1, 33.3, 27.0, 21.1; MS m/z 326.6 (MH⁺, 100%). Anal. calcd for C₁₈H₁₉N₃OS: C, 66.43; H, 5.88; N, 12.91.Found: C, 66.03; H, 6.04; N, 12.58%.

### Example 1-105

### N-(3-Methylphenyl)-4-(1-oxido-4-pyridinyl)-1,3-thiazol-2-amine (188)

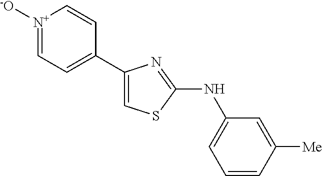

N-(3-Methylphenyl)-4-(1-oxido-4-pyridinyl)-1,3-thiazol-2-amine (188). MCPBA (50-55%, 710 mg, 2.1 mmol) was added to a solution of amine 5 (500 mg, 1.9 mmol) in DCM (30 mL) and the reaction mixture was stirred at 20° C. for 24 h. The mixture was diluted with DCM (50 mL), washed with sat. aq. NaHCO₃ solution (50 mL), washed with sat. aq. Na₂S₂O₅ solution (50 mL), and washed with brine (50 mL). The solution was dried, the solvent was evaporated and the residue was purified by chromatography, eluting with 5% DCM/MeOH, to give the N-oxide 188 (240 mg, 45%) as a yellow powder: mp (EtOAc/pet.ether) 162-164° C.; ¹H NMR δ 10.26 (s, 1 H, NH), 8.24 (dt, J=7.2, 2.5 Hz, 2 H, H-2′, H-6′) 7.86 (dt, J=7.2, 2.5 Hz, 2 H, H-3′, H-5′), 7.58-7.56 (m, 2 H, H-5, H-6″), 7.44 (br s, 1 H, H-2″), 7.23 (t, J=7.7 Hz, 1 H, H-5″), 6.81 (d, J=7.5 Hz, 1 H, H-4″), 2.32 (s, 3 H, CH₃); ¹³C NMR δ 163.6, 146.6, 140.9, 138.8 (2), 138.2, 130.8, 128.9, 122.6 (2), 122.3, 117.6, 114.2, 106.1, 21.3; MS m/z 284.6 (MH⁺, 100%). Anal. calcd for C₁₅H₁₃N₃OS: C, 63.58; H, 4.62; N, 14.83.Found: C, 63.08; H, 4.59; N, 14.86%.

### Example 1-106

### N-(3-Methylphenyl)-4-(4-quinolinyl)-1,3-thiazol-2-amine (192)

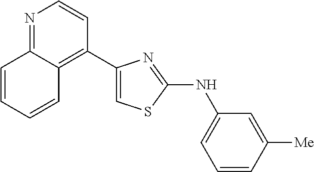

N-Methyl-N-(methyloxy)-4-quinolinecarboxamide (189). A suspension of quinoline 4-carboxylic acid (1.13 g, 6.53 mmol) and DMF (3 drops) in thionyl chloride (15 mL) was stirred at 40° C. for 3 h. Thionyl chloride was evaporated and the residue was dissolved in dry DCM (50 mL) and MeNHOMe.HC1 (0.96 g, 9.8 mmol) and Et₃N (2.7 mL, 19.6 mmol) was added. The mixture was stirred at 20° C. for 16 h. The resulting solution was diluted with DCM (100 mL) and washed with water (2×30 mL), washed with brine (30 mL), dried and the solvent evaporated. The residue was purified by column chromatography, eluting with EtOAc, to give amide 189 (1.36 g, 96%) as soft solid: ¹H NMR (CDCl₃) δ 8.97 (d, J=4.3 Hz, 1 H, H-2), 8.16 (d, J=8.5 Hz, 1 H, H-5), 7.87 (d, J=8.3 Hz, 1 H, H-8), 7.73-7.78 (m, 1 H, H-7), 7.56-7.62 (m, 1 H, H-6), 7.39 (d, J=4.3 Hz, 1 H, H-3), 3.47 (s, 3 H, OCH₃), 3.41 (s, 3 H, NCH₃); MS m/z 216.4 (MH⁺, 100%).

1-(4-Quinolinyl)ethanone (190). A solution of MeMgBr (3 M in hexanes, 3.1 mL, 9.4 mmol) was added slowly to a stirred solution of amide 189 (1.36 g, 6.3 mmol) in dry THF (50 mL) at 0° C. and the mixture was stirred at 0° C. for 3 h. The reaction mixture was quenched with saturated aqueous NH₄Cl solution (20 mL) and the mixture extracted with EtOAc (3×50 mL). The combined organic fraction was washed with water (40 mL) washed with brine (40 mL), dried and the solvent evaporated. The residue was purified by column chromatography, eluting with a gradient (50-100%) of EtOAc/pet. ether, to give ketone 190 (0.76 g, 70%) as an oil: ¹H NMR (CDCl₃) δ 9.03 (d, J=4.4 Hz, 1 H, H-2′), 8.45 (dd, J=8.5, 0.8 Hz, 1 H, H-5′), 8.17 (dd, J=8.5, 0.7 Hz, 1 H, H-8′), 7.78 (ddd, J=8.5, 6.9, 1.4 Hz, 1 H, H-7′), 7.65 (ddd, J=8.5, 6.9, 1.4 Hz, 1 H, H-6′), 7.62 (d, J=4.4 Hz, 1 H, H-3′), 2.75 (s, 3 H, H-2); MS m/z 172.4 (MH⁺, 100%).

2-Bromo-1-(4-quinolinyl)ethanone Hydrobromide (191). Br₂ (0.24 mL, 4.6 mmol) was added dropwise to a stirred a solution of ketone 190 (0.75 g, 4.4 mmol) in 30% HBr/HOAc (10 mL) at 15° C. The mixture was stirred at 20° C. for 1 h, then at 40° C. for 1 h and then at 75° C. for 1 h. The mixture was cooled to 20° C., diluted with Et₂O (40 mL) and stirred for 30 min at 0° C. The precipitate was filtered, washed with Et₂O (5 mL) and dried under vacuum to give the crude hydrobromide salt 191 (1.37 g, 95%) as an tan powder: ¹H NMR δ 9.05 (d, J=4.5 Hz, 1 H, H-2′), 8.23 (br d, J=8.5 Hz, 1 H, H-8′), 8.16 (br d, J=8.4 Hz, 1 H, H-5′), 8.08 (d, J=4.5 Hz, 1 H, H-3′), 7.87-7.92 (m, 1 H, H-7′), 7.75-7.80 (m, 1 H, H-6′), 5.80 (br s, 1 H, N.HBr), 5.10 (s, 2 H, H-2).

N-(3-Methylphenyl)-4-(4-quinolinyl)-1,3-thiazol-2-amine (192). A mixture of bromoketone 191 (0.40 g, 1.2 mmol) and 3-methylphenylthiourea (4) (0.20 g, 1.2 mmol) in EtOH (20 mL) was stirred at reflux temperature for 2 h. The mixture was cooled to 20° C., diluted with water (50 mL), the pH adjusted to ca. 8 with aqueous NH₃ and the mixture stirred at 5° C. for 2 h. The precipitate was filtered, washed with water (5 mL) and dried. The crude solid was purified by column chromatography, eluting with 50% EtOAc/pet. ether, to give amine 192 (0.26 g, 68%) as a tan powder: mp (EtOAc/Et₂O) 169-171° C.; ¹H NMR δ 10.33 (br s, 1 H, NH), 8.95 (d, J=4.5 Hz, 1 H, H-2′), 8.75 (dd, J=8.5, 0.9 Hz, 1 H, H-5′), 8.09 (dd, J=8.5, 0.8 Hz, 1 H, H-8′), 7.80 (ddd, J=8.5, 6.8, 1.4 Hz, 1 H, H-7′), 7.75 (d, J=4.5 Hz, 1 H, H-3′), 7.65 (ddd, J=8.5, 6.8, 1.4 Hz, 1 H, H-6′), 7.58 (br s, 1 H, H-2″), 7.44-7.48 (m, 2 H, H-5, H-6″), 7.20 (br t, J=7.7 Hz, 1 H, H-5″), 6.80 (br d, J=7.5 Hz, 1 H, H-4″), 2.29 (s, 3 H, CH₃); ¹³C NMR δ 163.4, 150.1, 148.4, 147.6, 140.9, 140.0, 138.1, 129.4, 129.2, 128.7, 126.4, 126.1, 125.2, 122.1, 120.4, 117.4, 114.1, 109.4, 21.2; MS m/z 318.5 (MH⁺, 100%). Anal. calcd for C₁₉H₁₅N₃S.¼(CH₃CH₂)₂O: C, 71.51; H, 5.25; N, 12.51.Found: C, 71.62; H, 5.25; N, 12.51%.

Alternative Preparation of 2-Bromo-1-(4-quinolinyl)ethanone Hydrobromide (191). A suspension of quinoline 4-carboxylic acid (0.74 g, 4.3 mmol) and DMF (2 drops) in thionyl chloride (15 mL) was stirred at 40° C. for 3 h. Thionyl chloride was evaporated and the residue was dissolved in dry THF (50 mL) and cooled to 0° C. A solution of TMSCH₂N₂ in hexanes (2.5 M, 5.3 mL, 10.7 mmol) was added and the mixture was stirred at 0° C. for 5 h. cHBr (9 mL) was added carefully and the mixture stirred at 20° C. for 16 h. The mixture was neutralized with saturated aqueous KHCO₃ and then extracted with EtOAc (3×50 mL). The combined organic fraction was washed with water (40 mL), washed with brine (40 mL), dried and the solvent evaporated. The residue was purified by column chromatography, eluting with a gradient (20-50%) of EtOAc/pet. ether, to give crude bromide 191 (0.76 g, 71%) as oil: ¹H NMR (CDCl₃) δ 9.05 (d, J=4.4 Hz, 1 H, H-2′), 8.34 (dd, J=8.6, 0.8 Hz, 1 H, H-8′), 8.20 (dd, J=8.4, 0.6 Hz, 1 H, H-5′), 7.81 (ddd, J=8.6, 6.9, 1.4 Hz, 1 H, H-7′), 7.68 (ddd, J=8.4, 6.9, 1.3 Hz, 1 H, H-6′), 7.62 (d, J=4.4 Hz, 1 H, H-3′), 4.52 (s, 2 H, H-2); MS m/z 251.4 (MH⁺, 100%). The bromide was converted to the hydrobromide salt and used directly.

### Example 1-107

### N-(3-Methylphenyl)-4-(2-phenyl-4-quinolinyl)-1,3-thiazol-2-amine (194)

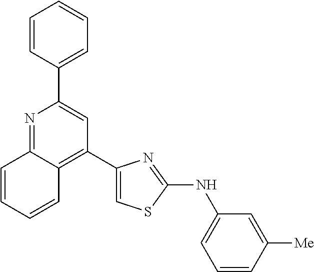

2-Bromo-1-(2-phenyl-4-quinolinyl)ethanone Hydrobromide (193). A suspension of 2-phenyl-4-quinolinecarboxylic acid (0.80 g, 3.2 mmol) and DMF (2 drops) in thionyl chloride (15 mL) was stirred at 40° C. for 3 h. Thionyl chloride was evaporated and the residue was dissolved in dry THF (50 mL) and cooled to 0° C. A solution of TMSCH₂N₂ in hexanes (2.5 M, 3.2 mL, 8.0 mmol) was added and the mixture was stirred at 0° C. for 5 h. cHBr (5 mL) was added carefully and the mixture stirred at 20° C. for 16 h. The mixture was neutralized with saturated aqueous KHCO₃ and then extracted with EtOAc (3×50 mL). The combined organic fraction was washed with water (40 mL), washed with brine (40 mL), dried and the solvent evaporated. The residue was purified by column chromatography, eluting with 5% EtOAc/pet. ether, to give bromide 193 (0.50 g, 48%) as an oil: ¹H NMR (CDCl₃) δ 8.30 (dd, J=8.6, 0.8 Hz, 1 H, H-8′), 8.25 (d, J=8.5 Hz, 1 H, H-5′), 8.15-8.19 (m, 2 H, H-2″, H-6″), 8.07 (s, 1 H, H-3′), 7.80 (ddd, J=8.4, 6.9, 1.4 Hz, 1 H, H-7′), 7.64 (ddd, J=8.4, 6.9, 1.3 Hz, 1 H, H-6′), 7.50-7.59 (m, 3 H, H-3″, H-4″, H-5″), 4.58 (s, 2 H, H-2); MS m/z 326.5/328.5 (MH⁺, 100%). The bromide was converted to the hydrobromide salt and used directly.

N-(3-Methylphenyl)-4-(2-phenyl-4-quinolinyl)-1,3-thiazol-2-amine (194). A mixture of bromoketone hydrobromide 193 (0.23 g, 0.56 mmol) and 3-methylphenylthiourea (4) (94 mg, 0.56 mmol) in EtOH (10 mL) was stirred at reflux temperature for 2 h. The mixture was cooled to 20° C., diluted with water (50 mL), the pH adjusted to ca. 8 with aqueous NH₃ and the mixture stirred at 5° C. for 2 h. The precipitate was filtered, washed with water (5 mL) and dried. The crude solid was purified by column chromatography, eluting with 50% EtOAc/pet. ether, to give amine 194 (0.18 g, 85%) as a tan powder: mp (EtOAc/Et₂O) 192-194° C.; ¹H NMR δ 10.36 (br s, 1 H, NH), 8.76 (dd, J=8.5, 0.8 Hz, 1 H, H-8′), 8.32-8.36 (m, 3 H, H-3′, H-2″, H-6″), 8.15 (dd, J=8.4, 0.6 Hz, 1 H, H-5′), 7.82 (ddd, J=8.5, 6.8, 1.4 Hz, 1 H, H-7′), 7.50-7.67 (m, 6 H, H-5, H-6′, H-3″, H-4″, H-5″, H-2″), 7.42 (br d, J=8.1 Hz, 1 H, H-8″), 7.21 (br t, J=7.8 Hz, 1 H, H-5″′), 6.80 (d, J=7.5 Hz, 1 H, H-4″), 2.31 (s, 3 H, CH₃); ¹³C NMR δ 163.3, 155.7, 148.4, 147.9, 141.1, 140.9, 138.5, 138.1, 129.6, 129.5, 129.4, 128.8, 128.7 (2), 127.1 (2), 126.2, 126.0, 124.4, 122.1, 117.7, 117.5, 114.1, 109.6, 21.2; MS m/z 394.6 (MW, 100%). Anal. calcd for C₂₅H₁₉N₃S: C, 76.31; H, 4.87; N, 10.68.Found: C, 76.01; H, 4.79; N, 10.59%.

### Example 1-108

### N-(3-Methylphenyl)-4-(4-pyridinyl)-1H-imidazol-2-amine (196)

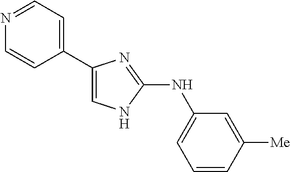

N-(3-Methylphenyl)-4-(4-pyridinyl)-1H-imidazol-2-amine (196). KOH (80 mg, 1.4 mmol) was added to a stirred suspension of N-(3-methylphenyl)guanidine nitrate 195 (Tavares, X. T. et. al. J. Med. Chem. 2004, 47, 4716.) (300 mg, 1.4 mmol) in absolute ethanol (30 mL), and the mixture was heated at 80° C. for 10 min. A solution of bromoketone hydrobromide 1 (393 mg, 1.4 mmol) and NEt₃ (0.4 mL, 2.8 mmol) in absolute ethanol (10 mL) was added within 5 min and the reaction mixture was stirred at 80° C. for 4 h. The mixture was then cooled to 20° C. and the solvent was evaporated. Dilute aqueous ammonia solution was added (15 mL), and the residue was extracted with EtOAc (3×50 mL). The combined organic phases were dried, filtered and the solvent was removed under reduced pressure. The residue was purified by column chromatography, eluting with a gradient (5-10%) of MeOH/DCM, to give imidazole 196 (40 mg, 12%) as a beige solid: mp (DCM/MeOH) 170-171° C.; ¹H NMR δ 8.45 (dd, J=4.6, 1.6 Hz, 2 H, H-2′, H-6′), 7.67 (s, 1 H, H-5), 7.61 (dd, J=4.5, 1.6 Hz, 2 H, H-3′, H-5′), 7.42 (t, J=7.7 Hz, 1 H, H-5″), 7.34 (br s, 1 H, H-2″), 7.30 (br d, J=7.7 Hz, 1 H, H-6″), 7.23 (d, J=7.5 Hz, 1 H, H-4″), 5.66 (br s, 1 H, NH), 2.38 (s, 3 H, CH₃), imidazole NH not observed; ¹³C NMR (CD₃OD) δ 151.5, 150.2 (2), 144.1, 141.6, 137.8, 135.3, 130.9, 130.3, 126.6, 123.1, 120.3 (2), 116.9, 21.4; MS m/z 251.5 (MH⁺, 100%).

### Example 1-109

### N-(3-Methylphenyl)-4-[2-(4-morpholinyl)-4-pyridinyl]-1,3-thiazol-2-amine (197)

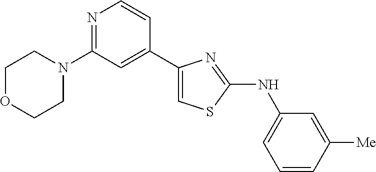

N-(3-Methylphenyl)-4-[2-(4-morpholinyl)-4-pyridinyl]-1,3-thiazol-2-amine (197). A mixture of 4-(2-bromo-4-pyridinyl)-N-(3-methylphenyl)-1,3-thiazol-2-amine (154) (0.16 g, 0.45 mmol) in morpholine (5 mL) was stirred at 100° C. for 48 h. The mixture was cooled to 20° C., diluted with water (80 mL), extracted with EtOAc (3×20 mL). The organic fraction was washed with water (10 mL), washed with brine (10 mL) and dried. The solvent was evaporated and the crude solid was purified by column chromatography, eluting with a gradient (20-50%) of EtOAc, to give amine 197 (0.10 g, 64%) as a white powder: mp (EtOAc/pet. ether) 190-191° C.; ¹H NMR [(CD₃)₂SO]δ 10.23 (s, 1 H, NH), 8.17 (d, J=5.1 Hz, 1 H, H-6′), 7.61 (s, 1 H, H-5), 7.58 (br s, 1 H, H-2″), 7.45 (br d, J=8.0 Hz, 1 H, H-6″), 7.49 (br s, 1 H, H-3′), 7.23 (br t, J=7.8 Hz, 1 H, H-5″), 7.18 (dd, J=5.1, 1.2 Hz, 1 H, H-5′), 6.80 (br d, J=7.5 Hz, 1 H, H-4″), 3.74 (br dd, J=5.1, 4.6 Hz, 4 H, 2×CH₂O), 3.51 (br dd, J=5.1, 4.6 Hz, 4 H, 2×CH₂N), 2.33 (s, 3 H, CH₃); ¹³C NMR [(CD₃)₂SO]δ 163.2, 159.9, 148.5, 148.1, 142.7, 141.0, 138.2, 128.9, 122.1, 117.6, 114.2, 110.5, 106.4, 102.9, 66.0 (2), 45.2 (2), 21.3; MS m/z 352.6 (MW, 100%). Anal. calcd for C₁₉H₂₀N₄OS: C, 64.75; H, 5.72; N, 15.90.Found: C, 64.74; H, 5.85; N, 15.91%.

### Example 1-110

### N-(3-Methylphenyl)-4-[2-(4-methyl-1-piperazinyl)-4-pyridinyl]-1,3-thiazol-2-amine (198)

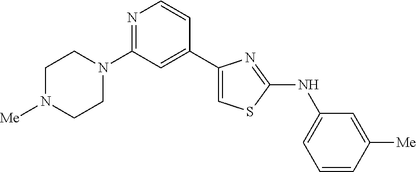

N-(3-Methylphenyl)-4-[2-(4-methyl-1-piperazinyl)-4-pyridinyl]-1,3-thiazol-2-amine (198). A mixture of 4-(2-bromo-4-pyridinyl)-N-(3-methylphenyl)-1,3-thiazol-2-amine (154) (0.16 g, 0.45 mmol) in 1-methylpiperazine (5 mL) was stirred at 100° C. for 16 h. The mixture was cooled to 20° C., diluted with water (80 mL), extracted with EtOAc (3×20 mL). The organic fraction was washed with water (10 mL), washed with brine (10 mL) and dried. The solvent was evaporated and the crude solid was purified by column chromatography, eluting with 5% MeOH/DCM, to give amine 198 (0.09 g, 57%) as a white powder: mp (EtOAc/pet. ether) 141-143° C.; ¹H NMR [(CD₃)₂SO]δ 10.23 (s, 1 H, NH), 8.15 (d, J=5.1 Hz, 1 H, H-6′), 7.62 (br s, 1 H, H-2″), 7.60 (s, 1 H, H-5), 7.42 (br d, J=8.0 Hz, 1 H, H-6″), 7.29 (br s, 1 H, H-3′), 7.22 (br t, J=7.8 Hz, 1 H, H-5″), 7.14 (dd, J=5.1, 1.0 Hz, 1 H, H-5′), 6.80 (br d, J=7.5 Hz, 1 H, H-4″), 3.55 (br dd, J=5.0, 4.7 Hz, 4 H, 2×CH₂N), 2.44 (br dd, J=5.0, 4.7 Hz, 4 H, 2×CH₂N), 2.32 (s, 3 H, CH₃), 2.24 (s, 3 H, CH₃); ¹³C NMR [(CD₃)₂SO]δ 163.2, 159.8, 148.6, 148.1, 142.6, 141.0, 138.1, 128.8, 122.1, 117.6, 114.2, 110.0, 106.3, 103.0, 54.4 (2), 45.8, 44.7 (2), 21.3; MS m/z 366.9 (MH⁺, 100%). Anal. calcd for C₂₀1^{˜}1₂₃N₅S: C, 65.72; H, 6.34; N, 19.16.Found: C, 65.51; H, 6.48; N, 18.86%.

### Example 1-111

### N,N-Dimethyl-4-{2-[(3-methylphenyl)amino]-1,3-thiazol-4-yl}-2-pyridinamine (199)

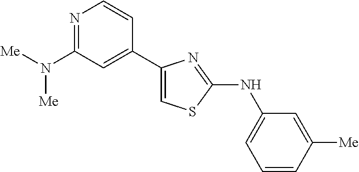

N,N-Dimethyl-4-{2-[(3-methylphenyl)amino]-1,3-thiazol-4-yl}-2-pyridinamine (199). A mixture of 4-(2-bromo-4-pyridinyl)-N-(3-methylphenyl)-1,3-thiazol-2-amine (154) (0.17 g, 0.45 mmol) in 40% aqueous dimethylamine (5 mL) was stirred at 80° C. for 16 h. The mixture was cooled to 20° C., diluted with water (80 mL), extracted with EtOAc (3×20 mL). The organic fraction was washed with water (10 mL), washed with brine (10 mL) and dried. The solvent was evaporated and the crude solid was purified by column chromatography, eluting with 40% EtOAc/pet. ether, to give amine 199 (0.08 g, 55%) as a tan powder: mp (EtOAc/pet. ether) 148-150° C.; ¹H NMR [(CD₃)₂SO]δ 10.22 (s, 1 H, NH), 8.11 (d, J=5.1 Hz, 1 H, H-6), 7.65 (br s, 1 H, H-2″), 7.55 (s, 1 H, H-5′), 7.40 (br d, J=8.0 Hz, 1 H, H-6″), 7.22 (br t, J=7.8 Hz, 1 H, H-5″), 7.14 (br s, 1 H, H-3), 7.05 (dd, J=5.1, 1.0 Hz, 1 H, H-5), 6.80 (br d, J=7.5 Hz, 1 H, H-4″), 3.09 [s, 6 H, N(CH₃)₂], 2.32 (s, 3 H, CH₃); ¹³C NMR [(CD₃)₂SO]δ 163.1, 159.8, 148.8, 148.0, 142.3, 141.0, 138.1, 128.8, 122.1, 117.6, 114.2, 108.4, 106.1, 101.9, 37.7 (2), 21.3; MS m/z 311.7 (MH⁺, 100%). Anal. calcd for C₁₇H₁₈N₄S: C, 65.78; H, 5.84; N, 18.05.Found: C, 65.99; H, 6.08; N, 17.74%.

### Example 2

### Identification of Novel Molecules Targeting VHL-Deficient Renal Cell Carcinoma

### Materials and Methods

Cell Culture and Cell Treatments: RCC4 parental and RCC4 with VHL-reintroduced (RCC4/VHL), SN12C and SN12C-CSCG-VHL shRNA, MEF ATG5+/+and ATG5−/− were maintained in DMEM supplemented with 10% FCS. Cells were plated at 70% confluency, and treated with 1.25 μM (RCC4) or 3.75 μM (SN12C) of STF-62247 (“compound 5”) overnight in presence or absence of 2 mM 3-methyladenine (“3-MA”), 1 μg/mlBrefeldin A, 15 μM LY294002, 2 μg/ml Tunicamycin, or 10 μM U0126 (Sigma Chemical Co).

Cell Viability and XTT assays. For cell viability, 100,000 cells were plated in a 12-well plate. The following day, 1.25 μM STF-62247 was added in the presence or absence of 1 mM 3-MA for 24 hours at 37° C. Cells were trypsinized and counted by trypan blue exclusion. For XTT assays, 5000 RCC4 with and without VHL cells or 2,500 SN12C with and without VHL shRNA cells were plated in 96-well plates. The following day, vehicle (DMSO), STF-62247 or each of 19 analogs were added to media by serial dilution. Four days later, the media was aspirated and XTT solution containing 0.3 mg/ml of XTT (Sigma Chemical Co) in Phenol Red-free media, 20% FCS and 2.65 mg/ml N-methyl dibenzopyrazine methyl sulfate (PMS) was added to the cells and incubated at 37° C. for 1-2 hours. Metabolism of XTT was quantified by measuring the absorbance at 450 nm on a plate reader.

Clonogenic Cell Survival Assay. RCC4, RCC4/VHL, SN12C and SN12C-VHL shRNA were plated at 300 cells per 60 mm tissue culture dish. The following day, vehicle or STF-62247 was added and cells were incubated in the presence of drug for 10 days in RCC4 and RCC4/VHL and 7-10 days for SN12C and SN12C-VHL shRNA. The colonies were stained with crystal violet, and quantified. Each experiment was performed at least three times in triplicate.

Western blot analysis. After treatments, cells were lysed in urea buffer (9M urea, 75 mM Tris (pH 7.5), 150 mM b-mercaptoethanol), placed on ice for 10 minutes and sonicated briefly (15 seconds). Extracts were centrifugated at 8,000×g for 10 minutes to eliminate insoluble material. Lysates were quantitated using Bio-Rad protein Assay (Bio-Rad Laboratories) and 20-30 μg of proteins were separated on a 7.5% or 15% bis-acrylamide gels and then transferred onto PVDF membranes. Proteins were visualized using HIF-2α (Novus Biological), HIF-1α, pVHL and BiP (BD Biosciences), LC3 (MBL), Atg5, or α-tubulin antibodies.

Quantification of Acidic Vesicle Organelles with Acridine Orange. In acridine orange-stained cells, the cytoplasm and nucleus are bright green and dim red, whereas acidic compartments are bright red. The intensity of the red fluorescence is proportional to the degree of acidity. Following 20 hours of treatment with STF-62247 or analogs, cells were stained with 1 μg/ml of acridine orange for 15 min. Cells were trypsinized and then analyzed by flow cytometry using FACScan cytometer and CellQuest software.

Immunofluorescence staining. Cells were seeded on glass coverslips and at subconfluence, STF-62247 was added at 1.25 μM for RCC4 and 3.75 μM for SN12C. After 20 hours, cells were fixed in 3.7% formaldehyde for 10 min at room temperature and washed in PBS. Then cells were permeabilized in 0.2% Triton X-100 for 5 min at room temperature and washed in PBS. The coverslips were incubated with primary antibody LC3 diluted 1:500 in PBS containing 0.2% BSA for 1 hour at room temperature. Cells were washed three times in PBS-BSA and the bound primary antibody was detected using a fluorescein-conjugated secondary antibody (ALEXA) diluted 1:500 in PBS-BSA and incubated for 1 hour. Coverslips were washed and mounted in mounting medium from Vector Laboratories according to the manufacturer's instructions. Cells were examined with a confocal microscope

Visualization of Monodansylcadaverine (MDC) vacuoles. Cells were incubated in the presence of drug for 20 hours and labeled with 50 μM of the autofluorescent marker MDC (Sigma Chemical Co) in PBS at 37° C. for 10 minutes. Then, cells are fixed with 3.7% formaldehyde for 10 min at room temperature and washed in PBS. MDC was observed by fluorescence microscopy (Nikon Eclipse E800).

In vivo studies. 2−3×10⁶ SN12C and SN12C-VHL shRNA cells were injected in male scid mice 4-6 weeks old. When tumor volume reached 150 mm³, mice were randomized into vehicle control group or STF-62247 treatment group. Mice were treated every day with 10-30 μM of STF-62247 intraperitoneally. Tumor growth was measured every 2-3 days after the drug treatment was started.

shRNA constructs, plasmids and retroviral infection. The CMV-EYFP fluorescent protein and the HIF-2α P531A/P847A double mutant were cloned into pBabe-puro vector. The sequence used for HIF-1α and HIF-2α shRNA were gaggaactaccattatat and ggagacggaggtggtctat respectively. Retrovirus were produced by transfecting plasmids into ΦNX-amphotropic cell lines according to manufacturer's instructions with Lipofectamine Plus (Invitrogen). The next day after transfection, cells were placed at 32° C. Retrovirus was harvested 48 and 72 hours after transfection. Puromycin was used to select for stable retroviral integrants.

Transient transfection of RCC. Cells were transfected with siRNA against AtgS, CHMP6, PAK2, Rab11a, OSBP3, ALG5, Atg7, Atg9 (SMART pool) and siControl2 non-targeting pool from Dharmacon using Dharmafect Reagent 1 for 48 hours. Cells were split for clonogenic assay, harvested for RNA or protein.

Screen in yeast and killing curves. Genotypes of the parental yeast strain BY4743, construction of the homozygous diploid deletion strains, and construction of the homozygous diploid deletion pool have been described previously (Thomas, G. V., et al., Nat. Med. 2006, 12, 122). A mutant pool of 4728 nonessential homozygous diploid deletion strains was used. The parental diploid strain BY4743 was used as control in survival and complementation assays. For clonogenic survival, equivalent numbers of early log-phase cells parental and deletion strains for ALG5, APS1, OSH3, CLA4, DRS2, DAPI, VPS20, VPS25, FIG4, SPT4 were suspended in yeast extract peptone dextrose (YPD) and treated with STF-62247 (0-100 μM) for 1 h while being shaken at 300 rpm at 30° C. Cells were then pelleted, washed once with buffer (10 mM Tris and 5 mM EDTA), pelleted, and resuspended in the same buffer. Cells were then plated at appropriate dilutions onto YPD solid medium to allow for accurate counting of surviving colonies (range, 50-250). Plates were incubated for 2 days at 30° C. before counting colonies. Full survival curves were performed in triplicates for each selected deletion strain.

Quantitative RT-PCR (qRT-PCR). RNA from cells transfected with siControl or siAtg was prepared using TRIzol (Invitrogen, Carlsbad, Calif.) according to the manufacturer's protocol. The reverse transcription was performed with Moloney Murine Leukemia Virus MMLV reverse transcriptase (Invitrogen) using two μ grams of RNA and 5 μM-random primers (Invitrogen) according to the manufacturer's instructions. Approximately 0.5% of each RT reaction was used for the quantitative RT (qRT)-PCR reactions containing 5 μl 2×SYBR Green master mix (ABI, Foster City, Calif.) and 200 nM forward and reverse primers specific of the different genes of interest in a volume of 10 μl. Detection and data analysis was carried out with the ABI PRISM 7900 sequence detection system using TBP primers as an internal control. PCR primer sequences are obtained from the Primer Bank database (see Primerbank web site, Harvard University) and synthesized in the Stanford Protein and Nucleic Acid Biotechnology Facility.

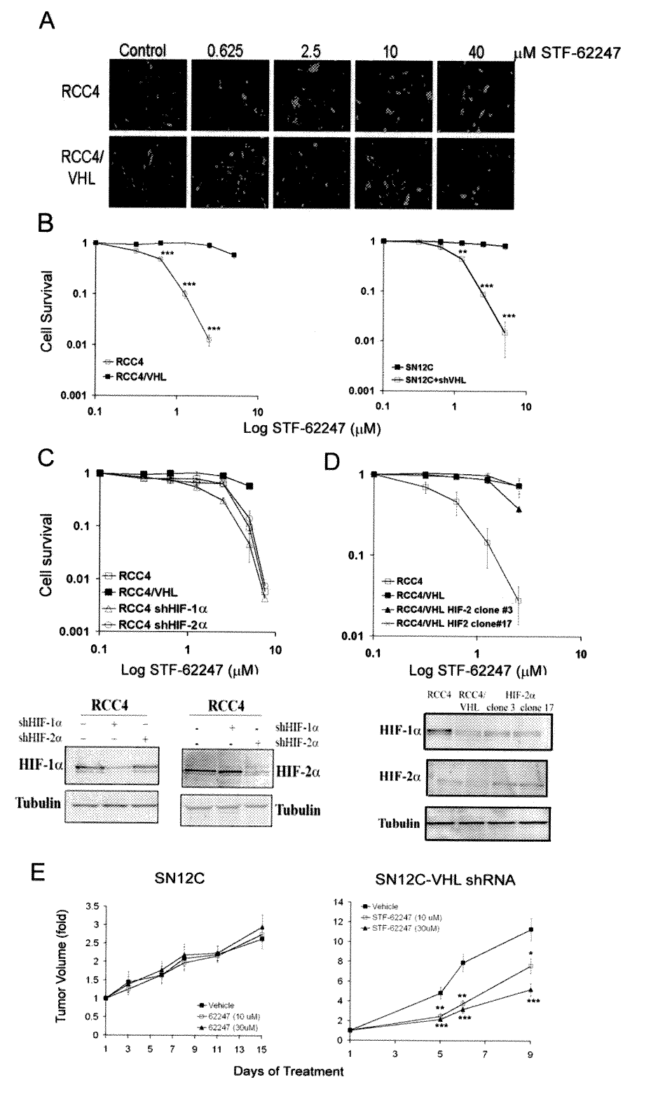

FIG. 1 shows that STF-62247 induces cytotoxicity and reduces tumor growth in VHL-deficient cells in HIF-independent manner. A. RCC4 and RCC4/VHL cells were labeled with EYFP and the effect of STF-62247 was monitored by fluorescence in each cell type separately. Cells are pseudocolored. B. Clonogenic assay in RCC4, RCC4/VHL, SN12C, SN12C-VHL shRNA cells in the presence of STF-62247 (0-30 μM). Each point of the cell survival is calculated by the average of three different experiments in triplicate as the percent of treated on untreated plate. The error bars represented the standard error of the mean (SEM). C. Upper Panel, clonogenic assay in RCC4 infected with HIF-1α or HIF-2α shRNA by retroviral infection in presence of drug in same conditions described above. Bottom panel, HIF-1α or HIF-2α protein expression evaluated by western blot in RCC4 cells with HIF-1α or HIF-2α shRNA. Tubulin is used as loading control. D. Upper Panel, clonogenic assay with STF-62247 in RCC4 where HIF-2α was stably introduced into VHL-positive cells by retroviral infection. Bottom panel, western blot for HIF-1α or HIF-2α in RCC4 VHL-positive cells. E. SN12C and SN12C-VHL shRNA (2−3×10⁶ cells) are injected in SCID mice. When the tumors reach 120 mm³, a daily treatment with vehicle or 10-30 μM of STF-62247 was administrated intraperitoneally. Tumor volume was measured every 2-3 days and data are represented in fold. Five tumors in each condition were analyzed where * p<0.05, **p<0.01 and ***p<0.005.

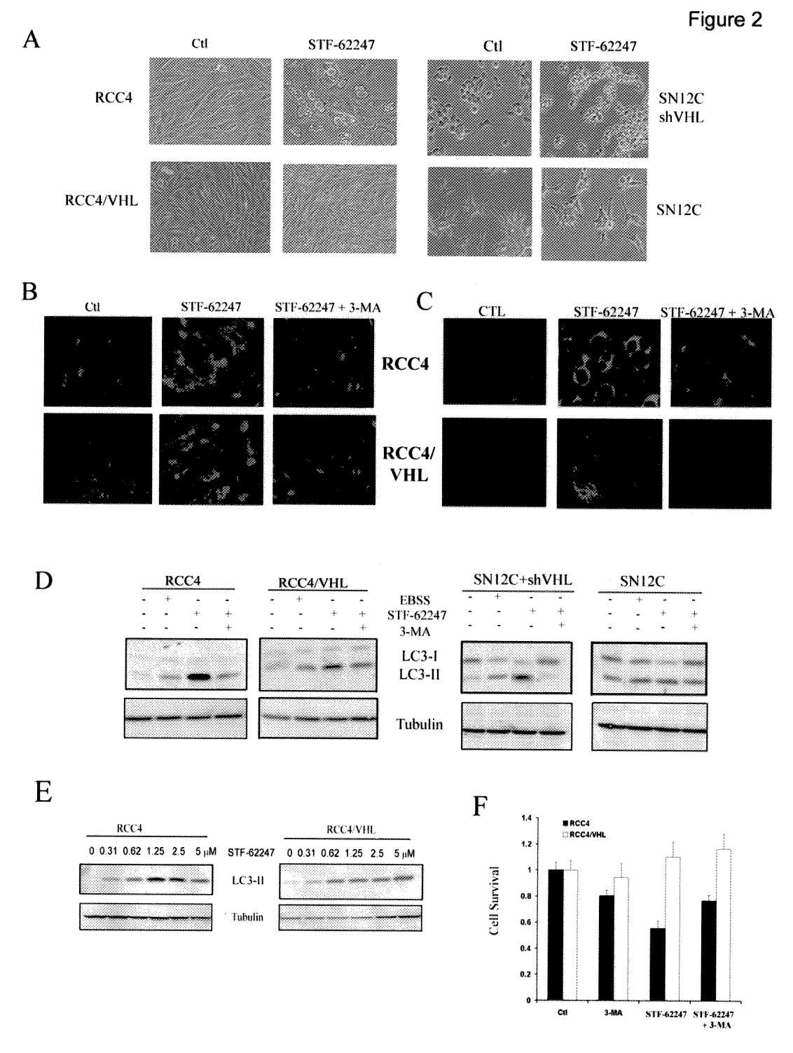

FIG. 2 shows the presence of autophagic vacuoles upon treatment with STF-62247 in RCCs. A. Phase contrast pictures of VHL-deficient RCC4 and SN12C-VHL shRNA cells and VHL-positive cells RCC4 and SN12C after 20 hrs of 1.25 μM STF-62247. B. Immunostaining for LC3 following STF-62247 treatment at 1.25 μM for 20 hrs with or without 2 mM of 3-MA in RCC4 with and without VHL cells. After treatment, cells were fixed, permeabilized and processed for the detection of punctuate staining by indirect immunofluorescence. C. Autophagic vacuoles stained with monodansylcadaverine (MDC). After STF-62247 treatment in the same conditions, MDC was added for the last 10 minutes. Cells were fixed, washed with PBS and observed directly under microscope. D. Western blot for LC3 in RCC4, RCC4/VHL, SN12C, and SN12C-VHL shRNA cells after STF-62247 treatment with or without 3-MA. Nutrient starvation (EBSS) is used as positive control. E. Western blot for LC3 in RCC4 and RCC4/VHL with different concentrations of STF-62247. Tubulin was used as loading control. F. Cell viability assay in VHL-deficient RCC4 and wild-type VHL cells treated with 1 mM of 3-MA, 1.25 μM of STF-62247 alone or in combination for 24 hrs. Viability is measured by trypan blue exclusion assay and is represented by the average of three different experiments in duplicate as the percent of treated on untreated cells. The error bars are represented as SEM.

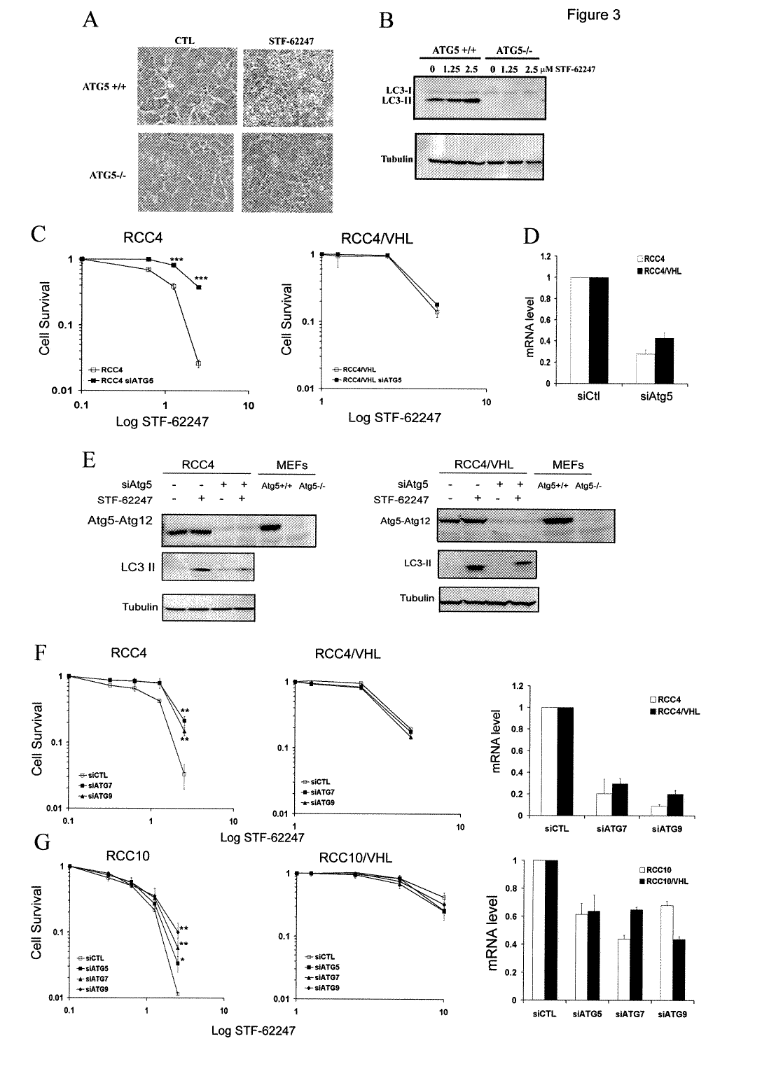

FIG. 3 shows the effects of STF-62247 on ATG5 in autophagic cell death. A. MEFs ATG5+/+ and ATG5−/− were treated in presence of 3 μM of STF-62247 and examined under light microscopy 20 hrs later. B. LC3 processing evaluated by Western blot following 24 hours of drug treatment in MEFs. C. Cell Survival measured by clonogenic assay with STF-62247 in RCC4 and RCC4/VHL cells transiently transfected with siRNA to AtgS. Colony formation was evaluated as described before. The error bars are represented as SEM. ***p<0.005 represents the difference between RCC4 and RCC4 with siAtg5 cells in presence of STF-62247. D. Western blot for LC3 and AtgS in RCC4 and RCC4/VHL cells transiently transfected with siRNA against AtgG5. Tubulin was used as loading control. E. mRNA level of AtgS quantified by qRT-PCR in RCC4 and RCC4/VHL cells transfected with siRNA to Atg5 for 48 hours. F, G. Left and middle panels, Clonogenic assay with STF-62247 in cells transfected with Atg7 and Atg9 siRNA in VHL-deficient RCC4, RCC10 and wild-type VHL cells. Colony formation was evaluated as described before. The error bars are represented as SEM. **p<0.01 represents the difference between siControl cells and siAtg cells in response to STF-62247. Right panels, mRNA levels of Atg7 and Atg9 (and Atg5 in RCC10 cells) quantified by qRT-PCR after 48 hours of transfection using siRNA to Atg7 and Atg9 (and Atg5 in RCC10 cells) in RCC4 and RCC10 cells with or without VHL.

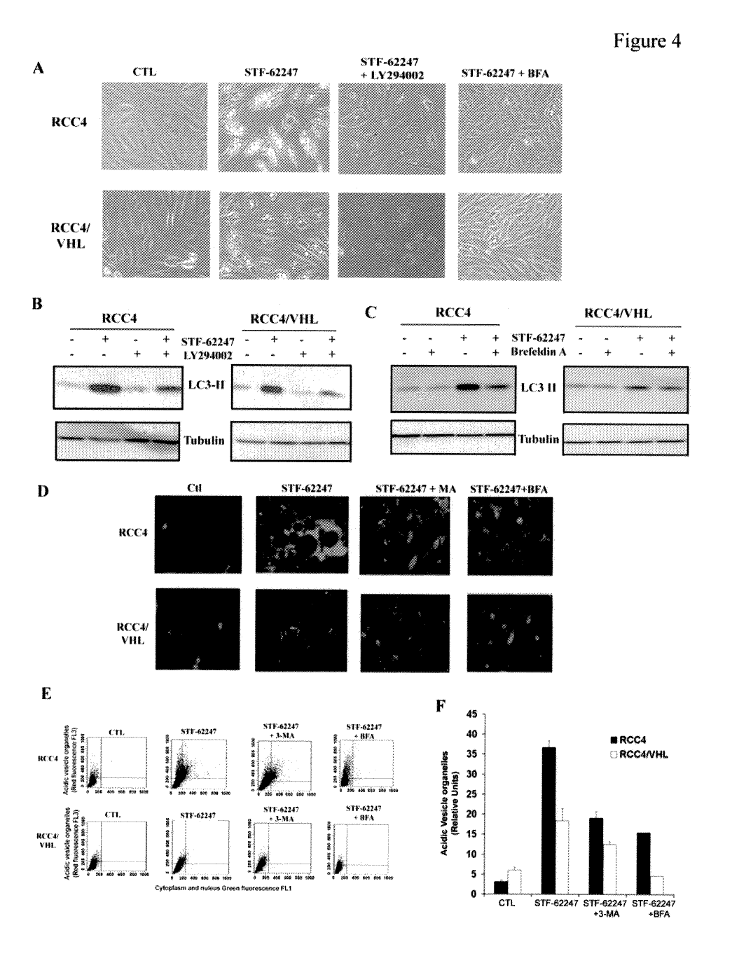

FIG. 4 shows the effects of STF-62247 on Golgi trafficking, PI3K, and vesicle acidification. A. VHL-deficient RCC4 and wild-type VHL were incubated in presence of 10 μM LY294002, or 1 μg/mlBrefeldin A with 1.25 μM of STF-62247 for 20 hrs and the presence of vacuoles was examined under light microscopy. B,C. LC3 processing was evaluated by Western blot after the treatment with Brefeldin A or LY294002 in presence or absence of STF-62247 as described above. Tubulin was used as loading control. D. Cells were treated with 1.25 μM of STF-62247 with or without 2 mM of 3-MA, 1 μg/ml of Brefeldin A for 20 hrs. For the last 15 min, acidic components were stained with 1 μg/ml of acridine orange and visualized under fluorescence microscope. E. FACS analysis to measure the acidic vesicle organelles (AVO) in cells treated in D. y axis represents the concentrated dye in the acidic vesicles, whereas the cytoplasm and the nucleus showed green fluorescence in x axis. F. Quantification of the FACS analysis. The data are calculated as the average of three different experiments as relative units of treated over untreated cells. The error bars are represented as the standard error of the mean (SEM).

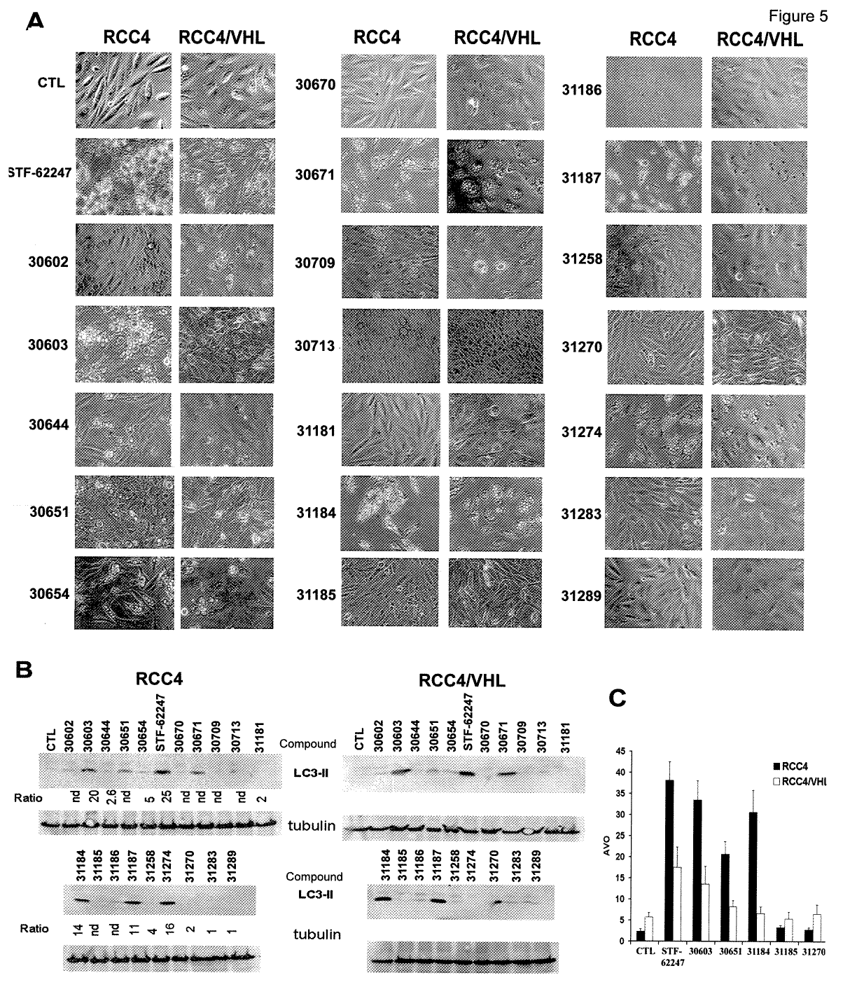

FIG. 5 shows an evaluation of autophagy in RCC treated with analogs of STF-62247. A. RCC4 and RCC4/VHL were treated with 1.25 μM of each 19 analogs for 20 hours and the presence of vacuoles was examined under light microscopy. B. Western blot for LC3 after treatment with each compound. Tubulin was used as loading control. Ratio is the relative from the IC50 in RCC4/VHL cells/IC50 in RCC4 cells. C. Acidic vesicle organelles (AVO) measured by FACS analysis in cells treated with 1.25 μM of STF-62247 or with compounds 21, 15, 25, 47, and 53 and stained with acridine orange.

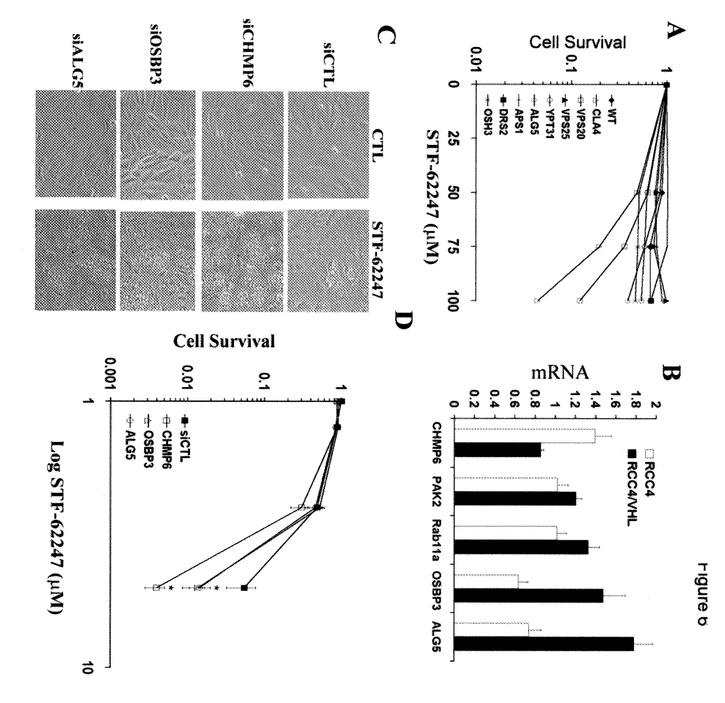

FIG. 6 shows the effects of STF-62247 on Golgi trafficking proteins. A. Killing curves for 8 deletion yeast strains. Wild-type, ALG5, APS1, CLA4, DRS2, OSH3, VPS20, VPS25, YPT31 were incubated with STF-62247 (0-100 μM) for 1 hour and colony formation was evaluated after two days. Each point of the survival is calculated by the average of two different experiments in triplicate and as the percent of treated on untreated plate. B. Quantitative real time PCR for CHMP6, PAK2, RAB11a, OSBP3 and ALG5 in RCC4 and RCC4/VHL. Histogram is calculated by the average of three different experiments in triplicate as the percent of RCC4/VHL on RCC4 cells. The error bars are represented as SEM and *p<0.05, **p<0.01. C. VHL cells were transiently transfected with siRNA for CHMP6, OSBP3 and ALG5 for 72 hours. The last 24 hours, 1.25 μM of STF-62247 was added and the vacuole formation was examined under light microscopy. D. Clonogenic assay with STF-62247 (0-30 μM) in RCC4/VHL cells transiently transfected in the same condition than in C. Colony formation was evaluated as described before. *p<0.05 represents the difference between VHL cells and VHL with siRNA in presence of 5 μM of STF-62247.

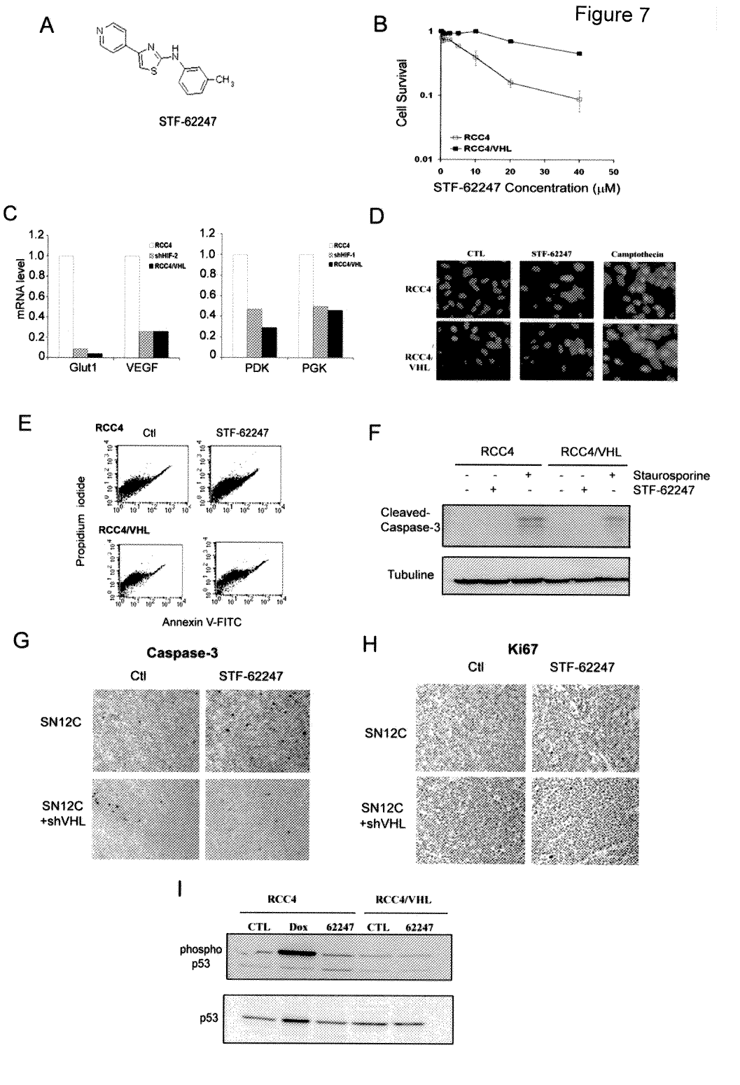

FIG. 7 shows A. Structure of STF-62247 (also termed “30661” and “compound 5”). B. XTT assay in RCC4 and RCC4/VHL in presence of STF-62247 (0-40 μM). Each point of the cell survival is calculated by the average of three different experiments in duplicate as the percent of treated on untreated plate. The error bars are represented as the standard error of the mean (SEM) C. qRT-PCR for Glutl, VEGF, PDF and PGK in RCC4 cells with or without HIF-1a, HIF-2a shRNA and in RCC4/VHL. D. Hoescht staining in RCC4 and RCC4/VHL cells with 1.25 μM of STF-62247 for 2 days. Camptothecin was used as positive control E. FACS analysis for apoptosis after an annexin V-FITC staining in presence or absence of propidium iodine (PI) in cells after a treatment in same conditions than in C. F. Immunoblot for active caspase-3 in RCC4 and RCC4/VHL following treatment with 1.25 μM of STF-62247 for 24 hours. Staurosporine was used as positive control (0.2 μM for 24 hours). G,H. Immunostaining of caspase-3 and Ki67 respectively in SN12C and SN12C+shVHL tumor samples untreated or treated with 30 μM of STF-62247.1. Immunoblot for p53 and SerlS phospho-p53 in RCC4 and RCC4/VHL cells after a treatment with 1.25 μM of STF-62247 or doxorubicin for 24 hours.

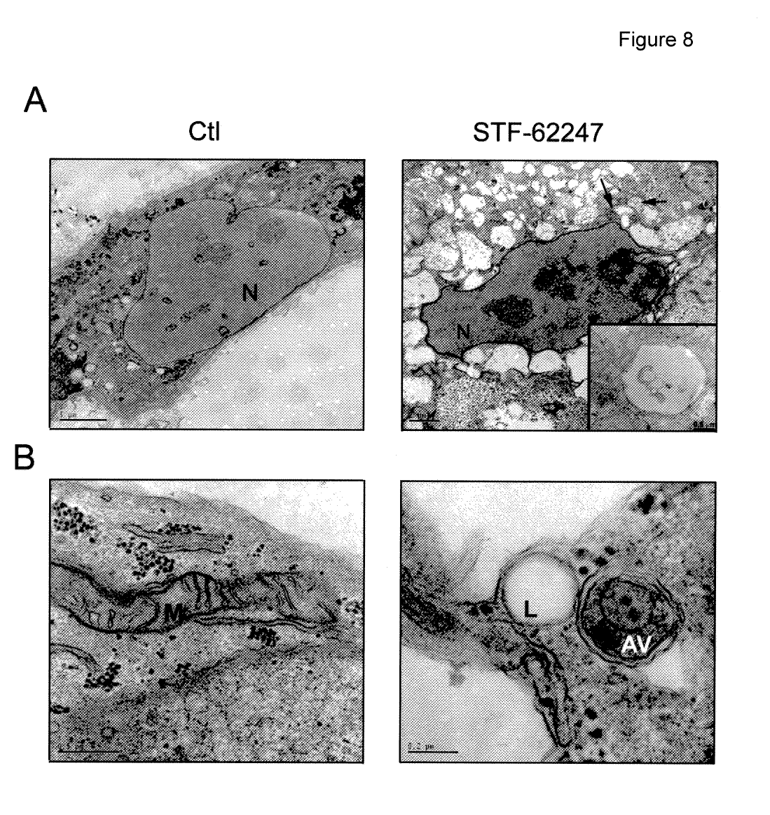

FIG. 8 shows A. Transmission electron microscopy in RCC4 cells treated with vehicle control (DMSO) or with 1.25 μM STF-62247 for 20 hrs. Arrows represent autophagic vacuoles during drug treatment. N. Nucleus. B. RCC4 cells treated with STF-62247 observed at higher magnification. M. Mitochondria L. Lysosome AV. Autophagic Vacuole

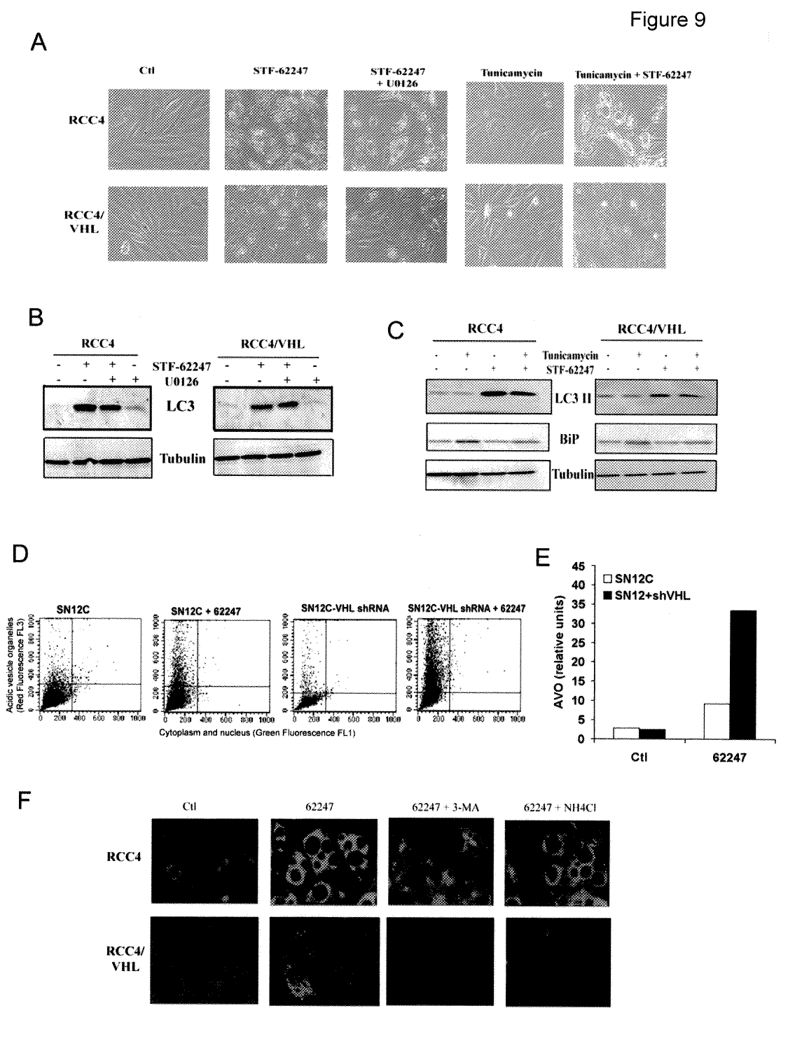

FIG. 9 shows A. VHL-deficient RCC4 and wild-type VHL incubated in the presence of 10 μM U0126, or 2 μg/ml tunicamycin with 1.25 μM of STF-62247 for 20 hrs and the presence of vacuoles was examined under light microscopy. B,C. LC3 processing was evaluated by Western blot after the treatment with U0126 or tunicamycin in presence or absence of STF-62247 as described above. Tubulin was used as loading control. D. FACS analysis to measure the acidic vesicle organelles after staining with acridine orange in SN12C and SN12C-VHL shRNA following treatment with STF-62247 for 24 hours. E. AVO quantification from the FACS analysis in A. F. Autophagic vacuoles stained with MDC. After STF-62247 treatment with or without 3-MA, NH₄Cl, MDC was added the last 10 minutes. Then, cells were fixed, washed with PBS and observed directly under microscope.

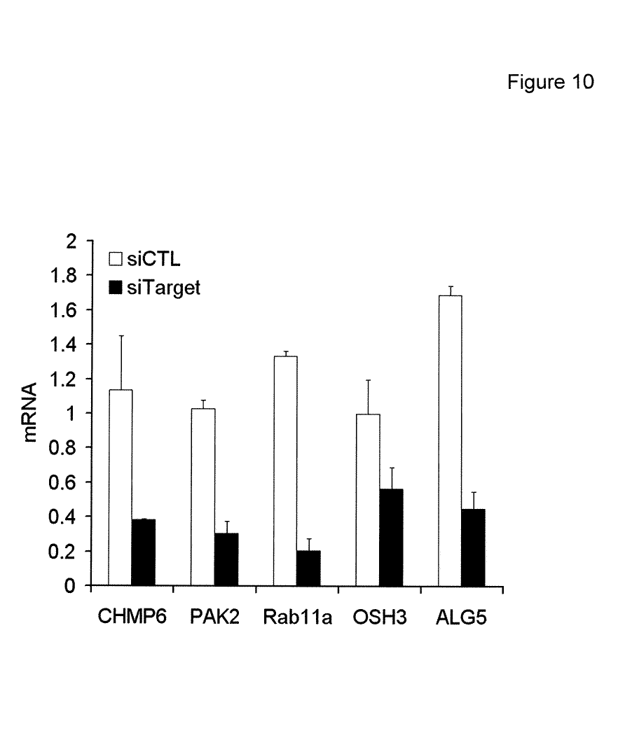

FIG. 10 shows knockdown of various genes using siRNA.

### Results

### STF-62247 Induces Cytotoxicity and Reduces Tumor Growth in VHL-Deficient Cells in a HIF-Independent Manner

In this study, the possibility of selectively targeting VHL-deficient cells using small molecule cytotoxins was evaluated. A library of 64,000 compounds was screened against wild-type VHL and VHL-deficient RCCs that were stably transfected with EYFP. The effect of small molecules on each cell type was monitored separately by fluorescence (FIG. 1A). The drug STF-62247 was identified in this screen (FIG. 7A) and decreases the viability of VHL-deficient cells in a short term assay (FIG. 7B). Moreover, STF-62247 specifically results in cytotoxicity in renal cells that have lost VHL as demonstrated by two genetic models: RCC4 cells deficient in VHL and their matched counterparts where wild-type VHL has been reintroduced, and SN12C cells stably expressing shRNA to VHL and SN12C cells with wild-type VHL (Thomas, G. V., et al., Nat. Med. 2006, 12, 122). Clonogenic assays demonstrated that STF-62247 is selectively toxic to VHL-deficient cells compared to their VHL wild-type counterparts (FIG. 1B).

Since VHL is an important negative regulator of HIF-α through its E3-ligase activity, VHL-null RCC4 cells expressing shRNA to HIF-1α or HIF2α were used to determine whether STF-62247 cytotoxicity was dependent on HIF (FIG. 1C bottom panel). The level of HIF target genes was quantified by qRT-PCR verifying functional knockdown of HIF (FIG. 7C). Clonogenic assays demonstrated that the reduction of either HIF-1α or HIF-2α in VHL-null cells did not affect cell death induced by STF-62247 (FIG. 1C upper panel). Also, a decrease level in ARNT (HIF-1β) using shRNA did not change the cell survival by STF-62247 (data not shown). Previous studies have indicated that HIF-2 is critical for tumor growth of RCCs (Kondo, K., et al., PLoS Biol. 2003, 1, E83; Zimmer, M., et al., Mol. Cancer. Res. 2004, 2, 89). To investigate the effect of HIF-2 in the sensitivity of RCC4 cells to STF-62247, a VHL cell line that stably expressed a normoxically stable, constitutively active HIF-2αmutant P531A N547A was generated. Two individual HIF-2α RCC4/VHL clones were randomly chosen that had elevated HIF-2α expression under normoxic conditions (FIG. 1D bottom panel). Results showed that cytotoxicity of STF-62247 was unaffected by the presence of HIF-2α expression under normoxic conditions when compared to RCC4/VHL cells. Thus, by using complementary approaches, the results indicate that STF-62247 induces cytotoxicity in VHL-null cells in a HIF-independent manner.

To evaluate the effect of STF-62247 on tumor growth in vivo, SN12C or SN12C-VHL shRNA were implanted subcutaneously in immune deficient mice. Daily treatment with STF-62247 significantly reduced tumor growth in VHL-deficient cells (FIG. 1E). This decrease in tumor growth was concentration-dependent. Importantly, drug treatment did not have any effect on the growth of SN12C tumor cells that have wild-type VHL. Together, these results show that STF-62247 selectively kills RCC cells with a loss of VHL in vitro and also significantly reduces tumor growth in cells deficient in VHL.

### Autophagy is Induced by STF-62247 Treatment

It was found that STF-62247 did not induce apoptosis in vitro in VHL-deficient cells as assayed by Hoechst (FIG. 7D), Annexin V staining (FIG. 7E) or caspase-3 (FIG. 7F). These results were further confirmed by immunostaining for caspase-3 in tumor sections (FIG. 7G). There was no difference in proliferation as assayed by Ki67 staining in response to STF-62247 in RCC deficient in VHL or with wild-type VHL (FIG. 7H). STF-62247 did not induce DNA damage as measured by the comet assay (data not shown). In addition, the phosphorylation of p53 on serine 15 and total p53 levels were unaffected by STF-62247 treatment, further supporting the lack of a DNA damage signal in cell killing induced by STF-62247 (FIG. 7I). It was observed that STF-62247-treated cells accumulated intracytoplasmic vacuoles characteristic of cells undergoing autophagy. Moreover, these vacuoles were larger in VHL-deficient RCC4 and SN12C-VHL shRNA cells than in wild-type VHL cells (FIG. 2A). Vacuole formation in RCC cells with and without VHL was monitored by time-lapse for 48 hours, showing that vacuoles appeared as soon as 3 hours after addition of STF-62247. Time-lapse microscopy also indicated that wild-type VHL prevents the formation of larger vacuoles present in VHL-deficient cells.

Induction of autophagy was confirmed by immunostaining and Western blotting for LC3. STF-62247 increased punctate staining of LC3, which is associated with processing of LC3 to its lipidated form (FIG. 2B). LC3-II, corresponding to the lipidated form found with the formation of double-membranes, was induced by STF-62247 treatment, indicating that STF-62247 stimulates autophagy (FIG. 2D). Transmission electron microscopy (TEM) was also used to monitor autophagy in response to STF-62247. There were large numbers of autophagic vacuoles present in STF-62247-treated cells, but not in untreated cells (FIG. 8A). The presence of double-membrane containing cellular organelles was observed in RCC4 treated cells at higher magnification (FIG. 8B).

The increase of the LC3-II form was abrogated by 3-methyladenine (3-MA), an autophagy inhibitor that inhibits PI3 kinase. It is noteworthy that although the stimulation of LC3 is higher in VHL-deficient cells treated with STF-62247, the increase was also observed in wild-type VHL cells, and therefore the presence of autophagosomes cannot by themselves explain the difference in toxicity observed between RCC cells with and without VHL. Cells were also stained with the autofluorescent compound monodansylcadaverine (MDC), which acts as a lysosomotropic agent and labels some of the acidic compartments that are observed after fusion with lysosomes (autolysosomes) (FIG. 2C). Staining with MDC in RCC4 cells was primarily observed around the larger vacuoles compared to the punctate staining of LC3, suggesting that these large vacuoles are associated with the acidic components of autolysosomes.

Since autophagy might be an early response to drug treatment, LC3 processing was monitored in cells exposed to increasing concentrations of drug. Results show that the LC3-lipidated form increases in a concentration-dependent manner, indicating that the autophagic process is tightly linked with drug cytotoxicity (FIG. 2E). To determine whether autophagy induced by STF-62247 was responsible for the reduced viability observed in VHL-deficient cells, the viability after 24 hours of cells treated with 3-MA to inhibit autophagy was measured. The viability of VHL-deficient cells was partially restored by 3-MA, indicating that STF-62247 induced autophagy leads to cell death in VHL-deficient cells (FIG. 2F).

### ATG5, ATG7 and ATG9 are Involved in Autophagosome Formation in STF-62247 Treated Cells

The autophagy-related gene 5 (AtgS) is involved in the formation of the autophagosome and the Atg12-AtgS ubiquitin-like conjugate has been previously reported to be necessary for LC3 modification (Kabeya, Y., et al., Embo J. 2000, 19, 5720; Mizushima, N., et al., J. Cell Biol. 2001, 152, 657). Thus, hypothically, if autophagy was important in STF-62247 cytotoxicity of VHL-deficient cells, ATG5−/− cells would be resistant to STF-62247-induced cell death. Significantly more autophagic vacuoles were found during STF-62247 treatment in ATG5+/+ cells compared to ATG5−/− cells (FIG. 3A). This effect correlates with the LC3 processing that was observed only in ATG5+/+ cells in response to STF-62247 and was concentration dependent (FIG. 3B). Importantly, it was confirmed that ATG5 is involved in STF-62247-induced toxicity in VHL-deficient RCC cells. Silencing ATG5 by siRNA protected RCC4 cells deficient in VHL to killing induced by STF-62247 as assessed by clonogenic assay (FIG. 3C). VHL-positive RCC4 cells were unaffected by ATG5 siRNA, suggesting that the cell death observed at higher concentrations in VHL-positive cells is independent of Atg5 and autophagy. A reduction in Atg12-AtgS levels by siRNA decreases LC3 formation by STF-62247, indicating a critical role for Atg5 in STF-62247 autophagic response in RCC VHL-deficient cells (FIG. 3D). The siRNA to Atg7 and Atg9 also prevents the STF-62247 cytotoxicity in RCC4 VHL-deficient cells, consistert with the essential role of the autophagy pathway in STF-62247 cytotoxicity (FIG. 3F). Atg7 acts as an E1 enzyme in the autophagy conjugation system where in combination with an E2 enzyme, conjugates the phosphatidyl ethanolamine to LC3I to form, LC3II. No lipidation of LC3 occurs in the brain of Atg7 deficient mice (Tanida, I., et al., J. Biol. Chem. 2002, 277, 13739). On the other hand, Atg9 has been reported to cycle between the trans-Golgi network and endosomes and its disruption impairs formation of autophagosomes (Young, A. R., et al., J. Cell Sci. 2006, 119, 3888). Protection against autophagic cell death induced by STF-62247 by disruption of Atg5, Atg7 and Atg9 is also demonstrated using VHL-deficient RCC10 and wild-type VHL RCC10 cells (FIG. 3G).

### PI3K and Golgi Trafficking are Required as Initial Signals in STF-62247-Induced Autophagy

Previous studies have implicated PI3K and ERK signaling as well as Golgi trafficking and endoplasmic reticulum (ER) stress in autophagy (Ogata, M., et al., Mol. Cell. Biol. 2006, 26, 9220; Yorimitsu, T., et al., J. Biol. Chem. 2006, 281, 30299). To evaluate a role for PI3K in response to STF-62247, the effects of the autophagy inhibitor 3-MA were compared to LY294002, another PI3K inhibitor. LY294002 significantly decreased large vacuole formation and the LC3-lipidation form in VHL-deficient cells during STF-62247 treatment to a level similar as 3-MA (FIG. 4 A-B and 2D), indicating a role for PI3K in STF-62247 signaling. In contrast, the MEK inhibitor U0126, which inhibits ERK activation, had little effect on autophagy induced by STF-62247 (FIG. 9A, 9B), indicating that MEK signaling is not involved in STF-62247 induced autophagy. Furthermore, it was found that Brefeldin A (BFA), a blocker of protein transport from the ER to the Golgi apparatus, abolished the formation of autophagic vacuoles and LC3-II stimulated by STF-62247 in VHL-deficient RCC cells. Without intending to be bound by theory, this suggests that Golgi trafficking is involved in the cytotoxicity of STF-62247 in cells that have loss VHL (FIG. 4 A-C). In contrast, no vacuole or LC3 processing was observed during STF-62247 treatment with tunicamycin, although it induced BiP (Grp78), a marker of ER stress (FIG. 9A, 9C). Taken together, but without intending to be bound by theory, the results suggest that Golgi trafficking and PI3K are part of the initial signal for the induction of autophagy in RCC cells treated with STF-62247.

### STF-62247 Increases Acidification in VHL-Deficient Cells

Although autophagosome formation in response to STF-62247 is independent of VHL status, how STF-62247 specifically kills cells that have lost VHL was investigated. A late step in the autophagic cell death process is the fusion of lysosomes with autophagosomes into autolysosomes, which can be detected by measuring their acidification with acridine orange staining Interestingly, we found acridine orange staining significantly increased in VHL-deficient RCC4 cells compared to their wild-type VHL counterparts (FIG. 4D). This observation was quantified and confirmed by FACS analyses and illustrates a significant difference in the acidification of the acidic vesicular organelles (AVOs) in RCC4 and SN12C-VHL shRNA cells (FIG. 4E-F and 9D-E) compared to wild-type VHL cells. Blocking autophagy with 3-MA or BFA resulted in a decrease in autolysosomal acidification, demonstrating that acidification induced by STF-62247 is linked to the autophagic pathway. In addition, MDC staining performed using NH₄Cl to block vesicle acidification attenuated the functional staining following drug treatment (FIG. 9F). However, even if the acidification level in VHL cells increases following STF-62247 treatment, this level of acidification remained significantly lower than in VHL-deficient cells. Without intending to be bound by theory, these results suggest that the fusion of autophagosomes with lysosomes is a key step leading to autophagic cell death in VHL-deficient cells.

### Structure-Activity Studies

Based on the STF-62247 structure, other compounds were designed and synthesized to evaluate their ability to induce selective cell death in VHL-deficient cells. Analysis of the high throughput screening (HTS) data indicated the importance of a 4-pyridyl unit linked to the 4-position of the thiazole, while the tested 3-pyridyl analogs were inactive. Without intending to be bound by theory, the importance of a thiazole NH suggests an H-bond contact in this region. Substituents at the 2-position on the aryl ring were not tolerated in the tested compounds. With these constraints in mind, we prepared a focused set of analogs of STF-62247 to probe structure-activity relationships (SAR) (Tables 1 and 2). The IC50 for these analogs shows that the STF-62247 has a low IC50 in VHL-deficient RCC4 cells and a high IC50 ratio (wild-type VHL/VHL-deficient cells). The compound 30603 with a methyl group in 4-position also has a high ratio but also a higher IC50 for both RCC4 and RCC4/VHL cells. In some cases, the IC50 could not be determined because of poor compound solubility.

To associate the cytotoxicity of these compounds with autophagy, the vacuole formation and LC3 processing were evaluated (FIG. 5A-B). For the tested compounds, those with an IC50 ratio VHL/VHLdeficient cells >5 correlated positively with the presence of large vacuoles in VHL-deficient cells and the processing of LC3. As observed with STF-62247 treatment, the LC3-lipidated form was present in both VHL wild type and deficient cell lines. To confirm that the cell death observed in VHL-deficient cells is associated with the acidification of autophagic vesicules, the level of AVOs was measured in response to 5 different analogs (FIG. 5C). The amount of AVOs was consistertly higher in VHL-deficient cells treated with compounds 30603, 31184, and 30651, three analogs where the IC50 was relatively low, but which induced significant vacuole formation and the LC3-lipidated form. In contrast, AVO staining was not influenced in response to compound 31185 and 31270, two analogs where no toxicity or autophagy were observed. These results with STF-62247 analogs confirm that the autophagic cell death observed in VHL-deficient cells is related to the acidification of the vacuoles. Without intending to be bound by theory, the strong relationship between autophagic cell death and vacuole acidification might explain why autophagy leads to cell death in VHL-deficient cells and not in wild-type cells.

TABLE 1
IC₅₀ values and selectivity ratios for compounds of the invention

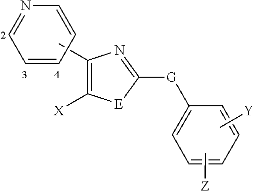

| | Cpd. | | | | | | | RCC4 | |
|---|---|---|---|---|---|---|---|---|---|
| SN # | No | pyridyl | E | X | Y | Z | G | μM | Ratio |
| 30602 | 3 | 4 | S | | H | | NH | 7 | >10 |
| 30661 | 5 | 4 | S | | 3-Me | | NH | 2.1 | 19 |
| STF- | | | | | | | | | |
| 62247 | | | | | | | | | |
| 32032 | 7 | 4 | S | | 3-Et | | NH | 2.5 | 9.6 |
| 31910 | 9 | 4 | S | | 3-iPr | | NH | 17 | 1.4 |
| 31938 | 11 | 4 | S | | 3-tBu | | NH | >40 | nd |
| 31185 | 13 | 4 | S | | 3-OH | | NH | 28.5 | >3 |
| 30651 | 15 | 4 | S | | 3-OMe | | NH | 3 | 17 |
| 30654 | 17 | 4 | S | | 3-Cl | | NH | 3 | 5 |
| 30670 | 19 | 4 | S | | 3-CF₃ | | NH | >40 | nd |
| 30603 | 21 | 4 | S | | 4-Me | | NH | 2.5 | 20 |
| 31274 | 23 | 4 | S | | 4-CH₂CH₂OH | | NH | 5 | 16 |
| 31184 | 25 | 4 | S | | 4-OH | | NH | 5.3 | 8 |
| 30644 | 27 | 4 | S | | 4-OMe | | NH | 14 | 3.6 |
| 31289 | 29 | 4 | S | | 4-OCH₂CH₂NMe₂ | | NH | 60 | 1.3 |
| 30671 | 31 | 4 | S | | 4-Cl | | NH | 38 | >2 |
| 30709 | 33 | 4 | S | | 4-F | | NH | 6 | >7 |
| 30715 | 35 | 4 | S | | 4-NO₂ | | NH | >40 | nd |
| 31283 | 36 | 4 | S | | 4-CH₂CH₂NMe₂ | | NH | 50 | 1.2 |
| 30723 | 37 | 4 | S | | 4-NH₂ | | NH | nd | nd |
| 31892 | 39 | 4 | S | | 3-OP(O)(OH)₂ | | NH | 29 | 1.1 |
| 31857 | 41 | 4 | S | | 4-OP(O)(OH)₂ | | NH | 3 | 6.3 |
| 31886 | 43 | 4 | S | | 3-Me | 4-Me | NH | 6.5 | 6.4 |
| 31849 | 45 | 4 | S | | 3-Me | 5-Me | NH | 4.5 | >9 |
| 31187 | 47 | 4 | S | | 3-CH₂CH₂CH₂-4 | | NH | 2.0 | 10 |
| 31258 | 49 | 4 | S | | 3-OH | 4-Me | NH | 12 | 4.6 |
| 31259 | 51 | 4 | S | | 3-OH | 4-OMe | NH | nd | nd |
| 31270 | 53 | 4 | S | | 3-Me | 4-OH | NH | 70 | 2.3 |
| 31181 | 55 | 4 | S | | 3-OMe | 4-OMe | NH | 24 | 1.9 |
| 31850 | 57 | 4 | S | | 3-OMe | 5-OMe | NH | >40 | nd |
| 31186 | 59 | 4 | S | | 3-OCH₂O-4 | | NH | >40 | nd |
| 30713 | 61 | 4 | S | | 3-Cl | 4-Cl | NH | 13.5 | >3 |
| 31851 | 63 | 4 | S | | 3,4,5-OMe₃ | | NH | >40 | nd |
| 31884 | 65 | 3 | S | | 3-Me | | NH | >40 | nd |
| 31888 | 66 | 3 | S | | 4-OH | | NH | >40 | nd |
| 31885 | 68 | 2 | S | | 3-Me | | NH | 37 | 0.9 |
| 31889 | 69 | 2 | S | | 4-OH | | NH | 36 | 0.7 |
| 32010 | 71 | 4 | S | Me | 3-Me | | NH | 10 | >4 |
| 32015 | 72 | 4 | S | Me | 3-CH₂CH₂CH₂-4 | | NH | 12 | 1.5 |
| 31961 | 73 | 4 | S | CH₂OH | | | NH | 30 | >1.3 |
| 31982 | 74 | 4 | S | CH₂OH | 3-Me | | NH | 7 | 2.4 |
| 31981 | 75 | 4 | S | CH₂OH | 4-Me | | NH | 14 | 1.2 |
| 31978 | 76 | 4 | S | CH₂OH | 3-CH₂CH₂CH₂-4 | | NH | 6.8 | 1.4 |
| 32009 | 77 | 4 | S | CHO | 3-Me | | NH | 15 | n.d. |
| 32014 | 78 | 4 | S | CH═CHCO₂Me | 3-Me | | NH | 2.5 | 2.8 |
| 32087 | 79 | 4 | S | CH₂CH₂CO₂Me | 3-Me | | NH | >40 | nd |
| 32098 | 81 | 4 | S | CH₂CH₂CH₂OH | 3-Me | | NH | 3.6 | 3.7 |
| 32109 | 82 | 4 | S | CH₂CH₂CH₂Nmorph | 3-Me | | NH | 5.8 | 1.6 |
| 32008 | 83 | 4 | S | CH₂NMe₂ | 3-Me | | NH | 16 | 2.3 |
| 32048 | 84 | 4 | S | CH₂NEt₂ | 3-Me | | NH | 13 | 1.4 |
| 32049 | 85 | 4 | S | CH₂Npiperidine | 3-Me | | NH | 13 | 1.9 |
| 32007 | 86 | 4 | S | CH₂Nmorpholine | 3-Me | | NH | 2.5 | 4.8 |
| 32013 | 88 | 4 | S | CO₂Et | 3-Me | | NH | >40 | n.d. |
| 32016 | 89 | 4 | S | CO₂Et | 3-CH₂CH₂CH₂-4 | | NH | >40 | n.d. |
| 32097 | 91 | 4 | S | CONH₂ | 3-Me | | NH | 19.2 | >2.0 |
| 32096 | 92 | 4 | S | CONMe₂ | 3-Me | | NH | 16 | >2.5 |
| 32091 | 93 | 4 | S | CONHCH₂CH₂NMe₂ | 3-Me | | NH | >40 | nd |
| 32093 | 94 | 4 | S | CONHCH₂CH₂CH₂Nmorph | 3-Me | | NH | >40 | nd |
| 31922 | 95 | 4 | S | | 3-Me | | NMe | 28 | 0.6 |
| 31923 | 96 | 4 | S | | 3-Me | | NEt | 40 | 0.6 |
| 31940 | 97 | 4 | S | | 3-Me | | NCH₂CH₂OH | >40 | nd |
| 31875 | 99 | 4 | S | | | | NHCO | >40 | nd |
| 31852 | 100 | 4 | S | | 3-Me | | NHCO | >40 | nd |
| 31872 | 101 | 4 | S | | 4-Me | | NHCO | >40 | nd |
| 31876 | 102 | 4 | S | | 4-OMe | | NHCO | >40 | nd |
| 31903 | 103 | 4 | S | | 3-Me | | NHCH₂ | >40 | nd |
| 32084 | 105 | 4 | O | | 3-Me | | NH | >40 | nd |
| 32680 | 107 | 4 | S | | 2-OH | | NH | 12.8 | >3.1 |
| 32523 | 109 | 4 | S | | 2-OMe | | NH | 33.4 | >1.2 |
| 32522 | 111 | 4 | S | | 2-Me | | NH | >40 | nd |
| 32685 | 113 | 4 | S | | 2-F | | NH | 14.3 | >2.8 |
| 32540 | 115 | 4 | S | | 2-Cl | | NH | 27.6 | >1.4 |
| 32591 | 117 | 4 | S | | 2-CF₃ | | NH | >40 | nd |
| 32594 | 119 | 4 | S | | 2-NO₂ | | NH | >40 | nd |
| 32185 | 121 | 4 | S | | 3-F | | NH | >40 | nd |
| 32291 | 123 | 4 | S | | 3-Br | | NH | 12.4 | >3.2 |
| 32623 | 125 | 4 | S | | 3-COCH₃ | | NH | 13 | >3.1 |
| 32518 | 127 | 4 | S | | 3-CN | | NH | >40 | nd |
| 32292 | 129 | 4 | S | | 3-NO₂ | | NH | >40 | nd |
| 32558 | 131 | 4 | S | | 4-COCH₃ | | NH | 3.8 | 3.1 |
| 32552 | 133 | 4 | S | | 4-CF₃ | | NH | >40 | nd |
| 32542 | 135 | 4 | S | | 4-CN | | NH | >40 | nd |
| 32682 | 137 | 4 | S | | 2-CH₃ | 6-CH₃ | NH | >40 | nd |
| 32678 | 139 | 4 | S | | 2-F | 6-F | NH | >40 | nd |
| 32842 | 196 | 4 | NH | | 3-CH₃ | | NH | | |

TABLE 2
IC₅₀ values and selectivity ratios for compounds of the invention

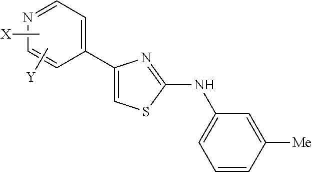

| | Cpd. | | | RCC4 | |
|---|---|---|---|---|---|
| SN # | No | X | Y | μM | Ratio |
| 32201 | 141 | 2-OMe | | 28.7 | 1.2 |
| 32181 | 145 | 2-Me | | >40 | nd |
| 32769 | 149 | 2-F | | 4.5 | >8.9 |
| 32684 | 150 | 2-Cl | | 2.2 | 3.8 |
| 32312 | 154 | 2-Br | | 3.9 | >10 |
| 32780 | 158 | 2-Cl | 6-Cl | 15 | 2.3 |
| 32715 | 159 | 2-C≡CH | | 17.8 | >2.2 |
| 32718 | 160 | 2-Et | | 6.4 | >6.3 |
| 32712 | 161 | 2-C≡CCH₂OH | | 9.3 | 1.6 |
| 32717 | 162 | 2-CH₂CH₂CH₂OH | | 5.2 | >7.7 |
| 32398 | 166 | 3-CH₃ | | 5.2 | >7.7 |
| 32770 | 170 | 3-F | | 2.0 | >20 |
| 32740 | 174 | 3-Cl | | 2.9 | >13.8 |
| 32346 | 178 | 3-Br | | 3.9 | >10 |
| 32758 | 181 | 3-NO₂ | | 8.2 | 4.1 |
| 32761 | 182 | 3-NH₂ | | >40 | nd |
| 32767 | 183 | 3-NHAc | | 33.3 | 1.0 |
| 32732 | 184 | 3-C≡CH | | 1.35 | 4.4 |
| 32734 | 185 | 3-Et | | 6.2 | 2.8 |
| 32733 | 186 | 3-C≡CCH₂OH | | 0.24 | 11.9 |
| 32735 | 187 | 3-CH₂CH₂CH₂OH | | 6.8 | >5.9 |
| 32764 | 188 | N⁺—O⁻ | | >40 | nd |
| 32208 | 192 | 4-quinolinyl | | 12.3 | 1.6 |
| 32311 | 194 | 2-phenyl-4-quinolinyl | | 25.4 | >1.6 |
| 32884 | 197 | 2-morpholine | | | |
| 32887 | 198 | 2-(4-Me-piperazine) | | | |
| 32892 | 199 | 2-NMe₂ | | | |

### Golgi Trafficking Proteins are Sensitive and a Target for the STF-62247.

To identify a target for STF-62247, a screen of the yeast deletion pool was performed to determine which genes affect STF-62247 sensitivity. It was found that proteins involved in Golgi trafficking and in morphogenesis were the most sensitive to STF-62247 (Table 3). To validate the data obtained from this screen, the sensitivity of several deleted yeast strains following STF-62247 treatment were evaluated (FIG. 6A). The killing curves for CLA4, VPS20, ALG5, OSH3, and YPT31 deletion strains show a significant toxicity in response to STF-62247. The ATG8 deletion strain was also evaluated and did not exhibit sensitivity to STF-62247 as expected based on human Atg studies (data not shown). The human orthologs for CLA4, VPS20, ALG5, OSH3 and YPT31 strains are PAK2, CHMP6, ALG5, OSBP3 and Rabll a respectively. In mammalian cells, it was found that the glucosyltransferase ALG5, the oxysterol binding protein 3 (OSBP3), and CHMP6, a key component of the endosomal sorting complex (ESCRT III) are VHL-regulated (FIG. 6B). These three proteins play a role in coordinating vesicular transport between the ER, the trans-Golgi network (TGN) and lysosomes. Moreover, the knockdown of these genes using siRNA (FIG. 10) increases the vacuole formation in response to STF-62247 (FIG. 6C) and cytotoxicity in VHL cells suggesting their loss partially phenocopies the loss of VHL (FIG. 6D). Taken together, the results demonstrate that the Trans-Golgi Network is a target of the STF-62247 and a drug-selective pathway synthetically lethal in VHL-deficient cells.

TABLE 3
Yeast screen in response to STF-62247

| | | Human | |
|---|---|---|---|
| ORF | Gene | homolog | Description |
| YPL170W | DAP1 | PGRMC1 | Heme-binding protein involved |
| | | | in regulation of cytochrome P450 |
| | | | protein Erg11p; damage response |
| | | | protein, related to mammalian |
| | | | membrane progesterone |
| | | | receptors; mutations lead to |
| | | | defects in telomeres, mitochondria, |
| | | | and sterol synthesis |
| YHR073W | OSH3 | OSBPL3 | Member of an oxysterol-binding |
| | | | protein family with seven |
| | | | members in S. cerevisiae; family |
| | | | members have overlapping, |
| | | | redundant functions in sterol |
| | | | metabolism and collectively |
| | | | perform a function essential |
| | | | for viability |
| YLR170C | APS1 | LOC653653 | Small subunit of the |
| | | | clathrin-associated adaptor complex |
| | | | AP-1, which is involved in protein |
| | | | sorting at the trans-Golgi network; |
| | | | homolog of the sigma subunit of the |
| | | | mammalian clathrin AP-1 complex |
| YAL026C | DRS2 | ATP8A2 | Aminophospholipid translocase |
| | | | (flippase) that maintains membrane |
| | | | lipid asymmetry in post-Golgi |
| | | | secretory vessicles; localizes to the |
| | | | trans-Golgi network; contributes to |
| | | | clathrin-coated vesicle formation and |
| | | | endocytosis; type 4 P-type ATPase |
| YER031C | YPT31 | RAB11A | GTPase of the Ypt/Rab family, |
| | | | involved in the exocytic |
| | | | pathway; mediates intra-Golgi |
| | | | traffic or the budding of post-Golgi |
| | | | vesicles from the trans-Golgi |
| YPL227C | ALG5 | ALG5p/ | UDP-glucose: dolichyl-phosphate |
| | | ALG5 | glucosyltransferase, involved |
| | | | in asparagine-linked glycosylation |
| | | | in the endoplasmic reticulum |
| YGR063C | SPT4 | SUPT4H1 | Protein that forms a complex with |
| | | | Spt5p and mediates both activation |
| | | | and inhibition of transcription |
| | | | elongation, and plays a role in pre- |
| | | | mRNA processing; in addition, |
| | | | Spt4p is involved in kinetochore |
| | | | function and gene silencing |
| YMR077C | VPS20 | CHMP6 | Myristoylated subunit of |
| | | | ESCRTIII, the endosomal sorting |
| | | | complex required for transport of |
| | | | transmembrane proteins into the |
| | | | multivesicular body pathway to |
| | | | the lysosomal/vacuolar lumen; |
| | | | cytoplasmic protein recruited |
| | | | to endosomal membranes |
| YJR102C | VPS25 | VPS25 | Component of the ESCRT-II |
| | | | complex, which is involved in |
| | | | ubiquitin-dependent sorting of |
| | | | proteins into the endosome |
| YNL298W | CLA4 | PAK2 | Cdc42p activated signal transducing |
| | | | kinase of the PAK (p21-activated |
| | | | kinase) family, involved in septin ring |
| | | | assembly and cytokinesis; directly |
| | | | phosphorylates septins Cdc3p |
| | | | and Cdc10p; other yeast PAK family |
| | | | members are Ste20p and Skm1p |
| YNL325C | Fig4 | KIAA0274 | Protein required for efficient mating, |
| | | | member of a family of eukaryotic |
| | | | proteins that contain a domain |
| | | | homologous to Sac1p |
| YBL078C | ATG8 | LC3 | Biological marker for autophagy |

All of the above-cited references and publications are incorporated by reference herein in their entireties.

Those skilled in the art will recognize, or be able to ascertain using no more than routine experimentation, numerous equivalents to the specific method and reagents described herein. Such equivalents are considered to be within the scope of this invention and are covered by the following claims.

1-17. (canceled)

18. A compound of Formula II: 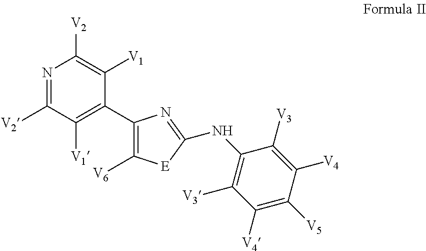
or a pharmaceutically acceptable salt, derivative, or prodrug thereof, wherein:
E is S, O, or N—V₇;
V₃ and V₄′ are both hydrogen;
V₁, V₁′, V₂, V₂′, V₃, V₄, V₅, V₆, and V₇ are independently hydrogen, halo, R, OH, OR, OC(O)H, OC(O)R, OC(O)NH₂, OC(O)NHR, OC(O)NRR, OP(O)(OH)₂, OP(O)(OR)₂, NO₂, NH₂, NHR, NRR, N′(—O⁻)RR, NHC(O)H, NHC(O)R, NRC(O)R, NHC(O)NH₂, NHC(O)NRR, NRC(O)NHR, N²C(O)NHR, SH, SR, S(O)H, S(O)R, SO₂R, SO₂NH₂, SO₂NHR, SO₂NRR, CF₃, CN, CO₂H, CO₂R, CHO, C(O)R, C(O)NH₂, C(O)NHR, C(O)NRR, CONHSO₂H, C(O)NHSO₂R, C(O)NRSO₂R, cyclic C₃-C₇ alkylamino, imidazolyl, piperazinyl, morpholinyl, piperidinyl, azepanyl, pyrrolidinyl or azetidinyl;
wherein each imidazolyl, piperazinyl, morpholinyl, piperidinyl, azepanyl, pyrrolidinyl and azetidinyl is optionally and independently substituted by one or more R¹, halo, OH, OR¹, OC(O)R¹, OC(O)NH₂, OC(O)NHR¹, OC(O)NR¹R¹, OP(O)(OH)₂, OP(O)(OR¹)₂, NO₂,NH₂, NHR¹, NR¹R¹, NHC(O)H. NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, CONHSO₂H, C(O)NHSO₂R¹ or C(O)NR¹SO₂R¹;
and wherein groups V₁ and V₂, groups V₃ and V₄, and groups V₄ and V₅, taken together with the atoms to which they are attached, optionally and independently form a cyclic structure having from 4 to 8 atoms in the ring;
each R is independently C₁₋₆ alkyl, C₂₋₆ alkenyl, C₂₋₆ alkynyl, C₃₋₇ cyclic alkyl, aryl, aralkyl, heteroaryl, or heteroaralkyl, and is optionally and independently substituted with halo, OH, R¹, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, C(O)NHSO₂R¹, C(O)NR¹SO₂R¹, cyclic C₃-C₇ alkylamino, imidazolyl, piperazinyl, morpholinyl, piperidinyl, azepanyl, pyrrolidinyl, or azetidinyl;
wherein each imidazolyl, piperazinyl, morpholinyl, piperidinyl, azepanyl, pyrrolidinyl and azetidinyl is optionally and independently substituted with one or more R¹, halo, OH, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, CONHSO₂H, C(O)NHSO₂R¹ or C(O)NR¹SO₇R¹;
and wherein each heteroaryl group contains one or more heteroatoms in its ring system, independently selected from O, N or S;
each R¹ is independently C₁₋₆ alkyl or C₂₋₆ alkenyl, optionally and independently substituted with halo, OH, OR², OC(O)R², OC(O)NH₂, OC(O)NHR², OC(O)NR²R², OP(O)(OH)₂, OP(O)(OR²)₂, NO₂ NH₂. NHR², NR²R², N⁺(—O⁻)R²R², NHC(O)R², NR²C(O)R², NHC(O)NH₂, NHC(O)NR²R², NR²C(O)NHR², SH, SR², S(O)H, S(O)R², SO₂R², SO₂NH₂, SO₂NHR², SO₂NR²R², CF₃, CN, CO₂H, CO₂R², CHO, C(O)R², C(O)NH₂, C(O)NHR², C(O)NR²R², C(O)NHSO₂R², C(O)NR²SO₂R², cyclic C₃-C₇ alkylamino, imidazolyl, piperazinyl, morpholinyl, piperidinyl, azepanyl, pyrrolidinyl or azetidinyl;
wherein each imidazolyl, piperazinyl, morpholinyl, piperidinyl, azepanyl, pyrrolidinyl and azetidinyl is optionally and independently substituted with one or more R², halo, OH, OR², OC(O)R², OC(O)NH₂, OC(O)NHR², OC(O)NR²R², OP(O)(OH)₂, OP(O)(OR²)₂, NO₂, NH₂, NHR², NR²R², N⁺(—O⁻)R²R², NHC(O)H, NHC(O)R², NR²C(O)R², NHC(O)NH₂, NHC(O)NR²R², NR²C(O)NHR², SH, SR², S(O)H, S(O)R², SO₂R², SO,NH₂, SO₂NHR², SO₂NR²R², CF₃, CN, CO₂H, CO₂R², CHO, C(O)R², C(O)NH₂, C(O)NHR², C(O)NR²R², CONHSO₂H, C(O)NHSO₂R² or C(O)NR²SO₂R²;
each R² is independently C₁₋₆ alkyl, C₂₋₆ alkenyl, OH, OMe, NO, NH₂, CF₃, CN, CO₂H or SH;
provided that V₁, V₁′, V₂, V₂¹, V₃, V₄, V₅, V₆, and V₇ are not all hydrogen; and
when E is S; V₁, V₁′, and V₂ are all hydrogen; and V₂ is Cl; then V₃ is not CH₃ when V₄ is Cl; and V₅ is not CH₃, OCH₃, or Cl; and
when E is S; V₁, V₁′, and V₂′ are all hydrogen; V₂ is H or NH₂; and V₄ is CF₃; then V₅ is not F; and
when E is S and V₁, V₁′, V₂, V₂′, V₃ and V₅ are all hydrogen, then V₄ is not COOH, COOCH₃, C(O)CH₃, CF₃, CH₃, OH, OCH₃, SCH₃, CN, O-phenyl, Cl, Br, or NO₂; and
when E is S and V₁, V V₂, V₂′, V₄ and V₅ are all hydrogen, then V₃ is not CH₃, CH₂CH₃, F, Cl, NO₂, CF₃, OCH₃, OCH₂CH₃, or a substituted 1,4-dihydropyridyl ring; and
when E is S and V₁, V₁′, V₂, V₂′, V₃ and V₄ are all hydrogen, then V₅ is not CH₃, CH₂CH₃, C(O)CH₃, CF₃, S(O)₂—NH₂, S(O)₂—NHR, N(CH₃)₂, N(CH₂CH₃)₂, N(i-Pr)phenyl, NO₂, OCH₂CH₃, NH—C(O)CH₃, NH₂, COOH, F, CI, Br, I, OH, OCH₃, O-phenyl, O—C₇₋₈-alkyl, or C(O)NHC(i-Bu)C(O)NHCH₂CN; and
when E is S and V₁, V₁′, V₂, V₂′, and V₅ are all hydrogen; then when V₃ is C₁, V₄ is not Cl; when V₃ is CH₃, then V₄ is not Cl; and V₃ and V₄, taken together with the ring to which they are attached, do not form 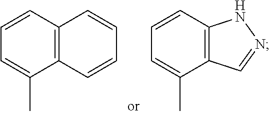
and
when E is S and V₁, V₁′, V₂, V₂′, and V₃ are all hydrogen; then when V₅ is O—C₅₋₈-alkyl, V₄ is not hydrogen, F, or CF₃; when V₅ is CH₃. V₄ is not CH₃; and V₄ and V₅, taken together with the ring to which they are attached, do not form 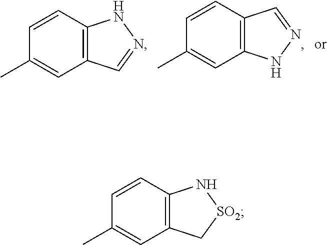
when E is S and V₁′, V₂′, V₃, V₄, and V₅ are all hydrogen, then V₁ and V₂, taken together with the ring to which they are attached, do not form

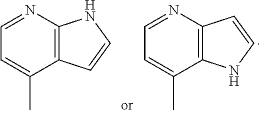

19. The compound of claim 18, wherein V₃, V₄, and V₅ are independently hydrogen; halo; R; OH; OR; CF₃; NO₂; NH₂; NHR; NRR; OP(O)(OH)₂; or OP(O)(OR)₂; and groups V₃ and V₄ and groups V₄ and V₅, taken together with the atoms to which they are attached, optionally and independently form a 5- or 6-membered ring structure.

20. The compound of claim 18, wherein V₃, V₄, and V₅ are independently hydrogen; halo; C₁₋₆ alkyl, optionally substituted with OH, NH₂, or N(CH₃)₂; OH; O—C₁₋₆ alkyl, optionally substituted with OH, NH₂, or N(CH₃)₂; CF₃; NO₂; NH₂; or OP(O)(OH)₂.

21. The compound of claim 18, wherein V₁ and V₂ are independently hydrogen, halo, R, OH, OR, CF₃, NO₂, NH₂, NHR, NRR, or, taken together with the atoms to which they are attached, form a 5- or 6-membered ring structure.

22. The compound of claim 18, wherein
V₁, V₁′, V₂, V₂′, are all hydrogen.

23. The compound of claim 18, wherein
E is S or O.

24. The compound of claim 23, wherein E is S.

25. A pharmaceutical composition comprising a compound of claim 1 and a pharmacuetically acceptable carrier.

26. (canceled)

27. A method of treating or preventing a disease, comprising administering to a mammalian host a therapeutically-effective amount of a compound of Formula II or a pharmaceutically acceptable salt, derivative, or prodrug thereof: 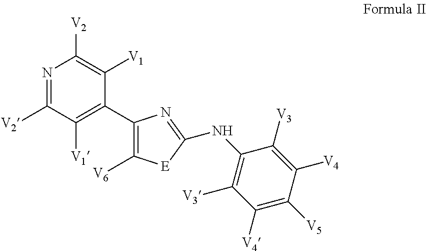
wherein:
E is S, O, or N—V₇;
V₃, V₃′, V₄′, and V₅ are all hydrogen;
V₁, V₁′, V₂, V₂′, V₄, V₆, and V₇ are independently hydrogen, halo, R, OH, OR, OC(O)H, OC(O)R, OC(O)NH, OC(O)NHR, OC(O)NRR, OP(O)(OH)₂, OP(O)(OR)₂, NO₂, NH₂, NHR, NRR, N(—O)RR, NHC(O)H, NHC(O)R, NRC(O)R, NHC(O)NH₂, NHC(O)NRR, NRC(O)NHR, N²C(O)NHR, SH, SR, S(O)H, S(O)R, SO₂R, SO₂NH₂, SO₂NHR, SO₂NRR, CF₃, CN, CO₂H, CO₂R, CHO, C(O)R, C(O)NH₂, C(O)NHR, C(O)NRR, CONHSO₂H, C(O)NHSO₂R, C(O)NRSO₂R, cyclic C₃-C₇ alkylamino, imidazolyl, piperazinyl, morpholinyl, piperidinyl, azepanyl, pyrrolidinyl or azetidinyl;
wherein each imidazolyl, piperazinyl, morpholinyl, piperidinyl, azepanyl, pyrrolidinyl and azetidinyl is optionally and independently substituted by one or more R¹, halo, OH, OW, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, CONHSO₂H, C(O)NHSO₂R¹ or C(O)NR¹SO₂R¹;
and wherein V₁ and V₂, taken together with the atoms to which they are attached, optionally form a cyclic structure having from 4 to 8 atoms in the ring;
each R is independently C₁₋₆ alkyl, C₂₋₆ alkenyl, C₂₋₆ alkynyl, C₃₋₇ cyclic alkyl, aryl, aralkyl, heteroaryl, or heteroaralkyl, and is optionally and independently substituted with halo, OH, R¹, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, C(O)NHSO₂R¹, C(O)NR¹SO₂R¹, cyclic C₃-C₇ alkylamino, imidazolyl, piperazinyl, morpholinyl, piperidinyl, azepanyl, pyrrolidinyl, or azetidinyl;
wherein each imidazolyl, piperazinyl, morpholinyl, piperidinyl, azepanyl, pyrrolidinyl and azetidinyl is optionally and independently substituted with one or more R¹, halo, OH, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, CONHSO₂H, C(O)NHSO₂R¹ or C(O)NR¹SO₇Z¹;
and wherein each heteroaryl group contains one or more heteroatoms in its ring system, independently selected from O, N or S;
each R¹ is independently C₁₋₆ alkyl or C₂₋₆ alkenyl, optionally and independently substituted with halo, OH, OR², OC(O)R², OC(O)NH₂, OC(O)NHR², OC(O)NR²R², OP(O)(OH)₂, OP(O)(OR²)₂, NO₂, NH₂, NHR², NR²R², N⁺(—O⁻)R²R², NHC(O)R², NR²C(O)R². NHC(O)NH₂, NHC(O)NR²R², NR²C(O)NHR², SH, SR². S(O)H, S(O)R², SO₂R², SO₂NH₂, SO₂NHR², SO₂NR²R², CF₃, CN, CO₂H, CO₂R², CHO. C(O)R², C(O)NH₂, C(O)NHR², C(O)NR²R², C(O)NHSO₂R², C(O)NR²SO₂R², cyclic C₃-C₇ alkylamino, imidazolyl, piperazinyl, morpholinyl, piperidinyl, azepanyl, pyrrolidinyl or azetidinyl;
wherein each imidazolyl, piperazinyl, morpholinyl, piperidinyl, azepanyl, pyrrolidinyl and azetidinyl is optionally and independently substituted with one or more R², halo, OH, OR², OC(O)R², OC(O)NH₂, OC(O)NHR², OC(O)NR²R², OP(O)(OH)₂, OP(O)(OR²)₂, NO₂, NH₂, NHR², NR²R², N⁺(—O⁻)R²R², NHC(O)H, NHC(O)R², NR²C(O)R², NHC(O)NH₂, NHC(O)NR²R², NR²C(O)NHR², SH, SR², S(O)H, S(O)R², SO₂R², SO₂NH₂, SO₂NHR², SO₂NR²R², CF₃, CN, CO₂H, CO₂R², CHO, C(O)R², C(O)NH₂, C(O)NHR², C(O)NR²R², CONHSO₂H, C(O)NHSO₂R² or C(O)NR²SO₂R²; and
each R² is independently C₁₋₆ alkyl, C₂₋₆ alkenyl, OH, OMe, NO₂, NH₂, CF₃, CN, CO₂H or SH;
provided that V₁, V₁′, V₂, V₂′, V₄, V₆, and V₇ are not all hydrogen; and
when E is S; V₁, V₁′, V₂′, and V₆ are all hydrogen; and V₂ is Cl; then V₄ is not Cl; and
when E is S and V₁, V₁′, V₂, V₂′, and V₆ are all hydrogen; then V₄ is not COOH, COOCH₃, C(O)CH₃, CF₃, CH₃, OH, OCH₃, SCH₃, CN, O-phenyl, Cl, Br, or NO₂; and
when E is S and V₁′, V₂′, V₄, and V₆ are all hydrogen; then V₁ and V₂, taken together with the ring to which they are attached, do not form

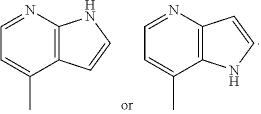

28. The method of claim 27, wherein in the compound of Formula II:
V₄ is hydrogen; halo; R; OH; OR; CF₃; NO₂; NH₂; NHR; NRR; OP(O)(OH)₂; or OP(O)(OR)₂.

29. The method of claim 27, wherein in the compound of Formula II:
V₄ is hydrogen: halo; C₁ alkyl, optionally substituted with OH, NH₂, or N(CH₃)₂; OH; O—C₁₋₆ alkyl, optionally substituted with OH. NH₂, or N(CH₃)₂: CF₃; NO₂; NH₂; or OP(O)(OH)₂.

30. The method of claim 27, wherein in the compound of Formula II:
V₆ is hydrogen; R; CHO; CO₂R; C(O)NH₂; C(O)NHR; or C(O)NRR.

31. The method of claim 27, wherein in the compound of Formula II:
V₆ is hydrogen; C₁₋₆ alkyl or C₂₋₄ alkenyl, optionally substituted with OH, OR¹, CO₂R¹, NH₂, NHR¹, or NR¹R¹; CHO; or CO₂R.

32. The method of claim 27, wherein in the compound of Formula II:
V₆ is hydrogen.

33-50. (canceled)

51. A method of treating or preventing a disease, comprising administering to a mammalian host a therapeutically-effective amount of a compound of Formula II or a pharmaceutically acceptable salt, derivative, or prodrug thereof: 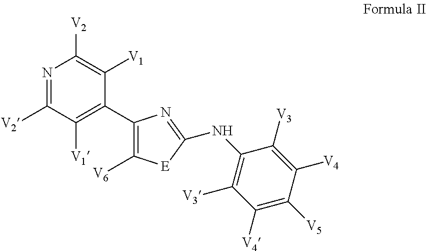
wherein:
E is S. O, or N—V₇;
V₁, V₁′, V₂, V₂′, V₃, V₃¹, V₄, V₄¹, V₅, V₆, and V₇ are independently hydrogen, halo, R, OH, OR, OC(O)H, OC(O)R, OC(O)NH₂, OC(O)NHR, OC(O)NRR, OP(O)(OH)₂, OP(O)(OR)₂, NO₂, NH), NHR, NRR, N NHC(O)H, NHC(O)R, NRC(O)R, NHC(O)NH₂, NHC(O)NRR, NRC(O)NHR, N²C(O)NHR, SH, SR, S(O)H, S(O)R, SO₂R, SO₂NH₂, SO₂NHR, SO₂NRR, CF₃, CN, CO₂H, CO₂R, CHO, C(O)R, C(O)NH₂, C(O)NHR, C(O)NRR, CONHSO₂H, C(O)NHSO₂R, C(O)NRSO₂R, cyclic C₃-C₇ alkylamino, imidazolyl, piperazinyl, morpholinyl, piperidinyl, azepanyl, pyrrolidinyl or azetidinyl;
wherein each imidazolyl, piperazinyl, morpholinyl, piperidinyl, azepanyl, pyrrolidinyl and azetidinyl is optionally and independently substituted by one or more R¹, halo, OH, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₁H, CO₂R¹, CHO, C(O)R¹, C(O)NH₂, C(O)NHR¹, C(O)NR¹R¹, CONHSO₂H, C(O)NHSO₂R¹ or C(O)NR¹SO₂R¹;
and wherein groups V₁ and V₂, groups V₃ and V₄, and groups V₄ and V₅, taken together with the atoms to which they are attached, optionally and independently form a cyclic structure having from 4 to 8 atoms in the ring;
each R is independently C₁₋₆ alkyl, C₂₋₆ alkenyl, C₂₋₆ alkynyl, C₃₋₇ cyclic alkyl, aryl, aralkyl, heteroaryl, or heteroaralkyl, and is optionally and independently substituted with halo, OH, R¹, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, C(O)NHSO₂R¹, C(O)NR¹SO₂R¹, cyclic C₃-C₇ alkylamino, imidazolyl, piperazinyl, morpholinyl, piperidinyl, azepanyl, pyrrolidinyl, or azetidinyl;
wherein each imidazolyl, piperazinyl, morpholinyl, piperidinyl, azepanyl, pyrrolidinyl and azetidinyl is optionally and independently substituted with one or more R¹, halo, OH, OR¹, OC(O)R¹, OC(O)NH₂, OC(O)NHR¹, OC(O)NR¹R¹, OP(O)(OH)₂, OP(O)(OR¹)₂, NO₂, NH₂, NHR¹, NR¹R¹, N⁺(—O⁻)R¹R¹, NHC(O)H, NHC(O)R¹, NR¹C(O)R¹, NHC(O)NH₂, NHC(O)NR¹R¹, NR¹C(O)NHR¹, SH, SR¹, S(O)H, S(O)R¹, SO₂R¹, SO₂NH₂, SO₂NHR¹, SO₂NR¹R¹, CF₃, CN, CO₂H, CO₂R¹, CHO, C(O)R¹, C(O)NH₂, C(O)NHR¹, C(O)NR¹R¹, CONHSO₂H, C(O)NHSO₂R¹ or C(O)NR¹SO₂R¹:
and wherein each heteroaryl group contains one or more heteroatoms in its ring system independently selected from O, N or S;
each R¹ is independently C₁₋₆ alkyl or C₂₋₆ alkenyl, optionally and independently substituted with halo, OH, OR², OC(O)R², OC(O)NH₂, OC(O)NHR², OC(O)NR²R², OP(O)(OH)₂, OP(O)(OR²)₂, NO₂, NH₂, NHR², NR²R², N⁺(—O⁻)R²R², NHC(O)R², NR²C(O)R², NHC(O)NH₂, NHC(O)NR²R², NR²C(O)NHR², SH, SR², S(O)H, S(O)R², SO₂R², SO₂NH₂, SO₂NHR², SO₂NR²R², CF₃, CN, CO₂H, CO₂R², CHO, C(O)R², C(O)NH₂, C(O)NHR², C(O)NR²R², C(O)NHSO₂R², C(O)NR²SO₂R², cyclic C₃-C₇ alkylamino, imidazolyl, piperazinyl, morpholinyl, piperidinyl, azepanyl, pyrrolidinyl or azetidinyl;
wherein each imidazolyl, piperazinyl, morpholinyl, piperidinyl, azepanyl, pyrrolidinyl and azetidinyl is optionally and independently substituted with one or more R², halo, OH, OR², OC(O)R², OC(O)NH₂, OC(O)NHR², OC(O)NR²R², OP(O)(OH)₂, OP(O)(OR²)₂, NO₂, NH₂, NHR², NR²R², N⁺(—O⁻)R²R², NHC(O)H, NHC(O)R², NR²C(O)R², NHC(O)NH₂, NHC(O)NR²R², NR²C(O)NHR², SH, SR², S(O)H, S(O)R², SO₂R², SO₂NH₂, SO₂NHR², SO₂NR²R², CF₃, CN, CO₂H, CO₂R², CHO, C(O)R², C(O)NH₂, C(O)NHR², C(O)NR²R², CONHSO₂H, C(O)NHSO₂R² or C(O)NR²SO₂R²;
each R² is independently C₁₋₆ alkyl, C₂₋₆ alkenyl, OH, OMe, NO₂, NH₂, CF₃, CN, CO₂H or SH;
and wherein the disease is caused by a defect in the von Hippel-Lindau gene.

52. The method of claim 51, wherein in the compound of Formula II:
V₃, V₄, and V₅ are independently hydrogen; halo; R; OH; OR; CF₃; NO₂; NH₂; NHR; NRR; OP(O)(OH)₂; or OP(O)(OR)₂; and groups V₃ and V₄ and groups V₄ and V₅, taken together with the atoms to which they are attached, optionally and independently form a 5- or 6-membered ring structure.

53. The method of claim 51, wherein in the compound of Formula II:
V₃, V₄, and V₅ are independently hydrogen; halo; C₁₋₆ alkyl, optionally substituted with OH, NH₂, or N(CH₃)₂; OH; O—C₁₋₆ alkyl, optionally substituted with OH. NH₂, or N(CH₃)₂; CF₃; NO₂; NH₂; or OP(O)(OH)₂.

54. The method of claim 51, wherein in the compound of Formula II:
V₆ is hydrogen; R; CHO; CO₂R; C(O)NH₂; C(O)NHR; or C(O)NRR.

55. The method of claim 51, wherein in the compound of Formula II:
V₆ is hydrogen; C₁₋₆ alkyl or C₂₋₄ alkenyl, optionally substituted with OH, OR¹, CO₂R¹, NH₂, NHR¹, or NR¹R¹; CHO; or CO₂R.

56-60. (canceled)

61. The method of claim 51, wherein the compound is selected from any one of the following compounds:
N-Phenyl-4-(4-pyridinyl)-1,3-thiazol-2-amine,
N-(3-Methylphenyl)-4-(4-pyridinyl)-1,3-thiazol-2-amine,
N-(3-Ethylphenyl)-4-(4-pyridinyl)-1,3-thiazol-2-amine,
N-(3-Isopropylphenyl)-4-(4-pyridinyl)-1,3-thiazol-2-amine,
N-(3-tert-Butylphenyl)-4-(4-pyridinyl)-1,3-thiazol-2-amine,
3-{[4-(4-Pyridinyl)-1,3-thiazol-2-yl]amino}phenol,
N-(3-Methoxyphenyl)-4-(4-pyridinyl)-1,3-thiazol-2-amine,
N-(3-Chlorophenyl)-4-(4-pyridinyl)-1,3-thiazol-2-amine,
4-(4-Pyridinyl)-N-[3-(trifluoromethyl)phenyl]-1,3-thiazol-2-amine,
N-(4-Methylphenyl)-4-(4-pyridinyl)-1,3-thiazol-2-amine,
2-(4-{[4-(4-Pyridinyl)-1,3-thiazol-2-yl]amino}phenyl)ethanol,
4-{[4-(4-Pyridinyl)-1,3-thiazol-2-yl]amino}phenol,
N-(4-Methoxyphenyl)-4-(4-pyridinyl)-1,3-thiazol-2-amine,
N-{4-[2-(Dimethylamino)ethoxy]phenyl}-4-(4-pyridinyl)-1,3-thiazol-2-amine,
N-(4-Chlorophenyl)-4-(4-pyridinyl)-1,3-thiazol-2-amine,
N-(4-Fluorophenyl)-4-(4-pyridinyl)-1,3-thiazol-2-amine,
N-(4-Nitrophenyl)-4-(4-pyridinyl)-1,3-thiazol-2-amine,
N-{4-[2-(Dimethylamino)ethyl]phenyl}-4-(4-pyridinyl)-1,3-thiazol-2-amine,
N¹-[4-(4-Pyridinyl)-1,3-thiazol-2-yl]-1,4-benzenediamine,
3-{[4-(4-Pyridinyl)-1,3-thiazol-2-yl]amino}phenyl Dihydrogen Phosphate,
4-{[4-(4-Pyridinyl)-1,3-thiazol-2-yl]amino}phenyl Dihydrogen Phosphate,
N-(3,4-Dimethylphenyl)-4-(4-pyridinyl)-1,3-thiazol-2-amine,
N-(3,5-Dimethylphenyl)-4-(4-pyridinyl)-1,3-thiazol-2-amine,
N-(2,3-Dihydro-1H-inden-5-yl)-4-(4-pyridinyl)-1,3-thiazol-2-amine,
2-Methyl-5-{[4-(4-pyridinyl)-1,3-thiazol-2-yl]amino}phenol,
2-Methoxy-5-{[4-(4-pyridinyl)-1,3-thiazol-2-yl]amino}phenol,
2-Methyl-4-{[4-(4-pyridinyl)-1,3-thiazol-2-yl]animno}phenol,
N-(3,4-Dimethoxyphenyl)-4-(4-pyridinyl)-1,3-thiazol-2-amine,
N-(3,5-Dimethoxyphenyl)-4-(4-pyridinyl)-1,3-thiazol-2-amine,
N-(1,3-Benzodioxol-5-yl)-4-(4-pyridinyl)-1,3-thiazol-2-amine,
N-(3,4-Dichlorophenyl)-4-(4-pyridinyl)-1,3-thiazol-2-amine,
4-(4-Pyridinyl)-N-(3,4,5-trimethoxyphenyl)-1,3-thiazol-2-amine,
N-(3-Methylphenyl)-4-(3-pyridinyl)-1,3-thiazol-2-amine,
4-{[4-(3-Pyridinyl)-1,3-thiazol-2-yl]amino}phenol,
N-(3-Methylphenyl)-4-(2-pyridinyl)-1,3-thiazol-2-amine,
4-{[4-(2-Pyridinyl)-1,3-thiazol-2-yl]amino}phenol,
5-Methyl-N-(3-methylphenyl)-4-(4-pyridinyl)-1,3-thiazol-2-amine,
N-(2,3-Dihydro-1H-inden-5-yl)-5-methyl-4-(4-pyridinyl)-1,3-thiazol-2-amine,
[2-(Phenylamino)-4-(4-pyridinyl)-1,3-thiazol-5-yl]methanol,
[2-[(3-Methylphenyl)amino]-4-(4-pyridinyl)-1,3-thiazol-5-yl]methanol,
[2-[(4-Methylphenyl)amino]-4-(4-pyridinyl)-1,3-thiazol-5-yl]methanol,
[2-(2,3-Dihydro-1H-inden-5-ylamino)-4-(4-pyridinyl)-1,3-thiazol-5-yl]methanol,
2-[(3-Methylphenyl)amino]-4-(4-pyridinyl)-1,3-thiazole-5-carbaldehyde,
Methyl (2E)-3-[2-[(3-Methylphenyl)amino]-4-(4-pyridinyl)-1,3-thiazol-5-yl]-2-propenoate,
Methyl 3-[2-[(3-Methylphenyl)amino]-4-(4-pyridinyl)-1,3-thiazol-5-yl]propanoate,
3-[2-[(3-Methylphenypamino]-4-(4-pyridinyl)-1,3-thiazol-5-yl]-1-propanol,
N-(3-Methylphenyl)-5-[3-(4-morpholinyl)propyl]-4-(4-pyridinyl)-1,3-thiazol-2-amine,
5-[(Dimethylamino)methyl]-N-(3-methylphenyl)-4-(4-pyridinyl)-1,3-thiazol-2-amine,
5-[(Diethylamino)methyl]-N-(3-methylphenyl)-4-(4-pyridinyl)-1,3-thiazol-2-amine,
N-(3-Methylphenyl)-5-(1-piperidinylmethyl)-4-(4-pyridinyl)-1,3-thiazol-2-amine,
N-(3-Methylphenyl)-5-(4-morpholinylmethyl)-4-(4-pyridinyl)-1,3-thiazol-2-amine,
Ethyl 2-[(3-methylphenyl)amino]-4-(4-pyridinyl)-1,3-thiazole-5-carboxylate,
Ethyl 2-(2,3-dihydro-1H-inden-5-ylamino)-4-(4-pyridinyl)-1,3-thiazole-5-carboxylate,
2-[(3-Methylphenyl)amino]-4-(4-pyridinyl)-1,3-thiazole-5-carboxamide,
N,N-Dimethyl-2-[(3-methylphenyl)amino]-4-(4-pyridinyl)-1,3-thiazole-5-carboxamide,
N-[2-(Dimethylamino)ethyl]-2-[(3-methylphenyl)amino]-4-(4-pyridinyl)-1,3-thiazole-5-carboxamide,
2-[(3-Methylphenyl)amino]-N-[3-(4-morpholinyl)propyl]-4-(4-pyridinyl)-1,3-thiazole-5-carboxamide,
N-Methyl-N-(3-methylphenyl)-4-(4-pyridinyl)-1,3-thiazol-2-amine,
N-Ethyl-N-(3-methylphenyl)-4-(4-pyridinyl)-1,3-thiazol-2-amine,
2-{3-Methyl[4-(4-pyridinyl)-1,3-thiazol-2-yl]anilino}ethanol,
N-(3-Methylphenyl)-4-(4-pyridinyl)-1,3-oxazol-2-amine,
2-{[4-(4-Pyridinyl)-1,3-thiazol-2-yl]amino}phenol,
N-(2-Methoxyphenyl)-4-(4-pyridinyl)-1,3-thiazol-2-amine,
N-(2-Methylphenyl)-4-(4-pyridinyl)-1,3-thiazol-2-amine,
N-(2-Fluorophenyl)-4-(4-pyridinyl)-1,3-thiazol-2-amine,
N-[2-Chlorophenyl]-4-(4-pyridinyl)-1,3-thiazol-2-amine,
4-(4-Pyridinyl)-N-[2-(trifluoromethyl)phenyl]-1,3-thiazol-2-amine,
N-[2-Nitrophenyl]-4-(4-pyridinyl)-1,3-thiazol-2-amine,
N-(3-Fluorophenyl)-4-(4-pyridinyl)-1,3-thiazol-2-amine,
N-(3-Bromophenyl)-4-(4-pyridinyl)-1,3-thiazol-2-amine,
1-(3-{[4-(4-Pyridinyl)-1,3-thiazol-2-yl]amino}phenyl)ethanone,
3-{[4-(4-Pyridinyl)-1,3-thiazol-2-yl]amino}benzonitrile,
N-(3-Nitrophenyl)-4-(4-pyridinyl)-1,3-thiazol-2-amine,
1-(4-{[4-(4-Pyridinyl)-1,3-thiazol-2-yl]amino}phenyl)ethanone,
4-(4-Pyridinyl)-N-[4-(trifluoromethyl)phenyl]-1,3-thiazol-2-amine,
4-{[4-(4-Pyridinyl)-1,3-thiazol-2-yl]amino}benzonitrile,
N-(2,6-Dimethylphenyl)-4-(4-pyridinyl)-1,3-thiazol-2-amine,
N-(2,6-Difluorophenyl)-4-(4-pyridinyl)-1,3-thiazol-2-amine,
N-{4-[2-(Methyloxy)-4-pyridinyl]-1,3-thiazol-2-yl}-N-(3-methylphenyl)amine,
N-(3-Methylphenyl)-4-(2-methyl-4-pyTidinyl)-1,3-thiazol-2-amine,
4-(2-Fluoro-4-pyridinyl)-N-(3-methylphenyl)-1,3-thiazol-2-amine,
4-(2-Chloro-4-pyridinyl)-N-(3-methylphenyl)-1,3-thiazol-2-amine,
4-(2-Bromo-4-pyridinyl)-N-(3-methylphenyl)-1,3-thiazol-2-amine,
N-[4-(2,6-Dichloro-4-pyridinyl)-1,3-thiazol-2-yl]-N-(3-methylphenyl)amine,
4-(2-Ethynyl-4-pyridinyl)-N-(3-methylphenyl)-1,3-thiazol-2-amine,
4-(2-Ethyl-4-pyridinyl)-N-(3-methylphenyl)-1,3-thiazol-2-amine,
3-(4-{2-[(3-Methylphenyl)amino]-1,3-thiazol-4-yl}-2-pyridinyl)-2-propyn-1-ol,
3-(4-{2-[(3-Methylphenyl)aminc]1,3-thiazol-4-yl}-2-pyridinyl)-1-propanol,
N-(3-Methylphenyl)-4-(3-methyl-4-pyridinyl)-1,3-thiazol-2-amine,
4-(3-Fluoro-4-pyridinyl)-N-(3-methylphenyl)-1,3-thiazol-2-amine,
4-(3-Chloro-4-pyridinyl)-N-(3-methylphenyl)-1,3-thiazol-2-amine,
4-(3-Bromo-4-pyridinyl)-N-(3-methylphenyl)-1,3-thiazol-2-amine,
N-(3-Methylphenyl)-4-(2-nitro-4-pyridinyl)-1,3-thiazol-2-amine,
N-[4-(2-Amino-4-pyridinyl)-1,3-thiazol-2-yl]-N-(3-methylphenyl)amine,
N-(4-{2-[(3-Methylphenyl)amino]-1,3-thiazol-4-yl}-2-pyridinyl)acetamide,
4-(3-Ethynyl-4-pyridinyl)-N-(3-methylphenyl)-1,3-thiazol-2-amine,
4-(3-Ethyl-4-pyridinyl)-N-(3-methylphenyl)-1,3-thiazol-2-amine,
3-(4-{2-[(3-Methylphenyl)amino]-1,3-thiazol-4-yl}-3-pyridinyl)-2-propyn-1-ol,
3-(4-{2-[(3-Methylphenyl)amino]-1,3-thiazol-4-yl}-3-pyridinyl)-1-propanol,
N-(3-Methylphenyl)-4-(1-oxido-4-pyridinyl)-1,3-thiazol-2-amine,
N-(3-Methylphenyl)-4-(4-quinolinyl)-1,3-thiazol-2-amine,
N-(3-Methylphenyl)-4-(2-phenyl-4-quinolinyl)-1,3-thiazol-2-amine,
N-(3-Methylphenyl)-4-(4-pyridinyl)-1H-imidazol-2-amine,
N-(3-Methylphenyl)-442-(4-morpholinyl)-4-pyridinyl)-1,3-thiazol-2-amine,
N-(3-Methylphenyl)-4-[2-(4-methyl-1-piperazinyl)-4-pyridinyl]-1,3-thiazol-2-amine, and
N,N-Dimethyl-4-[2-[(3-methylphenyl)amino]-1,3-thiazol-4-yl]-2-pyridinamine.

62-72. (canceled)
